(19) United States
(12) Patent Application Publication
(10) Pub. No.: US 2023/0144097 A1
(43) Pub. Date: May 11, 2023

(54) ACTIVE DNA TRANSPOSON SYSTEMS AND METHODS FOR USE THEREOF

(71) Applicant: Institute of Zoology, Chinese Academy of Sciences, Beijing (CN)

(72) Inventors: Haoyi WANG, Beijing (CN); Yong ZHANG, Beijing (CN); Shengjun TAN, Beijing (CN); Tongtong ZHANG, Beijing (CN)

(21) Appl. No.: 17/915,964

(22) PCT Filed: Mar. 30, 2021
(86) PCT No.: PCT/CN2021/084084

§ 371 Date: Sep. 29, 2022

(30) Foreign Application Priority Data
Mar. 30, 2020 (CN) PCT/CN2020/082087

(51) Int. Cl. C12N 15/85; C12N 15/10; C12N 15/90

(52) U.S. Cl. CPC C12N 15/85; C12N 15/1086; C12N 15/907; C12N 2800/22; C12N 2800/90

(57) ABSTRACT

Provided are engineered transposable elements, gene transfer systems comprising the engineered transposable elements, as well as methods and kits for using the same. The compositions, systems, and methods are useful for inserting a heterologous nucleic acid into a target nucleic acid in vitro or in a cell.

### CROSS REFERENCE TO RELATED APPLICATIONS

This application claims priority benefit of International Patent Application No. PCT/CN2020/082087 filed Mar. 30, 2020, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 182452000641SEQLIST.txt, date recorded: Mar. 29, 2021, size: 244 KB).

### FIELD

The present application relates generally to the field of genetics. More specifically, the present application relates to transposable elements and uses thereof.

### BACKGROUND

Typical methods for introducing DNA into a cell include DNA condensing reagents such as calcium phosphate, polyethylene glycol, and the like, lipid-containing reagents, such as liposomes, multi-lamellar vesicles, and the like, as well as virus-mediated strategies. However, such methods can have limitation. For example, there are size constraints associated with DNA condensing reagents and virus-mediated strategies. Further, the amount of nucleic acid that can be transfected into a cell is limited in virus strategies. Not all methods facilitate insertion of the delivered nucleic acid into cellular nucleic acid and while DNA condensing methods and lipid-containing reagents are relatively easy to prepare, the insertion of nucleic acid into viral vectors can be labor intensive. Virus-mediated strategies can be cell-type or tissue-type specific and the use of virus-mediated strategies can create immunologic problems when used in vivo.

One suitable tool in order to overcome these problems is by means of transposable elements. A transposable element (TE, transposon, or jumping gene) is a DNA sequence that can change its position in a nucleic acid, thereby creating or reversing mutations and altering sequences in a genome. Transposable elements represent a substantial fraction of many eukaryotic genomes. For example, around 50% of the human genome is derived from transposable element sequences and other genomes, for example, plants, may consist of substantially higher proportions of transposable element-derived DNA. Transposable elements are typically divided into two classes, class 1 and class 2. Class 1 is represented by the retrotransposons, including 1) long terminal repeat (LTR) retrotransposons, such as endogenous retroviruses (ERVs), and 2) non-LTR retrotransposons, such as long interspersed elements (LINEs) and short interspersed elements (SINEs). Class 2 TEs include 1) “cut-and-paste” DNA transposons, which are characterized by the terminal repeats (TRs, also known as terminal inverted repeats, TIRs) and are mobilized by a transposase, and 2) non-“cut-and-paste” DNA transposons, such as helitrons and polintons. While class 2 TEs are widespread and active in a variety of eukaryotes, not all of them are transpositionally active. Examples of recently active transposons include members of the hAT and piggyBac superfamilies with signs of mobilization in the past few million years. However, current options of active transposable elements available and suitable for gene discovery research and gene therapy are limited.

Therefore, there exists a need for new transposable elements suitable for introducing DNA into a cell, as well as methods and systems for efficient insertion of heterologous sequences of varying sizes into the nucleic acid of a cell or the insertion of DNA into the genome of a cell by means of transposable elements.

### BRIEF SUMMARY

The present application provides engineered transposable elements, gene transfer systems comprising the engineered transposable elements, as well as methods and kits for using the same. Also provided are methods of inserting a heterologous nucleic acid into a target nucleic acid in vitro or in a cell. The compositions, systems and methods described herein are useful for various applications including tagmentation, genome engineering and gene discovery research.

In one aspect, the present application provides an engineered transposable element comprising, from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof, and wherein the transposable element exhibits transposition activity that allows the heterologous nucleic acid to be inserted into the DNA of a cell. In some embodiments, the 5′TR comprises a nucleic acid sequence that has at least about 90% sequence identity to a nucleic acid sequence selected from the group consisting of SEQ ID NOs: 1-26 and 79-90. In some embodiments, the 5′TR comprises a nucleic acid sequence selected from the group consisting of SEQ ID NOs: 1-26 and 79-90. In some embodiments, the 3′TR comprises a nucleic acid sequence that has at least about 90% sequence identity to a nucleic acid sequence selected from the group consisting of SEQ ID NOs: 27-52 and 91-102. In some further embodiments, the 3′TR comprises a nucleic acid sequence selected from the group consisting of SEQ ID NOs: 27-52 and 91-102.

In some embodiments according to any one of the engineered transposable elements described above, the transposable element further comprises a 5′ target site duplication sequence (TSD) flanking the 5′ of the 5′TR or a 3′TSD flanking the 3′ of the 3′TR. In some embodiments, the nucleic acid sequences of 5′TSD and 3′TSD are the same sequence. In some embodiments, the 5′TSD comprises a nucleic acid sequence selected from the group consisting of SEQ ID NOs: 191-206, a variant thereof, or a fragment thereof, and the 3′TSD comprises a nucleic acid sequence selected from the group consisting of SEQ ID NOs: 191-206, a variant thereof, or a fragment thereof.

In some embodiments according to any one of the engineered transposable elements described above, the 5′TR comprises a nucleic acid sequence that has at least about 90% sequence identity to a nucleic acid sequence selected from the group consisting of SEQ ID NOs: 3, 8, 11, 12, 13, 16, 22, 23, 29 and 82, and the 3′TR comprises a nucleic acid sequence that has at least about 90% sequence identity to a nucleic acid sequence selected from the group consisting of SEQ ID NOs: 29, 34, 37, 38, 39, 42, 48, 49, 91 and 94.

In some embodiments according to any one of the engineered transposable elements described above, the engineered transposable element is derived from Tc1-8B_DR, Tc1-3_FR, Mariner2_AG, Tc1-1_Xt, Tc1-1_AG, Tc1-1_PM, Tc1-4_Xt, Tc1-15_Xt, Mariner-6_AMi, or Mariner-3_Crp. In some embodiments, the engineered transposable element is derived from Tc1-8B_DR, Tc1-3_FR, Mariner2_AG, Tc1-1_Xt, or Tc1-1_PM. In some embodiments, the 5′TR comprises a nucleic acid sequence of SEQ ID NO: 3, and the 3′TR comprises a nucleic acid sequence of SEQ ID NO: 29. In some embodiments, the 5′TR comprises a nucleic acid sequence of SEQ ID NO: 8, and the 3′TR comprises a nucleic acid sequence of SEQ ID NO: 34. In some embodiments, the 5′TR comprises a nucleic acid sequence of SEQ ID NO: 11, and the 3′TR comprises a nucleic acid sequence of SEQ ID NO: 37. In some embodiments, the 5′TR comprises a nucleic acid sequence of SEQ ID NO: 12, and the 3′TR comprises a nucleic acid sequence of SEQ ID NO: 38. In some embodiments, the 5′TR comprises a nucleic acid sequence of SEQ ID NO: 16, and the 3′TR comprises a nucleic acid sequence of SEQ ID NO: 42.

In some embodiments according to any one of the engineered transposable elements described above, the heterologous nucleic acid comprises a coding sequence. In some further embodiments, the heterologous nucleic acid further comprises a promoter operably linked to the coding sequence.

In some embodiments according to any one of the engineered transposable elements described above, the transposition activity of the transposable element is higher than that of a piggyBac (PB) transposon, a Sleeping Beauty (SB) transposon, and/or a TcBuster transposon.

In some embodiments according to any one of the engineered transposable elements described above, the cell is an animal cell, a plant cell, an algal cell, a fungal cell, a yeast cell, or a bacterial cell. In some embodiments, the cell is a mammalian cell. In some embodiments, the mammalian cell is selected from the group consisting of an immune cell (e.g., T cell), a hepatic cell, a tumor cell, a stem cell, a zygote, a muscle cell, and a skin cell. In some embodiments, the cell is a human cell. In some embodiments, the transposition activity of the transposable element is higher in a human embryonic kidney 293T (293T) cell than in a HeLa cell.

In some embodiments according to any one of the engineered transposable elements described above, the transposable element is present in a vector. In some further embodiments, the vector is a plasmid or a viral vector.

Another aspect of the present application provides a gene transfer system comprising: 1) an engineered transposable element according to any one of transposable elements described above; and 2) a transposase, or a nucleic acid encoding a transposase. In some embodiments, the transposase comprises an amino acid sequence selected from the group consisting of SEQ ID NOs: 53-78 and 103-114, or a variant thereof.

In yet another aspect, the present application provides a gene transfer system comprising: 1) an engineered transposable element; and 2) a transposase, or a nucleic acid encoding a transposase, wherein the transposable element comprises from 5′ to 3′: a 5′ terminal repeat sequence (5′TR), a heterologous nucleic acid, and a 3′ terminal repeat sequence (3′TR), wherein the transposable element exhibits transposition activity that allows the heterologous nucleic acid to be inserted into the DNA of a cell, and wherein the transposase comprises an amino acid sequence selected from the group consisting of SEQ ID NOs: 53-78 and 103-114, or a variant thereof. In some embodiments, the 5′TR comprises a nucleic acid sequence selected from the group consisting of SEQ ID NOs: 1-26 and 79-90, a variant thereof, or a fragment thereof, and wherein the 3′TR comprises a nucleic acid sequence selected from the group consisting of SEQ ID NOs: 27-52 and 91-102, a variant thereof, or a fragment thereof.

In some embodiments according to any one of the gene transfer systems described above, the transposable element comprises a 5′TR having a nucleic acid sequence that has at least about 90% sequence identity to a nucleic acid sequence selected from the group consisting of SEQ ID NOs: 3, 8, 11, 12, 13, 16, 22, 23, 29 and 82, and the 3′TR comprises a nucleic acid sequence that has at least about 90% sequence identity to a nucleic acid sequence selected from the group consisting of SEQ ID NOs: 29, 34, 37, 38, 39, 42, 48, 49, 91 and 94. In some embodiments, the engineered transposable element is derived from Tc1-8B_DR, Tc1-3_FR, Mariner2_AG, Tc1-1_Xt, Tc1-1_AG, Tc1-1_PM, Tc1-4_Xt, Tc1-15_Xt, Mariner-6_AMi, or Mariner-3_Crp. In some embodiments, the engineered transposable element is derived from Tc1-8B_DR, Tc1-3_FR, Mariner2_AG, Tc1-1_Xt, or Tc1-1_PM. In some embodiments, the 5′TR comprises a nucleic acid sequence of SEQ ID NO: 3, the 3′TR comprises a nucleic acid sequence of SEQ ID NO: 29, and the transposase comprises the amino acid sequence of SEQ ID NO: 55. In some embodiments, the 5′TR comprises a nucleic acid sequence of SEQ ID NO: 8, the 3′TR comprises a nucleic acid sequence of SEQ ID NO: 34, and the transposase comprises the amino acid sequence of SEQ ID NO: 60. In some embodiments, the 5′TR comprises a nucleic acid sequence of SEQ ID NO: 11, the 3′TR comprises a nucleic acid sequence of SEQ ID NO: 37, and the transposase comprises the amino acid sequence of SEQ ID NO:63. In some embodiments, the 5′TR comprises a nucleic acid sequence of SEQ ID NO: 12, the 3′TR comprises a nucleic acid sequence of SEQ ID NO: 38, and the transposase comprises the amino acid sequence of SEQ ID NO: 64. In some embodiments, the 5′TR comprises a nucleic acid sequence of SEQ ID NO: 16, the 3′TR comprises a nucleic acid sequence of SEQ ID NO: 42, the transposase comprises the amino acid sequence of SEQ ID NO: 68.

In some embodiments according to any one of the gene transfer systems described above, the gene transfer system comprises a nucleic acid encoding the transposase. In some further embodiments, the transposable element and the nucleic acid encoding the transposase are in separate vectors. In some further particular embodiments, the transposable element and the nucleic acid encoding the transposase are in the same vector.

In still another aspect, the present application provides a method of inserting a heterologous nucleic acid into a target nucleic acid, comprising: contacting the target nucleic acid with a transposable element according to any one of the engineered transposable elements described above or a gene transfer system according to any one of the gene transfer systems describe above. In some embodiments, the method is carried out in vitro. In some embodiments, the target nucleic acid is in a cell. In some embodiments, the target nucleic acid is genomic DNA.

In some embodiments according to any one of the methods described above, wherein the target nucleic acid is in a cell, the cell is an animal cell, a plant cell, an algal cell, a fungal cell, a yeast cell, or a bacterial cell. In some embodiments, the cell is a mammalian cell. In some embodiments, the mammalian cell is selected from the group consisting of an immune cell (e.g., T cell), a hepatic cell, a tumor cell, a stem cell, a zygote, a muscle cell, and a skin cell. In some embodiments, insertion of the heterologous nucleic acid inactivates a gene of the cell.

In some embodiments according to any one of the methods described above, the heterologous nucleic acid encodes a protein. In some embodiments, the protein is selected from the group consisting of a reporter protein, an engineered receptor, a cytokine, an antibiotic resistance protein, an antigen, and a therapeutic protein.

In some embodiments according to any one of the methods described above, the heterologous nucleic acid encodes a RNA. In some embodiments, the RNA is selected from the group consisting of a therapeutic RNA, a small interfering RNA (siRNA), a microRNA, a short hairpin RNA (shRNA), a long non-coding RNA (lincRNA), and a guide RNA (gRNA). In some embodiments, the heterologous nucleic acid encodes more than one molecule.

In some embodiments according to any one of the methods described above, the heterologous nucleic acid is no more than about 300 kilobases (kb) long, e.g., about 10 kb to about 300 kb, or about 100 basepairs (bp) to about 10 kb, or about 100 bp to about 5 kb, or about 100 bp to about 2 kb, or about 2 kb to about 300 kb long.

In some embodiments according to any one of the methods described above, the insertion is random.

In still another aspect, the present application provides a kit comprising a transposable element according to any one of the engineered transposable elements described above, or a gene transfer system according to any one of the gene transfer systems described above and instructions for inserting a heterologous nucleic acid in a target nucleic acid.

### BRIEF DESCRIPTION OF DRAWINGS

FIG. 1 shows a diagram illustrating the process of identifying active transposable elements (TEs).

FIG. 2 shows a summary of the identified 131 transposable elements (TEs) harboring an open reading frame (ORF) of no less than 300 amino acid (aa) long, a transposase (Tn) domain, and an average divergence value of no more than 25%. The pie chart illustrates the distribution of the five superfamilies for the identified 131 TEs.

FIG. 3 shows an exemplary set of binary constructs for use in screening active transposable elements. The top construct is the helper construct for transposase expression, comprising from 5′ to 3′: a cytomegalovirus (CMV) promoter, a transposase (Tn) gene, and a polyA (pA) signal. The bottom construct is the donor construct, comprising from 5′ to 3′: a phosphoglycerate kinase (PGK) promoter, a 5′ target site duplication (5′TSD) sequence, a 5′ terminal repeat (5′TR) sequence, a sequence encoding puromycin and enhanced GFP fusion (Puro-eGFP), a 3′ terminal repeat (3′TR) sequence, a 3′ target site duplication (3′TSD) sequence, and a polyA (pA) signal.

FIGS. 4A-4B demonstrate the transposition activity of the identified transposable elements with a divergence value no greater than 5% in HEK293T (293T) and HeLa cells as compared with control TEs including piggyBac, Hyper piggyBac and SB100X. FIG. 4A demonstrate the transposition activity of the identified transposable elements: 1_hAT-2_AG, 2_AgaP12, 3_P3_AG, 4_HAT2_CI, 5_HAT5_CI, 6_hAT-6_DR, 7_Chaplin1_DR, 8_Harbinger-4_XT, 9_HAT)_AG, 10_AgaP15, 11_IS4EU-1_DR, 12_POGO, 14_Tc1-8B_DR, 15_HOBO, 17_hAT-6_PM, 18_MARISP1, 19_hAT-7_XT, 20_BARI_DM, 21_Mariner-3_PM, 22_hAT-3_XT, 23_P1_AG, 24_IS4EU-2_DR, 25_Tc1-3_Xt, 26_Mariner-1_PM, 27_P2_AG, 28 hAT-5_DR, 29_Tc1-3_FR, 30_Mariner-4_XT 32_Tc1-5_Xt, 33_Tc1-10_Xt, and 34_hAT-1_D in HEK293T (293T) and HeLa cells as compared with control TEs including piggyBac, Hyper piggyBac, SB100X, TcBuster (TB), and negative control (NC). FIG. 4B demonstrate the transposition activity of the identified transposable elements: 35_Mariner2_AG, 36_Tc1-1_Xt, 37_Tc1-1_AG, 38_Mariner-1_XT, 39_Tc1DR3_Xt, 40_hAT-1B_PM, 41_hAT-3_PM, 42_hAT-6B_PM, 43_Tc1-1_PM, 44_TC1_XL, 45_Mariner-4_AMi, 46_Motis_hAT1, 47_hAT-7_PM, 48_TC1_FR4, 49_hAT-8_PM, 50_piggyBac1_Mm, 51_Tc1-11_Xt, 52_Tc1-16_Xt, 53_TC1-2_DM, 54_Tc1-4_Xt, 55_TC1_DM, 56_Tc1-15_Xt, 59_TC1_FR2, 60_Mariner-6B_AMi, 61_Tc1-9_Xt, 63_hAT-9_XT, 64_Mariner-5_XT, 65_S2_DM, 67_PROTOP, 68_Tc1-8_Xt and 69_Tc1-12 Xt in HEK293T (293T) and HeLa cells as compared with control TEs including piggyBac, Hyper piggyBac, SB100X, TcBuster (TB), and negative control (NC).

FIGS. 5A-5B demonstrate the transposition activity of the identified transposable elements with a divergence value greater than 5% in HEK293T (293T). FIG. 5A demonstrate the transposition activity of the identified transposable elements 70_Mariner-6 AMi, 71_hAT-3_Gav, 72_piggyBac2_Mm, 73_Mariner-2_XT, 74_hAT-4_Crp, 75_OposCharlie2, 76_hAT-1_AMi, 77_Mariner-2_AMi, 78_hAT-13_AMi, 80_hAT-12_AMi, 81_Mariner-7_Croc, 82_piggyBac-2_XT, 83_Mariner-3_AMi, 84_piggyBac1_CI, 86_piggyBac1-1_AMi, 87_Mariner-5_AMi, 89_Mariner-6_Crp, 90_Mariner-3_Crp, 91_hAT-3_AMi, 92_TC1_FR1, 93_hAT-5_Croc, 94_hAT-8_AMi, 95_PARIS, 96_Mariner-2_PM, 97_piggyBac-1_XT, 98_Tigger1, 99_Mariner-1_Cp, 101_S_DM, 102_hAT-12_Crp, 103_hAT-1_PM, 104_Senkusha1, 105_Tc1-2 PM, 106 Harbinger-2_AMi, 108_hAT-2_Gav and 110_Tigger2f in HEK293T (293T) FIG. 5B demonstrate the transposition activity of the identified transposable elements 111_Tc1-4_DR, 116_Tigger17, 117_Tigger3, 118_hAT-14_Crp, 119_Mariner-9_Crp, 124_hAT-11_AMi, 126_Mariner-10_Crp, 139_hAT-17_Croc, 140_hAT-4_AMi, 142_Tigger4, 144_Tigger7, 156_Tigger2, 180_hAT-19_Crp, 183_hAT-17B_Croc, 188_Tc1-13_Xt, 197_hAT-19B_Croc, 199_MarsTigger8, 212_Tigger5, 222_hAT-10_XT 237_hAT-6_AMi, 245_MarsTigger1c, 246_Tigger17c, 258 Harbinger-1_AMi, 260_Tc1-14_Xt, 271 Kanga1, 275_Arthur1, 295_Harbinger-1_Crp, 314_Zaphod3, 320 Joey1, 331_Zaphod, 342_Harbinger-3_AMi, 348_Harbinger-1B_Crp, 349_MARWOLEN1 and 351_Zaphod2 in HEK293T (293T)

FIGS. 6A-6B show the transposition (i.e., transfer) efficiency of the various identified TEs as compared with control TEs piggyBac, hyperpiggyBac and SB100X. FIG. 6A shows the transposition (transfer) efficiency of the various identified TEs as compared with control TEs piggyBac, hyperpiggyBac and SB100X in HEK293T (293T) cells. FIG. 6B shows the transposition (transfer) efficiencies of the various identified TEs as compared with control TEs piggyBac, hyperpiggyBac and SB100X in HeLa cells.

FIGS. 7A-7C shows a phylogenetic tree illustrating the phylogenetic relationship among the identified transposases belonging to different superfamilies and their control TEs. FIG. 7A shows a phylogenetic tree illustrating the phylogenetic relationship between the 14 identified hAT superfamily transposases 103_hAT-1_PM, 17_hAT-6_PM, 46 Myotis_hAT1, 28_hAT-5_DR, 22_hAT-3_XT, 41_hAT-3_PM, 47_hAT-7_PM, 180_hAT-19_Crp, 1_hAT-2_AG, 9_HAT)_AG, 139_hAT-17_Croc, 183_hAT-17B_Croc, 63_hAT-9 XT, 222_hAT-10_XT, and control TE TcBuster. FIG. 7B shows a phylogenetic tree illustrating the phylogenetic relationship between the 2 identified piggyBac superfamily transposases 82_piggyBac-2_XT: 86_piggyBac-1_AMi and control TE piggyBac. FIG. 7C shows a phylogenetic tree illustrating the phylogenetic relationship between the 22 identified TcMariner superfamily transposases and control TE SB100X.

FIGS. 8A-8B demonstrate the transposition activity of the identified transposable elements and top 5 active TEs among 131 candidates in HEK293T (293T) cells and HeLa cells as compared with control TEs including piggyBac, hyperpiggyBac and SB100X, and negative control (NC). FIG. 8A shows transposition (transfer) efficiencies of the various identified TEs as compared with control TEs including piggyBac, hyperpiggyBac and SB100X, and negative control (NC) in 293T cells. FIG. 8B shows transposition (transfer) efficiencies of the various identified TEs as compared with control TEs including piggyBac, hyperpiggyBac and SB100X, and negative control (NC) in HeLa cells.

FIGS. 9A-9B demonstrate the transposition activity of the identified transposable elements and top 5 active TEs among 131 candidates in Hct116 and K562 cells as compared with control TEs including piggyBac, hyperpiggyBac and SB100X, and negative control (NC). FIG. 9A shows transposition (transfer) efficiencies of the various identified TEs as compared with control TEs including piggyBac, hyperpiggyBac and SB100X, and negative control (NC) in Hct116 cells. FIG. 9B shows transposition (transfer) efficiencies of the various identified TEs as compared with control TEs including piggyBac, hyperpiggyBac and SB100X, and negative control (NC) in K562 cells.

FIG. 10A-10B demonstrate the transposition activity of the identified transposable elements 14-Tc1-8B_DR, 29-Tc1-3 FR, 35-Mariner2_AG, 36-Tc1-1_Xt, 37-Tc1-1_AG, 43-Tc1-1_PM, 52-Tc1-16_Xt, 54-Tc1-4_Xt, and 56-Tc1-15_Xt in primary T cells as compared with control TE SB100X. FIG. 10A shows the transposition assay results of primary T cells transfected with plasmids containing EF1α promoter and CopGFP gene, wherein presence of GFP positive T cells represent successful transposition in the cells. FIG. 10B shows the transposition assay results of primary T cells transfected with plasmids containing EF1α promoter and 019CAR-P2A-eGFP gene, wherein presence of GFP positive T cells represent successful transposition in the cells.

FIGS. 11A-11B demonstrate the transposition activity of the identified transposable elements SB100X and top 5 active TEs among 131 candidates based on different ratio of helper and transposon plasmids in 293T cells and HeLa cells as compared with control TE piggyBac and negative control (NC). FIG. 11A shows transposition (transfer) efficiencies of the various identified TEs as compared with control TE SB100X and negative control (NC) in 293T cells. FIG. 11B shows transposition (transfer) efficiencies of the various identified TEs as compared with control TE SB1001X and negative control (NC) in HeLa cells.

FIG. 12 demonstrate the cargo capacity of the identified transposable elements SB100X and top 5 active TEs among 131 candidates in 293T cells as compared with control TE piggyBac, piggyBac, hyperpiggyBac, SB100X. FIG. 12 shows transposition (transfer) efficiencies of the various identified TEs as compared with control TEs piggyBac, piggyBac, hyperpiggyBac, SB100X.

FIG. 13 demonstrate the consensus sequences at the genomic insertion loci in a 40 bp window around the target site in genome from TEs original species and stable transposition K562 cell line.

FIGS. 14A-14B demonstrate the insertion frequencies of the identified transposable elements including piggyBac, hyperpiggyBac, SB100X and top 5 active TEs among 131 candidates in the distance to nearest gene, FIG. 14A show the insertion frequencies in 0 bp-1 Mb windows and FIG. 14B show the fold enrichment than random insertion in 50 kb windows.

FIG. 15 demonstrate the fold enrichment than random insertion of the identified transposable elements including piggyBac, hyperpiggyBac, SB100X and top 5 active TEs among 131 candidates in the relative location in gene body from 5′ terminus to 3′terminus.

FIG. 16 demonstrate the fold enrichment than random insertion of the identified transposable elements including piggyBac, hyperpiggyBac, SB100X and top 5 active TEs among 131 candidates in the different genes with expression level, level 7 means the highest expression and level 0 means the lowest expression.

FIG. 17 demonstrate the fold enrichment than random insertion of the identified transposable elements including piggyBac, hyperpiggyBac, SB100X and top 5 active TEs among 131 candidates in the location around transcription start sites (TSS) in 5 kb windows.

FIG. 18 demonstrate the fold enrichment than random insertion of the identified transposable elements including piggyBac, hyperpiggyBac, SB100X and top 5 active TEs among 131 candidates in the different chromatin states.

### DETAILED DESCRIPTION

The present application provides engineered transposable elements, gene transfer systems comprising the engineered transposable elements, as well as methods and kits for using the same. The present application is based, at least in part, on the identification of novel transposable elements (e.g., Table 2) from a wide range of species from pan-genome bioinformatics analysis, as well as the surprising results that a number of the identified transposable elements are capable of transposition in human cells with high efficiency. The disclosed compositions, systems, and methods are useful for inserting heterologous nucleic acids into a target nucleic acid, including introducing heterologous DNA into the genome of a cell. The transposable elements and gene transfer systems described herein can be used for various applications, such as gene therapy and gene discovery research.

Accordingly, in one aspect, the present application provides an engineered transposable element comprising, from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof, and wherein the transposable element exhibits transposition activity that allows the heterologous nucleic acid to be inserted into the DNA of a cell.

In another aspect, the present application provides a gene transfer system comprising: 1) an engineered transposable element; and 2) a transposase, or a nucleic acid encoding a transposase, wherein the transposable element comprises from 5′ to 3′: a 5′ terminal repeat sequence (5′TR), a heterologous nucleic acid, and a 3′ terminal repeat sequence (3′TR), wherein the transposable element exhibits transposition activity that allows the heterologous nucleic acid to be inserted into the DNA of a cell. In some embodiments, the transposable element is an engineered transposable element. In some embodiments, the transposase comprises an amino acid sequence selected from the group consisting of SEQ ID NOs: 53-78 and 103-114, or a variant thereof.

### I. Definitions

As used herein, the term “transposon,” “transposable element” or “TE” refers to a polynucleotide that is able to excise from a first nucleic acid (i.e., donor nucleic acid, for example, a vector) and integrate into a target site (e.g., a second nucleic acid or genomic or extrachromosomal DNA in a cell). A transposon includes a nucleic acid sequence flanked by cis-acting nucleic acid sequences on the termini of the transposon. A nucleic acid sequence is “flanked by” cis-acting nucleic acid sequences if at least one cis-acting nucleic acid sequence is positioned 5′ to the nucleic acid sequence, and at least one cis-acting nucleic acid sequence is positioned 3′ to the nucleic acid sequence. Cis-acting nucleic acid sequences include at least one terminal repeat (TR, also known as an inverted terminal repeat (ITR) or a terminal inverted repeat (TIR)) at each end of the transposon, to which a transposase binds. A transposable element described herein may or may not contain an open reading frame (ORF) encoding a transposase.

As used herein, the term “transposition” refers to the change in location of a transposable element from a first nucleic acid (e.g., a vector) and integrate into a target site (e.g., a second nucleic acid or genomic or extrachromosomal DNA in a cell).

As used herein, the term “terminal repeats” or “TRs” refer to nucleic acid sequences at both ends of a transposable element and flanking a second nucleic acid sequence that can be transposed. The TR located 5′ (upstream) to the second nucleic acid sequence is referred to the 5′TR, and the TR located 3′ (downstream) to the second nucleic acid sequence is referred to the 3′TR. In class 2 transposable elements, the TRs are complements to each other.

As used herein, the term “target site duplication” or “TSD” refers to nucleic acid sequences that occur at insertion sites of transposable elements. TSDs may occur due to DNA repair of sticky ends caused by staggered cut of target DNA duplex by transposases. TSDs flank TRs in a transposable element. The TSD located 5′ to the 5′TR is the 5′TSD. The TSD located 3′ to the 3′TR is the 3′TSD.

As used herein, the term “transposase” refers to a polypeptide that catalyzes the excision of a transposon from a first nucleic acid (e.g., a vector) and integrate into a target site (e.g., a second nucleic acid or genomic or extrachromosomal DNA in a cell). In some embodiments, a transposase binds one or both terminal repeat sequences.

As used herein, a “left transposon fragment” or “LTF” refers to a fragment in a naturally occurring transposable element from the 5′TSD to the start codon of a transposase ORF sequence. As used herein, a “right transposon fragment” or “RTF” refers to a fragment in a naturally occurring transposable element from the stop codon of the transposase ORF sequence to the 3′TSD.

The terms “nucleic acid”, “polynucleotide”, and “nucleic acid sequence” are used interchangeably to refer to a polymeric form of nucleotides of any length, including deoxyribonucleotides, ribonucleotides, combinations thereof, and analogs thereof. “Oligonucleotide” and “oligo” are used interchangeably to refer to a short polynucleotide, having no more than about 50 nucleotides.

As used herein, a “heterologous nucleic acid” refers to a DNA or RNA sequence that is from a different origin than a reference nucleic acid sequence. For example, in the context of a transposable element, a heterologous nucleic acid is from a different origin than the terminal repeat sequences. For example, a nucleic acid sequence that has been isolated from an organism different from that of the terminal repeats is considered a heterologous nucleic acid with respect to the terminal repeats.

As used herein, the term “operably linked” refers to a nucleic acid sequence that is placed in a functional relationship with another nucleic acid sequence. For example, if a coding sequence is operably linked to a promoter sequence, this generally means that the promoter may promote transcription of the coding sequence. Operably linked means that the DNA sequences being linked are typically contiguous and, where necessary join two protein coding regions, contiguous and in reading frame. Since enhancers may function when separated from the promoter by several kilobases and intron sequences may be of variable lengths, some nucleic acid sequences may be operably linked but not contiguous.

“Percentage (%) sequence identity” with respect to a nucleic acid sequence is defined as the percentage of nucleotides in a candidate sequence that are identical with the nucleotides in the specific nucleic acid sequence, after aligning the sequences by allowing gaps, if necessary, to achieve the maximum percent sequence identity. “Percentage (%) sequence homology” with respect to a peptide, polypeptide or protein sequence is the percentage of amino acid residues in a candidate sequence that are identical substitutions to amino acid residues in the specific peptide or amino acid sequence, after aligning the sequences by allowing gaps, if necessary, to achieve the maximum percent sequence homology. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “vector” as used herein refers to a nucleic acid molecule capable of transporting another nucleic acid molecule to which it has been linked. Examples of vectors include but are not limited to bacteria, plasmids, phages, cosmids, episomes, viruses, and insertable DNA fragments, i.e., fragments capable of being inserted into a host cell genome by homologous recombination.

As used herein, the term “plasmid” refers to circular, double-stranded DNA capable of accepting a foreign DNA fragment and capable of replicating in prokaryotic or eukaryotic cells.

The terms “polypeptide”, and “peptide” are used interchangeably herein to refer to polymers of amino acids of any length. Thus, for example, the terms peptide, oligopeptide, protein, antibody, and enzyme are included within the definition of polypeptide. The polymer may be linear or branched, it may comprise modified amino acids, and it may be interrupted by non-amino acids. A protein may have one or more polypeptides. The terms also encompass an amino acid polymer that has been modified; for example, disulfide bond formation, glycosylation, lipidation, acetylation, phosphorylation, or any other manipulation, such as conjugation with a labeling component.

As used herein, a “variant” is interpreted to mean a polynucleotide or polypeptide that differs from a reference polynucleotide or polypeptide, respectively, but retains essential properties. A typical variant of a polynucleotide differs in nucleic acid sequence from another, reference polynucleotide. Changes in the nucleic acid sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, a “fragment” of a sequence refers to a portion of the sequence. For example, a fragment of a nucleic acid sequence refers to a portion of the nucleic acid sequence, and a fragment of an amino acid sequence refers to a portion of the amino acid sequence.

As used herein the term “genetic circuit”, “biological circuit”, or “synthetic circuit” refers to a set of biological components designed to perform logical functions. In general, an input is needed to activate a genetic circuit, which subsequently produces an output as a function of the input.

The term “engineer” as used herein refers to any manipulation that results in a detectable change in a polynucleotide or a polypeptide, wherein the manipulation includes, but is not limited to, inserting, deleting, and substituting a portion of the polynucleotide or amino acid sequence.

The term “transposition efficiency” as used herein refers to the efficiency of a transposable element to insert a heterologous nucleic acid into a population of target cells. For example, transposition efficiency can be determined by transfecting a plasmid comprising a transposable element comprising a reporter gene or a gene encoding a selectable marker, for example, an antibiotic resistance gene (e.g., puromycin), into a population of target cells, and determine the number of cells expressing the gene product encoded by the reporter gene or selectable marker, for example, by measuring the number of cells having antibiotic resistance.

The term “transfected” or “transformed” or “transduced” as used herein refers to a process by which exogenous nucleic acid is transferred or introduced into a host cell. A “transfected” or “transformed” or “transduced” cell is one, which has been transfected, transformed or transduced with exogenous nucleic acid. The term “transduction” and “transfection” as used herein include all methods known in the art using an infectious agent (such as a virus) or other means to introduce DNA into cells for expression of a protein or molecule of interest. Besides a virus or virus like agent, there are chemical-based transfection methods, such as those using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, delivery of plasmids, or transposons; particle-based methods, such as using a gene gun, magnectofection or magnet assisted transfection, particle bombardment; and hybrid methods, such as nucleofection.

The term “in vivo” refers to inside the body of the organism from which the cell is obtained. “Ex vivo” or “in vitro” means outside the body of the organism from which the cell is obtained.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise.

### II. Engineered Transposable Elements

The present application in one aspect provides an engineered transposable element comprising, from 5′ to 3′: a 5′ terminal repeat sequence (5′TR), a heterologous nucleic acid, and a 3′ terminal repeat sequence (3′TR). In some embodiments, the transposable element exhibits transposition activity that allows the heterologous nucleic acid to be inserted into a target nucleic acid (e.g., DNA) in vitro. In some embodiments, the engineered transposable element exhibits transposition activity that allows the heterologous nucleic acid to be inserted into a target nucleic acid in a cell (such as DNA in a plant of mammalian cell). In some embodiments, the transposable element comprises a nucleic acid sequence encoding the transposase. In some embodiments, the transposable element does not comprise a nucleic acid sequence encoding the transposase.

In some embodiments, the engineered transposable element comprises, from 5′ to 3′: a 5′ target site duplication sequence (5′TSD), a 5′TR, a heterologous nucleic acid, a 3′TR and a 3′TSD. In some embodiments, the transposable element exhibits transposition activity that allows the heterologous nucleic acid to be inserted into a target nucleic acid (e.g., DNA) in vitro. In some embodiments, the engineered transposable element exhibits transposition activity that allows the heterologous nucleic acid to be inserted into a target nucleic acid in a cell (such as DNA in a plant of mammalian cell). In some embodiments, the transposable element comprises a nucleic acid sequence encoding the transposase. In some embodiments, the transposable element does not comprise a nucleic acid sequence encoding the transposase.

In some embodiments, the present application provides novel transposable elements from a wide range of species that are capable of transposition in human cells with high efficiency. The transposable elements described herein provide direct experimental evidence for naturally active mammalian cut-and-paste DNA transposons.

A list of exemplary transposable elements with their corresponding left terminal fragment (LTF), right terminal fragment (RTF), transposase, 5′terminal repeat (TR), 3′TR, 5′ target site duplication (TSD) and 3′TSD sequences can be found in Tables 1-2 and the sequence listing. Table 1 lists 131 TEs identified from the bioinformatics analysis as disclosed in the present application, including 11 TEs (TE IDs: 4, 5, 7, 12, 18, 20, 22, 25, 26, 28 and 30) meeting the criteria of having a length of no more than 3000 bp, a MITE copy number greater than 10, and an average divergence smaller than 1%, suitable for use in efficient genome engineering. Table 2 lists experimentally validated active TEs using transposition assays in human cell lines HEK293T and Hela.

In some embodiments, the transposable elements are Class 2 transposable elements. Class 2 transposable elements may be classified into superfamilies based on the relatedness of the transposase and on shared structural features, including the terminal repeats (TRs) and the length of the target site duplications (TSDs) generated during integration flanking the TRs. It is contemplated that the transposable element of the present application may be from various suitable TE superfamilies and/or families. In some embodiments, the transposable element is from the hAT superfamily. In some embodiments, the transposable element is from the P superfamily. In some embodiments, the transposable element is from the PIF-Harbinger superfamily. In some embodiments, the transposable element is from the piggyBac superfamily. In some embodiments, the transposable element is from the TcMariner superfamily. In some embodiments, the 5′TR is a reverse complement of the 3′ TR. In some embodiments, the 5′TR is not a reverse complement of the 3′TR.

In some embodiments, the engineered transposable element comprises a 5′TR comprising a nucleic acid sequence that has at least about 50%, 60%, 70%, 80%, 90%, 95%, 99% or 100% sequence identity to the 5′TR of a transposable element of a superfamily selected from the group consisting of hAT, P, PIF-Harbinger, piggyBac, and TcMariner. In some embodiments, the engineered transposable element comprises a 3′TR comprising a nucleic acid sequence that has at least about 50%, 60%, 70%, 80%, 90%, 95%, 99% or 100% sequence identity to the 3′TR of a transposable element of a superfamily selected from the group consisting of hAT, P, PIF-Harbinger, piggyBac, and TcMariner.

In some embodiments, the engineered transposable element comprises a 5′TR comprising a nucleic acid sequence that has at least about 50%, 60%, 70%, 80%, 90%, 95%, 99% or 100% sequence identity to the 5′TR of a transposable element of a superfamily selected from the group consisting of hAT, P, PIF-Harbinger, piggyBac, and Tc1, and a 3′TR comprising a nucleic acid sequence that has at least about 50%, 60%, 70%, 80%, 90%, 95%, 99% or 100% sequence identity to the 3′TR of a transposable element of a superfamily selected from the group consisting of hAT, P, PIF-Harbinger, piggyBac, and TcMariner.

In some embodiments, the engineered transposable element comprises a 5′ TR, a 3′TR, a LTF, an RTF, a transposase, a 5′TSD, and/or a 3′TSD derived from any one of the TEs of Table 1. In some embodiments, the engineered transposable element comprises a 5′ TR, a 3′TR, a LTF, an RTF, a transposase, a 5′TSD, and/or a 3′TSD derived from any one of the TEs of Table 2. In some embodiments, the engineered transposable element is derived from Tc1-8B_DR, Tc1-3_FR, Mariner2_AG, Tc1-1_Xt, Tc1-1_AG, Tc1-1_PM, Tc1-4 Xt, Tc1-15_Xt, Mariner-6 AMi, or Mariner-3_Crp. In some embodiments, the engineered transposable element is derived from Tc1-8B_DR, Tc1-3_FR, Mariner2_AG, Tc1-1_Xt, or Tc1-1_PM.

In some embodiments, the engineered transposable element comprises a LTF comprising a nucleic acid sequence selected from the group consisting of SEQ ID NOs: 115-140 and 167-178, a variant thereof, or a fragment thereof.

In some embodiments, the engineered transposable element comprises a RTF comprising a nucleic acid sequence selected from the group consisting of SEQ ID NOs: 141-190, a variant thereof, or a fragment thereof.

In some embodiments, the engineered transposable element comprises a LTF having at least about any one of 80%, 85%, 90%, 91%, 92%, 93%, 94%, 95%, 96%, 97%, 98%, 99% or more sequence identity to a nucleic acid sequence selected from the group consisting of SEQ ID NOs: 115-140 and 167-178; and a RTF having at least about any one of 80%, 85%, 90%, 91%, 92%, 93%, 94%, 95%, 96%, 97%, 98%, 99% or more sequence identity to a nucleic acid sequence selected from the group consisting of SEQ ID NOs: 141-190. In some embodiments, the engineered transposable element comprises a LTF comprising a nucleic acid sequence selected from the group consisting of SEQ ID NOs: 115-140 and 167-178; and an RTF comprising a nucleic acid sequence selected from the group consisting of SEQ ID NOs: 141-190.

In some embodiments, the engineered transposable element comprises a 5′TR in a LTF comprising a nucleic acid sequence selected from the group consisting of SEQ ID NOs: 115-140 and 167-178._In some embodiments, the engineered transposable element comprises a 3′TR in a RTF comprising a nucleic acid sequence selected from the group consisting of SEQ ID NOs: 141-190.

In some embodiments, the engineered transposable element comprises a 5′TR in a LTF comprising a nucleic acid sequence selected from the group consisting of SEQ ID NOs: 115-140 and 167-178, a variant of the 5′TR, or a fragment of the 5′TR; and a 3′TR in a RTF comprising a nucleic acid sequence selected from the group consisting of SEQ ID NOs: 141-190, a variant of the 3′TR, or a fragment of the 3′TR.

In some embodiments, the engineered transposable element comprises a 5′TR having a nucleic acid sequence that has at least about 90% sequence identity to a nucleic acid sequence selected from the group consisting of SEQ ID NOs: 1-26 and 79-90. In some embodiments, the engineered transposable element of the present application comprises a 5′TR having a nucleic acid sequence that has at least about any one of 90%, 91%, 92%, 93%, 94%, 95%, 96%, 97%, 98%, 99%, or more sequence identity to a nucleic acid sequence selected from the group consisting of SEQ ID NOs: 1-26 and 79-90. In some embodiments, the 5′TR comprises a nucleic acid sequence selected from the group consisting of SEQ ID NOs: 1-26 and 79-90. In some embodiments, the engineered transposable element comprises a 3′TR that has complementary sequence as the 5′TR.

In some embodiments, the engineered transposable element comprises a 3′TR having a nucleic acid sequence that has at least about 90% sequence identity to a nucleic acid sequence selected from the group consisting of SEQ ID NOs: 27-52 and 91-102. In some embodiments, the engineered transposable element comprises a 3′TR having a nucleic acid sequence that has at least about any one of 90%, 91%, 92%, 93%, 94%, 95%, 96%, 97%, 98%, 99%, or more sequence identity to a nucleic acid sequence selected from the group consisting of SEQ ID NOs: 27-52 and 91-102. In some embodiments, the 3′TR comprises a nucleic acid sequence selected from the group consisting of SEQ ID NOs: 27-52 and 91-102. In some embodiments, the engineered transposable element comprises a 5′TR that has complementary sequence as the 3′TR.

In some embodiments, the engineered transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to a nucleic acid sequence selected from the group consisting of SEQ ID NOs: 1-26 and 79-90; and 2) a 3′TR having a nucleic acid sequence that has at least about 90% (e.g. at least about any one of 91%, 92%, 93%, 94%, 95%, 96%, 97%, 98%, 99%, or more) sequence identity to a nucleic acid sequence selected from the group consisting of SEQ ID NOs: 27-52 and 91-102.

In some embodiments, the engineered transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to a nucleic acid sequence selected from the group consisting of SEQ ID NOs: 1-26; and 2) a 3′TR having a nucleic acid sequence that has at least about 90% (e.g., at least about any one of 91%, 92%, 93%, 94%, 95%, 96%, 97%, 98%, 99%, or more) sequence identity to a nucleic acid sequence selected from the group consisting of SEQ ID NOs: 27-52.

In some embodiments, the engineered transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to a nucleic acid sequence selected from the group consisting of SEQ ID NOs: 79-90; and 2) a 3′TR having a nucleic acid sequence that has at least about 90% (e.g., at least about any one of 91%, 92%, 93%, 94%, 95%, 96%, 97%, 98%, 99%, or more) sequence identity to a nucleic acid sequence selected from the group consisting of SEQ ID NOs: 91-102.

Also contemplated herein are engineered transposable elements comprising a variant or a fragment of any one of the 5′TRs and/or 3′TRs, or LTF and/or RTF described herein, e.g., in Tables 1 and 2. In some embodiments, the variant comprises no more than about any one of 50, 40, 35, 30, 25, 20, 15, 10, 9, 8, 7, 6, 5, 4, 3, 2, or 1 nucleotide substitution(s). In some embodiments, the fragment comprises at least about any one of 5, 6, 7, 8, 9, 10, 15, 20, 25, 30, 35, 40, 45, 50, 60, 70, 80, 90, 100, 150, 200, 250, 300, 350, 400, 450, or 500 nucleotides.

In some embodiments, the engineered transposable element comprises a 5′TSD. In some embodiments, the engineered transposable element comprises a 3′TSD. In some embodiments, the engineered transposable element comprises both a 5′TSD and a 3′TSD. In some embodiments, the engineered transposable element does not comprise a 5′TSD. In some embodiments, the engineered transposable element does not comprise a 3′TSD. In some embodiments, the engineered transposable element does not comprise a 5′TSD or a 3′TSD. In some embodiments, the 5′TSD is identical to the 3′TSD. In some embodiments, the 5′TSD is different from the 3′TSD.

In some embodiments, the engineered transposable element comprises a nucleic acid sequence encoding a transposase. In some embodiments, the engineered transposable element does not comprise a nucleic acid sequence encoding a transposase. In some embodiments, the transposase is derived from the same species as the 5′TR and 3′TR sequences. In some embodiments, the transposase is a native transposase with respect to the 5′TR and 3′TR sequences. In some embodiments, the transposase is an engineered transposase based on a native transposase with respect to the 5′TR and 3′TR sequences.

Transposase catalyzes excision of a transposon from a donor polynucleotide (e.g., a vector) and subsequent integration of the transposon into a target nucleic acid, such as genomic or extrachromosomal DNA of a target cell. In some embodiments, a transposase binds a terminal repeat of a transposable element.

In some embodiments, the transposase comprises an amino acid sequence selected from the group consisting of SEQ ID NOs: 53-78 and 103-114, a variant thereof, or a fragment thereof.

The present application in some embodiments contemplate variants of the transposases listed in Tables 1-2 and the sequence listing. The recitation of a transposase variant refers to transposase polypeptides that are distinguished from a reference transposase polypeptide (e.g., a naturally occurring transposase polypeptide) by the addition, deletion, truncations, and/or substitution of at least one amino acid residue, which retain transposition activity. In certain embodiments, a transposase polypeptide variant is distinguished from a reference transposase polypeptide by one or more substitutions, which may be conservative or non-conservative, as known in the art. In certain embodiments, a variant transposase comprises an amino acid sequence having at least about any one of 80%, 85%, 90%, 91%, 92%, 93%, 94%, 95%, 96%, 97%, 98%, 99% or more sequence identity or similarity to a corresponding sequence of a reference transposase. In some embodiments, a variant transposase comprises no more than about any one of 50, 40, 35, 30, 25, 20, 15, 10, 9, 8, 7, 6, 5, 4, 3, 2, or 1 amino acid substitutions.

Functional fragments having amino acid deletions or variants having amino acid additions of the transposases described herein are also contemplated. In some embodiments, a transposase fragment is at least about any one of 50, 60, 70, 80, 90, 100, 150, 200, 250, 300, 350, 400, 450, 500, 600, 700 or more amino acid residues long. In certain embodiments, the amino acid additions or deletions occur at the C-terminal end and/or the N-terminal end of the reference transposase. In some embodiments, the amino acid additions or deletions occur at an internal position, such as a flexible loop of the reference transposase. In certain embodiments, the amino acid deletions (e.g., N-terminal and/or C-terminal truncation) comprises any one of about 1, about 2, about 3, about 4, about 5, about 6, about 7, about 8, about 9, about 10, about 15, about 20, about 25, about 30, about 35, about 40, about 45, 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175 or more amino acids, including all values and ranges in between these values. In some embodiments, a variant transposase comprises an N-terminal or C-terminal purification tag, selection marker (e.g., antibiotic resistance gene), or a reporter (e.g., a fluorescence reporter).

As noted above, transposase polypeptides of the invention may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleic acid sequence alterations are well known in the art. See, for example, Kunkel (1985, Proc. Natl. Acad. Sci. USA. 82: 488-492), Kunkel et al., (1987. Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In some embodiments, the transposase is codon-optimized as compared to the reference transposase. In some embodiments, the transposase is codon-optimized for expression in a mammalian cell, such as a human cell. In some embodiments, the transposase is codon-optimized for expression in a plant cell.

In some embodiments, the transposase comprises an amino acid sequence that has at least about 80% (e.g., at least about any one of 85%, 86%, 87%, 88%, 89%, 90%, 91%, 92%, 93%, 94%, 95%, 96%, 97%, 98%, 99%, or more) sequence identity to an amino acid sequence selected from the group consisting SEQ ID NOs: 53-78 and 103-114. In some embodiments, the transposase comprises an amino acid sequence selected from the group consisting SEQ ID NOs: 53-78 and 103-114.

In some embodiments, the transposable element comprises: 1) a 5′TR in a LTF comprising the nucleic acid sequence of SEQ ID NO: 115; and 2) a 3′TR in a RTF comprising the nucleic acid sequence of SEQ ID NO: 141 In some embodiments, the transposable element comprises: 1) a 5′TR comprising the nucleic acid sequence of TAGGC (SEQ ID NO: 1), a variant thereof, or a fragment thereof; and 2) a 3′TR comprising the nucleic acid sequence of GCCTA (SEQ ID NO: 27), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 1; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 27. In some embodiments, the transposable element comprises: 1) a 5′TR comprising the nucleic acid sequence of SEQ ID NO: 1; and 2) a 3′TR comprising the nucleic acid sequence of SEQ ID NO: 27. In some embodiments, the transposable element further comprises a 5′TSD comprising the nucleic acid sequence of GTATGGAC (SEQ ID NO: 191), and a 3′TSD comprising the nucleic acid sequence of SEQ ID NO: 191. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 115; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 141. In some embodiments, the transposable element is associated with a transposase comprising the amino acid sequence of SEQ ID NO: 53, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 53. In some embodiments, the transposable element comprises a nucleic acid sequence encoding the transposase. In some embodiments, the transposable element does not comprise a nucleic acid sequence encoding the transposase.

(transposase of hAT-2_AG)
SEQ ID NO: 53
MASNVATSSSIWDHFTRNGTKAKCRYCYNEIAFTK
GSTSNLKRHMKAKHPSISLERQHLASTTSPSTQNS
EPGPSCSSRPPNLISNFFKQPMNSETKRKLDMMLL
KLICKDCLPLSIVESEAFKTFVGCLNQNYDLPTRK
NVSNALLPSIYNEILVKVQGEVRNATSIALTTDGW
TNVNNTSFLGLTAHFIDNDYKLRSCLLECSEISLS
HSGQNIAAWIKEVIIKYEIQDKIVGIVTDNAANMK
SAARELEFNHVTCFAHSLHLIVKDATKKSIISTVD
EVKRIVMYFKKSPKATNELADTQSKFNLPNLKLKQ
DVPTRWNSTYDMLNRFYKNKIAIVACADKLNTLDP
IKIDWAILEHSLNALKIFDVATNMVSAEKNITVSH
VGLLSKMIIRKLNETDYTIPELGNLVTHLKEGVKK
RLEIYSNNQIIAKSMLLDPRIKKQGFHEEPLKYRE
TYELIIQELIPFQTPSMNAQEPHNIDNEANLLLGE
FITWVNNAECEIESPIELAKNELNSFLKIRNIDIK
NDPLEWWRIHSSKYPSIYALAKTIICIPGTSVPCE
RLFSKAGQIYSDKRSRLHPKKFKEIIFIQQNVDKF

In some embodiments, the transposable element comprises: 1) a 5′TR in a LTF comprising the nucleic acid sequence of SEQ ID NO: 116; and 2) a 3′TR in a RTF comprising the nucleic acid sequence of SEQ ID NO: 142. In some embodiments, the transposable element comprises: 1) a 5′TR comprising the nucleic acid sequence of TAG (SEQ ID NO: 2), a variant thereof, or a fragment thereof; and 2) a 3′TR comprising the nucleic acid sequence of CTA (SEQ ID NO: 28), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 2; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 28. In some embodiments, the transposable element comprises: 1) a 5′TR comprising the nucleic acid sequence of SEQ ID NO: 2; and 2) a 3′TR comprising the nucleic acid sequence of SEQ ID NO: 28. In some embodiments, the transposable element further comprises a 5′TSD comprising the nucleic acid sequence of ATGTGAAC (SEQ ID NO: 192), and a 3′TSD comprising the nucleic acid sequence of SEQ ID NO: 192. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 116; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 142. In some embodiments, the transposable element is associated with a transposase comprising the amino acid sequence of SEQ ID NO: 54, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 54. In some embodiments, the transposable element comprises a nucleic acid sequence encoding the transposase. In some embodiments, the transposable element does not comprise a nucleic acid sequence encoding the transposase.

(transposase of HAT1_AG)
SEQ ID NO: 54
MMAPTNATTSPVWDHFSPVETGAKCLYCLKVFKYT
KGTTSNLKRHLNLVHKTVPYLKQKQPIPQTINIDD
EAGPSAVNFQPSNQYFNSNMSIQGYLKKPINSETK
KVLDRMLLDLICKECLPFNLVESEIFKKFVYTLNP
NYIMPTRKSLSNALLPSVYNQEFEKAKEKLSTAKA
IAITSDGWTNLNQISFFALTGHYIDENCKLSSILI
ECSEFENPHSGRNIANWIQGTLNKFDIEDKIVAMV
TDNASNMKAASTELNFCHIPCFAHTLNLIVRDAIK
KSVLPVVEEVKRVVMLFKKSPKASQMLADTQKKLN
LDQLKMIQEVSTRWNSGYDMLNRFYKNKIALLSCA
DSLKMKISLESHDWEAIEQIVRVLKYFYSATNIVS
AQKYITISHVGLLCNVLLTKTSQFRNDEDIAENIQ
NLVALLIEGLQNKLKIYRSNEQILKSMILDPRIKQ
LGFQDDVEKFKNICESIISELLPLQKPAVEVEKVV
KKVSKDVDMLFGDLLKNKGAQNYKTPRQIAENELH
QYLSVENIDLENDPLLWWKEHQVLYPSLYTLAMST
LCIPGTSVPCERLFSKAGQIYSEKRSRLAPKKLQE
ILFIQQNA

In some embodiments, the transposable element comprises: 1) a 5′TR in a LTF comprising the nucleic acid sequence of SEQ ID NO: 117; and 2) a 3′TR in a RTF comprising the nucleic acid sequence of SEQ ID NO: 143. In some embodiments, the transposable element comprises: 1) a 5′TR comprising the nucleic acid sequence of SEQ ID NO: 3, a variant thereof, or a fragment thereof; and 2) a 3′TR comprising the nucleic acid sequence of SEQ ID NO: 29, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 3; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 29. In some embodiments, the transposable element comprises: 1) a 5′TR comprising the nucleic acid sequence of SEQ ID NO: 3; and 2) a 3′TR comprising the nucleic acid sequence of SEQ ID NO: 29. In some embodiments, the transposable element further comprises a 5′TSD comprising the nucleic acid sequence of TA (SEQ ID NO: 193), and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 117; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 143. In some embodiments, the transposable element is associated with a transposase comprising the amino acid sequence of SEQ ID NO: 55, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 55. In some embodiments, the transposable element comprises a nucleic acid sequence encoding the transposase. In some embodiments, the transposable element does not comprise a nucleic acid sequence encoding the transposase.

(5′TR of Tcl-8B_DR)
SEQ ID NO: 3
CAGCGGGGAAAATAAGTATTTGACACATCAGCATT
TTTATCAGTAAGGGGATTTCTAAGTGGGCTACTGA
CACAAAATTCCTACCAGATGTAGCCATCAAGCCAA
ATATTGAATTCATACAAAGAAATCAGAACATTTAA
GTATACAAGTTGAGTCATAATAAATAAAGTGAAAT
GACACAGGGAATAAGTATTGAACACA
(3′TR of Tcl-8B_DR)
SEQ ID NO: 29
TGTGTTCAATACTTATTCCCTGTGTCATTTCACTT
TATTTATTATGACTCAACTTGTATACTTAAATGTT
CTGATTTCTTTGTATGAATTCAATATTTGGCTTGA
TGGCTACATCTGGTAGGAATTTTGTGTCAGTAGCC
CACTTAGAAATCCCCTTACTGATAAAAATGCTGAT
GTGTCAAATACTTATTTTCCCCGCTG
(transposase of Tc1-8B_DR)
SEQ ID NO: 55
MMGKNKELSQDLRSLIVEKHFDGNGYRRISRMLNV
PVSTVGAIIRKWKKHKFTINRPRSGAPRKIPVRGV
QRIIRRVLQEPRTTRAELQEDLASAGTIVSKKTIS
NALNHHGIHARSPRKTPLLNKKHVEARLKFAKQHL
EKPVDYWETIVWSDESKIELFGSHSTHHVWRRNGT
AHHPKNTIPTVKFGGGSIMVWGCFSARGTGRLHII
EGRMNGEMYRDILDKNLLPSTRKLKMKRGWTFQQD
NDPKHKAKETMKWFQRKKIKLLEWPSQSPDLNPIE
NLWRELKIKVHKRGPRNLQDLKTVCVEEWARJTPE
QCRRLVSPYKRRLEAVITNKGFSTKY

In some embodiments, the transposable element comprises: 1) a 5′TR in a LTF comprising the nucleic acid sequence of SEQ ID NO: 118; and 2) a 3′TR in a RTF comprising the nucleic acid sequence of SEQ ID NO: 144. In some embodiments, the transposable element comprises: 1) a 5′TR comprising the nucleic acid sequence of SEQ ID NO: 4, a variant thereof, or a fragment thereof; and 2) a 3′TR comprising the nucleic acid sequence of SEQ ID NO: 30, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 4; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 30. In some embodiments, the transposable element comprises: 1) a 5′TR comprising the nucleic acid sequence of SEQ ID NO: 4; and 2) a 3′TR comprising the nucleic acid sequence of SEQ ID NO: 30. In some embodiments, the transposable element further comprises a 5′TSD comprising the nucleic acid sequence of TTAGAG (SEQ ID NO: 195), and a 3′TSD comprising the nucleic acid sequence of SEQ ID NO: 195. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 118; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 144. In some embodiments, the transposable element is associated with a transposase comprising the amino acid sequence of SEQ ID NO: 56, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 56. In some embodiments, the transposable element comprises a nucleic acid sequence encoding the transposase. In some embodiments, the transposable element does not comprise a nucleic acid sequence encoding the transposase.

(transposase of hAT-6_PM)
SEQ ID NO: 56
MAKRKKDDEYRTFQDEWTEEFAFVERAGSAVCLIC
NDKITSLKRSNVKRHFDTRHATFASKYPAGDSRKK
ACQELLSRVQASQQQLRVWTRQGDYNSASFAGSLA
IVRNGKPFTDGEYAKTFMLDVANELFDDLPNKDKI
IKRIQDMPLSARTVHDRTIVMANKVEETQVKDINA
APFFSLALDESTDVSHLSQFSVIARYAVGDTLREE
SLAVLPLKGSTRGEDLFKSFMEFAQEKSLPMDKLL
SVCTDGAPCMVGKNKGFVALLREHENRPILSFHCI
IIIQEALCAQMCDRQFGEVMSLVIRVINFIVARAL
NDRQFKTLLDEVVNNYPGLLLHSNVRWLSRGKVLS
RFAACLNEIRTFLEMKGVGHPELADTEWLLKFYYL
VDLTEHLNQLNVKMQGIGNTVLSLQQAVFAFENKL
ELFTTDLETGRLLHFEKLSQFKDACTASEPIQNLD
LHQLAGCTSSLLQSFKARFGEFREHTRLFKFITHP
NECSLNTADLNYIPGVSVRDFEAEVADLKASDMWV
NKFKSLNEDLERITRQKAELASKHMWTEMKKLQPE
DQLILKTWNALPVTYHTLQRVSIAVLTMFGSTYAC
EQSFSHLKNIKSNLRSRLTDESLNACMKLNITKYQ
PDYKDISKSMQHQKSH

In some embodiments, the transposable element comprises: 1) a 5′TR in a LTF comprising the nucleic acid sequence of SEQ ID NO: 119; and 2) a 3′TR in a RTF comprising the nucleic acid sequence of SEQ ID NO: 145. In some embodiments, the transposable element comprises: 1) a 5′TR comprising the nucleic acid sequence of CAGGGG (SEQ ID NO: 5), a variant thereof, or a fragment thereof; and 2) a 3′TR comprising the nucleic acid sequence of SEQ ID NO: 31, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 5; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 31. In some embodiments, the transposable element comprises: 1) a 5′TR comprising the nucleic acid sequence of SEQ ID NO: 5; and 2) a 3′TR comprising the nucleic acid sequence of CCCCTG (SEQ ID NO: 31). In some embodiments, the transposable element further comprises a 5′TSD comprising the nucleic acid sequence of CTGTATAG (SEQ ID NO: 196), and a 3′TSD comprising the nucleic acid sequence of SEQ ID NO: 196. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 119; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 145. In some embodiments, the transposable element is associated with a transposase comprising the amino acid sequence of SEQ ID NO: 57, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 57. In some embodiments, the transposable element comprises a nucleic acid sequence encoding the transposase. In some embodiments, the transposable element does not comprise a nucleic acid sequence encoding the transposase.

(transposase of hAT-3_XT)
SEQ ID NO: 57
MTSSQPAVKRKIDDEHRQFQEKWEMQYFFVEHRGI
PTCLICAEKVAVHKEYNLKRHYSTKHAEECAKYQG
DERAKRVASLKACLMRQQDFFKKATKENVASVQAS
YMVSEMIAKAGKPFTEGEFVKKCMLQVASIICPEK
KGQFSKISLSANTVAERISDMSSDIYHQLCEKAKC
FDAYSVALDESTDITGTAQLTIYVRGVDCNFELTE
ELLTIIPMHGQTTANEIFHHLCDAIENAGLPWKRF
VGIITDGAPSMTGRKNGLVALVKKKLEEEGIEEEA
IALHCIIHQQALCSKCLPCDNVMSVVVKCVNQIRS
RGLTHRRFRAFLEEMGSEYGDVLYFTEVRWLSRGN
VLKRFFELREEVKAFMEKNGKAVSELSDHKWLMDL
AFLVDITQRLNVLNKMLQGQGQLVSAAYDNVRAFS
TKLVLWKSQLSQTNLCHFPACKELVDAGIPFSGEK
YVDAIFKLEKEFDHRFADFKTHRATFQIFVDPFSF
DVQDAPPVLQMELIDLQCNSDIKAKFREMSGKADT
HVQFLRELPPSFPELSRMFKR

In some embodiments, the transposable element comprises: 1) a 5′TR in a LTF comprising the nucleic acid sequence of SEQ ID NO: 120; and 2) a 3′TR in a RTF comprising the nucleic acid sequence of SEQ ID NO: 146. In some embodiments, the transposable element comprises: 1) a 5′TR comprising the nucleic acid sequence of SEQ ID NO: 6, a variant thereof, or a fragment thereof; and 2) a 3′TR comprising the nucleic acid sequence of SEQ ID NO: 32, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 6; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 32. In some embodiments, the transposable element comprises: 1) a 5′TR comprising the nucleic acid sequence of SEQ ID NO: 6; and 2) a 3′TR comprising the nucleic acid sequence of SEQ ID NO: 32. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 120; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 146. In some embodiments, the transposable element is associated with a transposase comprising the amino acid sequence of SEQ ID NO: 58, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 58. In some embodiments, the transposable element comprises a nucleic acid sequence encoding the transposase. In some embodiments, the transposable element does not comprise a nucleic acid sequence encoding the transposase.

(5′TR of Tcl-3_Xt)
SEQ ID NO: 6
CAGTGGAGGAAATAATTATTTGACCCCTCACTGAT
TTTGTAAGTTTGTCCAATGACAAAGAAATGAAAAG
TCTCAGAACAGTATCATTTCAATGGTAGGTTTATT
TTAACAGTGGCAGATAGCACATCAAAAGGAAAATC
GAAAAAATAACTTTAAATAAAAGATAGCAACTGAT
TTGCATTTCATTGAGTGAAATAAGTTTTTGAACCC
(3′TR of Tcl-3_Xt)
SEQ ID NO: 32
GGGTTCAAAAACTTATTTCACTCAATGAAATGCAA
ATCAGTTGCTATCTTTTATTTAAAGTTATTTTTTC
GATTTTCCTTTTGATGTGCTATCTGCCACTGTTAA
AATAAACCTACCATTGAAATGATACTGTTCTGAGA
CTTTTCATTTCTTTGTCATTGGACAAACTTACAAA
ATCAGTGAGGGGTCAAATAATTATTTCCTCCACTG
(transposase of Tcl-3_Xt)
SEQ ID NO: 58
MGKTKELSKDVRDKIVDLHKAGMGYKTISKKLGEK
VTTVGAIVRKWKEHKMTINRPRSGAPRKISPRGVS
MILRKVKKIIPRTTREELVNDLKLAGTTVTKKTIG
NTLIIRNGLKSCRARKVPLLKKAHVQARLKFANEH
LNDSVSDWEKVLWSDETKIELFGINSTRCVWRKKN
AAYDPQNTVPTVKHGGGNTLLWGCFSAKGTGQLIR
JNGKMDGAMYREILNDNLLPSARKLKMGRGWVFQH
DNDPKHTAKATKEWLKKKHIKVMEWPSQSPDLNPI
ENLWRELKLRVAQRQPRNLRDLEMICKEEWTNIPP
KMCANLVINYKKRLTSVLANKGFSTKY

In some embodiments, the transposable element comprises: 1) a 5′TR in a LTF comprising the nucleic acid sequence of SEQ ID NO: 121; and 2) a 3′TR in a RTF comprising the nucleic acid sequence of SEQ ID NO: 147. In some embodiments, the transposable element comprises: 1) a 5′TR comprising the nucleic acid sequence of CAGGGGTGGCGAACC (SEQ ID NO: 7), a variant thereof, or a fragment thereof; and 2) a 3′TR comprising the nucleic acid sequence of GGTTCGCCACCCCTG (SEQ ID NO: 33), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 7; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 33. In some embodiments, the transposable element comprises: 1) a 5′TR comprising the nucleic acid sequence of SEQ ID NO: 7; and 2) a 3′TR comprising the nucleic acid sequence of SEQ ID NO: 33. In some embodiments, the transposable element further comprises a 5′TSD comprising the nucleic acid sequence of GTCTATAC (SEQ ID NO: 194), and a 3′TSD comprising the nucleic acid sequence of SEQ ID NO: 194. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 121; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 147. In some embodiments, the transposable element is associated with a transposase comprising the amino acid sequence of SEQ ID NO: 59, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 59. In some embodiments, the transposable element comprises a nucleic acid sequence encoding the transposase. In some embodiments, the transposable element does not comprise a nucleic acid sequence encoding the transposase.

(transposase of hAT-5_DR)
SEQ ID NO: 59
MAESGKRTAKRKYEDERRTFLSEWEDLYFFVERNG
KPFCLICQTSLSHFKASNLERHFTSLHSAVAREFP
KGSELRKHKVKTLKGQAEKQTQLFRKFTKHSETVT
LASYQLAWNIARAKKPYLEGEFVKKCLSDAVAILC
PENENLKRSVKDLQLSRHTVEQRISDIDNSVETHL
LSDLQKCQYFSIALDESCDVQDKPQLAIFVRFVSE
DCTIREELLDIVPLKDRTRGIDLKETLMTVVEKAN
LQLSKLTAIVTDGAPAMLGSERGLVGLCKADDRFP
AFWTFHCIIHQEHLVSKKJLNLDHIMKPVLEIVNF
VRTHALNHRQFKNLIDELDEDLPSDLLFHCAVRWL
SRGHVLSRFFELLNPVKLFLAEKHKEYPELHDPQW
ISDLAFLVDVLHYLNGLNVDLQGKLKMLPDLVQSV
FAFVNKLKLFKTHLQKRDYTHFPTLLKASGQEAEV
VKGSTARYATLLENLEQSFEERFSNLRQKRQQITF
LINPFTAESGCLKAPLVEDEAASQLEMIELSEDDR
LKSVLREGTVEFWKIVPVERYPNVKQAALKLLSMF
GSTYVCESLFSTLKLVKSKHRSVLTDTHVKELLRV
ATTEYEPDLKKIVETKECQVSH

In some embodiments, the transposable element comprises: 1) a 5′TR in a LTF comprising the nucleic acid sequence of SEQ ID NO: 122; and 2) a 3′TR in a RTF comprising the nucleic acid sequence of SEQ ID NO: 148. In some embodiments, the transposable element comprises: 1) a 5′TR comprising the nucleic acid sequence of SEQ ID NO: 8, a variant thereof, or a fragment thereof; and 2) a 3′TR comprising the nucleic acid sequence of SEQ ID NO: 34, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 8; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 34. In some embodiments, the transposable element comprises: 1) a 5′TR comprising the nucleic acid sequence of SEQ ID NO: 8; and 2) a 3′TR comprising the nucleic acid sequence of SEQ ID NO: 34. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 122; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 148. In some embodiments, the transposable element is associated with a transposase comprising the amino acid sequence of SEQ ID NO: 60, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 60. In some embodiments, the transposable element comprises a nucleic acid sequence encoding the transposase. In some embodiments, the transposable element does not comprise a nucleic acid sequence encoding the transposase.

(5′TR of Tcl-3_FR)
SEQ ID NO: 8
CAGTGAGAGTAAAAAGTATTTGATCCCTTGCTGAT
TTTGTTGGTTTGTCCACTAATAAAGACATGATCAT
TCTATACTTTTAATGGTAGATGTATTCTAACATGG
AGAGACAGAATATCAAAAAGAAAATC
(3′TR of Tcl-3_FR)
SEQ ID NO: 34
GATTTTCTTTTTGATATTCTGTCTCTCCATGTTAG
AATACATCTACCATTAAAAGTATAGAATGATCATG
TCTTTATTAGTGGACAAACCAACAAAATCAGCAAG
GGATCAAATACTTTTTACTCTCACTG
(transposase ofTcl-3 FR)
SEQ ID NO: 60
MGKTKELSQDLRDRIVDLHKSGMGYKNISKLLGIK
VTTIGAIVRKFKKYNMTINRPRSGAPQKISPRGVA
MIMRTVRNRPATTQQELVNDLQAAGTKVTRKTIGN
TLRRNGLKSCSARKVPLLKKAHVEARLKYANDHLK
DAQSDWEKVLWSDETKIELFGLNSTRHVWRKKNAA
YDPRNTVPTVKHGGGNIMFWGCFSAKGTGLLHRIT
GKMDGAMYRAVLRDNLLPSARKLKMGRGWVFQHDN
DPKHTAKATKEWLKKNIIIKVMEWPSQSPDLNPIE
NLWRELKVRVAKRQPTNLNDLERICKEEWAKIPPD
MCANLVVNYNKRLTAVLANNGFATKY

In some embodiments, the transposable element comprises: 1) a 5′TR in a LTF comprising the nucleic acid sequence of SEQ ID NO: 123; and 2) a 3′TR in a RTF comprising the nucleic acid sequence of SEQ ID NO: 149. In some embodiments, the transposable element comprises: 1) a 5′TR comprising the nucleic acid sequence of SEQ ID NO: 9, a variant thereof, or a fragment thereof; and 2) a 3′TR comprising the nucleic acid sequence of SEQ ID NO: 35, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 9; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 35. In some embodiments, the transposable element comprises: 1) a 5′TR comprising the nucleic acid sequence of SEQ ID NO: 9; and 2) a 3′TR comprising the nucleic acid sequence of SEQ ID NO: 35. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 123; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 149. In some embodiments, the transposable element is associated with a transposase comprising the amino acid sequence of SEQ ID NO: 61, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 61. In some embodiments, the transposable element comprises a nucleic acid sequence encoding the transposase. In some embodiments, the transposable element does not comprise a nucleic acid sequence encoding the transposase.

(5′TR of Tcl-5_Xt)
SEQ ID NO: 9
CAAACCGGATTCCAAAAAAGTTGGGACACTAAACA
AATTGTGAATAAAAACTGAACGCAATGATGTGGAG
GTGCCAACTTCTAATATTTTATTCAGAATAGAACA
TAAATCACGGAACAAAAGTTTAAACTGAGAAAATG
TACCATTTTAAGGGAAAAATATGTTGATTCAGAAT
TTCATGGTGTCAACAAATCCCAAAAAAGTTGGGAC
AAG
(3′TR of Tcl-5_Xt)
SEQ ID NO: 35
CTTGTCCCAACTTTTTTGGGATTTGTTGACACCAT
GAAATTCTGAATCAACATATTTTTCCCTTAAAATG
GTACATTTTCTCAGTTTAAACTTTTGTTCCGTGAT
TTATGTTCTATTCTGAATAAAATATTAGAAGTTGG
CACCTCCACATCATTGCGTTCAGTTTTTATTCACA
ATTTGTTTAGTGTCCCAACTTTTTTGGAATCCGGG
TTG
(transposase of Tcl-5_Xt)
SEQ ID NO: 61
MIGYKKSFSEWQCLSEAKMGRGSPIPTMLRRKIVE
QYQKGVTQRKIAKILHLSSSTVHNIIRRFRESGTI
SVRKGQGRKTILDARDLRALKRHCTTNRNATVKEI
TEWAQEYFQKPLSVNTIHRAIRRCQLKLYSAKKKP
FLSKIHKLRRFHWARDHLKWSVAKWKTVLWSDESRF
EVLF
GNLGRHVIRTKEDKDNPSCYQRSVQKPASLMVWGC
MSACGMGSLHVWKGSINAEKYIQVLEQHMLPSRRH
LFQGRPCIFQQDNARPHSASITTSWLRRRRIRVLK
WPVCSPDLSPIENIWRIIKRKVRQRRPKTIEQLEA
CIRQEWESIPIPKLEKLVSSVPRRLLSVVRRRGDA
TQW

In some embodiments, the transposable element comprises: 1) a 5′TR in a LTF comprising the nucleic acid sequence of SEQ ID NO: 124; and 2) a 3′TR in a RTF comprising the nucleic acid sequence of SEQ ID NO: 150. In some embodiments, the transposable element comprises: 1) a 5′TR comprising the nucleic acid sequence of SEQ ID NO: 10, a variant thereof, or a fragment thereof; and 2) a 3′TR comprising the nucleic acid sequence of SEQ ID NO: 36, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 10; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 36. In some embodiments, the transposable element comprises: 1) a 5′TR comprising the nucleic acid sequence of SEQ ID NO: 10; and 2) a 3′TR comprising the nucleic acid sequence of SEQ ID NO: 36. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 124; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 150. In some embodiments, the transposable element is associated with a transposase comprising the amino acid sequence of SEQ ID NO: 62, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 62. In some embodiments, the transposable element comprises a nucleic acid sequence encoding the transposase. In some embodiments, the transposable element does not comprise a nucleic acid sequence encoding the transposase.

(5′TR of Tcl-10_Xt)
SEQ ID NO: 10
CAGTGGCTTGCAAAAGTATTCGGCCCCCTTGAACT
TTTCCACATTTTGTCACATTACAGCCACAAACATG
AATCAATTTTATTGGAATTCCACGTGAAAGACCAA
TACAAAGTGGTGTACACGTGAGAAGTGGAACGAAA
ATCATACATGATTCCAAACATTTTTTACAAATAAA
TAACTGCAAAGTGGGGTGTGCGTAATTATTCAGCC
CCCT
(3′TR of Tcl-W_Xt)
SEQ ID NO: 36
AGGGGGCTGAATAATTACGCACACCCCACTTTGCA
GTTATTTATTTGTAAAAAATGTTTGGAATCATGTA
TGATTTTCGTTCCACTTCTCACGTGTACACCACTT
TGTATTGGTCTTTCACGTGGAATTCCAATAAAATT
GATTCATGTTTGTGGCTGTAATGTGACAAAATGTG
GAAAAGTTCAAGGGGGCCGAATACTTTTGCAAGCC
ACTG
(transposase of Tcl-10_Xt)
SEQ ID NO: 62
MKSKEHTRQVRDKVIEKFKAGLGYKKISKALNIPR
STVQAIIQKWKEYQTTVNLPRQGRPPKLTGKTRRA
LIRNAAKRPMVTLDKLQRSTAQVGESVHRTTISRA
LHKVGLYGRVARRKPLLTENHKKSRIQFATSIIVG
DTANMWKKVLWSDETKMELFGQNAKRYVWRKTNTA
HHSEHTIPTVKYGGGSIMLWGCFSSAGTGKLVRVD
GKMDGAKYRAILEENLLESAKDLRLGRRFTFQQDN
DPKHKARATMEWFKTKHIHVLEWPSQSPDLNPIEN
LWQDLKTAVHKRCPSNLTELELFCKEEWARISVSR
CAKLVETYPKRLAAVIAAKGGSTKY

In some embodiments, the transposable element comprises: 1) a 5′TR in a LTF comprising the nucleic acid sequence of SEQ ID NO: 125; and 2) a 3′TR in a RTF comprising the nucleic acid sequence of SEQ ID NO: 151. In some embodiments, the transposable element comprises: 1) a 5′TR comprising the nucleic acid sequence of TACAGTGTCGGACAAATC (SEQ ID NO: 11), a variant thereof, or a fragment thereof; and 2) a 3′TR comprising the nucleic acid sequence of GATTTGTCCGACACTGTA (SEQ ID NO: 37), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 11; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 37. In some embodiments, the transposable element comprises: 1) a 5′TR comprising the nucleic acid sequence of SEQ ID NO: 11; and 2) a 3′TR comprising the nucleic acid sequence of SEQ ID NO: 37. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 125; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 151. In some embodiments, the transposable element is associated with a transposase comprising the amino acid sequence of SEQ ID NO: 63, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 63. In some embodiments, the transposable element comprises a nucleic acid sequence encoding the transposase. In some embodiments, the transposable element does not comprise a nucleic acid sequence encoding the transposase.

(transposase of Mariner2_AG)
SEQ ID NO: 63
MGRSAHSTQQQRLDIKRLSAAGYSQRKIAEILGRS
KTFVYNALHSTGTKIPTGRPRKTSARDDARMTRLC
KADPFKSARAIRDELQLSVSDRTVQRRLFENNLVG
RNPRKVPLLRRCHVQARLQFAREHYDWAGENLNKW
RNVLWSDESKVNLVGSDGKRFVRRPKNTAYRPQYT
LKTVKHGGGNIMVWACFSWYGVGPIFWIKDIMDQH
RYLNIIQTVMLPHAEWEMPLKWQFMHDNDPKHTAK
AVKKWFVDQKIDVMNWPAQSPDLNPIENLWKIVKA
KLPPAGSRTKEKLWKHIENA

In some embodiments, the transposable element comprises: 1) a 5′TR in a LTF comprising the nucleic acid sequence of SEQ ID NO: 126; and 2) a 3′TR in a RTF comprising the nucleic acid sequence of SEQ ID NO: 152. In some embodiments, the transposable element comprises: 1) a 5′TR comprising the nucleic acid sequence of SEQ ID NO: 12, a variant thereof, or a fragment thereof; and 2) a 3′TR comprising the nucleic acid sequence of SEQ ID NO: 38, a variant thereof, or a fragment thereof. In some embodiments, the transposable element comprises: 1) a 5′TR having a nucleic acid sequence that has at least about 90% (e.g., at least about any one of 91%4, 92%, 93%, 94%, 95%, 96%, 97%, 98%, 99%, or more) sequence identity to the nucleic acid sequence of SEQ ID NO: 12; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 38. In some embodiments, the transposable element comprises: 1) a 5′TR comprising the nucleic acid sequence of SEQ ID NO: 12; and 2) a 3′TR comprising the nucleic acid sequence of SEQ ID NO: 38. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%4, 92%, 93%, 94%, 95%, 96%, 97%, 98%, 99%, or 100%) sequence identity to the nucleic acid sequence of SEQ ID NO: 126; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 152. In some embodiments, the transposable element is associated with a transposase comprising the amino acid sequence of SEQ ID NO: 64, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 64. In some embodiments, the transposable element comprises a nucleic acid sequence encoding the transposase. In some embodiments, the transposable element does not comprise a nucleic acid sequence encoding the transposase.

(5′TR of Tcl-1_Xt)
SEQ ID NO: 12
CAGTGGTGTGAAAAACTATTTGCCCCCTTCCTGAT
TTCTTATTCTTTTGCATGTTTGTCACAC
(3′TR of Tcl-1_Xt)
SEQ ID NO: 38
GTGTGACAAACATGCAAAAGAATAAGAAATCAGGA
AGGGGGCAAATAGTTTTTCACACCACTG
(transposase of Tcl-1_Xt)
SEQ ID NO: 64
MPRSKEIQEQMRTKVIEIYQSGKGYKAISKALGLQ
RTTVRAIIHKWQKIIGTVVNLPRSGRPTKITPRAQ
RQLIREATKDPRTTSKELQASLASIKVSVHDSTIR
KRLGKNGLHGRFPRRKPLLSKKNIKARLNFAKKHL
NDCQDFWENTLWTDETKVELFGRCVSRYIWRKSNT
AFQKKNIIPTVKYGGGSVMVWGCFAASGPGRLAVI
DGTMNSTVYQKILKENVRPSVRQLKLKRSWVLQQD
NDPKHTSKSTSEWLKKNKMKTLEWPSQSPDLNPIE
MLWHDLKKAVHARKPSNKAELQQFCKDEWAKIPPE
RCKRLVASYRKRLIAVIAAKGGPTSY

In some embodiments, the transposable element comprises: 1) a 5′TR in a LTF comprising the nucleic acid sequence of SEQ ID NO: 127; and 2) a 3′TR in a RTF comprising the nucleic acid sequence of SEQ ID NO: 153. In some embodiments, the transposable element comprises: 1) a 5′TR comprising the nucleic acid sequence of CACTGGTGGACAT (SEQ ID NO: 13), a variant thereof, or a fragment thereof; and 2) a 3′TR comprising the nucleic acid sequence of ATGTCCACCAGTG (SEQ ID NO: 39), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 13; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 39. In some embodiments, the transposable element comprises: 1) a 5′TR comprising the nucleic acid sequence of SEQ ID NO: 13; and 2) a 3′TR comprising the nucleic acid sequence of SEQ ID NO: 39. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 127; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 153. In some embodiments, the transposable element is associated with a transposase comprising the amino acid sequence of SEQ ID NO: 65, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 65. In some embodiments, the transposable element comprises a nucleic acid sequence encoding the transposase. In some embodiments, the transposable element does not comprise a nucleic acid sequence encoding the transposase.

(transposase of Tcl-1_AG)
SEQ ID NO: 65
MGRGKHCTPEERKHIQGLYRENVPIKTICKAFGRS
RTFVDNAIRSEATGKSTGRPRKTTADVDAQIVEMI
RADPFKTCTRIKQELGLQVSAKTVSRRLHAAGFCA
RRPRKVRKLLPHHVEARIRFAEEHLAASIFWWSKI
IFSDESRINLDGSDGIKYVWRFPNQAYHPKNTIKT
LSHGGGHVMVWGCFSWHGTGPLFRINGTLNSEGYR
KILSRKMLPYARQQFGDEEHYIFQHDNDSKHTSRT
VKCYLANQDVQVLPWPALSPDLNPIENLWSTLKRQ
LKNQPARSADDLWTRCKVMWERIPRSEC

In some embodiments, the transposable element comprises: 1) a 5′TR in a LTF comprising the nucleic acid sequence of SEQ ID NO: 128; and 2) a 3′TR in a RTF comprising the nucleic acid sequence of SEQ ID NO: 154. In some embodiments, the transposable element comprises: 1) a 5′TR comprising the nucleic acid sequence of ATATACAC (SEQ ID NO: 14), a variant thereof, or a fragment thereof; and 2) a 3′TR comprising the nucleic acid sequence of GTGTATAT (SEQ ID NO: 40), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 14; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 40. In some embodiments, the transposable element comprises: 1) a 5′TR comprising the nucleic acid sequence of SEQ ID NO: 14; and 2) a 3′TR comprising the nucleic acid sequence of SEQ ID NO: 40. In some embodiments, the transposable element further comprises a 5′TSD comprising the nucleic acid sequence of AT (SEQ ID NO: 198), and a 3′TSD comprising the nucleic acid sequence of SEQ ID NO: 198. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 128; and 2) a RTF comprising a nucleic acid sequence having at least about 90% (e.g., at least about any one of 91%, 92%, 93%, 94%, 95%, 96%, 97%, 98%, 99%, or 1000) sequence identity to the nucleic acid sequence of SEQ ID NO: 154. In some embodiments, the transposable element is associated with a transposase comprising the amino acid sequence of SEQ ID NO: 66, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 66. In some embodiments, the transposable element comprises a nucleic acid sequence encoding the transposase. In some embodiments, the transposable element does not comprise a nucleic acid sequence encoding the transposase.

(transposase of TclDR3_Xt)
SEQ ID NO: 66
MGKKGDLSNFERGMVVGARRAGLSISQSAQLLGFS
RTTISRVYKEWCEKGKTSSMRQSCGRKCLVDARGQ
RRMGRLIQADRRATLTEITTRYNRGMQQSICEATT
RTTLRRMGYNSRRPHRVPLISTTNRKKRLQFAQAH
QNWTVEDWKNVAWSDESRFLLRHSNGRVRIWRKQN
ENMDPSCLVTTVQAGGGGVMVWGMFSWHTLGPLVP
IGHRLNATAYLSIVSDHVHPFMTTMYPSSDGYFQQ
DNAPCHKARIISNWFLEHDNEFTVLKWPPQSPDLN
PIEHLWDVVERELRALDVHPTNLHQLQDAILSIWA
NISKECFQHLVESMPRRIKAVLKAKGGQTPY

In some embodiments, the transposable element comprises: 1) a 5′TR in a LTF comprising the nucleic acid sequence of SEQ ID NO: 129; and 2) a 3′TR in a RTF comprising the nucleic acid sequence of SEQ ID NO: 155. In some embodiments, the transposable element comprises: 1) a 5′TR comprising the nucleic acid sequence of CAGGGGTCACCAAACT (SEQ ID NO: 15), a variant thereof, or a fragment thereof; and 2) a 3′TR comprising the nucleic acid sequence of SEQ ID NO: 41, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 15; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 41. In some embodiments, the transposable element comprises: 1) a 5′TR comprising the nucleic acid sequence of SEQ ID NO: 15; and 2) a 3′TR comprising the nucleic acid sequence of AGTTTGGTGACCCCTG (SEQ ID NO: 41). In some embodiments, the transposable element further comprises a 5′TSD comprising the nucleic acid sequence of CTCTAGAC (SEQ ID NO: 199), and a 3′TSD comprising the nucleic acid sequence of SEQ ID NO: 199. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 129; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 155. In some embodiments, the transposable element is associated with a transposase comprising the amino acid sequence of SEQ ID NO: 67, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 67. In some embodiments, the transposable element comprises a nucleic acid sequence encoding the transposase. In some embodiments, the transposable element does not comprise a nucleic acid sequence encoding the transposase.

(transposase of hAT-3_PM)
SEQ ID NO: 67
MATKKRKVDAECRVFNEEWGVKYFFVETKDQKASC
VICSESVAVLKEYNLRRHYETKHLSTYSKFSGKLR
SEKYESMKRGLETQRNLFTRKFAENESVTRTSYKR
VHKMAERGKPFTDGNFIKECMMEAANDLCPERADL
FGSISLSASSVVRRTEELGENIVLQIREKARNLLW
YSLALDESTDLSSTSQLLVFIRGVNLDFQITEELA
SVCSMHGTTTGKDIFMEVQKTLQDYNLQWNQLRGV
TVDGGKRMAGVRKGLVGQLRTQLEDLQIPGALFIH
CIIHQQALCGKDLDISCVLKPVVSAVNFIRGHALN
HRQFQAFLEEMDSDFCDLPYIITAVRWLSCGKVLF
RFYKLRNEIDVFLTEKDRADPQLSDPTWLSKLSFL
VDITSIIMNELNLKLQGKDNLVCDLYRIIKGFRRK
LSLFEAQLEGENFSHFHCFKEFCATIAEDVNLDFP
KKIIRDLKKHFLKRFSDLDRIESDILLFQNPFDCN
LDDVPVELQLELIDLQANDLLKEKHREGKLVEFYR
CLPDVEFPKLKKFAAGMASVFGTTYVCEQTTSMAT
KKRKVDAECRVFNEEWGVKYFFVETKDQKASCVIC
SESVAVLKEYNLRRHYETKHLSKMKYVKSTHRTRL
TDEHLKAILLIGCSNSKPNIDDILKAKRQFHKSH

In some embodiments, the transposable element comprises: 1) a 5′TR in a LTF comprising the nucleic acid sequence of SEQ ID NO: 130; and 2) a 3′TR in a RTF comprising the nucleic acid sequence of SEQ ID NO: 156. In some embodiments, the transposable element comprises: 1) a 5′TR comprising the nucleic acid sequence of CAGGGGTCACCAAACT (SEQ ID NO: 16), a variant thereof, or a fragment thereof; and 2) a 3′TR comprising the nucleic acid sequence of AGTTTGGTGACCCCTG (SEQ ID NO: 42), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 16; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 42. In some embodiments, the transposable element comprises: 1) a 5′TR comprising the nucleic acid sequence of SEQ ID NO: 16; and 2) a 3′TR comprising the nucleic acid sequence of SEQ ID NO: 42. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 130; and 2) a RTF comprising a nucleic acid sequence having at least about 90% (e.g., at least about any one of 91%, 92%, 93%, 94%, 95%, 96%, 97%, 98%, 99%, or 1000) sequence identity to the nucleic acid sequence of SEQ ID NO: 156. In some embodiments, the transposable element is associated with a transposase comprising the amino acid sequence of SEQ ID NO: 68, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 68. In some embodiments, the transposable element comprises a nucleic acid sequence encoding the transposase. In some embodiments, the transposable element does not comprise a nucleic acid sequence encoding the transposase.

(transposase of Tcl-1_PM)
SEQ ID No: 68
MATKKRKVDAECRVFNEEWGVKYFFVETRDQKASC
VCSEVAVLREYNLRRHYEIKHLSTYSKFSGKLRSE
KYESMKRGLETQRNLFTRKFAENESVTRTSYKIVH
KMAERGKPFTDGNFIKECMMEAANDLCPERADLFG
SISLSASSVVRRTEELGENIVLQIREKARNLLWYS
LALDESTDLSSTSQLLVFIRGVNLDFQITEELASV
CSMHGTTTGKDIFMEVQKTLQDYNLQWNQLRGVTV
DGGKNMAGVRKGLVGQIRTQLEDLQIPGALFIHCI
IHQQALCGKDLDISCVLKPVVSAVNFIRGHALNHR
QFQAFLEEMDSDFCDLPYHTAVRWLSCGKVLFRFY
KLRNEIDVFLTEKDRADPQLSDPTWLSKLSFLVDI
TSHMNELNLKLQGKDNLVCDLYRIIKGFRRKLSLF
EAQLEGENFSHFHCFKEFCATIAEDVNLDFPKKII
RDLKKHFLKRFSDLDRIESDILLFQNPFDCNLDDV
PVELQLELIDLQANDLLKEKHREGKLVEFYRCLPD
VEFPKLKKFAAGMASVFGTTYVCEQTFSKMKYVKS
THRTRLTDEHLKAILLIGCSNSKPNIDDILKAKRQ
FHKSH

In some embodiments, the transposable element comprises: 1) a 5′TR in a LTF comprising the nucleic acid sequence of SEQ ID NO: 131; and 2) a 3′TR in a RTF comprising the nucleic acid sequence of SEQ ID NO: 157. In some embodiments, the transposable element comprises: 1) a 5′TR comprising the nucleic acid sequence of CAGGC (SEQ ID NO: 17), a variant thereof, or a fragment thereof; and 2) a 3′TR comprising the nucleic acid sequence of GCCTG (SEQ ID NO: 43), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 17; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 43. In some embodiments, the transposable element comprises: 1) a 5′TR comprising the nucleic acid sequence of SEQ ID NO: 17; and 2) a 3′TR comprising the nucleic acid sequence of SEQ ID NO: 43. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 131; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 157. In some embodiments, the transposable element is associated with a transposase comprising the amino acid sequence of SEQ ID NO: 69, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 69. In some embodiments, the transposable element comprises a nucleic acid sequence encoding the transposase. In some embodiments, the transposable element does not comprise a nucleic acid sequence encoding the transposase.

(transposase of Mariner-4_AMi)
SEQ ID NO: 69
MSAKRKSMTSPGGSAKKQRKAIDLEMKMKIINDYE
AGKKVKAIARDIELAHSTISTILKDKDRVKEAV
QASTGFKAIITRQRKG
LIHEMEKLLAIWFDDQIQKRMPMSLLIIQAKARSI
FETLKAREGEESTETFTASRGWFQRFRRRFNVHNR
SISGEAASADVEAAEKFVDQFDEHEKGGYRPEQIF
NVDETGLFWKKMPERSYIHKEAKAMPGFKAFKDRV
TLLLGGNVAGFKLKPFLIHRSENPRALKQVSKHTL
PVYYRANSKAWMTQALFEDWFINCFIPSVKHYCLE
KGVPFKIILLLDNAPGHPQHILDDLHPDVKVVYLP
KNTTAILQPMDQGAIATFKVYYLRATFSKAVAATE
SDEVTLRDFWKSYNILHCIKNIESAWEGVTEKCMQ
GIWKKCLKVFVNNFEGFDKDEHVDVINKKIVELAN
VLNLDVEVKDIEELVEYVEGELTNEDLIELEAQQH
LEEEEEEEEERMEEVQKKFTVNGLAGVFSKVNAAI
LELEGMDPNVERFTKVERQMNELLRCYCEIYEEKK
KKKRKLQSRPH

In some embodiments, the transposable element comprises: 1) a 5′TR in a LTF comprising the nucleic acid sequence of SEQ ID NO: 132; and 2) a 3′TR in a RTF comprising the nucleic acid sequence of SEQ ID NO: 158. In some embodiments, the transposable element comprises: 1) a 5′TR comprising the nucleic acid sequence of CAGTGATGGCGAACCT (SEQ ID NO: 18), a variant thereof, or a fragment thereof; and 2) a 3′TR comprising the nucleic acid sequence of AGGTTCGCCATCACTG (SEQ ID NO: 44), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 18; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 44. In some embodiments, the transposable element comprises: 1) a 5′TR comprising the nucleic acid sequence of SEQ ID NO: 18; and 2) a 3′TR comprising the nucleic acid sequence of SEQ ID NO: 44. In some embodiments, the transposable element further comprises a 5′TSD comprising the nucleic acid sequence of GTCTAGAG (SEQ ID NO: 197), and a 3′TSD comprising the nucleic acid sequence of SEQ ID NO: 197. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 132; and 2) a RTF comprising a nucleic acid sequence having at least about 90% (e.g., at least about any one of 91%, 92%, 93%, 94%, 95%, 96%, 97%, 98%, 99%, or 100/o) sequence identity to the nucleic acid sequence of SEQ ID NO: 158. In some embodiments, the transposable element is associated with a transposase comprising the amino acid sequence of SEQ ID NO: 70, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 70. In some embodiments, the transposable element comprises a nucleic acid sequence encoding the transposase. In some embodiments, the transposable element does not comprise a nucleic acid sequence encoding the transposase.

(transposase of Myotis_hAT1)
SEQ ID NO: 70
MENQKNKKARLNDKGSGSSSSRPFQDIWTEMYGIV
DRNNRSLCILCNETVVSRTWNVKRHFETNHCQLLK
KSIDERKEYISRQLHLYKSQSNSIIKFVRCSTNLT
SASYCIAHSIAQHGKALSDGEFIKETFLRCAPALF
HDMKNKDDIIKRISELPLSRNTIKDRIIDLNKNVQ
HQLKKDLNSCKYFSISLDETTDVTSNARLAIIARY
SDGLTMREELIKLESVPISTSGNEICKVVIKTFSD
LNIDISKIVSVTTDGAPNMVGKNVGFLKLFMEAIR
HPLVPFHCIIHQEVLCAKSGFSELNDLMSVVTKIV
NFIASRPLHKREFSALLQEVDSTYSGLLMFNNVRW
LSRGKVLERFVECFEEITVFIENKDLANFPQINDH
KWVSNLMFFTDLSVHIMNELNLKLQGFGKSIDVMF
GYIKSFESKLKIFKRDIETKTYKYFPRIKKYFEKA
TTTSVMESILMYQNIVNSLFEQFSERFDQFRSLEQ
TIKIIKYPDVVVYSTLELKDFQWMQIDDLEMQLAD
FQGSIWTHVFVDLRSKLENLERNRLNNEEECNYEQ
EILSAWNRVPDTFSDIKNLAMALLTIFSSTYFCET
LFSALNNIKTNKRNRLTDEVSGACLALKCTKYEPA
IFFLADKLQHQKSH

In some embodiments, the transposable element comprises: 1) a 5′TR in a LTF comprising the nucleic acid sequence of SEQ ID NO: 133; and 2) a 3′TR in a RTF comprising the nucleic acid sequence of SEQ ID NO: 159. In some embodiments, the transposable element comprises: 1) a 5′TR comprising the nucleic acid sequence of CAG (SEQ ID NO: 19), a variant thereof, or a fragment thereof; and 2) a 3′TR comprising the nucleic acid sequence of CTG (SEQ ID NO: 45), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 19; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 45. In some embodiments, the transposable element comprises: 1) a 5′TR comprising the nucleic acid sequence of SEQ ID NO: 19; and 2) a 3′TR comprising the nucleic acid sequence of SEQ ID NO: 45. In some embodiments, the transposable element further comprises a 5′TSD comprising the nucleic acid sequence of GTCTAGAC (SEQ ID NO: 200), and a 3′TSD comprising the nucleic acid sequence of SEQ ID NO: 200. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 133; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 159. In some embodiments, the transposable element is associated with a transposase comprising the amino acid sequence of SEQ ID NO: 71, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 71. In some embodiments, the transposable element comprises a nucleic acid sequence encoding the transposase. In some embodiments, the transposable element does not comprise a nucleic acid sequence encoding the transposase.

(transposase of bAT-7_PM)
SEQ ID NO: 71
MSGSRKRKVDNECRVFNTEWTTKYFFTEVQSKAVC
LICRETVAVFKEYNISRHFATKHANYASKQSTQER
AATAQRLAANLQTQQHFFIIRQTAIQESTTKASFL
VAFEIAKASKPFSEGEFVKECMVQTADILCPEIKS
KFEKVSLSRRTVTRRVELIDENIASQLNKKSDSFE
LYSLALDESTDVKDTAQLLIFIRGIDDSFAITEEF
LTMESLKGTTRGEDIYNQVSAVIERMKIPWSKLVN
VTTDGSPNLTGKNVGLLKRIQNKVKEENPDQDLIF
LHCIIHQESLCKSVLQLNHVVNPAVKLVNFIRARG
LQHRQFITFLEETDADHQDLLYHSRVRWLSLGKVL
QRVWELKEDIIAFLELMGKSDEFPELSDKNWLSDF
AFAVDIFSHMNELNVKLQGKDQFVHDMYKHVKAFK
SKLTLFSRQIANKSFAHIFPTLAMQEEAPRNAKKY
SKSLEDLHGEFCRRFSDFENIEQSLQLVSCPLSQD
SETAPQELQLELIDLQSDSVLKEKFNSVKLNDFYA
SLNRATFPNLRRTAQKMLTLFGSTYVCEQTFSVMN
ANKARHRSKLTDQHLRSILRIATTKITPDLDALAK
MGDQQHCSH

In some embodiments, the transposable element comprises: 1) a 5′TR in a LTF comprising the nucleic acid sequence of SEQ ID NO: 134; and 2) a 3′TR in a RTF comprising the nucleic acid sequence of SEQ ID NO: 160. In some embodiments, the transposable element comprises: 1) a 5′TR comprising the nucleic acid sequence of SEQ ID NO: 20, a variant thereof, or a fragment thereof; and 2) a 3′TR comprising the nucleic acid sequence of SEQ ID NO: 46, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 20; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 46. In some embodiments, the transposable element comprises: 1) a 5′TR comprising the nucleic acid sequence of SEQ ID NO: 20; and 2) a 3′TR comprising the nucleic acid sequence of SEQ ID NO: 46. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 134; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 160. In some embodiments, the transposable element is associated with a transposase comprising the amino acid sequence of SEQ ID NO: 72, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 72. In some embodiments, the transposable element comprises a nucleic acid sequence encoding the transposase. In some embodiments, the transposable element does not comprise a nucleic acid sequence encoding the transposase.

(5′TR of Tcl-11_Xt)
SEQ ID NO: 20
CAGTGGATATAAAAAGTCTACACACCCCTGGTAAA
ATGTCAGGTTCCTGTGCTGTACAAAAATGAGACAA
AGATAAATCATTTCAGAACTTTTTCCACCTTTAAT
GTGACCTATAAACTGTACCACTCAATTGAAAAACA
AACTGAAATCTTTTAGG
(3′TR of TcMl_Xt)
SEQ ID NO: 46
CCTAAAAGATTTCAGTTTGTTTTTCAATTGAGTGG
TACAGTTTATAGGTCACATTAAAGGTGGAAAAAGT
TCTGAAATGATTTATCTTTGTCTCATTTTTGTACA
GCACAGGAACCTGACATTTTACCAGGGGTGTGTAG
ACTTTTTATATCCACTG
(transposase of Tcl-11 Xt)
SEQ ID NO: 72
MVHRELPKHQRDLIVKRYQSGEGYKRISKALDIPW
NTVKTVIIKWRKYGTTVTLPRTGRPSKIDEKTRRK
LVREATKRPTATLKELQEYLASTGCVVHVTTISRI
LHMSGLWGRVARRKPFLTKKNIQARLHFAKTHLKS
PKSMWEKVLWSDETKVELFGHNSKKYVWRKNNTAH
HQKNTIPTVKIIGGGSIMLWGCFSSAGTGALVKIE
GIMNSSKYQSILAQNLQASARKLNMRRNFIFQHDN
DPKHTSKSTKEWLHRKKIKVLEWPSQSPDLNPIEN
LWGDLKRAVHRRCPRNLTDLECFCKEEWANLAKSK
CAMLIDSYPKRLSAVIKSKGASTKY

In some embodiments, the transposable element comprises: 1) a 5′TR in a LTF comprising the nucleic acid sequence of SEQ ID NO: 135; and 2) a 3′TR in a RTF comprising the nucleic acid sequence of SEQ ID NO: 161. In some embodiments, the transposable element comprises: 1) a 5′TR comprising the nucleic acid sequence of SEQ ID NO: 21, a variant thereof, or a fragment thereof; and 2) a 3′TR comprising the nucleic acid sequence of SEQ ID NO: 47, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 21; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 47. In some embodiments, the transposable element comprises: 1) a 5′TR comprising the nucleic acid sequence of SEQ ID NO: 21; and 2) a 3′TR comprising the nucleic acid sequence of SEQ ID NO: 47. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 135; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 161. In some embodiments, the transposable element is associated with a transposase comprising the amino acid sequence of SEQ ID NO: 73, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 73. In some embodiments, the transposable element comprises a nucleic acid sequence encoding the transposase. In some embodiments, the transposable element does not comprise a nucleic acid sequence encoding the transposase.

(5′TR of Tcl-16 Xt)
SEQ ID NO: 21
CAGTCATGGCCAAAATTGTTGGCACCCCAGAAATT
TTTCCAGAAAATCAAGTATTTCTCACAGAAAAGTA
TTGCAGTAACACATGTTTTGCTATACACATGTTTA
TTCCCTTTGTGTGTATTGGAACAGAACAAAAAAGG
GAGGAAAAAAAGCAAATTGGACATAATGTCACACA
AAACTCCAAAAATGGGCTGGACAAAATTATTGGCA
CCCTT
(3′TR of Tcl-16_Xt)
SEQ ID NO: 47
AAGGGTGCCAATAATTTTGTCCAGCCCATTTTTGG
AGTTTTGTGTGACATTATGTCCAATTTGCTTTTTT
TCCTCCCTTTTTTGTTCTGTTCCAATACACACAAA
GGGAATAAACATGTGTATAGCAAAACATGTGTTAC
TGCAATACTTTTCTGTGAGAAATACTTGATTTTCT
GGAAAAATTTCTGGGGTGCCAACAATTTTGGCCAT
GACTG
(transposase of Tcl-16_Xt)
SEQ ID NO: 73
MDNRKRRRELSEDLRTKIVEKYQQSQGYKSISRDL
DLPLSTVRNIIKKFATHGTVANLPGRGRKRKIDER
LQRRIVRMVDKQPQTSSKEIQAVLQAQGASVSART
IRRHLNEMKRYGRRPRRTPILTQRHKKARLQFAKM
YISKPQSFWTNVLWTDETKIELFGKAHHSTVYRKR
NEAYKEKNTVPTVKYGGGSMMFWGCFAASGTGCLE
CVQGIMKSEDYQRILGRTVEPSVRKLGLRPRSWVF
QQDNDPKHTSKSTQKWMATKRWRVLKWPAMSPDLN
PIEHLWRDLKIAVGKRRPSNKRDLEQFAKEEWSKI
PGERCKKLIDGYRKRLISVIFSKGCATKY

In some embodiments, the transposable element comprises: 1) a 5′TR in a LTF comprising the nucleic acid sequence of SEQ ID NO: 136; and 2) a 3′TR in a RTF comprising the nucleic acid sequence of SEQ ID NO: 162. In some embodiments, the transposable element comprises: 1) a 5′TR comprising the nucleic acid sequence of SEQ ID NO: 22, a variant thereof, or a fragment thereof; and 2) a 3′TR comprising the nucleic acid sequence of SEQ ID NO: 48, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 22; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 48. In some embodiments, the transposable element comprises: 1) a 5′TR comprising the nucleic acid sequence of SEQ ID NO: 22; and 2) a 3′TR comprising the nucleic acid sequence of SEQ ID NO: 48. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 136; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 162. In some embodiments, the transposable element is associated with a transposase comprising the amino acid sequence of SEQ ID NO: 74, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 74. In some embodiments, the transposable element comprises a nucleic acid sequence encoding the transposase. In some embodiments, the transposable element does not comprise a nucleic acid sequence encoding the transposase.

(5′TR of Tcl-4_Xt)
SEQ ID NO: 22
CAGTGAGGAACATAAGTATTTGAACACCCTGCGAT
TTTGCAAGTTCTCCCACTTAGAAATCATGGAGGGG
TCTGAAATTCACATTGTAGGTGCATTCCCACTGTG
AGAGACAGCATTTAAAAAAAAAATTCAGGAAATCA
CATTGTATGATTTTTAAAGAATGTATTTGTATTGC
ACTGCTGCACATAAGTATTTGAACACCTG
(3′TR of Tcl-4_Xt)
SEQ ID NO: 48
CAGGTGTTCAAATACTTATGTGCAGCAGTGCAATA
CAAATACATTCTTTAAAAATCATACAATGTGATTT
CCTGAATTTTTTTTTTAAATGCTGTCTCTCACAGT
GGGAATGCACCTACAATGTGAATTTCAGACCCCTC
CATGATTTCTAAGTGGGAGAACTTGCAAAATCGCA
GGGTGTTCAAATACTTATGTTCCTCACTG
(transposase of Tcl-4_Xt)
SEQ ID NO: 74
MGKTKELSKDTRDKIVDLHKAGKGYGAIAKQLGEN
RSTVGAIVRKWKRLKTTVSLPRTGAPCKISPRGVS
LMIRKVRNQPRTTREELVNDMKRAGTTVSKVTVGR
TLRRHGFKSCIARKVPLLKSSHVQARLKFANDHLD
DPEEAWEKVMWSDETKVELFGLNFTRRVWRKNKDE
LHPKNTIPTVKHGGGNIMLWGCFSAKGTGRLHCIK
ERMNGAMYCEIISNNLLPSVRALKMGRGWVFQHDN
DPKHTARITKEWLRKRHIKVLEWPSQSPDLNPIEN
LWRELKLRVAQRQPRNLTDLEEICVEEWAKIPVAV
CANLVKNYRKRLTSVIANKGFCTKY

In some embodiments, the transposable element comprises: 1) a 5′TR in a LTF comprising the nucleic acid sequence of SEQ ID NO: 137; and 2) a 3′TR in a RTF comprising the nucleic acid sequence of SEQ ID NO: 163. In some embodiments, the transposable element comprises: 1) a 5′TR comprising the nucleic acid sequence of CACTGCTCAAAAAAATAAAGGGAACAC (SEQ ID NO: 23), a variant thereof, or a fragment thereof; and 2) a 3′TR comprising the nucleic acid sequence of GTGTTCCCTTTATTTTTTTGAGCAGTG (SEQ ID NO: 49), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 23; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 49. In some embodiments, the transposable element comprises: 1) a 5′TR comprising the nucleic acid sequence of SEQ ID NO: 23; and 2) a 3′TR comprising the nucleic acid sequence of SEQ ID NO: 49. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 137; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 163. In some embodiments, the transposable element is associated with a transposase comprising the amino acid sequence of SEQ ID NO: 75, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 75. In some embodiments, the transposable element comprises a nucleic acid sequence encoding the transposase. In some embodiments, the transposable element does not comprise a nucleic acid sequence encoding the transposase.

(transposase of Tcl-15_Xt)
SEQ ID NO: 75
MRRSLQPTQMAQVVQLIQDGTSMRAVARRFAVSVS
VVSRAWRRYQETGQYIRRRGGGRRRATTQQQDRYL
RLCARRNRRSTARALQNDLQRATNVHVSAQTVRNR
LHEGGMRARRPQVGVVLTAQHRAGRLAFAREHQDW
QIRHWRPVLFTDESRFTLSTCDRRDRVWRRRGERS
AACNILQHDRFGSGSVMVWGGISLGGRTALHVLAR
GSLTAIRYRDEILRPLVRPYAGAVGPGFLLMQDNA
RPHVAGVCQQFLQDEGIDAMDWPARSPDLNPIEHI
WDIMSRSIHQRHVAPQTVQELVDALVQVWEEIPQE
TIRHLIRSMPRRCREVIQARGGHTHY

In some embodiments, the transposable element comprises: 1) a 5′TR in a LTF comprising the nucleic acid sequence of SEQ ID NO: 138; and 2) a 3′TR in a RTF comprising the nucleic acid sequence of SEQ ID NO: 164. In some embodiments, the transposable element comprises: 1) a 5′TR comprising the nucleic acid sequence of CACTGCTCAAAAAAATTAGAGGAACACTT (SEQ ID NO: 24), a variant thereof, or a fragment thereof; and 2) a 3′TR comprising the nucleic acid sequence of AAGTGTTCCTCTAATTTTTTTGAGCAGTG (SEQ ID NO: 50), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 24; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 50. In some embodiments, the transposable element comprises: 1) a 5′TR comprising the nucleic acid sequence of SEQ ID NO: 24; and 2) a 3′TR comprising the nucleic acid sequence of SEQ ID NO: 50. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 138; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 164. In some embodiments, the transposable element is associated with a transposase comprising the amino acid sequence of SEQ ID NO: 76, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 76. In some embodiments, the transposable element comprises a nucleic acid sequence encoding the transposase. In some embodiments, the transposable element does not comprise a nucleic acid sequence encoding the transposase.

(transposase of TC1_FR2)
SEQ ID NO: 76
MVRYLDATEVAQVVQLLQDGTSIRAVARRFAVSPS
TVSRAWRRFQETGSYSRRAGQGRRRSLNPQQDRYL
LLCARRNRMSTARALQNDLQQATGVNVSDQTIRNR
LHEGGLRARRPVVGPVLTARIIRRARLAFAIDHQN
WQLRHWRPVLFTDESRFNLSTCDRRERVWRCRGER
YAACNIIQHDRFGGGSVMVWGGISLEGRTDLYRLD
NGTLTAIRYRDEILGPIVRTYAGAVGPGFLLVHDN
ARPHVARVCRQFLEDEGIDTIDWPPRSPDLNPIEH
LWDIMFRSIRRRQVDPQTVQELSDALVQIWEEIPQ
DTIRRLIRSMPRRCQACVQARGGHTNY

In some embodiments, the transposable element comprises: 1) a 5′TR in a LTF comprising the nucleic acid sequence of SEQ ID NO: 139; and 2) a 3′TR in a RTF comprising the nucleic acid sequence of SEQ ID NO: 165. In some embodiments, the transposable element comprises: 1) a 5′TR comprising the nucleic acid sequence of TAG (SEQ ID NO: 25), a variant thereof, or a fragment thereof; and 2) a 3′TR comprising the nucleic acid sequence of CTA (SEQ ID NO: 51), a variant thereof, or a fragment thereof. In some embodiments, the transposable element comprises: 1) a 5′TR having a nucleic acid sequence that has at least about 901% (e.g., at least about any one of 91%, 92%, 93%, 94%, 95%, 96%, 97%, 98%, 99%, or more) sequence identity to the nucleic acid sequence of SEQ ID NO: 25; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 51. In some embodiments, the transposable element comprises: 1) a 5′TR comprising the nucleic acid sequence of SEQ ID NO: 25; and 2) a 3′TR comprising the nucleic acid sequence of SEQ ID NO: 51. In some embodiments, the transposable element further comprises a 5′TSD comprising the nucleic acid sequence of ATCATCAT (SEQ ID NO: 201), and a 3′TSD comprising the nucleic acid sequence of SEQ ID NO: 201. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 139; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 165. In some embodiments, the transposable element is associated with a transposase comprising the amino acid sequence of SEQ ID NO: 77, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 77. In some embodiments, the transposable element comprises a nucleic acid sequence encoding the transposase. In some embodiments, the transposable element does not comprise a nucleic acid sequence encoding the transposase.

(transposase of hAT-9_XT)
SEQ ID NO: 77
MPGKGRPPRRGTRGRAAMISCGPRKLPSFPKALTL
NSQNAEEVVDWLTQHTPSSTVSNFATTSSSSSAMG
TPRTTSSTTAAPSSLESEELFTHEFVELSDAQPLL
PEEDEGDEDVTPDIIIQAGNTTEMDIRCDEVPAAA
VFCELSEEIDASEENDDEEIDILWVPTRREQEEDS
SDGETESQRGRRRIRLRRSRDSSQGTVGQQIIESA
PVVSQPTIIPPTSTVTARKPTSKGSAVWHFFNVCA
SDKSAVICNECSQKLSLGKPNTIILGTTAMRRIIM
NAKIIKALWEQIILKGSSQTLSIIPPSAPASYCST
SAVLDPSQPPSTPPSTLTTSSCSSAPSQVSVRAMF
ERKKPMPPSHPLARRLTAGESALLARQLLPYQLVD
SEAFHQFVAIGTPQWKVPSRNFFSQKGIPHLYQHV
QSQVTASLSFSVGAKVHMTTDTWSSKHGQGRYVTY
TAHWVNLVMAGKQGMRGSTTVELVSPPRVACGSAT
TSTPPLLSNSSSKSSSNSSSNLASSSASSSSAAVS
SSTPVHPQLHIGYSTCQVRRCHAVLGMTGLESRNH
TGSALLSSLHSQADRWLTPHQLKIGKVVCDNGSNL
LAALRLGNLTHVPCMAHVLNLIVQRFVSKYPGLQD
ILRQSRKVSGHFRRSYTAMARLADIQRRIINLPVR
RLICDSQTRWNSTLMMFERLLQQQRAVNTSTEYLF
ELGGRTGSAELGIFFPRYWVLMRDACRLMRPFDEV
TNMVSRTEGTISDLIPFAFFLERAVRRVTDDAVDQ
RDQEHDFWAESPERSQAPAATQGEVSEVESEEEGG
FVEAEDQQEQASRGACGHLLWSPGLVRGWGEETVV
DEDVVLDNEEGEMDTSASNLVRMGSFMLSCLLKDP
RIKRLKEKDLYWYATLLDPRYKHKVAEMLPTYHBC
SERMLHFQTSLQNMLYNAFKGDVTPHSRGRGAGNP
PTSTPARTMHFGHSVTSDMQSFFSPRQRQDPSGST
LRERLDRQVADYLALTADIDTLRSDEPLDYWVRRL
DLWPELSQFAINLLSCPASSVLSERTFSAAGGIVT
EKRTRLGIIKSVDYLTFIKMNEAWISEGYCTPEDL
FPEDLF

In some embodiments, the transposable element comprises: 1) a 5′TR in a LTF comprising the nucleic acid sequence of SEQ ID NO: 140; and 2) a 3′TR in a RTF comprising the nucleic acid sequence of SEQ ID NO: 168. In some embodiments, the transposable element comprises: 1) a 5′TR comprising the nucleic acid sequence of CCGTATTTTCCGCACTATAAGGCGCACC (SEQ ID NO: 26), a variant thereof, or a fragment thereof; and 2) a 3′TR comprising the nucleic acid sequence of GGTGCGCCTTATAGTGCGGAAAATACGG (SEQ ID NO: 52), a variant thereof, or a fragment thereof. In some embodiments, the transposable element comprises: 1) a 5′TR having a nucleic acid sequence that has at least about 90% (e.g., at least about any one of 91%4, 92%, 93%, 94%, 95%, 96%, 97%, 98%, 99%, or more) sequence identity to the nucleic acid sequence of SEQ ID NO: 26; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 52. In some embodiments, the transposable element comprises: 1) a 5′TR comprising the nucleic acid sequence of SEQ ID NO: 26; and 2) a 3′TR comprising the nucleic acid sequence of SEQ ID NO: 52. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%4, 92%, 93%, 94%, 95%, 96%, 97%, 98%, 99%, or 100%) sequence identity to the nucleic acid sequence of SEQ ID NO: 140; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 168. In some embodiments, the transposable element is associated with a transposase comprising the amino acid sequence of SEQ ID NO: 78, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 78. In some embodiments, the transposable element comprises a nucleic acid sequence encoding the transposase. In some embodiments, the transposable element does not comprise a nucleic acid sequence encoding the transposase.

(transposase of Mariner-5_XT)
SEQ ID NO: 78
MAPKKRHAYEAQFKLQAASYAVVNGNRAAAKEFNI
NESMVRKWRKQENELRQVKKTKQSFRGNKARWPQL
EDQLEQWVIEQRTAGRSVSTVTIRLKATTIAQDLE
IEHFQGGPSWCFRFMKRRHLSIRARTTVAQQLPAD
YKEKMAIFRTYCSNKITDKKIQPNHITNMDEVPLT
FDIPVNHTVEIKGTSTVSIRTTGHEKSAFTVVLSC
HGNGQKLPPMVIFKRKTLPKEKFPAGVIVKANQKG
WMDEEKMREWLREVYVKRPDGFFHTSPSLLICDSM
RAHLTATVKKQVKQMNSELAIIPGGLTKELQPLDV
GVNRAFKVKLRTAWERWMTDGEHTFTKTGKQRRAS
YATICEWIVDAWAKVSAITVVRAFAKTGIIAEQPP
GNDTGNETDSDNDEREPGMFDGEIAQLFNSDTEDE
DFDGFVGED

In some embodiments, the transposable element comprises: 1) a 5′TR in a LTF comprising the nucleic acid sequence of SEQ ID NO: 167; and 2) a 3′TR in a RTF comprising the nucleic acid sequence of SEQ ID NO: 179. In some embodiments, the transposable element comprises: 1) a 5′TR comprising the nucleic acid sequence of CCGTATTTTCTC (SEQ ID NO: 79), a variant thereof, or a fragment thereof; and 2) a 3′TR comprising the nucleic acid sequence of GAGAAAATACGG (SEQ ID NO: 91), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 79; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 91. In some embodiments, the transposable element comprises: 1) a 5′TR comprising the nucleic acid sequence of SEQ ID NO: 79; and 2) a 3′TR comprising the nucleic acid sequence of SEQ ID NO: 91. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 167; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 179. In some embodiments, the transposable element is associated with a transposase comprising the amino acid sequence of SEQ ID NO: 103, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, %%, 97%, 98%, 99% or more) sequence identity to the amino acid sequence of SEQ ID NO: 103. In some embodiments, the transposable element comprises a nucleic acid sequence encoding the transposase. In some embodiments, the transposable element does not comprise a nucleic acid sequence encoding the transposase.

(transposase of Mariner-6_AMi)
SEQ ID NO: 103
MPKRKSYTFDFKLSVIDYAKKHGNRAAGREFSVDE
KSVREWRKEEDILKTLNPRKRARRGKHCKWPTLEA
NLKEWVVAQRESNKVVSTIAIRQKARVMANEMKIT
DFGGGCNWVHKFMHRNNLAVRSRTTVGQKLPDDWE
EKLTKFRDFVRKECRVHNLCPADIINMDEVPISFD
VPATRTVDQRGKKTIAISTTGHERTNFTVVLACTA
SGVKLKPMVIFKRVTMPREKIPNGVAVICNKKGWM
NEDIMKEWTERCFRTRQGGFFAPKSLLIFDAMAAH
KHSSVQKQINKAGAHIAVIPGGLTCKLQPLDISVN
HSFKCFMRKEWEKWMSQGVHSFTPSGKQRRATYTE
VCNWIVAAWRAVKPSSIINGFFKAGILDGASGEES
DYTDSEDYADELDDAAFVDAYFGESSYDTDFEGFP
ASEKEEAPNWV

In some embodiments, the transposable element comprises: 1) a 5′TR in a LTF comprising the nucleic acid sequence of SEQ ID NO: 168; and 2) a 3′TR in a RTF comprising the nucleic acid sequence of SEQ ID NO: 180. In some embodiments, the transposable element comprises: 1) a 5′TR comprising the nucleic acid sequence of CCCTTT (SEQ ID NO: 80), a variant thereof, or a fragment thereof; and 2) a 3′TR comprising the nucleic acid sequence of AAAGGG (SEQ ID NO: 92), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 80; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 92. In some embodiments, the transposable element comprises: 1) a 5′TR comprising the nucleic acid sequence of SEQ ID NO: 80; and 2) a 3′TR comprising the nucleic acid sequence of SEQ ID NO: 92. In some embodiments, the transposable element further comprises a 5′TSD comprising the nucleic acid sequence of TTAA (SEQ ID NO: 202), and a 3′TSD comprising the nucleic acid sequence of SEQ ID NO: 202. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 168; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 180. In some embodiments, the transposable element is associated with a transposase comprising the amino acid sequence of SEQ ID NO: 104, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 104. In some embodiments, the transposable element comprises a nucleic acid sequence encoding the transposase. In some embodiments, the transposable element does not comprise a nucleic acid sequence encoding the transposase.

(transposase ofpiggyBac-2_XT)
SEQ ID NO: 104
MAKRFYSAEEAAAHCMASSSEEFSGSDSEYVPPAS
ESDSSTEESWCSSSTVSALEEPMEVDEDVDDLEDQ
EAGDRADAAAGGEPAWGPPCNFPPEIPPFTTVPGV
KVDTSNFEPINFFQLFMTEAILQDMVLYTNVYAEQ
YLTQNPLPRYARAHAWHPTDIAEMKRFVGLTLAMG
LIKANSLESYWDTTTVLSIPVFSATMSRNRYQLLL
RFLHFNNNATAVPPDQPGHDRLHKLRPLIDSLSER
FAAVYTPCQNICIDESLLLFKGRLQFRQYIPSKRA
RYGIKFYKLCESSSGYTSYFLIYEGKDSKLDPPGC
PPDLTVSGKIVWELISPLLGQGFHLYVDNFYSSIP
LFTALYCLDTPACGTINRNRKGLPRALLDKKLNRG
ETYALRKNELLAIKFFDKKNVFMLTSIHDESVIRE
QRVGRPPKNKPLCSKEYSKYMGGVDRTDQLQHYYN
ATRKTRAWYKKVGIYLIQMALRNSYIVYKAAVPGP
KLSYYKYQLQILPALLFGGVEEQTVPEMPPSDNVA
RLIGKIIFIDTLPPTPGKQRPQKGCKVCRKRGIRR
DTRYYCPKCPRNPGLCFKPCFEIYHTQLHY

In some embodiments, the transposable element comprises: 1) a 5′TR in a LTF comprising the nucleic acid sequence of SEQ ID NO: 169; and 2) a 3′TR in a RTF comprising the nucleic acid sequence of SEQ ID NO: 181. In some embodiments, the transposable element comprises: 1) a 5′TR comprising the nucleic acid sequence of CCTTCATACG TTCCCATG (SEQ ID NO: 81), a variant thereof, or a fragment thereof; and 2) a 3′TR comprising the nucleic acid sequence of CATGAGAACG GATGAGGG (SEQ ID NO: 93), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 81; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 93. In some embodiments, the transposable element comprises: 1) a 5′TR comprising the nucleic acid sequence of SEQ ID NO: 81; and 2) a 3′TR comprising the nucleic acid sequence of SEQ ID NO: 93. In some embodiments, the transposable element further comprises a 5′TSD comprising the nucleic acid sequence of SEQ ID NO: 202, and a 3′TSD comprising the nucleic acid sequence of SEQ ID NO: 202. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 169; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 181. In some embodiments, the transposable element is associated with a transposase comprising the amino acid sequence of SEQ ID NO: 105, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 105. In some embodiments, the transposable element comprises a nucleic acid sequence encoding the transposase. In some embodiments, the transposable element does not comprise a nucleic acid sequence encoding the transposase.

(transposase of piggyBac-1_AMi)
SEQ ID NO: 105
MRRQRIFSAEACLAQVTGDDSDTSTDEILSQGSED
ADFVPDMGDCSEQEDHASTEASSSSDEEELVPQAE
VMPTLPVGKGRGPARGRRGRPRRATAGRGAEPLPP
PPENIADDDTFRGRNGRVWGKSPQLIHHRRTAKDI
VHESAGPTRESNKDTVVETFHILFLSQDILTVIIR
ETNREAERRFREWNDSHPENRKEWKKLDETELKAY
IGLLLLSGLYHGGHEALEELWEQRTGRPCFRATMS
LKRFRQITNYLRFDNKLTREERRAVDKLAAFRDIW
TQFVEQLRKWYIPGIHLTVDEQLIAFRGRCPFRQY
MPSKPAKYGLKIWWCCDAATSFPLSGDIYLGKQPE
APRQGNLGTQVIEKLVHPWYKTGRNIVGDNFFTST
DLAERLLQQDLTYVGTIRKNKADIPNEMQANRRRE
TFSSIFAFDGELTLVSYVPKKSKVVLLLSSMHHDL
SVGGEQRKPDIIHDYNKNKGGVDNLDHLVRMTTCR
RKINRWPMTLFFNMLDVAAIASLIIWLGHNANWKQ
RSHLRRRRFFLQELGEQLIDGQIKRRMMDTRNLSK
SVKSGMTALNYKLPQRTPVEPSASMSGRCAFCPRT
DDRKTRKRCEDCKRFACVH

In some embodiments, the transposable element comprises: 1) a 5′TR in a LTF comprising the nucleic acid sequence of SEQ ID NO: 170; and 2) a 3′TR in a RTF comprising the nucleic acid sequence of SEQ ID NO: 182. In some embodiments, the transposable element comprises: 1) a 5′TR comprising the nucleic acid sequence of CAGTAGAACC CCG (SEQ ID NO: 82), a variant thereof, or a fragment thereof, and 2) a 3′TR comprising the nucleic acid sequence of CGGGGTTCTACTG (SEQ ID NO: 94), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 82; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 94. In some embodiments, the transposable element comprises: 1) a 5′TR comprising the nucleic acid sequence of SEQ ID NO: 82; and 2) a 3′TR comprising the nucleic acid sequence of SEQ ID NO: 94. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 170; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 182. In some embodiments, the transposable element is associated with a transposase comprising the amino acid sequence of SEQ ID NO: 106, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 106. In some embodiments, the transposable element comprises a nucleic acid sequence encoding the transposase. In some embodiments, the transposable element does not comprise a nucleic acid sequence encoding the transposase.

(transposase of Mariner-3_Crp)
SEQ ID NO: 106
MRRPSQMELRAAWVPKQDSGFSLSVATVFKMSASN
TDSTSASASTRKRKAITLEEKLEVVKRYERGEKTK
EIRCATGLSESTLRTIRDNAEKIKESCKSATRLST
ARVSLTRSAIMEKMERMLTTWIEHQNQEHVPLSTQ
VIQAKARSIYEVLKRDDPEAKPFNASAGWFDRFKA
RHGFHNLKLTGEAAAADKSAADKFPSILQAAIEEG
SYDPRQVFNLDETGLFWKRMPSRTFISVEERTAPG
FKAAKDRCTLLLGANAAGDFKLKPLMVYHAENPRA
LKGYAKGHLPVYWRANRKGWVTGSLFEDYFSSLLH
LELRAYCDRQNLPFKILLLLDNAPGHPPSLGDLSE
NIKVLFLPPNTTSLIQPMDQGVIRAFKSYYLRRTF
KKLINETESKEKANVKEFWKAFNIKMAIDIIGDAW
NDVTQQCLNGVWKNIWPAVVNDFRGFAADEFISDA
RHEIVEMAKSVGFEQVDEENVAELLDSHREELSNE
DLLELDRERHEEEEPMEENERPSPRVLTMKGMADA
FKLLDGYLAFFDENDPNRERSAKVARLMKEANACY
TELYREKRRCSVQSSLDKFFRKRDSTISTQSTDSQ
PSTSGGEPATSGGEISPPSFDVFDLDEDSAPH

In some embodiments, the transposable element comprises: 1) a 5′TR in a LTF comprising the nucleic acid sequence of SEQ ID NO: 171; and 2) a 3′TR in a RTF comprising the nucleic acid sequence of SEQ ID NO: 183. In some embodiments, the transposable element comprises: 1) a 5′TR comprising the nucleic acid sequence of CAGTGGTTCT TAACCT (SEQ ID NO: 83), a variant thereof, or a fragment thereof; and 2) a 3′TR comprising the nucleic acid sequence of AGGTTAAGAA CCACTG (SEQ ID NO: 95), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 83; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 95. In some embodiments, the transposable element comprises: 1) a 5′TR comprising the nucleic acid sequence of SEQ ID NO: 83; and 2) a 3′TR comprising the nucleic acid sequence of SEQ ID NO: 95. In some embodiments, the transposable element further comprises a 5′TSD comprising the nucleic acid sequence of CTCTAGAG (SEQ ID NO: 203), and a 3′TSD comprising the nucleic acid sequence of SEQ ID NO: 203. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 171; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 183. In some embodiments, the transposable element is associated with a transposase comprising the amino acid sequence of SEQ ID NO: 107, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 107. In some embodiments, the transposable element comprises a nucleic acid sequence encoding the transposase. In some embodiments, the transposable element does not comprise a nucleic acid sequence encoding the transposase.

(transposase of hAT-1_PM)
SEQ ID NO: 107
MSGKRRKWSDEYVQHGFSCITERDGSERPICMICN
AKLSNSSLAPAKLKEHFLKLHGDGEYKNTTLAEFR
VKRARFDERATLPALGFVPVNKPILTASYEVAYLI
AKQGKPHTIGETLVKPAALKMANLILGTAAEGKLS
QIPLSNDTVSDRIEDMSKDILAQVVADLISSPAKF
SLQLDETTDVSDLSQLAVFVRYVKDDMIKEEFLFC
KPLTTTTKAADVKKLVDDFFRDNNLSWDMVSAVCT
DGAPVMLGRKSGFGALVKADAPHIIVTHCILHRHA
LATKTLPPKMAEVLKTVVECVNYVRKSALRHRIFS
ELCKEMGSEFEVLLLHSNIRWLSRGQVLNRVFAMR
VELALFLQEHQHCHADCFKNPEFILTLAYMADTFA
ALNHLNQQMQGGGVNIMEAEEHLKAFQKKLPLWKR
RTENNNFANFPLLDDCASKIEDVSGIGDISVSTEL
KQTIATHLDELAKSLDGYFPTRESYPAWVRQPFTF
AVETADVNDEFLDEITEIQQSQVQQQLFRTTTLST
FWCRQMNKYPVIAKKALDFFIPFVTTYLCEQSFSR
MLDIKTKKRNRLCCENDMRVALAKVKPRISELISQ
RQQQKSH

In some embodiments, the transposable element comprises: 1) a 5′TR in a LTF comprising the nucleic acid sequence of SEQ ID NO: 172; and 2) a 3′TR in a RTF comprising the nucleic acid sequence of SEQ ID NO: 184. In some embodiments, the transposable element comprises: 1) a 5′TR comprising the nucleic acid sequence of TAGGGCTGTG CGAAA (SEQ ID NO: 84), a variant thereof, or a fragment thereof; and 2) a 3′TR comprising the nucleic acid sequence of TTTCGCACAG CCCTA (SEQ ID NO: 96), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 84; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 96. In some embodiments, the transposable element comprises: 1) a 5′TR comprising the nucleic acid sequence of SEQ ID NO: 84; and 2) a 3′TR comprising the nucleic acid sequence of SEQ ID NO: 96. In some embodiments, the transposable element further comprises a 5′TSD comprising the nucleic acid sequence of SEQ ID NO: 196, and a 3′TSD comprising the nucleic acid sequence of SEQ ID NO: 1%. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 172; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 184. In some embodiments, the transposable element is associated with a transposase comprising the amino acid sequence of SEQ ID NO: 108, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 108. In some embodiments, the transposable element comprises a nucleic acid sequence encoding the transposase. In some embodiments, the transposable element does not comprise a nucleic acid sequence encoding the transposase.

(transposase of hAT-17_Croc)
SEQ ID NO: 108
MTVSSTPISLPMLSLPVPEEELELDLDLSAIAREI
LGKEGESSEFVLHTPQVSPRARSPSPEGTSEEAEV
EVAEASGSAESAPPVAVPLPAEEGEKAASAHAPAP
QKRAGSVVWDHFELADDPTYAICLHCRAKTSRGKG
TKHMSTTGMLMHLRRHHPLALAPPQPGTSGSVPKG
KSPSRSKAPVPPKQRQATLEQWGKGGQKGGRVARA
SEITQSIGEMLAVDGQPFSLVERPGFRRLMALVAP
SYQVPARTTFSRTVVPSLYEACREYLREELRKAGP
QVALHFTSDIWSSRGGDHAYLSLTGHWCDQSGRRW
ALLQAEVMDESHTATETMGAMNRLVQGWLVGQGEL
TRGFMVTDNGANMVKAVRDANFVGIRCVAHKFHLI
VRDALEGDRAAGDGASTTSKLISKCRKVAGYFHRS
IKGGKMLRDKQAELNIPQHKIMQDVETRWNSTYLM
LERLVEQQKAIHEMALLGEIGISGPLNRAEWDTIS
QILVVLKPFLEATETLSAGDALLSQVIPVVRELQS
QMESFQGINVPGWGKPLSPDVQALVRRLKEGIRKR
LDPLRSSTVHVLAATCDPRVKGSVCGSQSLNQWTE
VLVKRVREAERRRRGDVEEGDPLSRASTPSTSQSP
PPPQALPMWAKGMASMVGSRGTRPHHQAGSAQALV
AAYLAEDVEPLLCDPLAYWASRSQMWPDLATVARE
HLSCPPTSVPSERVFSIAGDVVTPHRTSLDPGLVE
QLVFLKVNLPLIGFPKIQVQTE

In some embodiments, the transposable element comprises: 1) a 5′TR in a LTF comprising the nucleic acid sequence of SEQ ID NO: 173; and 2) a 3′TR in a RTF comprising the nucleic acid sequence of SEQ ID NO: 185. In some embodiments, the transposable element comprises: 1) a 5′TR comprising the nucleic acid sequence of CAGGTTGAG (SEQ ID NO: 85), a variant thereof, or a fragment thereof; and 2) a 3′TR comprising the nucleic acid sequence of CTCAACCTG (SEQ ID NO: 97), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 85; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 97. In some embodiments, the transposable element comprises: 1) a 5′TR comprising the nucleic acid sequence of SEQ ID NO: 85; and 2) a 3′TR comprising the nucleic acid sequence of SEQ ID NO: 97. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 173; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 185. In some embodiments, the transposable element is associated with a transposase comprising the amino acid sequence of SEQ ID NO: 109, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 109. In some embodiments, the transposable element comprises a nucleic acid sequence encoding the transposase. In some embodiments, the transposable element does not comprise a nucleic acid sequence encoding the transposase.

(transposase of Tigger4)
SEQ ID NO: 109
MSKRPADTPMGNSDKKKRKHLCLSIAQKVKLLEKL
DSGVSVKRLTEEYGVGMTTIYDLKKQKDKLLKFYA
ESDEQKLMKNRKTLHKAKNEDLDRVLKEWIRQRRS
EHMPLNGMLIMKQAKIYHDELKIEGNCEYSTGWLQ
KFKKRHGIKFLKICGDKASADHEAAEKFIDEFAKV
IADENLTPEQVYNADETSLFWRYCPRKTLTTADET
APTGIKDAKDRITVLOCANAAGTHKCKLAVIGKSL
RPRCFQGVNFLPVHYYANKKAWITRDIFSDWFHKH
FVPAARAHCREAGLDDDCKILLFLDNCSAHPPAEI
LIKNNVYAMYFPPNVTSLIQPCDQGILRSMKSKYK
NTFLNSMLAAVNRGVGVEGFQKEFSMKDAIYAVAN
AWNTVTKDTVVHAWHNLWPATMFSDDDEQGGDFEG
FRMSSEKKMMSDLLTYAKNIPSESVSKLEEVDIEE
VFNIDNEAPVVHSLTDGEIAEMVLNQGDRDNSDDE
DDVVNTAEKVPIDDMVKMCDGLIEGLEQRAFITEQ
EIMSVYKIKERLLRQKPLLMR

In some embodiments, the transposable element comprises: 1) a 5′TR in a LTF comprising the nucleic acid sequence of SEQ ID NO: 173; and 2) a 3′TR in a RTF comprising the nucleic acid sequence of SEQ ID NO: 185. In some embodiments, the transposable element comprises: 1) a 5′TR comprising the nucleic acid sequence of CAGTAGTCCC CCCTTATCCG CGG (SEQ ID NO: 86), a variant thereof, or a fragment thereof; and 2) a 3′TR comprising the nucleic acid sequence of CCGCGGATAA GGGGGGACTA CTG (SEQ ID NO: 98), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 86; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 98. In some embodiments, the transposable element comprises: 1) a 5′TR comprising the nucleic acid sequence of SEQ ID NO: 86; and 2) a 3′TR comprising the nucleic acid sequence of SEQ ID NO: 98. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 173; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 185. In some embodiments, the transposable element is associated with a transposase comprising the amino acid sequence of SEQ ID NO: 110, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 110. In some embodiments, the transposable element comprises a nucleic acid sequence encoding the transposase. In some embodiments, the transposable element does not comprise a nucleic acid sequence encoding the transposase.

(transposase of Tigger4)
SEQ ID NO: 110
MAPKRKSSDAGNSDMPKRSRKVLPLSEKVKVLDLI
RKEKKSYAEVAKIYGKNESSIREIVKKEKEIRASF
AVAPQTAKVTATVRDKCLVKMEKALNLWVEDMNRK
RVPIDGNVLRQKALSLYEDFSKGSPETSDTKPFTA
SKGWLHRFRNRFGLKNIKITGEAASANEEAAATFP
AELKKLIKEKGYHPKQVFNCDETGLFWKKMPNRTY
IHKSAKEAPGHKTWKDRLTLVLCGNAAGHMIKPGV
VYRAKNPRALKNKNKNYLPVFWQHNQKAWVIAILF
MEWFHQCFIPEVKKYLEEEGLLFKVLLIIDNAPGH
PESVCYENENVEVVFLPPNTTSLLQPLDQGIIRFV
KATYTRLVFDRIRSAIDADPNLDIMQCWKSFTIAD
AITFIKAAMDELKPETVNACWKNLWSEVVNDFKGF
PGIDGEVRKIIHAARQVGGEGFADMLDEEVEEHIE
GIIREVLTNEELEELVESSTEEEEDEEETEAEPAM
WTLPKFAEVFRIAQTLKDKIMEYDPRMERSIKVTR
MITEGLQPLQQHFDELKRKRQQLPITMFFQKISAK
NLQLSRIPNHRHRLLLTSNHRHRHGSMIQDHPKQM
ILLLTYRQKVNSSLTLRHNAYVIHLTSSHHVGIVS
SHIITRRRVSTVQ

In some embodiments, the transposable element comprises: 1) a 5′TR in a LTF comprising the nucleic acid sequence of SEQ ID NO: 175; and 2) a 3′TR in a RTF comprising the nucleic acid sequence of SEQ ID NO: 187. In some embodiments, the transposable element comprises: 1) a 5′TR comprising the nucleic acid sequence of TAGGG (SEQ ID NO: 87), a variant thereof, or a fragment thereof; and 2) a 3′TR comprising the nucleic acid sequence of CCCTA (SEQ ID NO: 99), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 87; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 99. In some embodiments, the transposable element comprises: 1) a 5′TR comprising the nucleic acid sequence of SEQ ID NO: 87; and 2) a 3′TR comprising the nucleic acid sequence of SEQ ID NO: 99. In some embodiments, the transposable element further comprises a 5′TSD comprising the nucleic acid sequence of TTTATAAT (SEQ ID NO: 204), and a 3′TSD comprising the nucleic acid sequence of SEQ ID NO: 204. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 175; and 2) a RTF comprising a nucleic acid sequence having at least about 90% (e.g., at least about any one of 91%, 92%, 93%, 94%, 95%, 96%, 97%, 98%, 99%, or 1000) sequence identity to the nucleic acid sequence of SEQ ID NO: 187. In some embodiments, the transposable element is associated with a transposase comprising the amino acid sequence of SEQ ID NO: 111, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 111. In some embodiments, the transposable element comprises a nucleic acid sequence encoding the transposase. In some embodiments, the transposable element does not comprise a nucleic acid sequence encoding the transposase.

(transposase of BAT-19_Crp)
SEQ ID NO: 111
MNAKKSSKTAKYVSALDRARQYPAGTLHADGGRLF
CTSCNVTLDVSRKTSIDRHLESEAHMKRKAAAEAE
GQSKKQATVSSLFKRTTESSLARREAAFSLVEAFA
AANIPLEKLDHPKLRDYLNQNVPNAGSFPRANKLR
QDYLPAVIASHVQSTKAALAEYHSFSIVADEATDP
QDNYVLHILFVPGSWTDRDLPVFQTEPVHLTSVNF
STVSQAIVKVIVSYGVDFNKISAFLSDNAMYMCKA
FSDVLQGMLPNAVHVTCNAHILSLVSDTWRTQFPE
VDSLVSSFKKAFKHCPGRKVRYRESVASQSGDSHG
TVPLPPEPVLTRWNSWFNAVRYHAKHMQHYQQFVS
EELEITPSTQCMLKLRDLLGRGDIQDQVTFVADNA
TRFMELLTWFEGRQAVIHHAHNKLMDLISWVEDRA
SQELSSCPRENQRRRDLFQAIAVKLNQYYRYDPSL
PPRFRQPAAPFLSAVRILDPNQVPLLSCDPQLLKA
IPGWGKEHDNERAAYVNFVKECQMPVPVPVFWDSV
CDRFPHLYALARRCLTIPTNSVDAERAISVYGQVF
TPQRQRMSPATAAGCSMLAFNT

In some embodiments, the transposable element comprises: 1) a 5′TR in a LTF comprising the nucleic acid sequence of SEQ ID NO: 176; and 2) a 3′TR in a RTF comprising the nucleic acid sequence of SEQ ID NO: 188. In some embodiments, the transposable element comprises: 1) a 5′TR comprising the nucleic acid sequence of TAGG (SEQ ID NO: 88), a variant thereof, or a fragment thereof; and 2) a 3′TR comprising the nucleic acid sequence of CCTA (SEQ ID NO: 100), a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 88; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 100. In some embodiments, the transposable element comprises: 1) a 5′TR comprising the nucleic acid sequence of SEQ ID NO: 88; and 2) a 3′TR comprising the nucleic acid sequence of SEQ ID NO: 100. In some embodiments, the transposable element further comprises a 5′TSD comprising the nucleic acid sequence of CTATATAG (SEQ ID NO: 205), and a 3′TSD comprising the nucleic acid sequence of SEQ ID NO: 205. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 176; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 188. In some embodiments, the transposable element is associated with a transposase comprising the amino acid sequence of SEQ ID NO: 112, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 112. In some embodiments, the transposable element comprises a nucleic acid sequence encoding the transposase. In some embodiments, the transposable element does not comprise a nucleic acid sequence encoding the transposase.

(transposase of hAT-17B_Croc)
SEQ ID NO: 112
MTVSSTPISSPSVSLPVPEEELDLEAGDLSALAKE
ILGTEGESEFVLHTPQSSPRARSPSPEGTSEEVEV
VEASGSAESAPPVVVPLPAEEGDKAGSPPSTQKRG
GSVVWDHFEVADDPKYAICQHCRRQISRGKEVKHF
TTTAMLLHLRRQHPLALA
PPQPGTSGSVPKGKSPSRSKAPVPTKQRQATLERW
GKAADKGGRVARASEVTQSIGEMLALDGQPFSLVE
RPGFRRLMALVAPSYQVPARTTFSRTVVPSLYEAC
REYLREELRKAGPQVALHFTSDIWSSRGGDHAYLS
LTGHWCDQSGRRWALLQAEVMDESHTAREIMVAMN
RMVQGWLVGQGELTRGFMVTDNGANMVKAVRDANF
VGIRCVAHKLHLIVRDALEGDRAAGDGAVTTTQLI
SKCRKVAGYFHRSIKGGKMLRDKQAELSVPQHKIM
QDVETRWNSTYLMLERLVEQQKAIHEMALLGEIGI
SGPLNKAEWDTISQILVVLKPFLEATETLSAGDAL
LSQVIPVVRELENQMEKFQGIDVPGWGKPLSPEVQ
ALVKRLKEGIKKWLDPLRSSTVHVLAGMCDPRVKG
SMCTGSTKTLDHWTEVLVNRVREAEGQRRGDVEEG
DPLSRASTPSTSQSPPRQPLPMWAKGMASMLGSRR
TRPQPRAGSAEASVATYLAEDVEPLDCDPLAYWAT
RSQMWQDLATVAREHLSCPPTSVPSERVFSIAGDV
VTPHRTSLDPGLV

In some embodiments, the transposable element comprises: 1) a 5′TR in a LTF comprising the nucleic acid sequence of SEQ ID NO: 177; and 2) a 3′TR in a RTF comprising the nucleic acid sequence of SEQ ID NO: 189. In some embodiments, the transposable element comprises: 1) a 5′TR comprising the nucleic acid sequence of SEQ ID NO: 89, a variant thereof, or a fragment thereof; and 2) a 3′TR comprising the nucleic acid sequence of SEQ ID NO: 101, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 89; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 101. In some embodiments, the transposable element comprises: 1) a 5′TR comprising the nucleic acid sequence of SEQ ID NO: 89; and 2) a 3′TR comprising the nucleic acid sequence of SEQ ID NO: 101. In some embodiments, the transposable element further comprises a 5′TSD comprising the nucleic acid sequence of ATTAATAG (SEQ ID NO: 206), and a 3′TSD comprising the nucleic acid sequence of SEQ ID NO: 206. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 177; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 189. In some embodiments, the transposable element is associated with a transposase comprising the amino acid sequence of SEQ ID NO: 113, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 113. In some embodiments, the transposable element comprises a nucleic acid sequence encoding the transposase. In some embodiments, the transposable element does not comprise a nucleic acid sequence encoding the transposase.

(transposase ofhAT-10_XT)
SEQ ID NO: 113
MTGRGRGRKGGMGGKGGEGGRKGGVGGEGGRKGGV
GGEGGVGGKGGLGGRSRGAGCGQEPSTRAMPSLGN
IGQQPQQQSQQQQQQLQQQQVAGRSGATGSRKTLP
DFFATTSRPIQ
SQQAEAVMDWLTQATSSAADSSEQQEVASPASSVI
RGMLSPLLFDPEVDFSPDTQDLFDDQTGMGGAFLS
DEEEVYVAQHTTSAVGDVGQGPLMAGQQADEPDVD
VDAGSDLGEDVEDDGDRSWVPAQEDNSSSSEGELC
VVVSDADEEPAPKKQKTPPTTARGGRQQGSTAPGS
TGSGRMGRDLQPSGPQPSTAGKVVARTSAVWAFFT
CQSQDPSVAVCHLCRQKVRRGQTGSHMGTSALSSH
MKRHHRMTWEQHQSGRAPGGSGASCPPPPIPQGTG
ASSPIPVAREEDSEAHSWRRPLCSSASSAAPSVSS
SSLLPSAQPLSRQVSLPQFLGRKAPLSSSHPQVQR
LNACLAKLLAVQLLPYQLVDAAPFRQLMACAAPNW
RIPSRHYFARKAVPALHQQVVDNVSLSLDYAVGGR
VHCTTDTWTSRHGQGRYISYTAHWVTLMSAGEGAG
SRTPLRLEVPPRGVKGKPHTATSSSSSSTMDEPPL
KRPRSYASVQYKRCQAVLQLLCLGERSHTAPELHA
AFQTQVQRWFTPRQLQAGNVVCDNGRNLLAALHLG
RLTHVPCFAHVLNLVVQHFLKSYTGLGVVLEKARR
VCGHFRRSPTASASLSRMQRQHSLPPHRLICDLPT
RWNSTLHMVERLVEQRWAVSNYLLEHSARGTQGAN
LGYFSTEQWQQMRQLCRVLAPFEQATRFVSRDNAC
VSDIIPLVFLLKRTLDGLLEEGDVPEEEECRPRRQ
VEGAARHDEEDMPNEEDEGEEDWVPAEQGEQGPRH
PTTPAVVRGWEDTEQGEEAGDDLLHLQGSQEEVGR
GHLFYMAAHMQTCLRSDSRICAIKDREDYWVATMI
DPRYKEKMAQFLVPSQRERRVGQLKRALCAKLMEA
FPQPDTQASTTPHIQQRQESSSSSSSAARGGGNLM
DVWRSFFEPRQAAAGLVSTQSHHQQRLDNMVADYM
GSVSVQDAMHTDDDPMNFWVSRLDQWPELAQYALE
VLACPPASVLSERVFSAAGGVVTEKRTRLSTGSVD
RLSFIKMNEAWISGDIHVPIADIRD

In some embodiments, the transposable element comprises: 1) a 5′TR in a LTF comprising the nucleic acid sequence of SEQ ID NO: 178; and 2) a 3′TR in a RTF comprising the nucleic acid sequence of SEQ ID NO: 190. In some embodiments, the transposable element comprises: 1) a 5′TR comprising the nucleic acid sequence of SEQ ID NO: 90, a variant thereof, or a fragment thereof; and 2) a 3′TR comprising the nucleic acid sequence of SEQ ID NO: 102, a variant thereof, or a fragment thereof. In some embodiments, the transposable element comprises: 1) a 5′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 90; and 2) a 3′TR having a nucleic acid sequence that has at least about 90% (e.g., at least about any one of 91%, 92%, 93%, 94%, 95%, 96%, 97%, 98%, 99%, or more) sequence identity to the nucleic acid sequence of SEQ ID NO: 102. In some embodiments, the transposable element comprises: 1) a 5′TR comprising the nucleic acid sequence of SEQ ID NO: 90; and 2) a 3′TR comprising the nucleic acid sequence of SEQ ID NO: 102. In some embodiments, the transposable element further comprises a 5′TSD comprising the nucleic acid sequence of SEQ ID NO: 193, and a 3′TSD comprising the nucleic acid sequence of SEQ ID NO: 193. In some embodiments, the transposable element does not comprise 5′TSD and/or 3′TSD. In some embodiments, the transposable element comprises a LTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 178; and 2) a RTF comprising a nucleic acid sequence having at least about 90% (e.g., at least about any one of 91%, 92%, 93%, 94%, 95%, 96%, 97%, 98%, 99%, or 100%) sequence identity to the nucleic acid sequence of SEQ ID NO: 190. In some embodiments, the transposable element is associated with a transposase comprising the amino acid sequence of SEQ ID NO: 114, or a variant thereof. In some embodiments, the transposase comprises an amino acid sequence having at least about 80% (e.g., at least about any one of 85%, 86%, 87%, 88%, 89%, 90%, 91%, 92%, 93%, 94%, 95%, 96%, 97%, 98%, 99% or more) sequence identity to the amino acid sequence of SEQ ID NO: 114. In some embodiments, the transposable element comprises a nucleic acid sequence encoding the transposase. In some embodiments, the transposable element does not comprise a nucleic acid sequence encoding the transposase.

(5′TR of MARWOLEN1)
SEQ ID NO: 90
TACAGTGGCGAGCAAAACTGAGTGCATGTTCACAA
CTCTCACTTTGGCCATCAATAAAAACACAACCGCT
TATGCAAATTCAATAATTTTTTTTATATTATTAAT
CTTAATTAATTTATAACTTATTTTATGAGAAAAAA
CAAATTTAGCATATATATTCAAAGACTTAGAATAA
AAAAACTAAATAAACTCACATAAACTCAATTTTGC
ACGTTT
(3′TR of MARWOLEN1)
SEQ ID NO: 102
AAACGTGCAAAATTGAGTTTATGTGAGTTTATTTA
GTTTTTTTATTCTAAGTCTTTGAATATATATGCTA
AATTTGTTTTTTCTCATAAAATAAGTTATAAATTA
ATTAAGATTAATAATATAAAAAAAATTATTGAATT
TGCATAAGCGGTTGTGTTTTTATTGATGGCCAAAG
TGAGAGTTGTGAACATGCACTCAGTTTTGCTCGCC
ACTGTA
(transposase of MARWOLENI)
SEQ ID NO: 114
MRKLSGEIQNNIVSLTESGNSTRQIAERLGISQSA
VVSIQKRRKLTPKPQVSGRKKLLKDSDARLMMSEM
RKNKNLTPKGACLAINKNVSEWTARRALQDIGYMS
IVKKNKPALSDKNVKARLKFAKDHKNWTIDDWKRV
VWSDESKFNRFQSDGKQYCWIRPGDRVQRHHVKQT
VKHGGGNIMVWGCFTWWHIGPLQLVEGIMKKEDYL
RILQTNLPNYFDKCAYPEKDIIFQQDGDPKIITAK
IVKEWIGKQHFQLMEWPAQSPDLNPIENLWSIVKR
RLGQYDSAPKNMGDLWERVAVEWSRIPQDILRNLV
ESMPKRVTEVTVNKGLWTKY

### Heterologous Nucleic Acid

The engineered transposable elements described herein are suitable for transposing a variety of heterologous nucleic acids. In some embodiments, the heterologous nucleic acid is a DNA. In some embodiments, the heterologous nucleic acid is double-stranded. In some embodiments, the heterologous nucleic acid comprises one or more modified nucleotides. In some embodiments, the heterologous nucleic acid is not modified.

The heterologous nucleic acid in the transposable element may be of various suitable lengths. In some embodiments, the heterologous nucleic acid is at least about any one of 1 kb, 2 kb, 10 kb, 20 kb, 30 kb, 40 kb, 50 kb, 60 kb, 70 kb, 80 kb, 90 kb, 100 kb, 150 kb, 200 kb, 250 kb, 300 kb, 350 kb, 400 kb, 450 kb, 500 kb, 600 kb, 700 kb, 800 kb, 900 kb, 1000 kb or more in length. In some embodiments, the heterologous nucleic acid is no more than about any one of 1000 kb, 900 kb, 800 kb, 700 kb, 600 kb, 500 kb, 450 kb, 400 kb, 350 kb, 300 kb, 250 kb, 200 kb, 150 kb, 100 kb, 90 kb, 80 kb, 70 kb, 60 kb, 50 kb, 40 kb, 30 kb, 20 kb, 10 kb, 5 kb, 2 kb, or 1 kb in length. In some embodiments, the heterologous nucleic acid has a length in any one of the ranges from about 100 bp to about 1 kb, about 1 kb to about 2 kb, about 2 kb to about 5 kb, about 5 kb to about 10 kb, about 100 bp to about 5 kb, about 100 bp to about 2 kb, about 2 kb to about 10 kb, about 1 kb to about 10 kb, about 10 kb to about 20 kb, about 20 kb to about 50 kb, about 50 kb to about 100 kb, about 1 kb to about 100 kb, about 150 kb to about 200 kb, about 200 kb to about 300 kb, about 300 kb to about 400 kb, about 400 kb to about 500 kb, about 500 kb to about 600 kb, about 600 kb to about 700 kb, about 700 kb to about 800 kb, about 800 kb to about 900 kb, about 900 kb to about 1000 kb, about 10 kb to about 100 kb, about 100 kb to about 500 kb, about 500 kb to about 1000 kb, or about 10 kb to about 500 kb. In some embodiments, the heterologous nucleic acid is from about 10 kb to about 300 kb nucleotides long. In some embodiments, the heterologous nucleic acid is from about 100 bp to about 300 kb nucleotides long.

The heterologous nucleic acid may comprise comprises one or more coding sequences, including any one of 1, 2, 3, 4, 5, 6, 10 or more coding sequences. Any suitable coding sequence may be used in the present application, and the coding sequence may encode any suitable biological product of interest. In some embodiments, the coding sequence encodes an RNA molecule. In some embodiments, the coding sequence encodes a polypeptide, such as a protein. In some embodiments, the heterologous nucleic acid comprises a first coding sequence encoding a first protein and a second coding sequence encoding a second protein. In some embodiments, the heterologous nucleic acid comprises a first coding sequence encoding a first RNA and a second coding sequence encoding a second RNA. In some embodiments, the heterologous nucleic acid comprises a first coding sequence encoding a protein and a second coding sequence encoding a RNA.

In some embodiments, the coding sequence encodes a therapeutic protein. In some embodiments, the coding sequence encodes a therapeutic antibody, including monoclonal antibody, multispecific antibody, and antibody fragments. In some embodiments, the coding sequence encodes a cytokine. In some embodiments, the coding sequence encodes an antigen. In some embodiments, the coding sequence encodes a therapeutic agent useful in gene therapy. Exemplary therapeutic proteins useful for gene therapy include, but are not limited to, adenosine deaminase, the enzymes affected in lysosomal storage diseases, apolipoprotein E, brain derived neurotropihic factor (BDNF), bone morphogenetic protein 2 (BMP-2), bone morphogenetic protein 6 (BMP-6), bone morphogenetic protein 7 (BMP-7), cardiotrophin 1 (CT-1), CD22, CD40, ciliary neurotrophic factor (CNTF), CCL1-CCL28, CXCL1-CXCL17, CXCL1, CXCL2, CX3CL1, vascular endothelial cell growth factor (VEGF), dopamine, erythropoietin, Factor IX, Factor VIII, epidermal growth factor (EGF), estrogen, FAS-ligand, fibroblast growth factor 1 (FGF-1), fibroblast growth factor 2 (FGF-2), fibroblast growth factor 4 (FGF-4), fibroblast growth factor 5 (FGF-5), fibroblast growth factor 6 (FGF-6), fibroblast growth factor 1 (FGF-7), fibroblast growth factor 1 (FGF-10), Flt-3, granulocyte colony-stimulating factor (G-CSF), granulocyte macrophage stimulating factor (GM-CSF), growth hormone, hepatocyte growth factor (HGF), interferon alpha (IFN-a), interferon beta (IFN-b), interferon gamma (IFNg), insulin, glucagon, insulin-like growth factor 1 (IGF-1), insulin-like growth factor 2 (IGF-2), interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 15 (IL-15), interleukin 17 (IL-17), interleukin 19 (IL-19), macrophage colony-stimulating factor (M-CSF), monocyte chemotactic protein 1 (MCP-1), macrophage inflammatory protein 3a (MIP-3a), macrophage inflammatory protein 3b (MIP-3b), nerve growth factor (NGF), neurotrophin 3 (NT-3), neurotrophin 4 (NT-4), parathyroid hormone, platelet derived growth factor AA (PDGF-AA), platelet derived growth factor AB (PDGF-AB), platelet derived growth factor BB (PDGF-BB), platelet derived growth factor CC (PDGF-CC), platelet derived growth factor DD (PDGF-DD), RANTES, stem cell factor (SCF), stromal cell derived factor 1 (SDF-1), transforming growth factor alpha (TGF-a), transforming growth factor beta (TGF-b), tumor necrosis factor alpha (TNF-a), Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16, Sonic hedgehog, Desert hedgehog, and Indian hedgehog.

In some embodiments, the coding sequence encodes an engineered receptor, such as a chimeric antigen receptor (CAR) or an engineered T-cell receptor (TCR).

As used herein, “Chimeric antigen receptor” or “CAR” refers to genetically engineered receptors, which graft one or more antigen specificity onto cells, such as T cells. CARs are also known as “artificial T-cell receptors,” “chimeric T cell receptors,” or “chimeric immune receptors.” In some embodiments, the CAR comprises an extracellular variable domain of an antibody specific for a tumor antigen, and an intracellular signaling domain of a T cell or other receptors, such as one or more costimulatory domains. “CAR-T” refers to a T cell that expresses a CAR.

In some particular embodiments, the coding sequence encodes a chimeric antigen receptor (CAR). Many chimeric antigen receptors are known in the art and may be suitable for use in the present application. CARs can also be constructed with a specificity for any cell surface marker by utilizing antigen binding fragments or antibody variable domains of, for example, antibody molecules. Any methods for producing a CAR may be used herein. See, for example, U.S. Pat. Nos. 6,410,319, 7,446,191, 7,514,537, 9,765,342B2, WO 2002/077029, WO2015/142675, US2010/065818, US 2010/025177, US 2007/059298, and Berger C. et al., J. Clinical Investigation 118:1 294-308 (2008), which are hereby incorporated by reference.

471 “T cell receptor” or “TCR” as used herein refers to endogenous or recombinant T cell receptor comprising an extracellular antigen binding domain that binds to a specific antigenic peptide bound in an MHC molecule. In some embodiments, the TCR comprises a TCRα polypeptide chain and a TCR β polypeptide chain. In some embodiments, the TCR specifically binds a tumor antigen. “TCR-T” refers to a T cell that expresses a recombinant TCR. The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

In some particular embodiments, the coding sequence encodes an engineered T-cell receptor (TCR). In some embodiments, the engineered TCR is specific for a tumor antigen. In some embodiments, the tumor antigen is derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for producing TCRs have been described, for example, in U.S. Pat. No. 5,830,755, and Kessels et al. Immunotherapy through TCR gene transfer. Nat. Immunol. 2, 957-961 (2001).

In some embodiments, the coding sequence encodes a selectable marker. A “selectable marker” is a gene, the expression of which creates a detectable phenotype and which facilitates detection of host cells having the heterologous nucleic acid encoding the selectable marker inserted in a target nucleic acid (e.g., genomic DNA). In some embodiments, the selectable marker confers resistance to an antibiotic agent, such as puromycin. Additional non-limiting examples of selectable markers include drug resistance genes and nutritional markers. For example, the selectable marker can be a gene that confers resistance to an antibiotic selected from the group consisting of: ampicillin, kanamycin, erythromycin, chloramphenicol, gentamycin, kasugamycin, rifampicin, spectinomycin, D-Cycloserine, nalidixic acid, streptomycin, or tetracycline. Other non-limiting examples of selection markers include adenosine deaminase, aminoglycoside phosphotransferase, dihydrofolate reductase, hygromycin-B-phosphotransferase, thymidine kinase, and xanthine-guanine phosphoribosyltransferase. Examples of selectable markers suitable for mammalian cells also include DHFR, thymidine kinase, metallothionein-I and -II, preferably primate metallothionein genes, adenosine deaminase, ornithine decarboxylase, etc. In some particular embodiments, the heterologous nucleic acid comprises a coding sequence of a puromycin resistance gene.

In some embodiments, the coding sequence is a reporter gene. A “reporter gene” is a gene that encodes a detectable product so that detection of the reporter gene product can be used to evaluate the function of a nucleic acid of interest. A reporter gene may be fused to any suitable nucleic acid of interest (e.g. promoter, a gene of interest, a selectable marker, and/or terminal repeats of a transposable element) to allow one to detect whether the nucleic acid of interest is expressed or altered (e.g. excised by a transposase) under a given set of conditions. Non-limiting examples of reporter genes include: 3-galactosidase, 3-glucuronidase, glutathione-S-transferase (GST), horseradish peroxidase (HRP), luciferase, chloramphenicol acetyltransferase (CAT), secreted alkaline phosphatase (SEAP), green fluorescent protein (GFP, e.g., eGFP), red fluorescent protein (RFP), HcRed, DsRed, cyan fluorescent protein (CFP), yellow fluorescent protein (YFP), catechol 2,3-oxygenase (xylE), and autofluorescent proteins including blue fluorescent protein (BFP). In some embodiments, the heterologous nucleic acid comprises a coding sequence encoding an enhanced green fluorescent protein (eGFP). In some embodiments, the coding sequence encodes more than one biological products, or the coding sequence may encode a fusion protein. In some embodiments, the heterologous nucleic acid of the present application comprises a coding sequence encoding a puromycine resistance—enhanced green fluorescent protein (eGFP) fusion protein.

In some embodiments, the coding sequence encodes a transposase.

In some embodiments, the coding sequence encodes a polypeptide useful in genome editing. Genome editing may be accomplished by using nucleases, which create specific double-strand breaks (DSBs) at desired locations in the genome, and harness the cell's endogenous mechanisms to repair the induced break by homology-directed repair (HDR) (e.g., homologous recombination) or by nonhomologous end joining (NHEJ). Any suitable nuclease may be introduced into a cell to induce genome editing of a target DNA sequence including, but not limited to, CRISPR-associated protein (Cas, e.g., Cas9) nucleases, zinc finger nucleases (ZFNs, e.g. FokI), transcription activator-like effector nucleases (TALENs, e.g., TALEs), meganucleases, and variants thereof (Shukla et al. (2009) Nature 459: 437-441; Townsend et al (2009) Nature 459: 442-445). In some embodiments, the coding sequence encodes a Cas9 polypeptide.

In some embodiments, the coding sequence encodes an RNA molecule. The RNA molecule may be a protein-coding RNA such as messenger RNA (mRNA), or a non-protein coding RNA, including, but not limited to, transfer RNA (tRNA) and ribosomal RNA (rRNA), small RNA such as microRNA (miRNA), small interfering RNA (siRNA), short hairpin RNA (shRNA), or piwi-interacting RNA (piRNA), and long non-coding RNA (lincRNA). Certain types of small RNA, such as microRNA and siRNA, are important in the process RNA interference (RNAi). RNAi is a process of genetic regulation in which a target gene that would otherwise normally express is suppressed from expression due to interference of small RNAs through post-transcriptional degradation or inhibition of translation. For detailed description of RNAi techniques, see, e.g., U.S. Pat. Nos. 5,034,323; 6,326,527; 6,452,067; 6,573,099; 6,753,139; and 6,777,588. In some embodiments, the coding sequence encodes a regulatory RNA. In some embodiments, the coding sequence encodes an RNAi molecule. In some embodiments, the coding sequence encodes a shRNA. In some embodiments, the coding sequence encode an miRNA.

In some embodiments, the coding sequence encodes an RNA molecule that is useful in genome editing. Examples of such RNA molecules include, but are not limited to, CRISPR RNA (crRNA), trans-activating crRNA (tracrRNA), guide RNA (gRNA), and single guide RNA (sgRNA).

In some embodiments, the heterologous nucleic acid further comprises one or more regulatory elements regulating expression of the coding sequence. Regulatory elements are contemplated for use with the methods and constructs described herein. The term “regulatory element” is intended to include promoters, enhancers, internal ribosomal entry sites (IRES), and other expression control elements (e.g., transcription termination signals, such as polyadenylation (poly-A) signals and poly-U sequences). Such regulatory elements are described, for example, in Goeddel, Gene Expression Technology: Methods in Enzymology 185, Academic Press, San Diego, Calif. (1990).

Promoters are an important regulatory element that directs expression pattern of the coding sequence. In some embodiments, the heterologous nucleic acid comprises a promoter operably linked to the coding sequence. Any suitable promoters may be used in the present application. In some embodiments, the promoter is an endogenous promoter. In some embodiments, the promoter is a heterologous promoter. Varieties of promoters have been explored for gene expression in mammalian cells, and any of the promoters known in the art may be used in the present application. Promoters may be roughly categorized as constitutive promoters or regulated promoters, such as inducible promoters. In some embodiments, the heterologous nucleic acid comprises a coding sequence (e.g., transposase-coding sequence) operably linked to a constitutive promoter. In some embodiments, the heterologous nucleic acid comprises a coding sequence (e.g., transposase-coding sequence) operably linked to an inducible promoter.

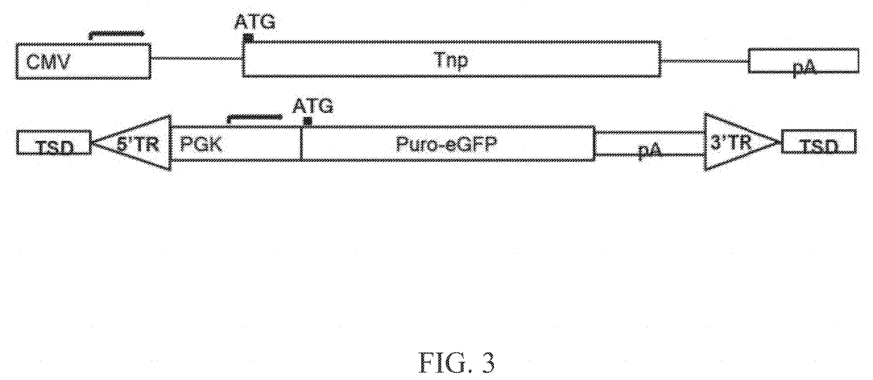

Constitutive promoters allow a heterologous nucleic acid to be expressed constitutively in the host cells. Exemplary constitutive promoters contemplated herein include, but are not limited to, cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the promoter in the heterologous nucleic acid is a CAG promoter. Exemplary engineered transposable elements comprising a heterologous nucleic acid sequence encoding a transposase or a selectable marker/reporter driven by a constitutive promoter are shown in FIG. 3, wherein the promoter is a CMV promoter or a PGK promoter.

It is contemplated that using a promoter with a moderate or weak expression pattern, as opposed to a strong expression promoter (e.g. the CMV promoter), of the coding sequence may be desired for certain applications, such as in certain gene therapies, in order to avoid or reduce events of transposase-based autoregulation, collectively referred to as overproduction inhibition (OPI).

Regulated promoters, such as inducible promoters, allow a heterologous nucleic acid to be expressed under certain circumstances, such as in a specific developmental stage, or a specific tissue type or subcellular location. Various types of regulated promoters are known in the art, including inducible, tissue-specific, cell-type-specific, or cell cycle-specific, see e.g., Sambrook and Russell, 2001. Inducible promoters belong to the category of regulated promoters. An inducible promoter can be induced by one or more conditions, such as a physical condition, microenvironment of the engineered mammalian cell, or the physiological state of the engineered mammalian cell, an inducer (i.e., an inducing agent), or a combination thereof.

In some embodiments, it may be desirable to use a promoter to express the coding sequence in only a subset of cell types, cell lineages, or tissues or during specific stages of development. Examples include, but are not limited to: an B29 promoter (B cell expression), a runt transcription factor (CBFa2) promoter (stem cell expression), an CD14 promoter (monocytic cell expression), an CD43 promoter (leukocyte and platelet expression), an CD45 promoter (hematopoietic cell expression), an CD68 promoter (macrophage expression), an endoglin promoter (endothelial cell expression), a fms-related tyrosine kinase 1 (FLT1) promoter (endothelial cell expression), an integrin, alpha 2b (ITGA2B) promoter (megakaryocyte expression), an intracellular adhesion molecule 2 (ICAM-2) promoter (endothelial cell expression), an interferon beta (IFN-β) promoter (hematopoietic cell expression), a β-globin LCR (erythroid cell expression), a globin promoter (erythroid cell expression), a β-globin promoter (erythroid cell expression), an α-globin HS40 enhancer (erythroid cell expression), an ankyrin-1 promoter (erythroid cell expression), and a Wiskott-Aldrich syndrome protein (WASP) promoter (hematopoietic cell expression).

Aspects of the methods described herein may make use of terminator sequences. A terminator sequence includes a section of nucleic acid sequence that marks the end of a gene or an operon during transcription. This sequence mediates transcriptional termination by providing signals in the newly synthesized mRNA that trigger processes to release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art. In some embodiments of the present application, the terminator sequence is a polyadenylation (poly-A) signal.

In some embodiments, the heterologous nucleic acid comprises at least one restriction endonuclease recognized site, e.g. restriction site, serving as a site for insertion of an exogenous nucleic acid. A variety of restriction sites are known in the art and include, but are not limited to: HindIII, PstI, SalI, AccI, HincII, XbaI, BamHI, SmaI, XmaI, KpnI, SacI, EcoRI, and the like. In some embodiments, the restriction site is a multiple cloning site (MCS, also known as a polylinker), i.e. a closely arranged series or array of sites recognized by a plurality of different restriction enzymes, such as those listed above. In other embodiments, the heterologous nucleic acid of the present application comprises recombinase recognition sites, such as LoxP, FRT, or AttB/AttP sites, which are recognized by the Cre, Flp, and PhiC31 recombinases, respectively.

In some embodiments, the heterologous nucleic acid comprises a tag sequence. A tag sequence can be used to identify a molecule, or provide a site for capture of a molecule, e.g., by hybridization.

In some embodiments, the heterologous nucleic acid comprises a barcode sequence. “Barcode sequence” refers to a nucleic acid having a sequence, which can be used to identify and/or distinguish one or more first molecules to which the nucleic acid barcode is conjugated from one or more second molecules. Nucleic acid barcode sequences are typically short, e.g., about 5 to 20 bases in length, and may be conjugated to one or more target molecules of interest or amplification products thereof. Nucleic acid barcode sequences may be single or double stranded.

In some embodiments, the heterologous nucleic acid comprises a unique molecular identifier (UMI). The term “unique molecular identifier” or “UMI” as used herein refers to nucleic acid sequence, which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a nucleic acid barcode sequence and a UMI are incorporated into a nucleic acid target molecule or an amplification product thereof. Generally, a UMI is used to distinguish between molecules of a similar type within a population or group, whereas a nucleic acid barcode sequence is used to distinguish between populations or groups of molecules. In some embodiments, where both a UMI and a nucleic acid barcode sequence are utilized, the UMI is shorter in sequence length than the nucleic acid barcode sequence. In some embodiments, where both a UMI and a nucleic acid barcode sequence are utilized, the UMI is incorporated into the target nucleic acid or an amplification product thereof prior to the incorporation of the nucleic acid barcode sequence. In some embodiments, where both a UMI and a nucleic acid barcode sequence are utilized, the nucleic acid barcode sequence is incorporated into the UMI or an amplification product thereof subsequent to the incorporation of the UMI into a target nucleic acid or an amplification product thereof.

### Transposition Activity

In some embodiments, the transposable element of the present application exhibits transposition activity in vitro or in a cell.

Transposition activity can be detected by a number of techniques known to an ordinary skilled person in the art. Examples of assays for measuring the excision of a transposable element from a vector, the integration of a transposon into the genomic or extrachromosomal DNA of a cell, and the ability of transposase to bind to an inverted repeat may be found in, for instance, Ivies et al. Cell, 91, 501-510 (1997), WO 98/40510 (Hackett et al.), WO 99/25817 (Hackett et al.), and WO00/68399 (Mclvor et al.).

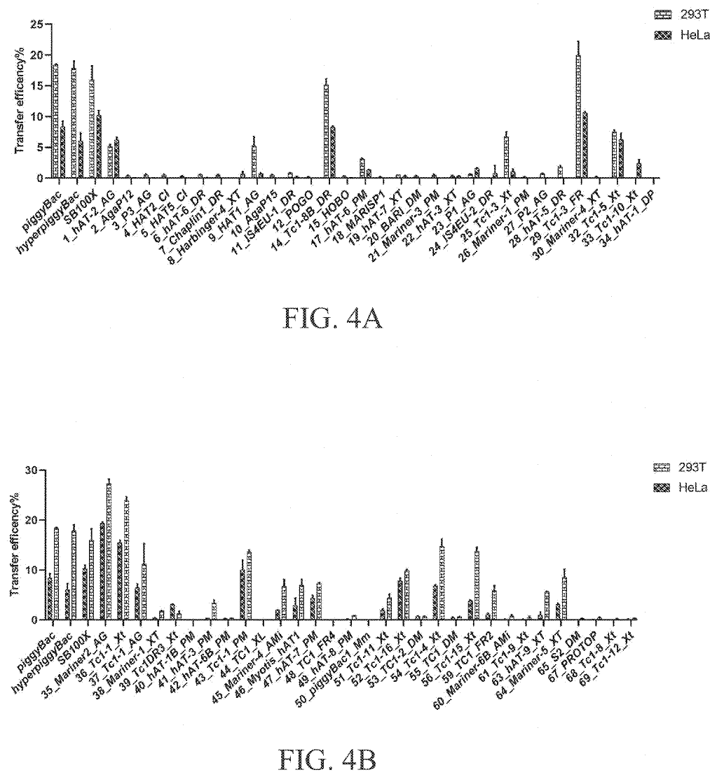

In some embodiments, the transposition assay is based on trans-complementation of two components in a transposable element system, with one component containing a selectable marker/reporter gene (donor) flanked by terminal repeats, and another component that expresses the transposase that recognizes and binds to the terminal repeats to perform transposition (helper). By way of example, FIG. 3 shows an exemplary set of binary constructs for use in screening active transposable elements. The top construct is the helper construct for transposase expression, comprising from 5′ to 3′: a cytomegalovirus (CMV) promoter, a transposase (Tn) gene, and a poly(A) signal. The bottom construct is the donor construct, comprising from 5′ to 3′: a phosphoglycerate kinase (PGK) promoter, a 5′ target site duplications (TSD) sequence, a 5′ terminal repeat (5′TR) sequence, a sequence encoding puromycin and enhanced GFP fusion (Puro-eGFP), a 3′ terminal repeat (3′TR) sequence, a 3′ target site duplications (TSD) sequence, and a poly(A) signal. In the transposition assay, the donor plasmid is co-transfected with the helper or control plasmids into cultured mammalian cells (e.g. human 293T. HeLa, or Hct116 cells), and the number of cell clones that are resistant to the puromycin due to chromosomal integration and expression of the puromycin resistance gene serves as an indicator of the efficiency of gene transfer, as demonstrated by methylene blue staining. For suspension cells, such as K562 and primary T cells, transposition activity can be assessed based on GFP reporter positive cells after electroporation. FIGS. 4A-6B, show the transposition efficiencies of the evaluated TEs as compared to controls based on colony counting of methylene blue staining results of the transposition assay. Transposition efficiency in such assays is also referred to as “transfer efficiency.”

In some embodiments, the transfer efficiency of the engineered transposable element is at least about any one of 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or higher. In some embodiments, the transfer efficiency of the engineered transposable element is determined in a human cell, such as human 293 T, HeLa, Hct116, K562, or primary T cells.

In some embodiments, the transposition activity of the engineered transposable element is higher than that of a piggyBac (PB) transposon, a Sleeping Beauty (SB) transposon, and/or a TcBuster (TB) transposon. In some embodiments, the transposition activity of the engineered transposable element is higher than that of a piggyBac (PB) transposon, for example, by about any one of 10%, 20%, 30%, 40%, 50%, 60%, 70%, 80%, 90%, 100%, 150%, 2×, 3×, 5×, 10× or more as determined by a reporter-based transposition assay (e.g., as described in Example 2) in a mammalian cell. In some embodiments, the transposition activity of the engineered transposable element is higher than that of a Sleeping Beauty (SB) transposon, for example, by about any one of 10%, 20%, 30%, 40%, 50%, 60%, 70%, 80%, 90%, 100%, 150%, 2×, 3×, 5×, 10× or more as determined by a reporter-based transposition assay (e.g., as described in Example 2) in a mammalian cell. In some embodiments, the transposition activity of the engineered transposable element is higher than that of a TcBuster (TB) transposon, for example, by at least about any one of 10%, 20%, 30%, 40%, 50%, 60%, 70%, 80%, 90%, 100%, 150%, 2×, 3×, 5×, 10× or more as determined by a reporter-based transposition assay (e.g., as described in Example 2) in a mammalian cell. In some embodiments, the transposition activity of the engineered transposable element is higher than that of a PB transposon and that of a SB transposon. In some embodiments, the transposition activity of the engineered transposable element is higher than that of a PB transposon and that of a TB transposon. In some embodiments, the transposition activity of the engineered transposable element is higher than that of a TB transposon and that of a SB transposon. In some embodiments, the transposition activity of the engineered transposable element is higher than that of a PB transposon, that of a SB transposon and that of a TB transposon.

In some embodiments, the transposition activity of the engineered transposable element is assessed in a mammalian cell. In some embodiments, the mammalian cell is a HeLa cell. In some embodiments, the mammalian cell is a human embryonic kidney 293T (293T). In some embodiments, the mammalian cell is a K562 cell. In some embodiments, the mammalian cell is a Hct116 cell. In some embodiments, the mammalian cell is a human T cell, such as primary T cell from a donor. In some embodiments, the engineered transposable element has higher transposition activity in a 293T cell than in a HeLa cell, such as at least about any one of 10%, 20%, 30%, 40%, 50%, 60%, 70%, 80%, 90%, 100%, 150%, 2×, 3×, 5×, 10× or more transposition activity in a 293T cell than in a HeLa cell.

### Cells

The engineered transposable elements described herein have transposition activity in a variety of cells, and can be used to insert heterologous nucleic acid into a target nucleic acid in any suitable cell.

In some embodiments, the cell is an isolated cell. In some embodiments, the cell is in cell culture. In some embodiments, the cell is ex vivo. In some embodiments, the cell is obtained from a living organism, and maintained in a cell culture. In some embodiments, the cell is a single-cellular organism. Cells may be classified into different types based on their sources, tissues of origin, morphologies, functions, histological markers, expression profiles, or the like.

In some embodiments, the cell is a prokaryotic cell. In some embodiments, the cell is a bacterial cell or derived from a bacterial cell. In some embodiments, the cell is an archaeal cell or derived from an archaeal cell. In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell. In some embodiments, the cell is related to an animal species from which the 5′TR, 3′TR and/or the transposase are derived from. In some embodiments, the cell is not related to an animal species from which the 5′TR, 3′TR and/or the transposase are derived from.

In some embodiments, the cell is a bacterium, a yeast cell, a fungal cell, an algal cell, a plant cell, or an animal cell. In some embodiments, the cell is a cell isolated from natural sources, such as a tissue biopsy. In some embodiments, the cell is a cell isolated from an in vitro cultured cell line. In some embodiments, the cell is a genetically engineered cell. In some embodiments, the cell is a seed cell that undergoes proliferation, differentiation, or both in the core.

In some embodiments, the cell is an animal cell from an organism selected from the group consisting of cattle, sheep, goat, horse, pig, deer, chicken, duck, goose, rabbit, and fish.

In some embodiments, the cell is a plant cell from an organism selected from the group consisting of maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, and a biomass crop.

In some embodiments, the cell is a mammalian cell, including cells from humans, domestic and farm animals, and zoo, sports, or pet animals, such as dogs, horses, cats, cows, etc. In some embodiments, the cell is a human cell. In some embodiments, the human cell is a human embryonic kidney 293T (HEK293T or 293T) cell or a HeLa cell.

In some embodiments, the cell is derived from a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

In some embodiments, the cell is derived from a cell line. A wide variety of cell lines are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)).

In some embodiments, the cell comprises an adherent cell. In some embodiments, the cell comprises a differentiated adherent cell. In some embodiments, the cell comprises an undifferentiated adherent cell. In some embodiments, the cell comprises a pluripotent stem cell. In some embodiments, the cell comprises a non-adherent cell.

In some embodiments, the cell is derived from an epithelial, muscular, nervous, or connective tissue, or any combination thereof. In some embodiments, the cell is derived from a tissue selected from the group consisting of liver, gastrointestinal, pancreatic, kidney, lung, tracheal, vascular, skeletal muscle, cardiac, skin, smooth muscle, connective tissue, corneal, genitourinary, breast, reproductive, endothelial, epithelial, fibroblast, neural, Schwann, adipose, bone, bone marrow, cartilage, pericytes, mesothelial, endocrine, stromal, lymph, blood, endoderm, ectoderm, mesoderm and combinations thereof. In some embodiments, the cell is derived from a tissue selected from the group consisting of connective tissue (for example, loose connective tissue, dense connective tissue, elastic tissue, reticular connective tissue and adipose tissue), muscle tissue (for example, skeletal muscle, smooth muscle and cardiac muscle), urogenital tissue, gastrointestinal tissue, lung tissue, bone tissue, nerve tissue and epithelial tissue (for example, a single layer of epithelial and stratified epithelium), endoderm-derived tissue, mesoderm-derived tissue and ectoderm-derived tissue, or any combination thereof. In some embodiments, the cell is derived from a tumor.

In some embodiments, the cell is selected from the group consisting of liver cell, gastrointestinal cell, pancreatic cell, kidney cell, lung cell, tracheal cell, vascular cell, skeletal muscle cell, cardiac cell, skin cell, smooth muscle cell, connective tissue cell, corneal cell, genitourinary cell, breast cell, reproductive cell, endothelial cell, epithelial cell, fibroblast, neural cell, Schwann cell, adipose cell, bone cell, bone marrow cell, cartilage cell, pericyte, mesothelial cell, cell derived from endocrine tissue, stromal cell, stem cell, progenitor cell, lymph cell, blood cell, endoderm-derived cell, ectoderm-derived cell, mesoderm-derived cell, undifferentiated cell (such as stem cell, or progenitor cell), tumor cell, iPS cell, and combinations thereof.

In some embodiments, the cell is an immune cell, such as T cells, B cells, Natural killer (NK) cells, dendritic cells (DCs) and macrophages. In some embodiments, the cell is a human T cell obtained from a patient or a donor. In some embodiments, the cell is an immune cell selected from the group consisting of a cytotoxic T cell, a helper T cell, a natural killer (NK) T cell, an iNK-T cell, an NK-T like cell, a αβT cell, a γδT cell, a tumor-infiltrating T cell and a dendritic cell (DC)-activated T cell. In some embodiments, the cell is an immune cell modified using the engineered transposable element or gene transfer system of the present application. In some embodiments, the modified immune cell is a CAR-T cell. In some embodiments, the modified immune cell is a TCR-T cell.

In some embodiments, the cell of the present application is a mammalian cell. In some embodiments, the mammalian cell is a human HKT293 cell or HeLa cell. In some further embodiments, the transposition activity of the transposable element is higher in 293T cells than in HeLa cells. In some embodiments, the mammalian the mammalian cell is selected from the group consisting of an immune cell, a hepatic cell, a tumor cell, a stem cell, a zygote, a muscle cell, and a skin cell.

In some embodiments, the cell is a stem cell or progenitor cell. Cells can include stem cells (e.g., adult stem cells, embryonic stem cells, iPS cells) and progenitor cells (e.g., cardiac progenitor cells, neural progenitor cells, etc.). Cells can include mammalian stem cells and progenitor cells, including rodent stem cells, rodent progenitor cells, human stem cells, human progenitor cells, etc.

In some embodiments, the cell is a diseased cell. A diseased cell can have altered metabolic, gene expression, and/or morphologic features. A diseased cell can be a cancer cell, a diabetic cell, and an apoptotic cell. A diseased cell can be a cell from a diseased subject.

In some embodiments, the cell of the present application belongs to a target cell type that is useful in gene therapy. Illustrative target cell types include hematopoietic stem cells, hematopoietic progenitor cells, myeloid progenitors, lymphoid progenitors, thrombopoietic progenitors, erythroid progenitors, granulopoietic progenitors, monocytopoictic progenitors, megakaryoblasts, promegakaryocytes, megakaryocytes, thrombocytes/platelets, proerythroblasts, basophilic erythroblasts, polychromatic erythroblasts, orthochromatic erythroblasts, polychromatic erythrocytes, erythrocytes (red blood cells or RBCs), basophilic promyelocytes, basophilic myelocytes, basophilic metamyelocytes, basophils, neutrophilic promyelocytes, neutrophilic myelocytes, neutrophilic metamyelocytes, neutrophils, eosinophilic promyelocytes, eosinophilic myelocytes, macrophages, dendritic cells, lymphoblasts, prolymphocytes, natural killer (NK)-cells, small lymphocytes, T-lymphocytes, B-lymphocytes, plasma cells, and lymphoid dendritic cells. In preferred embodiments, the target cell type is one or more erythroid cells, e.g., proerythroblast, basophilic erythroblast, polychromatic erythroblast, orthochromatic erythroblast, polychromatic erythrocyte, and erythrocyte (RBC).

### Vectors

In some embodiments, the engineered transposable element and/or the nucleic acid sequence encoding the transposase is present in one or more vectors.

Various suitable vectors may be used in the present application. In some embodiments, the vector is a plasmid vector, a cosmid vector, an artificial chromosome (for example a bacterial artificial chromosome, a yeast artificial chromosome or a mammalian artificial chromosome), a viral vector such as a bacteriophage, baculovirus, retrovirus, lentivirus, adenovirus, Vaccinia virus, semliki forest virus or adeno-associated virus (AAV) vector, all of which are well known and can be purchased from commercial sources. In general, a suitable vector contains an origin of replication functional in at least one organism, a promoter sequence, convenient restriction endonuclease sites, and one or more selectable markers.

In some embodiments, the vector is a plasmid. In some embodiments, the plasmid can be transformed into bacteria to store or to amplify, and can be transfected into a mammalian cell.

Methods of introducing vectors into a mammalian cell are known in the art. The vectors can be transferred into a host cell by physical, chemical, and/or biological methods. It is contemplated that various vector types and vector delivery methods may be used, either alone or in combination, for the present application.

Physical methods for introducing the vector into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the heterologous nucleic acid into a host cell include the use of DNA and RNA vectors. Viral vectors have become the most widely used method for inserting genes into mammalian, e.g., human cells.

Chemical means for introducing the vector into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, the vector is a viral vector. Examples of viral vectors include, but are not limited to, adenoviral vectors, adeno-associated virus (AAV) vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals. In some embodiments, delivery of a transposable element using viral vectors may be useful for gene therapy in combining the high efficiency of gene delivery by the viral vectors and the stability of gene expression enabled by the transposable element. Reference may be made to, for example, Yant, Stephen R., et al. “Transposition from a gutless adeno-transposon vector stabilizes transgene expression in vivo.” Nature biotechnology 20.10 (2002): 999-1005.

### III. Gene Transfer Systems

Another aspect of the present application provides a gene transfer system comprising: 1) an engineered transposable element (such as any one of the transposable elements describe herein); and 2) a transposase, or a nucleic acid encoding a transposase.

In some embodiments, there is provided a gene transfer system comprising: 1) an engineered transposable element, comprising from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; and 2) a transposase. In some embodiments, the transposable element exhibits transposition activity that allows the heterologous nucleic acid to be inserted into the DNA of a cell (e.g., mammalian cell or plant cell). In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a gene transfer system comprising: 1) an engineered transposable element, comprising from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; and 2) a nucleic acid (e.g., DNA or RNA) encoding a transposase. In some embodiments, the transposable element exhibits transposition activity that allows the heterologous nucleic acid to be inserted into the DNA of a cell (e.g., mammalian cell or plant cell). In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a gene transfer system comprising: 1) an engineered transposable element, comprising from 5′ to 3′: a 5′ terminal repeat sequence (5′TR), a heterologous nucleic acid, and a 3′ terminal repeat sequence (3′TR); and 2) a transposase comprising an amino acid sequence selected from the group consisting of SEQ ID NOs: 53-78 and 103-114, or a variant thereof. In some embodiments, the transposable element exhibits transposition activity that allows the heterologous nucleic acid to be inserted into the DNA of a cell (e.g., mammalian cell or plant cell). In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a gene transfer system comprising: 1) an engineered transposable element, comprising from 5′ to 3′: a 5′ terminal repeat sequence (5′TR), a heterologous nucleic acid, and a 3′ terminal repeat sequence (3′TR); and 2) a nucleic acid (e.g., DNA or RNA) encoding a transposase comprising an amino acid sequence selected from the group consisting of SEQ ID NOs: 53-78 and 103-114, or a variant thereof. In some embodiments, the transposable element exhibits transposition activity that allows the heterologous nucleic acid to be inserted into the DNA of a cell (e.g., mammalian cell or plant cell). In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a gene transfer system comprising: 1) an engineered transposable element; and 2) a transposase, wherein the transposable element comprises from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; wherein the transposase comprises an amino acid sequence selected from the group consisting of SEQ ID NOs: 53-78 and 103-114, or a variant thereof. In some embodiments, the transposable element exhibits transposition activity that allows the heterologous nucleic acid to be inserted into the DNA of a cell (e.g., mammalian cell or plant cell). In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a gene transfer system comprising a gene transfer system comprising: 1) an engineered transposable element; and 2) a nucleic acid (e.g., DNA or RNA) encoding a transposase, wherein the transposable element comprises from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; wherein the transposase comprises an amino acid sequence selected from the group consisting of SEQ ID NOs: 53-78 and 103-114, or a variant thereof. In some embodiments, the transposable element exhibits transposition activity that allows the heterologous nucleic acid to be inserted into the DNA of a cell (e.g., mammalian cell or plant cell). In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 3, a variant thereof, or a fragment thereof, wherein the 3′TR comprises the nucleic acid sequence of SEQ ID NO: 29, a variant thereof, or a fragment thereof; and 2) a transposase comprising the amino acid sequence of SEQ ID NO: 55, or a nucleic acid encoding the transposase.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 8, a variant thereof, or a fragment thereof, wherein the 3′TR comprises the nucleic acid sequence of SEQ ID NO: 34, a variant thereof, or a fragment thereof; and 2) a transposase comprising the amino acid sequence of SEQ ID NO: 60, or a nucleic acid encoding the transposase.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 11, a variant thereof, or a fragment thereof, wherein the 3′TR comprises the nucleic acid sequence of SEQ ID NO: 37, a variant thereof, or a fragment thereof; and 2) a transposase comprising the amino acid sequence of SEQ ID NO: 63, or a nucleic acid encoding the transposase.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 12, a variant thereof, or a fragment thereof, wherein the 3′TR comprises the nucleic acid sequence of SEQ ID NO: 38, a variant thereof, or a fragment thereof; and 2) a transposase comprising the amino acid sequence of SEQ ID NO: 64, or a nucleic acid encoding the transposase.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 13, a variant thereof, or a fragment thereof, wherein the 3′TR comprises the nucleic acid sequence of SEQ ID NO: 39, a variant thereof, or a fragment thereof; and 2) a transposase comprising the amino acid sequence of SEQ ID NO: 65, or a nucleic acid encoding the transposase.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 16, a variant thereof, or a fragment thereof, wherein the 3′TR comprises the nucleic acid sequence of SEQ ID NO: 42, a variant thereof, or a fragment thereof; and 2) a transposase comprising the amino acid sequence of SEQ ID NO: 68, or a nucleic acid encoding the transposase.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 22, a variant thereof, or a fragment thereof, wherein the 3′TR comprises the nucleic acid sequence of SEQ ID NO: 48, a variant thereof, or a fragment thereof; and 2) a transposase comprising the amino acid sequence of SEQ ID NO: 74, or a nucleic acid encoding the transposase.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 23, a variant thereof, or a fragment thereof, wherein the 3′TR comprises the nucleic acid sequence of SEQ ID NO: 49, a variant thereof, or a fragment thereof; and 2) a transposase comprising the amino acid sequence of SEQ ID NO: 75, or a nucleic acid encoding the transposase.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 79, a variant thereof, or a fragment thereof, wherein the 3′TR comprises the nucleic acid sequence of SEQ ID NO: 91, a variant thereof, or a fragment thereof; and 2) a transposase comprising the amino acid sequence of SEQ ID NO: 103, or a nucleic acid encoding the transposase.

In some embodiments, there is provided a gene transfer system comprising: 1) an engineered transposable element comprising, from 5′ to 3′: a 5′ terminal repeat sequence (5′TR), a heterologous nucleic acid, and a 3′ terminal repeat sequence (3′TR), wherein the 5′TR comprises the nucleic acid sequence of SEQ ID NO: 82, a variant thereof, or a fragment thereof, wherein the 3′TR comprises the nucleic acid sequence of SEQ ID NO: 94, a variant thereof, or a fragment thereof; and 2) a transposase comprising the amino acid sequence of SEQ ID NO: 106, or a nucleic acid encoding the transposase.

The gene transfer systems described herein comprises a transposase. The transposase may be present as a polypeptide. Alternatively, the transposase is present as a polynucleotide that includes a coding sequence encoding a transposase. The polynucleotide can be RNA, for instance an mRNA encoding the transposase, or DNA, for instance a coding sequence encoding the transposase. When the transposase is present as a coding sequence encoding the transposase, in some embodiments, the coding sequence may be present in the same vector that includes the transposable element, i.e., in cis. In some embodiments, the gene transfer system comprises a first vector comprising the engineered transposable element, and a second vector comprising the transposase coding sequence, i.e., in trans.

In some embodiments, the gene transfer system comprises: 1) a vector comprising an engineered transposable element (such as any one of the engineered transposable elements described herein); and 2) a transposase.

In some embodiments, the gene transfer system comprises: 1) an engineered transposable element (such as any one of the engineered transposable elements described herein); and 2) a nucleic acid encoding a transposase, wherein the nucleic acid is DNA. In some embodiments, the engineered transposable element and the nucleic acid are present in a single vector. In some embodiments, the engineered transposable element and the nucleic acid are present in separate vectors.

In some embodiments, the gene transfer system comprises: 1) an engineered transposable element (such as any one of the engineered transposable elements described herein); and 2) a nucleic acid encoding a transposase, wherein the nucleic acid is RNA. In some embodiments, the engineered transposable element and the nucleic acid are present in a single vector. In some embodiments, the engineered transposable element and the nucleic acid are present in separate vectors.

There are many potential and suitable combinations of intracellular delivery methods for the engineered transposable element and the transposase or nucleic acid encoding the transposase in the gene transfer systems described herein. The transposase may be delivered as a DNA, RNA, or protein. The engineered transposable element and the transposase may be delivered together or separately. For example, both the transposon and the transposase gene can be contained together in the same recombinant viral genome; a single infection delivers both parts of the system such that expression of the transposase directs cleavage of the transposon from the recombinant viral genome for subsequent integration into a cellular chromosome. In another example, the transposase and the transposable element can be delivered separately by a combination of viruses and/or non-viral systems such as lipid-containing reagents. In these cases, either the transposable element and/or the transposase gene can be delivered by a recombinant virus. In some embodiments, the transposase directs liberation of the transposon from its donor DNA for integration into a target location.

The transposase can be provided to the cell as protein or as nucleic acid encoding the transposase protein. Nucleic acid encoding the transposase protein can take the form of DNA or RNA. The protein can be introduced into the cell alone, or in a vector, such as a plasmid or a viral vector. Further, the nucleic acid encoding the transposase protein can be stably or transiently incorporated into the genome of the cell to facilitate temporary or prolonged expression of the transposase protein in the cell. Further, promoters or other expression control regions can be operably linked with the nucleic acid encoding the transposase protein to regulate expression of the protein in a quantitative or in a tissue-specific manner. In some embodiments, the transposase protein contains a DNA-binding domain, a catalytic domain (having transposase activity), and/or a nuclear localization signal (NLS).

As such, various methods and materials may be used in delivering the gene transfer system of the present application to a cell.

By way of example, one approach is to use plasmid vectors to deliver the gene transfer system. By way of example, the system may consist of two plasmids, one helper plasmid carrying the transposase expression cassette and one donor plasmid carrying the transposable element. After transfection, both plasmids find way to the nucleus, allowing production of transposase-encoding RNA from the helper plasmid and subsequent excision of the transposable element from the donor plasmid facilitated by transposase subunits imported into the nucleus. This approach can be further refined by placing both the transposase gene and the transposable element on a single plasmid, originally referred to as helper-independent transposable element-transposase vectors. Alternatively, transfected in vitro-transcribed mRNA may serve as a rich source of transposase, eliminating the risk of creating cells with prolonged expression of the transposase.

Accordingly, in some embodiments, the transposable element is present in a first vector (a donor vector), and a nucleic acid encoding a transposase is present in a second vector (a helper vector). In some embodiments, the first vector and the second vector are used to co-transfect a cell for transposition.

Another approach is to use viral vectors for the delivery of the gene transfer system. Although viral vectors could have immunogenic or carcinogenic issues, the high delivery efficiencies may be desirable in certain applications. The components of the gene transfer system may be carried and delivered by viral capsids, providing otherwise episomal vectors—like adenoviral or herpes simplex virus-based vectors—the ability to integrate genes and establish long-term transgene expression. The viral coat may provide vector stability, tissue-specific transposable element delivery and transport across the cellular membrane, while the transposable element facilitates viral vector integration according to the characteristic integration profile of the transposable element. Methods and techniques of viral vector-based delivery are known in the art. For example, viral vector-based transfer of the Sleeping Beauty transposon system was first demonstrated in mouse liver with adenoviral vectors, and recent studies have demonstrated the applicability of this approach in larger animals. Adeno-associated viral vectors have also been adapted as carriers of the Sleeping Beauty system. See. e.g., Yant, Stephen R., et al. “Transposition from a gutless adeno-transposon vector stabilizes transgene expression in vivo.” Nature biotechnology 20.10 (2002): 999-1005, Hausl, Martin A., et al. “Hyperactive sleeping beauty transposase enables persistent phenotypic correction in mice and a canine model for hemophilia B.” Molecular Therapy 18.11 (2010): 1896-1906, and Zhang, Wenli, et al. “Hybrid adeno-associated viral vectors utilizing transposase-mediated somatic integration for stable transgene expression in human cells.” PloS one 8.10 (2013).

As discussed above, the gene transfer system of the present application may be delivered into a host cell by physical, chemical, biological methods, or a combination thereof. It is contemplated that various delivery vectors and methods may be used for the present application, either alone or in combination, in order to achieve desirable results for certain applications. Those skilled in the art are thereby enabled to best utilize the various delivery methods and techniques with various modifications as are suited to the particular use contemplated, for example, gene therapy. For gene therapy to be practical, one should achieve stable integration of a therapeutic transgene in the genome of an afflicted tissue to provide a long-term and cost-effective treatment. For example, to achieve a high-efficiency and low-immunogenic gene transfer into patients, one may combine the use of synthetic compounds and plasmids for delivering DNA into a cell. Liposomes and other nanoparticles may be sufficient for this task. For instance, two plasmids can be delivered to the patient: one that provides expression of the transposase (a helper plasmid), and another that provides the transposable element containing a therapeutic transgene (the donor plasmid). These DNAs can be complexed with liposomes and administered via parenteral injection. Upon entering a cell, the transposase may bind to the transposable element in the donor plasmid, excise it, and then integrate it into the genome. Such insertions will be stable and permanent. The helper and donor plasmids may eventually be lost by cellular- and host-defense mechanisms, but any genome-integrated transposable element, containing the therapeutic transgene, will be stable and permanent modifications. The transient nature of these plasmids also curtails excessive transposition, and thus minimizes the risk of carcinogenesis.

Further details and exemplary transposable element delivery methods and techniques may be found in, e.g., Skipper, Kristian Alsbjerg, et al. “DNA transposon-based gene vehicles-scenes from an evolutionary drive.” Journal of biomedical science 20.1 (2013): 92.

### IV. Methods

The present application further provides methods of inserting a heterologous nucleic acid into a target nucleic acid, comprising: contacting the target nucleic acid with an engineered transposable element comprising the heterologous nucleic acid according to any one of the engineered transposable elements described herein or a gene transfer system comprising the heterologous nucleic acid according to any one of the gene transfer systems described herein. The method may be carried out in vitro, or in a cell.

### In Vivo Methods

In some embodiments, there is provided a method of inserting a heterologous nucleic acid into a target nucleic acid in vino, comprising contacting the target nucleic acid with an engineered transposable element comprising: 1) an engineered transposable element; and 2) a transposase, wherein the transposable element comprises from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; wherein the transposase comprises an amino acid sequence selected from the group consisting of SEQ ID NOs: 53-78 and 103-114, or a variant thereof. In some embodiments, the target nucleic acid is a circular DNA. In some embodiments, the target nucleic acid is a linear DNA. In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

The methods described herein are useful for in vitro transposition, including in a cell-free system. See, for example, Goryshin, Igor Yu, and William S. Reznikoff. “Tn5 in vitro transposition.” Journal of Biological Chemistry 273.13 (1998): 7367-7374. Transposable elements exhibiting in vitro transposition activity may be useful for next-generation sequence (NGS) library construction, including, for example, tagmentation methods.

In some embodiments, there is provided a method of preparing a plurality of barcoded nucleic acids from a target nucleic acid, comprising contacting the target nucleic acid with an engineered transposable element comprising: 1) an engineered transposable element; and 2) a transposase, wherein the transposable element comprises from 5′ to 3′: a 5′ terminal repeat sequence (5′TR), a heterologous nucleic acid comprising a barcode sequence, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; wherein the transposase comprises an amino acid sequence selected from the group consisting of SEQ ID NOs: 53-78 and 103-114, or a variant thereof, thereby provided a plurality of barcoded nucleic acids. In some embodiments, the target nucleic acid is genomic DNA. In some embodiments, the target nucleic acid is cDNA. In some embodiments, the target nucleic acid is amplified DNA. In some embodiments, the heterologous nucleic acid further comprises a primer sequence. In some embodiments, the method further comprises amplifying the plurality of barcoded nucleic acids to provide a nucleic acid sequencing library. In some embodiments, the method further comprises sequencing the nucleic acid sequencing library. In some embodiments, the method comprises contacting the target nucleic acid with a plurality of engineered transposable element, wherein each engineered transposable element comprises a unique barcode sequence. In some embodiments, the nucleic acid sequencing libraries prepared using the in vitro methods described herein preserve contiguity information in a target nucleic acid sequence. In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, tagmentation methods are provided using any one of the transposases and/or TR sequences described herein. Tagmentation methods using Tn5 transposon are known in the art, for example, in U.S. Pat. No. 9,080,211B2, which is incorporated herein by reference in its entirety. The tagmentation methods described herein uses transposome complex compositions.

In some embodiments, there is provided a transposome complex composition, comprising: a transposase comprising an amino acid sequence selected from the group consisting of SEQ ID NOs: 53-78 and 103-114, or a variant thereof and a heterologous nucleic acid comprising one or two TR sequences and a tag sequence. In some embodiments, transposome complex comprises a single heterologous nucleic acid that forms a hairpin. In some embodiments, the hairpin comprises a cleavable site.

In some embodiments, the transposome complex comprises two of the transposases bound to two heterologous nucleic acids. In some embodiments, there is provided a transposome complex composition, comprising: a first transposase bound to a first heterologous nucleic acid comprising a 5′TR sequence and a first tag sequence, and a second transposase bound to a second heterologous nucleic acid comprising a 3′TR sequence and a second tag sequence. In some embodiments, the 5′TR comprises a nucleic acid sequence selected from the group consisting of SEQ ID NOs: 1-26 and 79-90, a variant thereof, or a fragment thereof, and wherein the 3′TR comprises a nucleic acid sequence selected from the group consisting of SEQ ID NOs: 27-52 and 91-102, a variant thereof, or a fragment thereof. In some embodiments, the first tag sequence is different from the second tag sequence. In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a method for preparing a library of nucleic acid (e.g., DNA) fragments having first and second tag sequences for a target nucleic acid, comprising contacting the target nucleic acid with a plurality of transposome complexes comprising: (1) a first transposome complex comprising a first transposase and a first heterologous nucleic acid comprising a TR sequence and a first tag sequence; and (2) a second transposome complex comprising a second transposase and a second heterologous nucleic acid comprising a TR sequence and a second tag sequence, wherein the first tag sequence is different from the second tag sequence, and wherein the transposase comprises an amino acid sequence selected from the group consisting of SEQ ID NOs: 53-78 and 103-114, or a variant thereof; wherein the first heterologous nucleic acid and the second heterologous nucleic acid are inserted into the target nucleic acid and the target nucleic acid is fragmented into a multitude of nucleic acid fragments comprising one of the first or the second nucleic acids attached to each 5′ end of the nucleic acid fragments; thereby providing the library of nucleic acid fragments. In some embodiments, the transposome complex of (1) comprises two of the first heterologous nucleic acids, and the transposome complex of (2) comprises two of the second heterologous nucleic acids. In some embodiments, the 5′TR comprises a nucleic acid sequence selected from the group consisting of SEQ ID NOs: 1-26 and 79-90, a variant thereof, or a fragment thereof, and wherein the 3′TR comprises a nucleic acid sequence selected from the group consisting of SEQ ID NOs: 27-52 and 91-102, a variant thereof, or a fragment thereof. In some embodiments, the method further comprises amplifying the nucleic acid fragments. In some embodiments, the method further comprises sequencing the nucleic acid fragments or amplicons thereof. In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, the engineered transposable element or transposome complex inserts into the target nucleic acids in a random fashion, i.e., without bias for specific sequence motifs. In some embodiments, the engineered transposable element or transposome complex inserts into the target nucleic acids more randomly than a PB, SB, or TB transposon.

In some embodiments, the engineered transposable element or transposome complex preferentially inserts into sterically free regions of a target nucleic acid, such as open chromatin region, regions free from binding by nucleosome or other DNA binding proteins. The in vitro methods described herein can thus be used to prepare nucleic acid sequencing libraries for assays to study epigenomics (e.g., chromatin remodeling or DNA methylation), including, but not limited to, Assay for Transposase-Accessible Chromatin with high-throughput sequencing (ATAC-seq), Cleavage Under Targets and Tagmentation (CUT&TAG), Assay for Transposase-Accessible Chromatin and DNA methylation (ATAC-Me), and Transposase-mediated analysis of chromatin looping (Trac-looping). ATAC-seq, CUT&TAG, ATAC-Me and Trac-looping assays using Tn5 transposon have been described, for example, in Buenrostro, Jason D., et al. “Transposition of native chromatin for fast and sensitive epigenomic profiling of open chromatin, DNA-binding proteins and nucleosome position.” Nature methods (2013) 10(2): 1213; Kaya-Okur H S, et al., “CUT&Tag for efficient epigenomic profiling of small samples and single cells.” Nature Communications (2019), 10(1):1-10; Barnett K R et al. “ATAC-Me Captures Prolonged DNA Methylation of Dynamic Chromatin Accessibility Loci during Cell Fate Transitions.” Molecular Cell, 2020; and Lai B. et al. “Trac-looping measures genome structure and chromatin accessibility,” Nature methods, 2018, 15(9): 741, which are incorporated herein by reference in their entirety. In some embodiments, the sequencing library preparation is for use in Assay for Transposase-Accessible Chromatin with high-throughput sequencing (ATAC-seq).

In some embodiments, the engineered transposable element or transposome complex can be used to tag regions of a target nucleic acid (e.g., a genomic DNA) that are spatially in proximity. Two regions that are spatially in proximity with each other may contain the same pair of tag sequences at the ends. The in vitro methods described herein can thus be used to prepare fluorescently labeled probes useful for in situ hybridization to chromatin interaction boundaries in genomic DNA, for example, in a transposase-based fluorescence in situ hybridization (FISH). Tn5-based FISH methods have been described, for example, in Zhang X. et al. “Imaging chromatin interactions at sub-kilobase resolution via Tn5-FISH,” bioRxiv, 2019:601690, which is incorporated by reference in its entirety.

### Methods in Cell

In some embodiments, there is provided a method of inserting a heterologous nucleic acid into a target nucleic acid in a cell, comprising contacting the target nucleic acid with an engineered transposable element comprising: 1) an engineered transposable element; and 2) a transposase, wherein the transposable element comprises from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; wherein the transposase comprises an amino acid sequence selected from the group consisting of SEQ ID NOs: 53-78 and 103-114, or a variant thereof. In some embodiments, the target nucleic acid is genomic DNA. In some embodiments, the target nucleic acid is extrachromosomal DNA. In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a method of inserting a heterologous nucleic acid into a target nucleic acid in a mammalian cell, comprising contacting the target nucleic acid with an engineered transposable element comprising: 1) an engineered transposable element; and 2) a transposase, wherein the transposable element comprises from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; wherein the transposase comprises an amino acid sequence selected from the group consisting of SEQ ID NOs: 53-78 and 103-114, or a variant thereof. In some embodiments, the mammalian cell is a human cell. In some embodiments, the mammalian cell is an animal cell, such as a rodent cell. In some embodiments, the mammalian cell is an immune cell, such as T cell. In some embodiments, the method is carried out ex vivo. In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a method of inserting a heterologous nucleic acid into a target nucleic acid in a plant cell, comprising contacting the target nucleic acid with an engineered transposable element comprising: 1) an engineered transposable element; and 2) a transposase, wherein the transposable element comprises from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; wherein the transposase comprises an amino acid sequence selected from the group consisting of SEQ ID NOs: 53-78 and 103-114, or a variant thereof. In some embodiments, the plant cell is a cell of a crop plant. In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

The transposable elements or gene transfer systems described herein may be introduced into one or more cells using any of a variety of techniques known in the art such as, but not limited to, microinjection, combining the nucleic acid fragment with lipid vesicles, such as cationic lipid vesicles, particle bombardment, electroporation, DNA condensing reagents (e.g., calcium phosphate, polylysine or polyethyleneimine) or incorporating the nucleic acid fragment into a viral vector and contacting the viral vector with the cell. Where a viral vector is used, the viral vector can include any of a variety of viral vectors known in the art including viral vectors selected from the group consisting of a retroviral vector, an adenovirus vector or an adeno-associated viral (AAV) vector.

It is contemplated that the heterologous nucleic acid may contain multiple operons or the heterologous nucleic acid may encode more than one biological products. In some embodiments, the heterologous nucleic acid encodes a genetic circuit. An exemplary genetic circuit is a collection of parts that undergo transcription and/or translation to produce mRNA or proteins, respectively (each an “output” of the part). The part output can interact with other parts (for example to regulate transcription or translation) or can interact with other molecules in the cell (e.g., small molecules, DNA, RNA or proteins that are present in the cellular environment). For example, a circuit can be a metabolic pathway or a genetic cascade, which can be naturally occurring or non-naturally occurring, artificially engineered. Each part in the circuit can include a set of components or genetic modules, e.g., a promoter, ribosome binding site (RBS), coding sequence (CDS) and/or terminator. These components may be interconnected or assembled in different ways to implement different parts, and the resultant parts may be combined in different ways to create different circuits or pathways. In addition to these parts, the circuit may contain additional molecular species that are present in a cell or in the cell's environment that the components interact with.

Accordingly, in some embodiments, there is provided a method of inserting a heterologous nucleic acid into a target nucleic acid in a cell, comprising: contacting the cell with an engineered transposable element comprising: 1) an engineered transposable element; and 2) a transposase, wherein the transposable element comprises from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; wherein the transposase comprises an amino acid sequence selected from the group consisting of SEQ ID NOs: 53-78 and 103-114, or a variant thereof, wherein the heterologous nucleic acid encodes a genetic circuit. In some embodiments, the target nucleic acid is genomic DNA. In some embodiments, the target nucleic acid is an extrachromosomal DNA. In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

Genetic circuits can be useful for gene therapy. Methods and techniques of designing and using genetic circuits are known in the art. Further reference may be made to, for example, Brophy, Jennifer A N, and Christopher A. Voigt. “Principles of genetic circuit design.” Nature methods 11.5 (2014): 508.

The methods are applicable for any suitable cell type. In some embodiments, the cell is a bacterium, a yeast cell, a fungal cell, an algal cell, a plant cell, or an animal cell. In some embodiments, the cell is a cell isolated from natural sources, such as a tissue biopsy. In some embodiments, the cell is a cell isolated from an in vitro cultured cell line. In some embodiments, the cell is a genetically engineered cell. In some embodiments, the cell is a seed cell that undergoes proliferation, differentiation, or both in the core.

In some embodiments, the cell is an animal cell from an organism selected from the group consisting of cattle, sheep, goat, horse, pig, deer, chicken, duck, goose, rabbit, and fish.

In some embodiments, the cell is a plant cell from an organism selected from the group consisting of maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, and a biomass crop.

In some embodiments, the cell is a mammalian cell. In some embodiments, the cell is a human cell. In some embodiments, the human cell is a human embryonic kidney 293T (HEK293T or 293T) cell or a HeLa cell. In some embodiments, the mammalian the mammalian cell is selected from the group consisting of an immune cell, a hepatic cell, a tumor cell, a stem cell, a zygote, a muscle cell, and a skin cell.

In some embodiments, the cell is an immune cell selected from the group consisting of a cytotoxic T cell, a helper T cell, a natural killer (NK) T cell, an iNK-T cell, an NK-T like cell, a γδT cell, a tumor-infiltrating T cell and a dendritic cell (DC)-activated T cell. In some embodiments, the method produces a modified immune cell, such as a CAR-T cell or a TCR-T cell.

In some embodiments, the heterologous nucleic acid is inserted into the genome of the cell. In some further embodiments, insertion of the heterologous nucleic acid inactivates a gene of the cell. In some embodiments, the heterologous nucleic acid encodes a protein or an RNAi molecule.

In addition, the heterologous nucleic acid may encode an RNA molecule that is useful in genome editing. Examples of such RNA molecules include, but are not limited to, CRISPR RNA (crRNA), trans-activating crRNA (tracrRNA), guide RNA (gRNA), and single guide RNA (sgRNA).

In some embodiments, the heterologous nucleic acid encodes a biological product selected from the group consisting of a reporter protein, an antigen-specific receptor, a therapeutic protein, an antibiotic resistance protein, an RNAi molecule, a cytokine, a kinase, an antigen, an antigen-specific receptor, a cytokine receptor, and a suicide polypeptide. For example, the heterologous nucleic acid can encode a receptor specific to a tumor-associated antigen. A T-cell engineered via the method is capable of recognizing and specifically killing the tumor cells expressing the tumor-associated antigen. In another example, the heterologous nucleic acid encodes a hygromycin-resistance protein so that a hygromycin-resistance cell line can be established. Alternatively, the heterologous nucleic acid may not possess any biological function, and can be used to interrupt the function of another gene by inserting itself into an essential gene, thereby interrupting its function.

In some particular embodiments, the heterologous nucleic acid encodes a therapeutic protein that is useful for gene therapy. In some embodiments, the heterologous nucleic acid encodes a therapeutic antibody. In some embodiments, the heterologous nucleic acid encodes an engineered receptor, such as a chimeric antigen receptor (CAR), or an engineered TCR.

In some embodiments, the heterologous nucleic acid comprises one or more multiple cloning sites (MCSs) to facilitate insertion of a polynucleotide of interest (“cargo gene”).

In some embodiments, the method is carried out ex vivo. In some embodiments, the transduced or transfected cell (e.g., mammalian cell) is propagated ex vivo after introduction of the heterologous nucleic acid into the cell. In some embodiments, the transduced or transfected cell is cultured to propagate for at least about any of 1 day, 2 days, 3 days, 4 days, 5 days, 6 days, 7 days, 10 days, 12 days, or 14 days. In some embodiments, the transduced or transfected cell is cultured for no more than about any of 1 day, 2 days, 3 days, 4 days, 5 days, 6 days, 7 days, 10 days, 12 days, or 14 days. In some embodiments, the transduced or transfected cell is further evaluated or screened to select the engineered cell.

Reporter genes or selectable markers may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the heterologous nucleic acid in the cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

The present application contemplates methods of generating isogenic lines of cells of mammalian cells for the study of genetic variations. The present application also contemplates genome modification of microbes, cells, plants, animals or synthetic organisms for the generation of biomedically, agriculturally, and industrially useful products. The methods may be used as a biological research tool, for understanding the genome, e.g. gene knockout or knock-in studies.

Also provided are cells modified by a heterologous nucleic acid using any one of the methods described herein, and organisms (such as animals, plants, or fungi) comprising or produced from such cells.

### Target Nucleic Acid

The methods described herein are suitable for inserting a heterologous nucleic acid into a variety of target nucleic acids. In some embodiments, the target nucleic acid is a DNA. In some embodiments, the target nucleic acid is single-stranded. In some embodiments, the target nucleic acid is double-stranded. In some embodiments, the target nucleic acid comprises both single-stranded and double-stranded regions. In some embodiments, the target nucleic acid is linear. In some embodiments, the target nucleic acid is circular. In some embodiments, the target nucleic acid comprises one or more modified nucleotides, such as methylated nucleotides, damaged nucleotides, or nucleotides analogs. In some embodiments, the target nucleic acid is not modified. In some embodiments, the target nucleic acid is bound to one or more proteins, such as nucleosomes.

The target nucleic acid may be of any length, such as about at least any one of 100 bp, 200 bp, 500 bp, 1000 bp, 2000 bp, 5000 bp, 10 kb, 20 kb, 50 kb, 100 kb, 200 kb, 500 kb, 1 Mb, or longer. In some embodiments, the target nucleic acid is no more than about any one of 500 kb, 200 kb, 100 kb, 50 kb, 40 kb, 30 kb, 20 kb, 10 kb, 5 kb, 2 kb, 1 kb, 500 bp, 200 bp or less. In some embodiments, the target nucleic acid is about any one of 100 bp-500 bp, 500 bp-1 kb, 100 bp-1 kb, 1 kb-2 kb, 100 bp-5 kb, 100 bp-10 kb, 100 bp-20 kb, 1 kb-5 kb, 1 kb-10 kb, 1 kb-20 kb, 20 kb-100 kb, or 100 kb-1 Mb. The target nucleic acid may also comprise any sequence. In some embodiments, the target nucleic acid is enriched for particular sequences, which are hotspots for transposition by the engineered transposable element or gene delivery system described herein. In some embodiments, the target nucleic acid is AT-rich, such as having at least about any one of 40%, 45%, 50%, 55%, 60%, 65%, or higher AT content. In some embodiments, the target nucleic acid is not AT-rich. In some embodiments, the target nucleic acid is not enriched for particular hotspot sequences, as the engineered transposable element or gene delivery system described herein has no preference to insert the heterologous nucleic acid into a particular sequence or sequence motif. In some embodiments, the target nucleic acid has one or more secondary structures or higher-order structures. In some embodiments, the target nucleic acid is not in a condensed state, such as in a chromatin.

In some embodiments, the target nucleic acid is present in a cell. In some embodiments, the target nucleic acid is present in the nucleus of the cell. In some embodiments, the target nucleic acid is endogenous to the cell. In some embodiments, the target nucleic acid is a genomic DNA. In some embodiments, the target nucleic acid is a chromosomal DNA. In some embodiments, the target nucleic acid is a protein-coding gene or a functional region thereof, such as a coding region, or a regulatory element, such as a promoter, enhancer, a 5′ or 3′ untranslated region, etc. In some embodiments, the target nucleic acid is a non-coding gene, such as transposon, miRNA, tRNA, ribosomal RNA, ribozyme, or lincRNA. In some embodiments, the target nucleic acid is a plasmid.

In some embodiments, the target nucleic acid is exogenous to a cell. In some embodiments, the target nucleic acid is a viral nucleic acid, such as viral DNA. In some embodiments, the target nucleic acid is a horizontally transferred plasmid. In some embodiments, the target nucleic acid is integrated in the genome of the cell. In some embodiments, the target nucleic acid is not integrated in the genome of the cell. In some embodiments, the target nucleic acid is a plasmid in the cell. In some embodiments, the target nucleic acid is present in an extrachromosomal array.

In some embodiments, the target nucleic acid is an isolated nucleic acid, such as an isolated DNA. In some embodiments, the target nucleic acid is present in a cell-free environment. In some embodiments, the target nucleic acid is an isolated vector, such as a plasmid. In some embodiments, the target nucleic acid is an isolated linear DNA fragment.

### V. Kits and Articles of Manufacture

The present application also provides kits and articles of manufacture comprising any one of the transposable elements, or gene transfer systems describe herein. In some embodiments, the kit comprises instructions for inserting a heterologous nucleic acid into a target nucleic acid (e.g., using any one of the methods described herein). In some embodiments, the kit is for inserting a heterologous nucleic acid into a target nucleic acid in vitro. In some embodiments, the kit is for inserting a heterologous nucleic acid into a target nucleic acid in a cell, such as mammalian cell or plant cell. The kits and articles of manufacture described herein can be used for modification of a target nucleic acid in vitro or ex vivo, in genetic research, and in gene therapy.

In some embodiments, there is provided a kit comprising an engineered transposable element, comprising from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof, and wherein the transposable element exhibits transposition activity that allows the heterologous nucleic acid to be inserted into the DNA of a cell (e.g., mammalian cell or plant cell). In some embodiments, the heterologous nucleic acid comprises one or more multiple cloning sites (MCSs) to facilitate insertion of a polynucleotide of interest (“cargo gene”). In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, there is provided a kit comprising a gene transfer system comprising: 1) an engineered transposable element; and 2) a transposase, or a nucleic acid encoding a transposase, wherein the transposable element comprises from 5′ to 3′: a 5′ terminal repeat sequence (5′TR), a heterologous nucleic acid, and a 3′ terminal repeat sequence (3′TR), wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; wherein the transposase comprises an amino acid sequence selected from the group consisting of SEQ ID NOs: 53-78 and 103-114, or a variant thereof, and wherein the transposable element exhibits transposition activity that allows the heterologous nucleic acid to be inserted into the DNA of a cell (e.g., mammalian cell or plant cell). In some embodiments, the heterologous nucleic acid comprises one or more multiple cloning sites (MCSs) to facilitate insertion of a polynucleotide of interest (“cargo gene”). In some embodiments, the engineered transposable element is derived from any one of the TEs of Table 2. In some embodiments, the engineered transposable element is derived from Tc1-8B_DR, Tc1-3_FR, Mariner2_AG, Tc1-1_Xt, or Tc1-1_PM.

In some embodiments, the kit comprises one or more reagents for use in any one of the methods described herein. Reagents may be provided in any suitable container. For example, the kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the kit comprises culturing media, buffers, reagents and the like to allow propagation or induction of a cell modified using an engineered transposable element or gene transfer system described herein. In some embodiments, the kit comprises buffers, reagents and the like for isolating and/or preparing modified target nucleic acids using an engineered transposable element or gene transfer system described herein. In some embodiments, the kit comprises primers and reagents for preparation of sequencing libraries using an engineered transposable element or gene transfer system described herein.

The kits are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### EXAMPLES

The examples below are intended to be purely exemplary of the invention and should therefore not be considered to limit the invention in any way. The following examples and detailed description are offered by way of illustration and not by way of limitation.

### Example 1: Identification of Candidate Active Transposable Elements

This example describes the in silico identification of candidate active transposable elements (TEs, transposons) across species. There exists a large amount of transposons in various species. However, only a few number of transposons have been characterized that exhibit transposition activity in mammalian cells. Therefore, there is a need for a methodology to systematically identify candidate active transposons that could be useful as agents for genome engineering and gene therapy. This example concentrates on identification of transposons with terminal inverted repeats (TIRs, also known as terminal repeats-TRs), but the methodology can be used to identify any other types of transposons.

### Materials and Methods

Repbase is the most commonly used transposon database, which holds a collection of 38,000 transposon sequences from a wide range of eukaryotic species (Bao, W., K. K. Kojima, and O. Kohany, Repbase Update, a database of repetitive elements in eukaryotic genomes. Mob DNA, 2015, 6: p. 11). These prototypic sequences are mostly consensus sequences, which are reconstructed for each transposon family to approximate the sequence of its ancestral active status. Thus, the consensus sequences can be used to experimentally reconstruct active transposons for transgenesis and gene therapy, such as Sleeping Beauty (Ivics, Z., et al., Molecular reconstruction of Sleeping Beauty, a Tc1-like transposon from fish, and its transposition in human cells. Cell, 1997, 91(4): p. 501-10). As an initial step of this project, all consensus sequences were downloaded from Repbase (version Repbase24.02) for candidate transposon screening.

Because the consensus sequences do not necessarily contain an intact transposase gene (especially for those ancient transposons that have highly degenerated) and may not function as an active transposon in other systems, such as mammalian cells, several parameters were used to evaluate the identified TE transposition activity in their original species, including important domains encoded by transposase, average sequence divergence between the consensus and family members, copy number, conserved terminal inverted repeats (TR) sequences, and conserved target site duplications (TSDs) flanking the TEs. To obtain the abovementioned information, genome sequences of 100 animals were downloaded from UCSC Genome Browser (Haeussler, M., et al., The UCSC Genome Browser database: 2019 update. Nucleic Acids Res, 2019, 47(D1): p. D853-D858). The genome sequences were masked for repeats using the consensus sequences from Repbase. An active transposon is defined such that: 1) the candidate transposon matches the consensus sequence from start to end; and 2) the length of the candidate transposon reaches 90% length of the consensus transposon to ensure no significant deletions within the transposon.

### Results

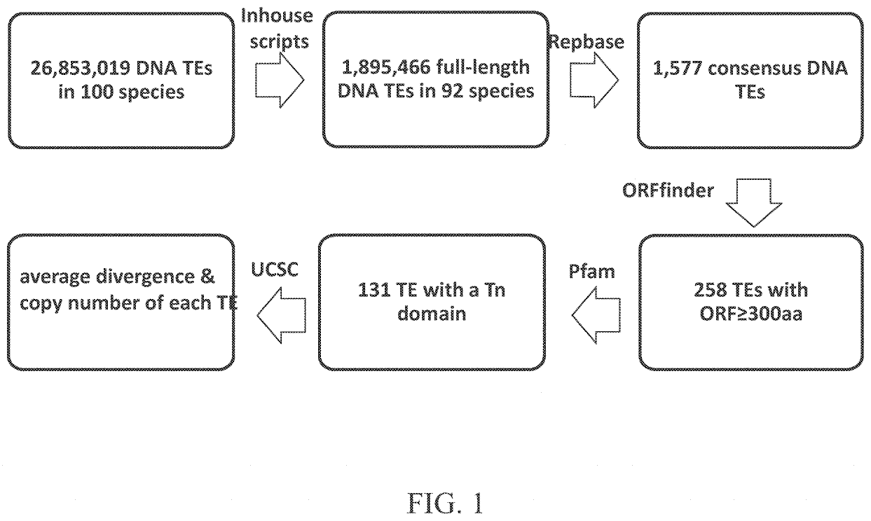

FIG. 1 illustrates a flow chart of the bioinformatics pipeline, as well as the number of candidate active transposable elements at each stage of the pipeline.

In total, 26,853,019 copies of DNA transposons (mostly TIR transposons) were identified from the 100 animal genomes. A large number of the copies were fragments degenerated from the active transposons. The number of full-length DNA transposon copies was 1,895,466 in total, which were mapped to 1,577 consensus DNA transposons in Repbase.

These transposons were then examined to see if they contain a transposase gene. ORFfinder was used to detect open reading frames (ORFs), and the length cutoff value was set to 300 amino acids. The protein sequences were then used to search for important domains against a library of Pfam HMM by PfamScan (Madeira, F., et al., The EMBL-EBI search and sequence analysis tools APIs in 2019. Nucleic Acids Res, 2019, 47(W1): p. W636-W641). After this filtering step, 131 transposons having a Tn domain remained in the pipeline.

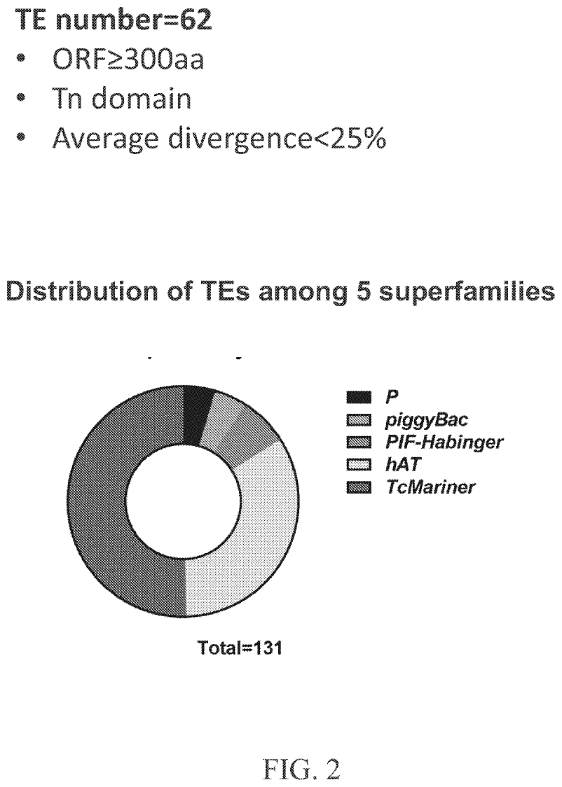

Next, the copy number of each transposon was determined using RepeatMasker (Smit, A F A, Hubley, R & Green, P. RepeatMasker Open-4.0.2013-2015). The average sequence divergence value was calculated between the consensus transposon and its family members in each species, and this divergence value can range from 0 to 24.4% in different species. As shown in Table 1, 62 identified TEs have an average divergence value no greater than 5%, and 69 identified TEs have an average divergence value greater than 5%. These 131 active transposons were found to distribute among five superfamilies (FIG. 2)

Among the 62 identified transposons in Table 1, a total of 11 transposons were found to meet the following stricter criteria, rendering them highly suitable for use in genome engineering: 1) the length of the transposon is less than 3000 bp; 2) the number of miniature inverted-repeat transposable elements (MITEs) within the transposon is greater than 10; and 3) the average divergence value of the transposon is less than 1% (TE IDs: 4, 5, 7, 12, 18, 20, 22, 25, 26, 28 and 30 as shown in Table 1).

Taken together, these data show 131 candidate active transposons identified via a pan-genome bioinformatics analysis of 100 genomes. Thus, this example demonstrates successful development of a robust bioinformatics pipeline for identifying candidate active transposable elements.

### Example 2: Validation of Candidate Active Transposable Elements

This example describes the experimental validation of the candidate active transposable elements identified in Example 1.

### Materials and Methods

### DNA Synthesis and Plasmid Construction

The mammalian codon-optimized transposase ORFs flanked by EcoRI and NotI were synthesized, and cloned into CMV-hyPBase vector (K. Yusa, et al., A hyperactive piggyBac transposase for mammalian applications. Proc Natl Acad Sci USA, 2011, 108(4): p. 1531-6). These vectors were the helper plasmids, for helping transposase expression under a human CMV promoter. Transposon donor plasmids comprise left and right transposon fragments that flank an antibiotic resistance gene. As used herein, a left transposon fragment (LTF) refers to a fragment from the 5′TSD to the start codon of the transposase ORF sequence of a TE sequence. As used herein, a right transposon fragment (RTF) refers to a fragment from the stop codon of the transposase ORF sequence to the 3′TSD of a TE sequence. Generally, the TR sequences are located within the left and right transposon fragments. For instance, the 5′TR sequence is located within the LTF sequence, and the 3′TR sequence is located within the RTF sequence. LTF and RTF sequences used in this experiment were synthesized by Qinglan Biotechnology Inc., and cloned into pMV vectors. 5′ and 3′ multiple cloning sites (MCSs) were also synthesized within the transposon fragments for cargo gene cloning. TSD sequences were located on the outermost sides. The donor plasmids for transposition screening in mammalian cells, carrying a P2A-linked puromycin resistance gene and an enhanced GFP gene expressed by PGK promoter. FIG. 3 shows an exemplary set of helper and donor constructs for use in validating active transposable elements.

### Transposition in Mammalian Cells

Four mammalian cell lines, HEK293T (also known as 293T), HeLa, Hct116 and K562, were used to screen for active transposons. 293T and HeLa cell lines were maintained in DMEM medium and supplemented with 10% fetal bovine serum and 1% penicillin/streptomycin. Hct116 and K562 cell lines were maintained in RPMI 1640 medium and supplemented with 10% fetal bovine serum and 1% penicillin/streptomycin. In addition to these cell lines, CD3⁺ T cell were isolated using the EasySep Human T Cell Enrichment Kit, following collection of mononuclear cells by histopaque-1077 (Sigma-Aldrich) gradient separation, and CD3′ T cell were cultured in and cultured in X-Vivo 15 mediums (Lonza), supplemented with 5% (v/v) heat-inactivated fetal bovine serum, 2 mM L-glutamine and 1 mM sodium pyruvate.

For the transposition assay, 1.2×10⁵ HEK293T cells, 0.7×10⁵ HeLa cells or 1.0×10⁵ Hct116 cells were seeded into individual wells of 24-wells plate 18 h before transfection. Either 200 ng of helper plasmid and 100 ng of donor plasmid, or 100 ng of donor plasmid alone were delivered into each cell line using Lipo3000. Two days after transfection, the number of cells was counted and the transfection efficiency was measured by FACS. Then 1/100^{th} transfected HEK293T, 1/10^{th} transfected HeLa cells or 1/10^{th} transfected Hct116 cells were transferred into 100-mm plates for puromycin (0.5 μg/ml) selection for 10 days (HEK-293T cell line) or 14 days (HeLa cell line and Hct116 cell line).

Following puromycin selection, the cells were washed once with 5 ml cold PBS, then fixed by 4% PFA for 15 min, followed by staining with 0.2% methylene blue (in PBS) for 1 h (Wu et al., piggyBac is a flexible and highly active transposon as compared to Sleeping Beauty, Tol2, and Mos1 in mammalian cells. PNAS, 2006, 103: p. 15008-15013). Finally, the residual non-specific staining was washed off with PBS. Individual stained colonies were counted by Image J software. According to previously counted total number of transfected cells and transfection efficiency, the transposition efficiency was calculated.

For suspension cells, like K562 cell line and CD3+ T cells, transposition activity was assessed based on the percent of GFP positive cells on the 14^{th}, days after electroporation, when plasmids were been diluted to an extremely small proportion.

### Construction of the TEs Insertion Library and Bioinformatics Analysis

Genome DNA was isolated from stable transposition K562 cells using a DNeasy Blood and Tissue Kit (Qiagen, Germany), sheared to an average length of 600 bp using a Covaris M220 ultrasonicator (Covaris, USA). DNA sample was end-repaired, linker ligation and amplified by nested PCRs was purified and sequenced on an Illumina HiSeq sequencer. Then, TEs integration sites were compared to random insertion sites among primary sequence upstream and downstream region flanking the vector integration site, distance to nearest gene and TSS, and different chromatin states.

### Results

The validation project comprised three rounds of experiments.

For the initial round, the 11 TEs with a divergence value less than 1% and a copy number of MITE greater than 10 (i.e., TE IDs: 4, 5, 7, 12, 18, 20, 22, 25, 26, 28 and 30) were first validated. Without wishing to be bound by any theory, it is postulated that a lower divergence value and a higher MITE copy number indicate a higher likelihood of a transposon having been recently active in a species (see R. Mitra, et al., Functional characterization of piggyBat from the bat Myotis lucifugus unveils an active mammalian DNA transposon. Proc Natl Acad Sci USA. 2013, 110(1): p. 234-9).

During plasmid construction, transposon TR sequences from two different sources were found available, one from the consensus sequence provided by the database, and the other from alignment of autonomous and MITE sequences. To test whether the different TR sequence sources would affect assay results, two sets of donor plasmids were designed using TR sequences from the different sources for the transcription assays, respectively.

Results showed that for the 11 candidate TEs tested in the initial validation, three TEs were found to be active ones. Tc1-3_Xt (TE ID 25) was found to be active in both HEK293T and HeLa cells, with a transfer efficiency about 9.6% in HEK293T cells and 3.6% in HeLa cells, equivalent to about half of that of piggyBac (18.4% in HEK293T cells and 8.3% in HeLa cells). Two TEs were found to be active in only one of the two cell lines tested: hAT-3_XT (TE ID 22) was about 1% active in HeLa cells and hAT-5_DR (TE ID 28) was about 2% active in HEK-293T cells. Because no significant differences in transposition efficiency were observed between the two donor plasmid sets having differently sourced TR sequences, later experiments only relied on the TR sequences sourced from consensus sequences in the database.

For the following round of experiments, 48 candidate TEs selected from Table 1 were evaluated for transposition activity in 293T and HeLa cell lines (among them, 16 TEs were tested in 293T cells only). With a surprisingly high success rate, 22 TEs were found to be active from the 48 candidate TEs tested. Among them, eight TEs exhibited a transposition efficiency about equal or even higher than piggyBac: Tc1-8B_DR (TE ID 14), Tc1-3 FR (TE ID 29), Mariner2_AG (TE ID 35), Tc1-1_Xt (TE ID 36), Tc1-1_AG (TE ID 37), Tc1-1_PM (TE ID 43), Tc1-4_Xt (TE ID 54), and Tc1-15_Xt (TE ID 56). In reference, piggyBac had a transposition efficiency of 17.44% in 293T cells and 10.25% in HeLa cells.

For several active TEs, because the transposition efficiency was much higher than expected, the standard dilution of cells was not enough to keep the surviving colonies separate on the assay plate for an accurate counting of the individual colonies. As a result, the number of stained colonies as counted by the image software was underestimated. For those TEs, even though the staining of the assay plate clearly suggested a much higher transposition efficiency, the calculated transposition efficiency based on the underestimated colony counting was only an underestimation of the actual transposition efficiency.

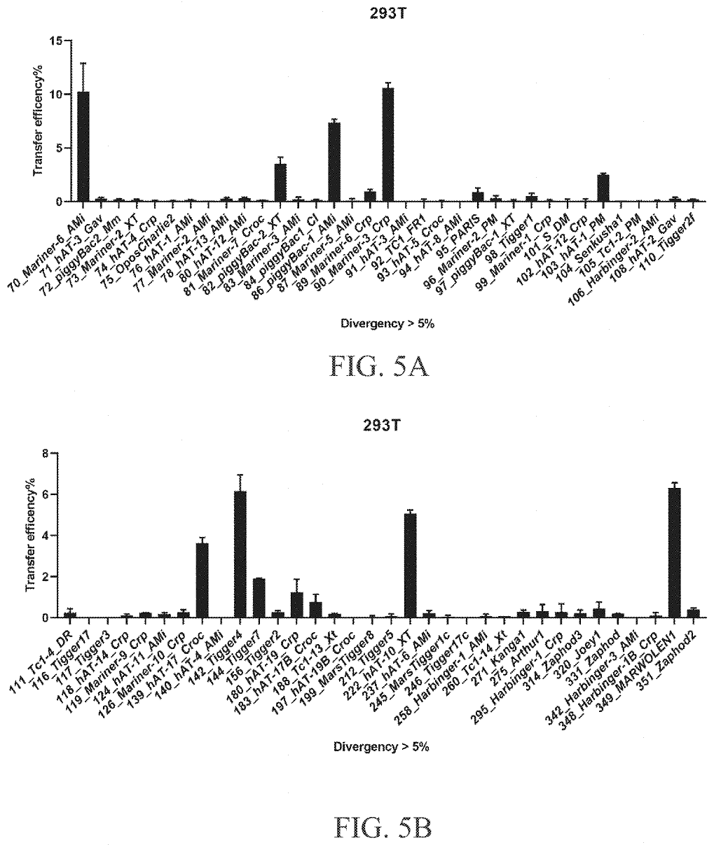

In summary, from two rounds of experiments, 59 candidate TEs were evaluated for transposition activity. FIGS. 4A-5B show the transposition efficiencies of the evaluated TEs as compared to control TEs. A total of 25 TEs were found to have activity in human cell lines.

Among the 25 active TEs, nine TEs belong to the hAT superfamily and 16 TEs belong to the TcMariner superfamily. Eight active TEs had a transposition activity comparable to or even higher than that of piggyBac. These eight highly efficient active TEs all belong to the TcMariner superfamily, suggesting more active transposons distributed in this superfamily. Further, nine of the 25 active TEs are from tropical clawed frog. In addition, no apparent relationships were observed between the transposition activity and the divergence value or the MITE copy number of a TE.

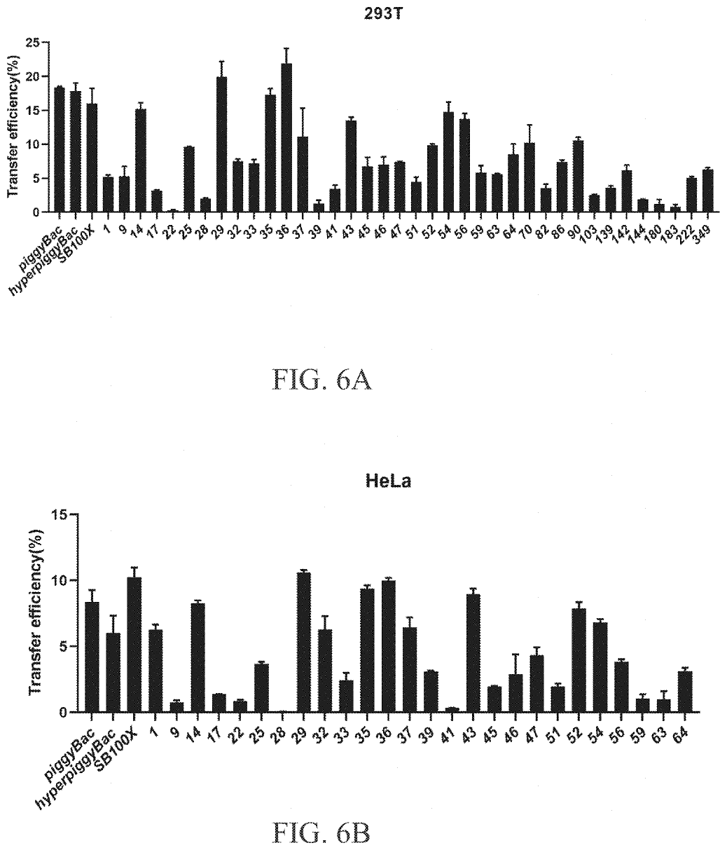

In a 3rd round of experiments, the remaining 72 candidate TEs selected from Table 1 were evaluated for transposition activity in 293T and HeLa cell lines. Among the candidate TEs, 69 TEs with a divergence value greater than 5% were tested in 293T cells only. 13 TEs were found to be active from the 72 candidate TEs tested. Results are shown in FIGS. 6A-6B.

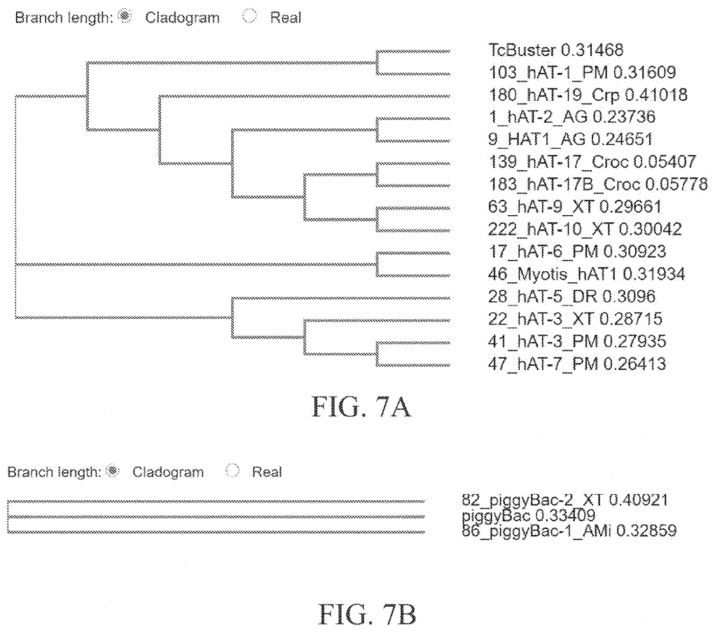

Table 2 summarizes validated active TEs from the above experiments, including a total of 38 TEs were validated active in both 293T cell line and HeLa cell line, or only in one of the two cell lines. Phylogenetic trees of the identified TEs belonging to different superfamilies based on the transposase sequences are shown in FIGS. 7A-7C. Percentage sequence similarities among the various transposases within each superfamily are shown in Tables 3-5.

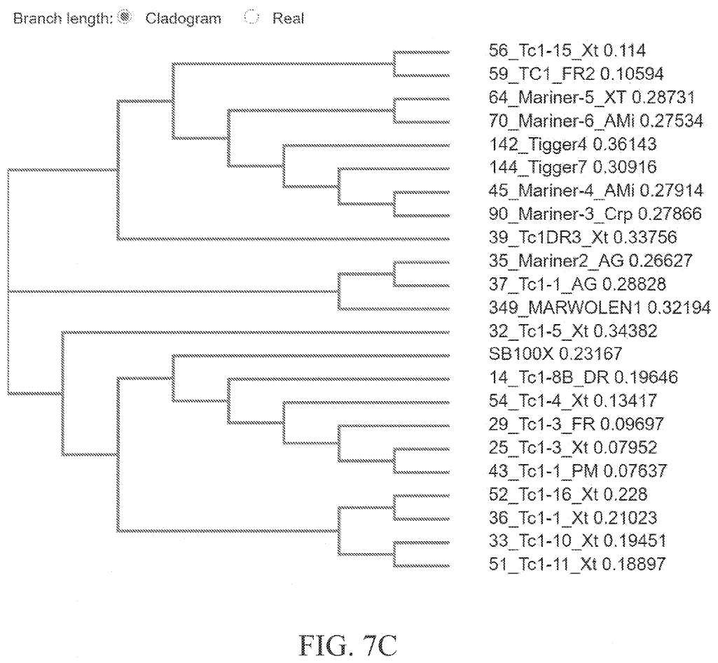

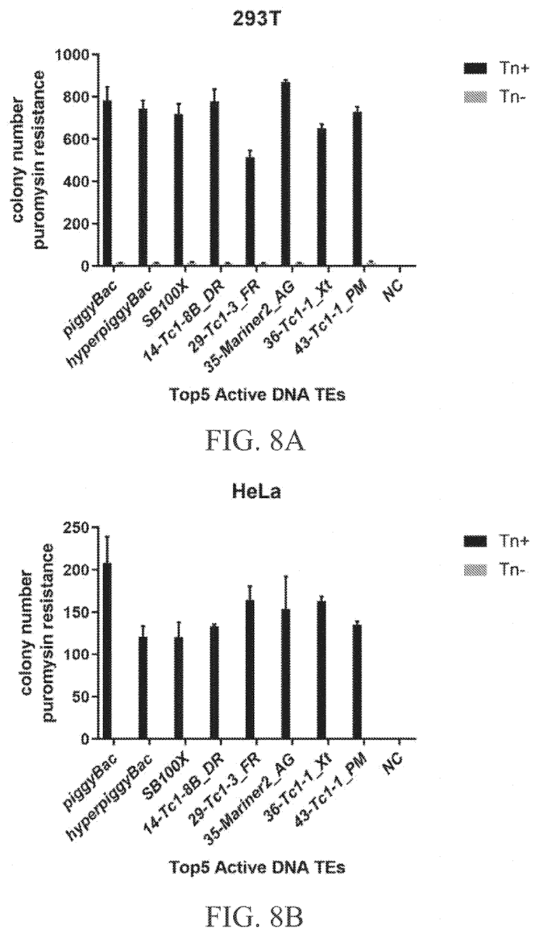

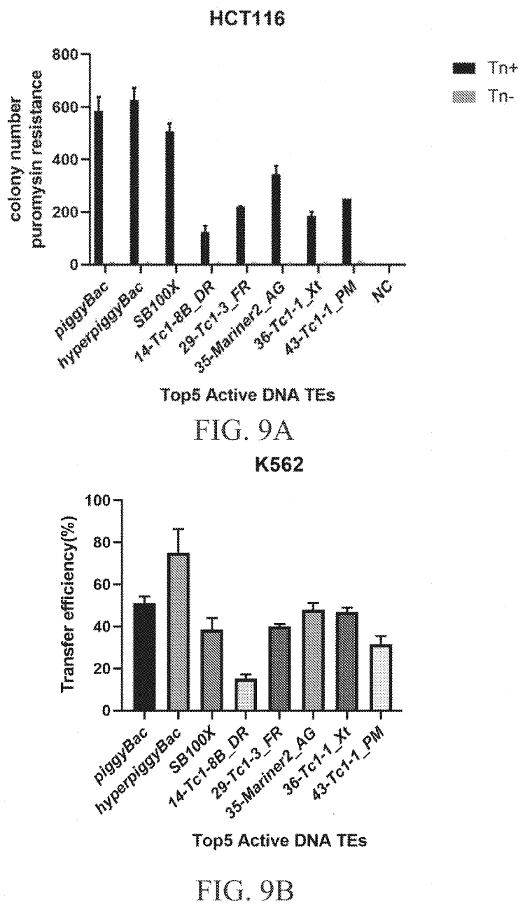

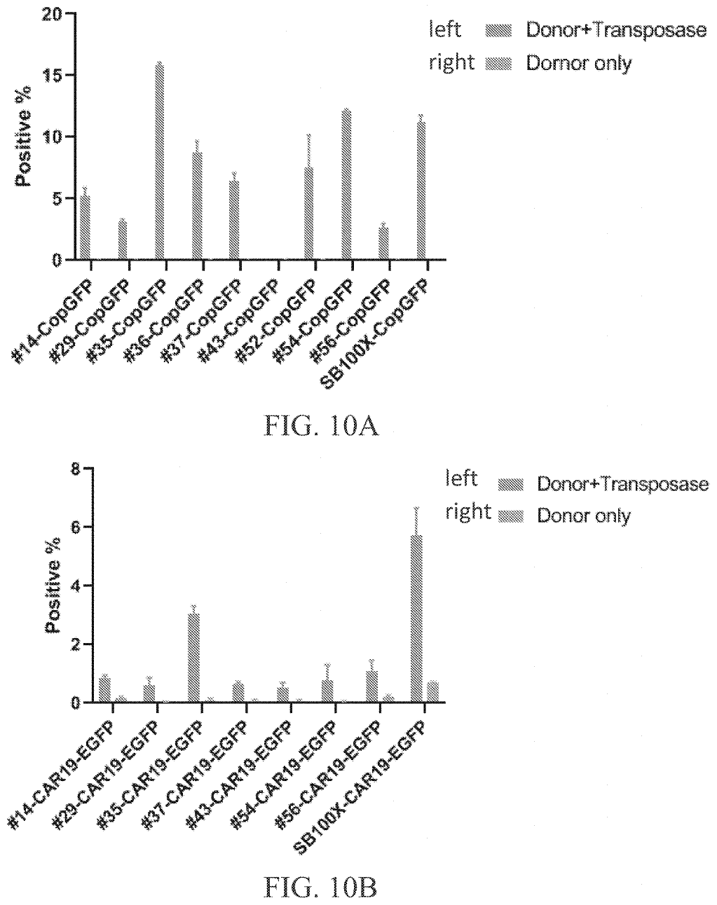

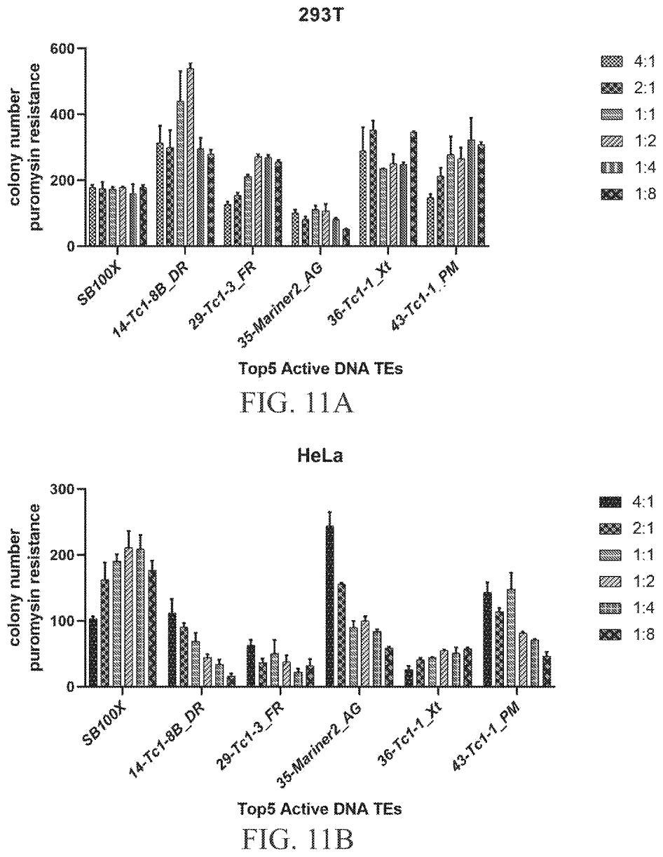

Notably, 5 DNA TEs (Tc1-8B_DR (TE ID 14), Tc1-3_FR (TE ID 29), Mariner2_AG (TE ID 35), Tc1-1_Xt (TE ID 36), Tc1-1_PM (TE ID 43) show superior transposition activity than SB100X, which has been optimized many times. The top 5 active TEs also contain higher transposition activity among HEK293T cells, Hela cells and HCT116 cells (FIGS. 5A-9B), and FIGS. 10A-10B show transposition activity of TEs in primary T cells. The phenomenon known as overproduction inhibition (OPI) didn't occur in the 3 TEs (Tc1-8B_DR (TE ID 14), Tc1-3_FR (TE ID 29), Tc1-1_PM (TE ID 43). FIGS. 11A-11B show increased transposition activity with optimized ratio of helper plasmid encoding transposase to transposon plasmid. FIG. 12 shows the cargo capacity, including 2 kb, 5 kb, 10 kb and 20 kb gene length, of top5 active TEs, compared to identified control TEs, piggyBAC, hyperPiggyBac and SB100X.

Comparison of the active TEs with the inactive TEs among the 131 TE candidates show that active TEs exhibited lower average diversity, slightly longer predicted TIR sequence and a significant increase in autonomous TEs numbers. These differences are helpful differentiating features that can be used in the bioinformatics pipeline for identifying active TEs, including both preliminary and large-scale screening settings.

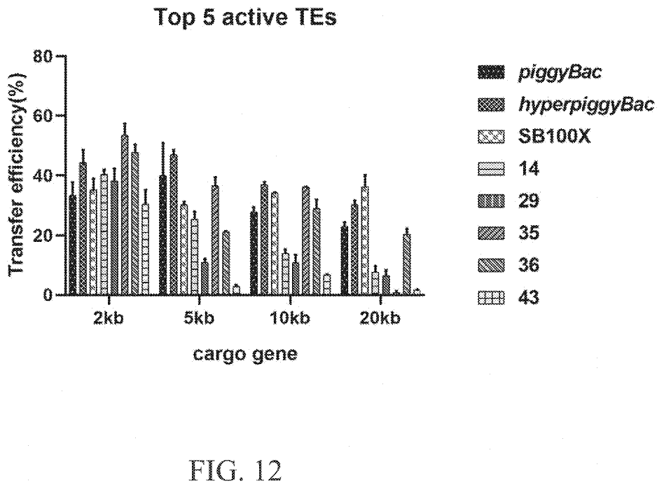

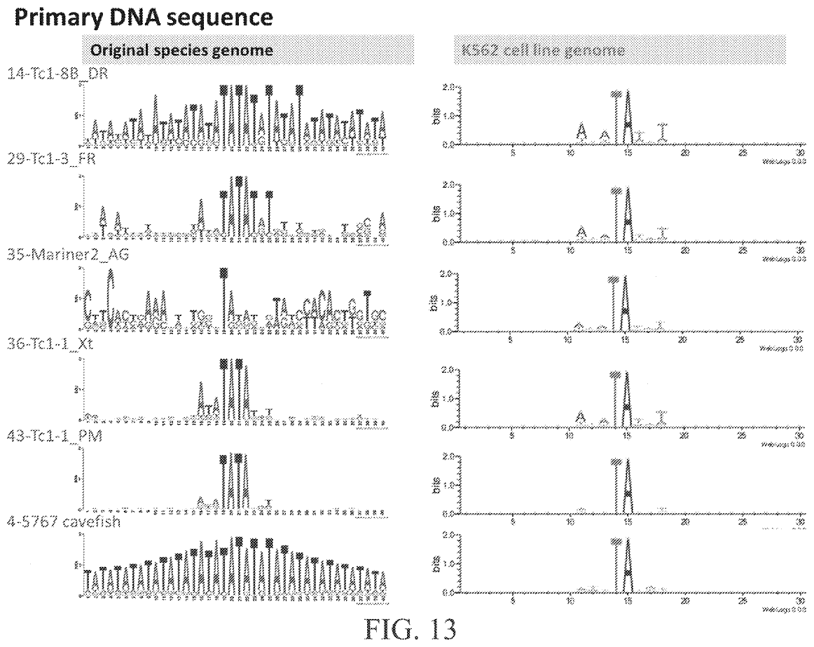

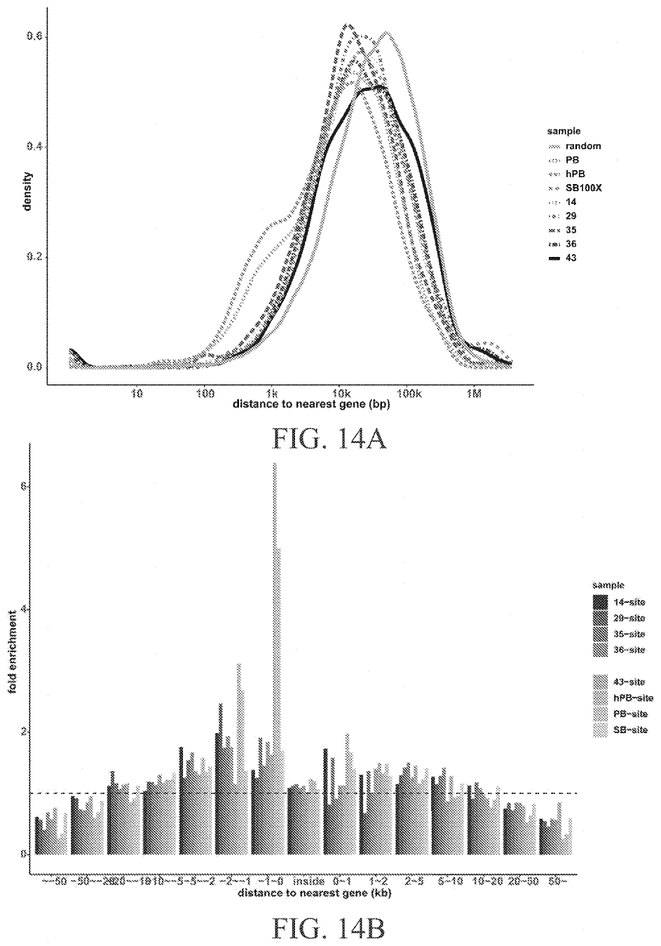

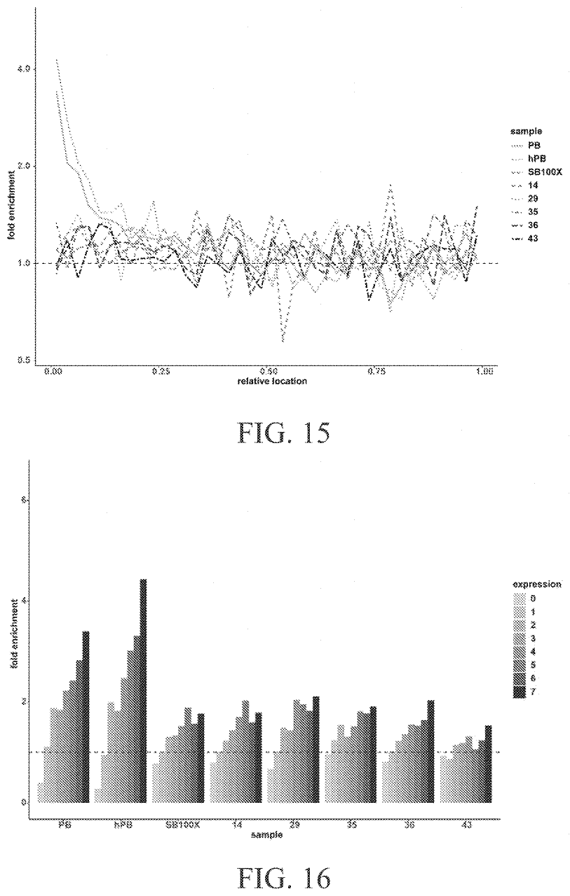

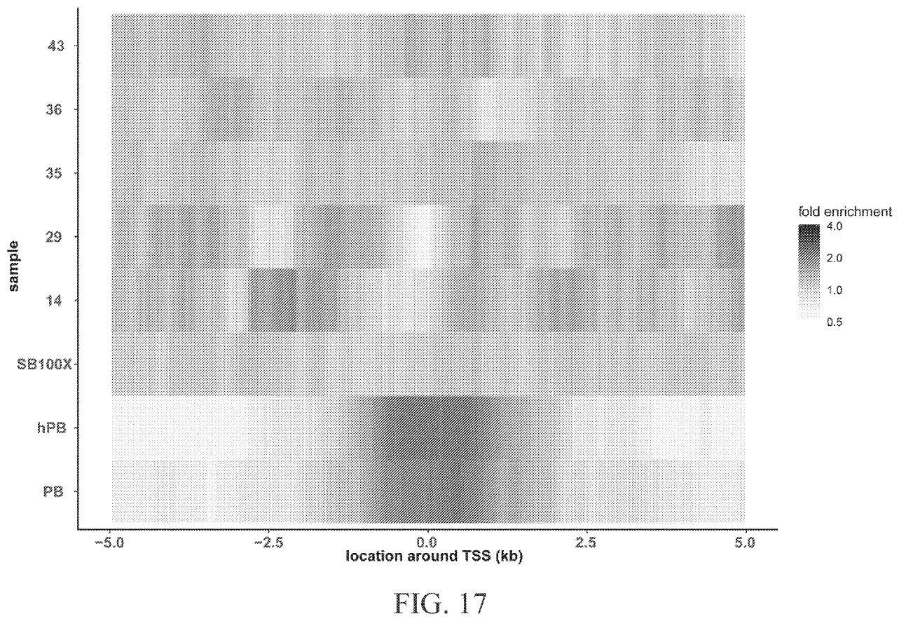

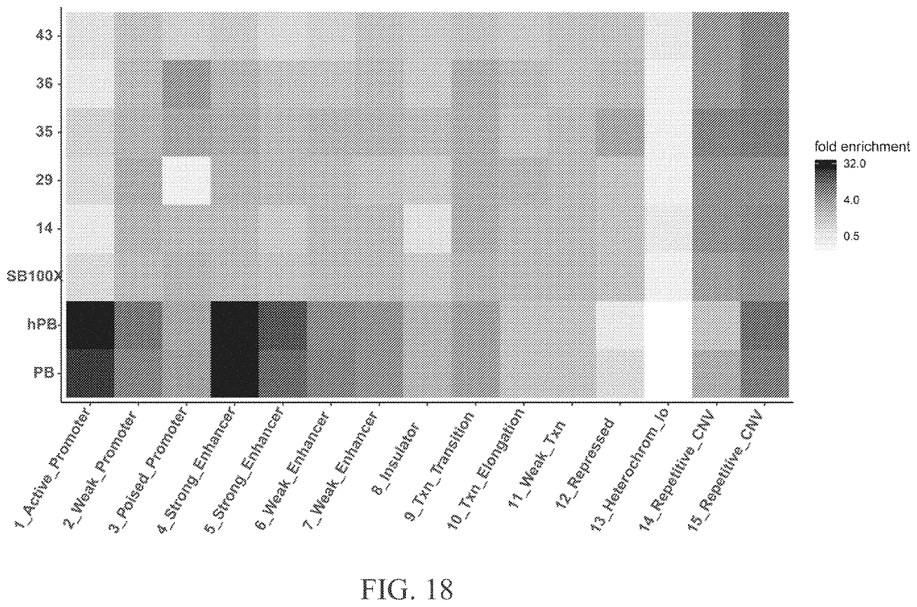

The retrieved integration sites at transposon integration sites revealed the highly preferred TA target site dinucleotides for the top 5 active TEs (FIG. 13). The target site dinucleotide of TEs belong to Tc/Mariner superfamily and is very conservative. FIGS. 14A-18 show frequencies of integration into genomic features, including distance to genes and transcription start sites (TSS), different chromatin states, by comparing computer-generated random data and control TEs, piggyBAC. The data show that all of the top 5 active TEs have very low preference for integration near gene sequences and no preference in the upstream and downstream sequences of genes and TSS. With respect to gene expression levels and chromatin states, the top 5 active TEs also show nearly random integration patterns, and they tend to not insert in hyperactive expression regions.

Taken together, these data exhibited successful establishment of a robust bioinformatics pipeline for identifying candidate active transposable elements, and successful experimental validation of the identified TEs. The transposition efficiencies and integration patterns of the various TEs suggest that these TEs may be useful in genome engineering applications and gene therapies.

TABLE 1
Candidate transposable elements (TEs) identified from the bioinformatics pipeline.

| | | | | | | | | | | | | | | | |
|---|---|---|---|---|---|---|---|---|---|---|---|---|---|---|---|
| | TE | | | | | | | | | | | | | | |
| | name/ | | | | | | | | | Auto, | | Total | | | |
| TE | Repbase | Super | | Length | Diver- | | | | 5′ TSD, | copy | MITE | copy | | | NCBI |
| ID | ID | family | Species | (bp) | gence | 5′TR | 3′TR | Transposase | 3′ TSD | # | copy # | # | LTF | RTF | LD |

Divergence < 5%

| | | | | | | | | | | | | | | | |
|---|---|---|---|---|---|---|---|---|---|---|---|---|---|---|---|
| 1 | hAT- | hAT | A. gambiae | 4,80 | 0.00% | SEQ ID | SEQ ID | SEQ ID | SEQ ID | 1 | 14 | 15 | SEQ ID | SEQ ID | |
| | 2_AG | | | 3 | | NO: 1 | NO: 27 | NO: 53 | NO: 191 | | | | NO: 115 | NO: 141 | |
| 2 | AgaP1 | P | A. gambiae | 5,64 | 0.00% | AgaP12:1- | AgaP12:562 | AgaP12:529 | gtegaaa | 1 | 15 | 16 | AgaP12:1- | AgaP12:2 | 2445005 |
| | 2 | | | 9 | | 26 | 4-5649 | -2346 | c | | | | 528 | 347-5649 | _L2 |
| 3 | P3_AG | P | A. gambiae | 4,39 | 0.00% | P3_AG:1-29 | P3_AG:436 | P3_AG:732- | gtcataag | 4 | 2 | 6 | P3_AG:1- | P3_AG:22 | 2445005 |
| | | | | 4 | | | 6-4394 | 2237 | | | | | 731 | 38-4394 | _L3 |
| 4 | HAT2_ | hAT | C. intestin | 2,67 | 0.00% | HAT2_CI:1- | HAT2 Cl:2 | HAT2_CI:4 | ATAAT | 1 | 53 | 54 | HAT2CI: | HAT2CI: | 2445005 |
| | CI | | alis | 7 | | 12 | 666-2677 | 22-2236 | AAT | | | | 1-421 | 2237-2677 | _L4 |
| 5 | HAT5_ | hAT | C. | 2,21 | 0.00% | HATS CI:1- | ITAT5 CI:2 | HATS CI:2 | ATAG | 1 | 14 | 15 | HATS CI: | HAT5 CI: | 2445005 |
| | CI | | mtestmalis | 1 | | 17 | 195-2211 | 56-2082 | GTAT | | | | 1-255 | 2083-2211 | _L5 |
| 6 | hAT- | hAT | D. rerio | 3,52 | 0.00% | hAT- | hAT- | hAT- | ctatgtat | 1 | 485 | 486 | hAT- | hAT- | 2445005 |
| | 6_DR | | | 9 | | 6_DR:1-12 | 6_DR:3518- | 6_DR:225- | | | | | 6_DR:1- | 6_DR:129 | _L6 |
| | | | | | | | 3529 | 1295 | | | | | 224 | 6-3529 | |
| 7 | Chaplin | hAT | D. rerio | 2,92 | 0.00% | Chaplin1_D | Chaplin1_D | Chaplin1_D | GTCTA | 1 | 281 | 282 | Chaplin1_ | Chaplini | 2445005 |
| | 1_DR | | | 0 | | R:1-16 | R:2905- | R:314-2251 | TAG | | | | DRA-313 | DR:2252- | _L7 |
| | | | | | | | 2920 | | | | | | | 2920 | |
| 8 | Harbin | PIF- | X. | 5,44 | 0.00% | Harbinger- | Harbinger- | Harbinger- | TTAAC | 1 | 374 | 375 | Harbinger- | Harbinger- | 2445005 |
| | ger- | Harbin | tropicalis | 2 | | 4_XT:1-30 | 4_XT:5413- | 4_XT:974- | TCTAA | | | | 4_XT:1- | 4_XT:173 | _L8 |
| | 4_XT | ger | | | | | 5442 | 1729 | | | | | 973 | 0-5442 | |
| 9 | HAT1_ | hAT | A. gambiae | 3,70 | 0.10% | SEQ ID | SEQ ID | SEQ ID | SEQ ID | 3 | 20 | 23 | SEQ ID | SEQ ID | |
| | AG | | | 2 | | NO: 2 | NO: 28 | NO: 54 | NO: 192 | | | | NO: 116 | NO: 142 | |
| 10 | AgaP1 | P | A. gambiae | 5,46 | 0.10% | AgaP15:1- | AgaP1.5:543 | AgaP1.5:971 | atatgaac | 2 | 96 | 98 | AgaP15:1- | AgaP15:2 | 2445005 |
| | 5 | | | 2 | | 31 | 2-5462 | -2425 | | | | | 970 | 426-5462 | _L10 |
| 11 | IS4EU- | PIF- | D. rerio | 5,00 | 0.10% | IS4EU- | IS4EU- | IS4EU- | TA | 1 | 2 | 3 | IS4EU- | IS4EU- | 2445005 |
| | 1_DR | Harbin | | 4 | | 1_DR:1-11 | 1_DR:4994- | 1_DR:1006- | | | | | 1_DR:1- | 1_DR:209 | _L11 |
| | | ger | | | | | 5004 | 2094 | | | | | 1005 | 5-5004 | |
| 12 | POGO | TcMari | D. | 2,12 | 0.10% | POGO:1-21 | POGO:2101 | POGO:340- | TA | 5 | 43 | 48 | POGO:1- | POGO:14 | 2445005 |
| | | ner | melano | 1 | | | -2121 | 1458 | | | | | 3.39 | 59-2121 | _L12 |
| | | | gaster | | | | | | | | | | | | |
| 14 | Tc1- | TcMari | D. rerio | 1,59 | 0.20% | SEQ ID | SEQ ID | SEQ ID | SEQ ID | 22 | 1 | 23 | SEQ ID | SEQ ID | |
| | 8B_DR | ner | | 7 | | NO: 3 | NO: 29 | NO: 55 | NO: 193 | | | | NO: 117 | NO: 143 | |
| 15 | HOBO | hAT | D. | 3,01 | 0.20% | HOBO:1-12 | HOBO:3005 | HOBO:316- | gttcacac | 1 | 59 | 60 | HOBO:1- | HOBO:22 | 2445005 |
| | | | persimilis | 6 | | | -3016 | 2250 | | | | | 315 | 51-3016 | _L15 |
| 17 | hAT- | hAT | P. | 2,39 | 0.20% | SEQ ID | SEQ ID | SEQ ID | SEQ ID | 21 | 4 | 25 | SEQ ID | SEQ ID | |
| | 6_PM | | marinus | 1 | | NO: 4 | NO: 30 | NO: 56 | NO: 195 | | | | NO: 118 | NO: 144 | |
| 18 | MARIS | TcMari | S. | 1,67 | 0.20% | MARISPI:1 | MARISP1:1 | MARISP1:3 | TA | 2 | 92 | 94 | MARISPI | MARISPI | 2445005 |
| | P1 | ner | purpurat | 6 | | -38 | 639-1676 | 21-1343 | | | | | :1-320 | :1344- | _L18 |
| | | | us | | | | | | | | | | | 1676 | |
| 19 | hAT- | hAT | x. | 2,10 | 0.20% | N/A | N/A | hAT- | ttatacte | 1 | 1 | 2 | hAT- | hAT- | 2445005 |
| | 7_XT | | tropicalis | 2 | | | | 7_XT:143- | | | | | 7_XT:1- | 7_XT:192 | _L19 |
| | | | | | | | | 1921 | | | | | 142 | 2-2102 | |
| 20 | BARI_ | TcMari | D. | 1,72 | 0.30% | BARLDM: | BARI_DM: | BARLDM: | AT | 2 | 28 | 30 | BARI D | BARI D | 2445005 |
| | DM | ner | persimihs | 7 | | 1-28 | 1701-1728 | 379-1398 | | | | | M:1-378 | M:1399- | _L20 |
| | | | | | | | | | | | | | | 1728 | |
| 21 | Marine | TcMari | P | 1,71 | 0.30% | N/A | N/A | Mariner- | A | 1 | 0 | 1 | Mariner- | Mariner- | 2445005 |
| | r- | ner | marinus | 7 | | | | 3_PM:3- | | | | | 3_PM:1-2 | 3_PM:171 | _L21 |
| | 3_PM | | | | | | | 1715 | | | | | | 6-1717 | |
| 22 | hAT- | hAT | x. | 2,92 | 0.30% | SEQ ID | SEQ ID | SEQ ID | SEQ ID | 5 | 326 | 331 | SEQ ID | SEQ ID | |
| | 3_XT | | tropicalis | 2 | | NO: 5 | NO: 31 | NO: 57 | NO: 196 | | | | NO: 119 | NO: 145 | |
| 23 | P1_AG | P | gambiae | 4,94 | 0.40% | P1_AG:1-11 | P1_AG:493 | PI_AG:781- | GGTG | 2 | 0 | 2 | P1_AG:1- | P1_AG:2.3 | 2445005 |
| | | | | 6 | | | 6-4946 | 2301 | CTGG | | | | 780 | 02-4946 | _L23 |
| 24 | IS4EU- | PIF- | D. rerio | 5,36 | 0.40% | IS4EU- | IS4EU- | IS4EU- | ATCAT | 3 | 0 | 3 | IS4EU- | IS4EU- | 2445005 |
| | 2_DR | Harbin | | 6 | | 2_DR:1-13 | 2_DR:5354- | 2_DR:1363- | TTATA | | | | 2_DR:1- | 2_DR:217 | _L24 |
| | | ger | | | | | 5366 | 2172 | | | | | 1362 | 3-5366 | |
| 25 | Tc1- | TcMari | X. | 1,61 | 0.40% | SEQ ID | SEQ ID | SEQ ID | SEQ ID | 1,268 | 106 | 1,374 | SEQ ID | SEQ ID | |
| | 3_Xt | ner | tropicalis | 4 | | NO: 6 | NO: 32 | NO: 58 | NO: 193 | | | | NO: 120 | NO: 146 | |
| 26 | Marine | TcMari | P. | 2,46 | 0.50% | Mariner- | Mariner- | Mariner- | TA | 5 | 61 | 66 | Mariner- | Mariner- | 2445005 |
| | r- | ner | marinus | 0 | | 1_PM:1-12 | 1_PM:2450- | 1_PM:287- | | | | | 1_PM:1- | 1_PM:209 | _L26 |
| | 1_PM | | | | | | 2461 | 2098 | | | | | 286 | 9-2461 | |
| 27 | P2_AG | P | gambiae | 4,19 | 0.60% | P2_AG:1-27 | P2_AG:417 | P2_AG:128 | GTTTC | 1 | 50 | 51 | P2_AG:1- | P2_AG:23 | 2445005 |
| | | | | 6 | | | 0-4196 | 5-2322 | TAG | | | | 1284 | 23-4196 | _L27 |
| 28 | hAT- | hAT | D. rerio | 2,49 | 0,60% | SEQ ID | SEQ ID | SEQ ID | SEQ ID | 4 | 21 | 25 | SEQ ID | SEQ ID | |
| | 5_DR | | | 6 | | NO: 7 | NO: 33 | NO: 59 | NO: 194 | | | | NO: 121 | NO: 147 | |
| 29 | Tc1- | TcMari | T. | 1,59 | 0.70% | SEQ ID | SEQ ID | SEQ ID | SEQ ID | 6 | 4 | 10 | SEQ ID | SEQ ID | |
| | 3_FR | ner | rubripes | 5 | | NO: 8 | NO: 34 | NO: 60 | NO: 193 | | | | NO: 122 | NO: 148 | |
| 30 | Marine | TcMari | Ai | 2,61 | 0.70% | Mariner- | Mariner- | Mariner- | TA | 1 | 229 | 230 | Mariner- | Mariner- | 2445005 |
| | r-4_XT | ner | tropicalis | 3 | | 1X1:1-19 | 4_XT:2595- | 4_XT:1030- | | | | | 4_XT:1- | 4_XT:233 | _L30 |
| | | | | | | | 2613 | 2331 | | | | | 1029 | 2-2613 | |
| 32 | Tc1- | TcMari | X. | 1,59 | 1.10% | SEQ ID | SEQ ID | SEQ ID | SEQ ID | 377 | 98 | 475 | SEQ ID | SEQ ID | |
| | 5_Xt | ner | tropicalis | 4 | | NO: 9 | NO: 35 | NO: 61 | NO: 193 | | | | NO: 123 | NO: 149 | |
| 33 | Tc1- | TcMar- | X. | 1606 | 1.30% | SEQ ID | SEQ ID | SEQ ID | SEQ ID | 1,330 | 323 | 1,653 | SEQ ID | SEQ ID | |
| | 10_Xt | Tcl | tropicalis | | | NO: 10 | NO: 36 | NO: 62 | NO: 193 | | | | NO: 124 | NO: 150 | |
| 34 | hAT- | hAT | D. | 2,69 | 1.40% | hAT- | hAT- | hAT- | atgcacat | 2 | 50 | 52 | hAT- | hAT- | 2445005 |
| | 1_DP | | persimitis | 9 | | 1 DP:1-8 | 1 DP:2692- | 1 DP:417- | | | | | 1 DP:1- | 1 DP:220 | _L34 |
| | | | | | | | 2699 | 2207 | | | | | 416 | 8-2699 | |
| 35 | Marine | TcMari | A. | 2,00 | 1.50% | SEQ ID | SEQ ID | SEQ ID | SEQ ID | 8 | 2 | 10 | SEQ ID | SEQ ID | |
| | r2_AG | ner | gambiae | 0 | | NO: 11 | NO: 37 | NO: 63 | NO: 193 | | | | NO: 125 | NO: 151 | |
| 36 | Tc1- | TcMari | X. laevis | 1,63 | 1.50% | SEQ ID | SEQ ID | SEQ ID | SEQ ID | 2 | 272 | 274 | SEQ ID | SEQ ID | |
| | 1_Xt | ner | | 4 | | NO: 12 | NO: 38 | NO: 64 | NO: 193 | | | | NO: 126 | NO: 152 | |
| 37 | Tc1- | TcMari | A. | 1,41 | 1.60% | SEQ ID | SEQ ID | SEQ ID | SEQ ID | 30 | 52 | 82 | SEQ ID | SEQ ID | |
| | i_AG | ner | gambiae | 3 | | NO: 13 | NO: 39 | NO: 65 | NO: 193 | | | | NO: 127 | NO: 153 | |
| 38 | Marine | TcMari | X. | 1,76 | 1.60% | Mariner- | Mariner- | Mariner- | TA | 3 | 0 | 3 | Mariner- | Mariner- | 2445005 |
| | r-1_XT | ner | tropicalis | 9 | | 1_XT:1-3 | 1_XT:1767- | 1_XT:81- | | | | | 1_XT:1- | 1_XT:143 | _L38 |
| | | | | | | | 1769 | 1436 | | | | | 80 | 7-1769 | |
| 39 | TclDR | TcMari | X. | 1,23 | 1.70% | SEQ ID | SEQ ID | SEQ ID | SEQ ID | 23 | 103 | 126 | SEQ ID | SEQ ID | |
| | 3_Xt | ner | tropicalis | 1 | | NO: 14 | NO: 40 | NO: 66 | NO: 198 | | | | NO: 128 | NO: 154 | |
| 40 | hAT- | hAT | P. | 2,44 | 1.80% | AT- | AT- | AT- | CTCTA | 13 | 336 | 349 | AT- | hAT- | 2445005 |
| | 1B_PM | | marinus | 6 | | IB_PM:1- | 1B_PM:243 | IB_PM:111 | GAG | | | | IB_PM:1- | IB_PM:22 | _L40 |
| | | | | | | 13 | 5-2447 | 6-2210 | | | | | 1115 | 11-2447 | |
| 41 | hAT- | hAT | P. | 2,98 | 1.90% | SEQ ID | SEQ ID | SEQ ID | SEQ ID | 10 | 149 | 159 | SEQ ID | SEQ ID | |
| | 3_PM | | marinus | 4 | | NO: 15 | NO: 41 | NO: 67 | NO: 199 | | | | NO: 129 | NO: 155 | |
| 42 | hAT- | hAT | P. | 2,55 | 1.90% | hAT- | hAT- | hAT- | CTCTA | 24 | 178 | 202 | hAT- | hAT- | 2445005 |
| | 613_PM | | marinus | 8 | | 6B_PM:1- | 6B_PM:254 | 6B_PM:112 | CAC | | | | 6B_PM:1- | 6B_PM:23 | _L42 |
| | | | | | | 16 | 3-2558 | 0-2337 | | | | | 1119 | 38-2558 | |
| 43 | Tc1- | TcMari | P. | 1,60 | 2.00% | SEQ ID | SEQ ID | SEQ ID | SEQ ID | 6,205 | 1,258 | 7,463 | SEQ ID | SEQ ID | |
| | 1_PM | ner | marinus | 7 | | NO: 16 | NO: 42 | NO: 68 | NO: 193 | | | | NO: 130 | NO: 156 | |
| 44 | TC1_X | TcMari | X. laevis | 1,35 | 2.00% | TCI XL:1- | TCI XL:12 | TCI_XL:25 | TA | 220 | 52 | 272 | TCI XL:1 | TCI XL:1 | 2445005 |
| | L | ner | | 6 | | 132 | 26-1356 | 6-1185 | | | | | -255 | 186-1356 | _L44 |
| 45 | Marine | TcMari | mississip | 2,12 | 2.10% | SEQ ID | SEQ ID | SEQ ID | SEQ ID | 4 | 245 | 249 | SEQ ID | SEQ ID | |
| | r- | ner | piensis | 1 | | NO: 17 | NO: 43 | NO: 69 | NO: 193 | | | | NO: 131 | NO: 157 | |
| | 4_AMi | | | | | | | | | | | | | | |
| 46 | Myotis | hAT | M. | 2,92 | 2.10% | SEQ ID | SEQ ID | SEQ ID | SEQ ID | 74 | 18,867 | 18,94 | SEQ ID | SEQ ID | |
| | hATl | | lucifugus | 1 | | NO: 18 | NO: 44 | NO: 70 | NO: 197 | | | 1 | NO: 132 | NO: 158 | |
| 47 | hAT- | hAT | P. | 2,49 | 2.20% | SEQ ID | SEQ ID | SEQ ID | SEQ ID | 26 | 343 | 369 | SEQ ID | SEQ ID | |
| | 7_PM | | marinus | 9 | | NO: 19 | NO: 45 | NO: 71 | NO: 200 | | | | NO: 133 | NO: 159 | |
| 48 | TC1_F | TcMari | T | 1,63 | 2.30% | TCI_FR4:1- | TC1_FR4:1 | TC1_FR4:3 | TA | 14 | 8 | 22 | TC1_FR4: | TC1_FR4: | 2445005 |
| | R4 | ner | rubripes | 1 | | 27 | 605-1631 | 57-1403 | | | | | 1-356 | 1404-1631 | _L48 |
| 49 | hAT- | hAT | P. | 3,78 | 2.30% | hAT- | hAT- | hAT- | CTCTA | 8 | 38 | 46 | hAT- | hAT- | 2445005 |
| | 8_PM | | marinus | 1 | | 8_PM:1-16 | 8_PM:3766- | 8_PM:2365- | CAG | | | | 8_PM:1- | 8_PM:361 | _L49 |
| | | | | | | | 3781 | 3615 | | | | | 2364 | 6-3781 | |
| 50 | piggyB | piggyB | M. | 2,52 | 2.50% | piggyBac1 | piggyBac1 | piggyBac1 | TTAA | 1 | 1,255 | 1,256 | piggyBac 1 | piggyBac1 | 2445005 |
| | acl_M | ac | murinus | 7 | | Mm:1-13 | Mm:2515- | Mm:1037- | | | | | Mtn:]- | Mm:234 | _L50 |
| | m | | | | | | 2527 | 2347 | | | | | 1036 | 8-2527 | |
| 51 | Tc1- | TcMari | X. | 1633 | 2.50% | SEQ ID | SEQ ID | SEQ ID | SEQ ID | 121 | 112 | 233 | SEQ ID | SEQ ID | |
| | 11_Xt | ner | tropicaiis | | | NO: 20 | NO: 46 | NO: 72 | NO: 193 | | | | NO: 134 | NO: 160 | |
| 52 | Tc1- | TcMari | X | 1635 | 2.80% | SEQ ID | SEQ ID | SEQ ID | SEQ ID | 66 | 61 | 127 | SEQ ID | SEQ ID | |
| | 16_Xt | ner | tropicaiis | | | NO: 21 | NO: 47 | NO: 73 | NO: 193 | | | | NO: 135 | NO: 161 | |
| 53 | TC1- | TcMari | D. | 1,64 | 2.90% | TC1- | TCI- | TCI- | TA | 3 | 9 | 12 | TCI- | TC1- | 2445005 |
| | 2_DM | ner | meianoga | 4 | | 2_DM:1-21 | 2_DM:1624 | 2_DM:356- | | | | | 2_DM:1- | 2_DM:137 | _L53 |
| | | | ster | | | | -1644 | 1378 | | | | | 355 | 9-1644 | |
| 54 | Tc1- | TcMari | X | 1596 | 3.30% | SEQ ID | SEQ ID | SEQ ID | SEQ ID | 61 | 71 | 132 | SEQ ID | SEQ ID | |
| | 4_Xt | ner | tropicaiis | | | NO: 22 | NO: 48 | NO: 74 | NO: 193 | | | | NO: 136 | NO: 162 | |
| 55 | TCID | TcMari | D. | 1,66 | 3.50% | TC1_DM:1- | TCI_DM:16 | TC1_DM:35 | TA | 4 | 21 | 25 | TC1_DM: | TCI_DM: | 2445005 |
| | M | ner | meianoga | 6 | | 26 | 41-1666 | 9-1354 | | | | | 1-358 | 1355-1666 | _L55 |
| | | | ster | | | | | | | | | | | | |
| 56 | Tc1- | TcMari | X. | 1581 | 3.50% | SEQ ID | SEQ ID | SEQ ID | SEQ ID | 60 | 76 | 136 | SEQ ID | SEQ ID | |
| | 15_Xt | ner | tropicaiis | | | NO: 23 | NO: 49 | NO: 75 | NO: 193 | | | | NO: 137 | NO: 163 | |
| 59 | Tc1_F | TcMari | T | 1602 | 3.80% | SEQ ID | SEQ ID | SEQ ID | SEQ ID | 15 | 36 | 51 | SEQ ID | SEQ ID | |
| | R2 | ner | rubripes | | | NO: 24 | NO: 50 | NO: 76 | NO: 193 | | | | NO: 138 | NO: 164 | |
| 60 | Marine | TcMari | A | 1,96 | 4.40% | Mariner- | Mariner- | Mariner- | TA | 3 | 202 | 205 | Mariner- | Mariner- | 2445005 |
| | r- | ner | mississip | 1 | | 6B_AMi:1- | 6B_AMi:19 | 6B_AMi:39 | | | | | 6B_AMi:1 | 6B_AMi:1 | _L60 |
| | 6B_AMi | | piensis | | | 25 | 37-1961 | 0-1649 | | | | | -389 | 650-1961 | |
| 61 | Tc1- | TcMari | X. | 1,65 | 4.40% | Tc1-9_Xt: | Tc1- | Tc1- | TA | 14 | 25 | 39 | Tc1- | Tc1- | 2445005 |
| | 9_Xt | ner | tropicalis | 4 | | 1-225 | 9_Xt:1430- | 9_Xt:362- | | | | | 9_Xt:1- | 9_Xt:1400 | _L61 |
| | | | | | | | 1654 | 1399 | | | | | 361 | -1654 | |
| 63 | hAT- | hAT | X. | 5716 | 4.60% | SEQ ID | SEQ ID | SEQ ID | SEQ ID | 124 | 5,189 | 5,313 | SEQ ID | SEQ ID | |
| | 9_XT | | tropicalis | | | NO: 25 | NO: 51 | NO: 77 | NO: 201 | | | | NO: 139 | NO: 165 | |
| 64 | Marine | TcMari | X | 1840 | 4.60% | SEQ ID | SEQ ID | SEQ ID | SEQ ID | 2 | 4 | 6 | SEQ ID | SEQ ID | |
| | r-5_XT | ner | tropicalis | | | NO: 26 | NO: 52 | NO: 78 | NO: 193 | | | | NO: 140 | NO: 166 | |
| 65 | S2_DM | TcMari | D. | 1,73 | 4.70% | S2_DM:1- | S2_DM:169 | S2_DM:403 | TA | 2 | 2 | 4 | S2_DM:1- | S2_DM:1 | 2445005 |
| | | ner | meianoga | 5 | | 46 | 0-1735 | -1386 | | | | | 402 | 387-1735 | _L65 |
| | | | ster | | | | | | | | | | | | |
| 67 | PROT | p | D. | 1,10 | 4.90% | PROTOP:1- | PROTOP:44 | PROTOP:11 | GTCCT | 8 | 461 | 469 | PROTOP: | PROTOP: | 2445005 |
| | OP | | meianoga | 5 | | 31 | 50-4480 | 94-3788 | GG | | | | 1-1193 | 3789-4480 | _L67 |
| | | | ster | | | | | | | | | | | | |
| 68 | Tc1- | TcMari | a | 1,65 | 4.90% | Tc1-8_Xt:1- | Tc1- | Tc1- | TA | 17 | 81 | 98 | Tc1- | Tc1- | 2445005 |
| | 8_Xt | ner | tropicalis | 0 | | 31 | 8_Xt:1620- | 8_Xt:400- | | | | | 8_Xt:1- | 8_Xt:1423 | _L68 |
| | | | | | | | 1650 | 1422 | | | | | 399 | -1650 | |
| 69 | Tc1- | TcMari | X. | 1,62 | 5.00% | Tc1- | Tc1- | Tc1- | TA | 5 | 22 | 27 | Tc1- | Tc1- | 2445005 |
| | 12_Xt | ner | tropicalis | 6 | | 12_Xt:1-74 | 12_Xt:1554- | 12_Xt:390- | | | | | 12_Xt:1- | 12_Xt:141 | E69 |
| | | | | | | | 1626 | 1409 | | | | | 389 | 0-1626 | |

Divergence > 5%

| 70 | Mari | TcMar- | A. | 2479 | 5.10% | SEQ ID | SEQ ID | | | | | | | | |
|---|---|---|---|---|---|---|---|---|---|---|---|---|---|---|---|
| | ner- | Tc2 | mississip | | | NO: 79 | NO: 91 | NO: 103 | NO: 193 | | | | NO: 167 | NO: 179 | |
| | 6_AMi | | piensis | | | | | | | | | | | | |
| 71 | hAT- | hAT | mississip | 3,32 | 5.50% | hAT- | hAT- | hAT- | CTTGT | 7 | 118 | 125 | hAT- | hAT- | 2445005 |
| | 3_Gm | | piensis | 5 | | 3_Gav:1-10 | 3_Gav:3316 | 3_Gav:1577 | ATC | | | | 3_Gav:1- | 3_Gav:30 | _L71 |
| | | | | | | | -3325 | -3028 | | | | | 1576 | 29-3325 | |
| 72 | piggyB | piggyB | M. | 2,21 | 5.70% | N/A | N/A | piggyBac2 | CTATA | 0 | 0 | 0 | piggyBac2 | piggyBac2 | 2445005 |
| | ac2_M | ac | murinus | 1 | | | | Mm:277- | TAG | | | | Mm:1- | Mm:208 | _L72 |
| | m | | | | | | | 2085 | | | | | 276 | 6-2211 | |
| 73 | Marine | TcMari | X. | 2,43 | 5.70% | Mariner- | Mariner- | Mariner- | TCTAT | 1 | 306 | 307 | Mariner- | Mariner- | 2445005 |
| | r-2_XT | ner | tropicalis | 8 | | 2_XT:1-27 | 2_XT:2412- | 2_XT:764- | GAC | | | | 2_XT:1- | 2_XT:212 | _L73 |
| | | | | | | | 2438 | 2128 | | | | | 763 | 9-2438 | |
| 74 | hAT- | hAT | A. | 3,50 | 5.80% | hAT- | hAT- | hAT- | TA | 3 | 9 | 12 | 4_Crp:1- | 4_Crp:324 | 2445005 |
| | 4_Crp | | mississip | 8 | | 4_Crp:1-14 | 4_Crp:3495- | 4_Crp:603- | | | | | 602 | 0-3508 | _L74 |
| | | | piensis | | | | 3508 | 3239 | | | | | | | |
| 75 | Opos | hAT | M. | 2,99 | 5.80% | OposCharlie | OposCharlie | OposCharlie | GTCAT | 17 | 1172 | 1189 | OposCharl | OposCharl | 2445005 |
| | Charlie | | domestic | 6 | | 2:1-16 | 2:2981-2996 | 2:744-2681 | AAG | | | | ie2:1-743 | ie2:2682- | _L75 |
| | 2 | | | | | | | | | | | | | 2996 | |
| 76 | hAT- | hAT | A. | 3,50 | 6.00% | hAT- | hAT- | hAT- | ITTCC | 16 | 43 | 59 | hAT- | hAT- | 2445005 |
| | 1_AMi | | mississip | | | 1_AMi:1-9 | 1_AMi:3499 | 1_AMi:1102 | TTG | | | | 1_AMi:1- | 1_AMi:29 | _L76 |
| | | | piensis | | | | -3507 | -2943 | | | | | 1101 | 44-3507 | |
| 77 | Mariner | TcMari | A | 2,26 | 6.00% | Mariner- | Mariner- | Mariner- | TA | 1 | 99 | 100 | Mariner- | Mariner- | 2445005 |
| | 2_AMi | ner | mississip | 0 | | 2_AMi:1-13 | 2_AMi:2248 | 2_AMi:587- | | | | | 2_AMi:1- | 2_AMi:20 | _L77 |
| | | | piensis | | | | -2260 | 2065 | | | | | 586 | 66-2260 | |
| 78 | hAT- | hAT | A. | 3.53 | 6.40% | hAT- | hAT- | hAT- | TA | 24 | 186 | 210 | hAT- | AT- | 2445005 |
| | 13_ | | mississip | 1 | | 13_AMi:1- | 13_AMi:350 | 13_AMi:125 | | | | | 13_AMi:1 | 13_AMi:3 | _L78 |
| | AMii | | piensis | | | 29 | 4-3531 | 5-3087 | | | | | -1254 | 088-35.31 | |
| 80 | hAT- | hAT | A. | 4,34 | 6.60% | hAT- | hAT- | hAT- | TTAA | 2 | 14 | 16 | hAT- | hAT- | 2445005 |
| | 12_AM | | mississip | 8 | | 12_AMi:1- | 12_AMi:433 | 12_AMi:139 | | | | | 12_AMi:1 | 12_AMi:3 | _L80 |
| | i | | piensis | | | | 2-4348 | 2-3989 | | | | | -1391 | 990-4348 | |
| 81 | Mari | TcMari | A. | 2,33 | 6.70% | Mariner- | Mariner- | Mariner- | TA | 6 | 347 | 353 | Mariner- | Mariner- | 2445005 |
| | ner- | ner | mississip | 5 | | 7_Croc:1-17 | 7_Croc:231 | 7_Croc:195- | | | | | 7_Croc:1- | 7_Croc:19 | _L81 |
| | 7_Croc | | piensis | | | | 9-2335 | 1982 | | | | | 194 | 83-2335 | |
| 82 | piggyB | PiggyB | tropicalis | 8451 | 6.70% | SEQ ID | SEQ ID | SEQ ID | SEQ ID | 0 | 19 | 19 | SEQ ID | SEQ ID | |
| | ac- | ac | | | | NO: 80 | NO: 92 | NO: 104 | NO: 202 | | | | NO: 168 | NO: 180 | |
| | 2_XT | | | | | | | | | | | | | | |
| 83 | Mari | TcMari | A. | 2,52 | 6.80% | Mariner- | Mariner- | Mariner- | TA | 0 | 190 | 190 | Mariner- | Mariner- | 2445005 |
| | ner- | ner | mississip | 2 | | 3_AMi:1-24 | 3_AMi:2499 | 3_AMi:345- | | | | | 3_AMi:1- | 3_AMi:21 | _L83 |
| | 3_AMi | | piensis | | | | -2522 | 2138 | | | | | 344 | 39-2522 | |
| 84 | piggvB | piggyB | C. | 2,21 | 6.80% | piggyBac1_ | piggy Bad | piggyBac1_ | GCTGC | 2 | 2 | A | piggyBac1 | piggyBac1 | 2445005 |
| | ael_CI | ac | intestinali | 6 | | CI:1-13 | CI:2204- | CE263-1984 | AGG | | | | CI:1-262 | CI:1985- | _L84 |
| | | | | | | | 2216 | | | | | | | 2216 | |
| 86 | piggyB | PiggyB | A. | 3224 | 7.00% | SEQ ID | SEQ ID | SEQ ID | SEQ ID | 20 | 112 | 132 | SEQ ID | SEQ ID | |
| | ac- | ac | mississip | | | NO: 81 | NO: 93 | NO: 105 | NO: 202 | | | | NO: 169 | NO: 181 | |
| | 1_AMi | | piensis | | | | | | | | | | | | |
| 87 | Marine | TcMari | A. | 2,29 | 7.00% | Mariner- | Mariner- | Mariner- | GTTGT | 2 | 2105 | 2107 | Mariner- | Mariner- | 2445005 |
| | r- | ner | mississip | 4 | | 5_AMi:1-21 | 5_AMi:2274 | 5_AMi:535- | AAC | | | | 5_AMi:1- | 5_AMi:20 | _L87 |
| | 5_AMi | | piensis | | | | -2294 | 2076 | | | | | 534 | 77-2294 | |
| 89 | Marine | TcMari | A. | 2,24 | 7.30% | Mariner- | Mariner- | Mariner- | TA | 5 | 715 | 720 | Mariner- | Mariner- | 2445005 |
| | r- | ner | mississip | 9 | | 6_Crp:1-24 | 6_Crp:2226- | 6_Crp:240- | | | | | 6_Crp:1- | 6_Crp:205 | _L89 |
| | 6_Crp | | piensis | | | | 2249 | 2051 | | | | | 239 | 2-2249 | |
| 90 | Marine | TeMar- | A. | 2292 | 7.50% | SEQ ID | SEQ ID | SEQ ID | SEQ ID | 9 | 25 | 34 | SEQ ID | SEQ ID | |
| | r- | Tigger | mississip | | | NO: 82 | NO: 94 | NO: 106 | NO: 193 | | | | NO: 170 | NO: 182 | |
| | 3_Crp | | piensis | | | | | | | | | | | | |
| 91 | hAT- | hAT | mississip | 3,56 | 7.60% | hAT- | hAT- | hAT- | ACTGT | 2 | 41 | 43 | hAT- | hAT- | 2445005 |
| | 3_AMi | | piensis | 5 | | 3_AMi:1-14 | 3_AMi:3552 | 3_AMi:1179 | TCTAT | | | | 3_AMi:1- | 3_AMi:30 | _L91 |
| | | | | | | | -3565 | -3029 | | | | | | 30-3565 | |
| 92 | TC1_F | TcMari | T. | 1,22 | 7.80% | TCI FRL:1- | TCI FR1:1 | TCI FR1:1 | CCTTG | 6 | 23 | 29 | TC1_FR1: | TCI FRI: | 2445005 |
| | RI | ner | rubripes | 0 | | 24 | 197-1220 | 48-1188 | AAG | | | | 1-147 | 1189-1220 | E92 |
| 93 | hAT- | hAT | mississip | 3,51 | 7.90% | hAT- | hAT- | hAT- | CTATA | 2 | 327 | 329 | hAT- | hAT- | 2445005 |
| | 5_Croc | | piensis | 9 | | 5_Croc:1-10 | 5_Croc:351 | 5_Croc:140 | TAG | | | | 5_Croc:1- | 5_Croc:29 | _L93 |
| | | | | | | | 0-3519 | 3-2.905 | | | | | 1402 | 06-3519 | |
| 94 | hA_T- | hAT | mississip | 3,33 | 7.90% | hAT- | hAT- | hAT- | TA | 0 | 57 | 57 | hAT- | hAT- | 2445005 |
| | 8_AMi | | piensis | 2 | | 8_AMi:1-16 | 8_AMi:33T7 | 8_AMi:1159 | | | | | 8_AMi:1- | 8_AMi:31 | _L94 |
| | | | | | | | -3332 | -3183 | | | | | 1158 | 84-3332 | |
| 95 | PARIS | TcMari | D. virilis | 1,73 | 7.90% | PARIS:1- | PARIS:1489 | PARIS:394- | CTGTA | 2 | 6 | 8 | PARIS:1- | PARIS:14 | 2445005 |
| | | ner | | 0 | | 242 | -1730 | 1440 | TAG | | | | 393 | 41-1730 | _L95 |
| 96 | Mari | TcMari | P. | 2,39 | 7.90% | Mariner- | Mariner- | Mariner- | TA | 3 | 226 | 229 | Mariner- | Mariner- | 2445005 |
| | ner- | ner | marinus | 0 | | 2_PM:1-23 | 2_PM:2368- | 2_PM:288- | | | | | 2_PM:1- | 2_PM:168 | _L96 |
| | 2_PM | | | | | | 2390 | 1682 | | | | | 287 | 3-2390 | |
| 97 | piggyB | piggyB | X | 6,16 | 8.30% | piggyBac- | piggyBac- | piggyBac- | CTGTA | 1 | 301 | 302 | piggyBac- | piggyBac- | 2445005 |
| | ac- | ac | tropicalis | 9 | | 1_XT:1-14 | 1_XT:6156- | 1_XT:1579- | TAG | | | | 1_XT:1- | 1_XT:337 | _L97 |
| | 1_XT | | | | | | 6169 | 3372 | | | | | 1578 | 3-6169 | |
| 98 | Tigger | TcMari | T. | 2,41 | 8.50% | Tiggerl:1- | Tiggerl:240 | Tiggerl:425 | CTAAT | 353 | 244 | 597 | Tiggerl:1- | Tiggerl:1 | 2445005 |
| | 1 | ner | manatus | 8 | | 13 | 6-2418 | -1789 | TAG | | | | 424 | 790-2418 | _L98 |
| 99 | Mari | TcMari | mississip | 2,88 | 8.80% | Mariner- | Mariner- | Mariner- | TTTAT | 2 | 290 | 292 | Mariner- | Mariner- | 2445005 |
| | ner- | ner | piensis | 1 | | 1_Crp:1-13 | 1_Crp:2869- | 1_Crp:823- | AAT | | | | 1_Crp:1- | 1_Crp:261 | _L99 |
| | 1_Crp | | | | | | 2881 | 2613 | | | | | 822 | 4-2881 | |
| 101 | S_DM | TcMari | D. | 1,73 | 9.10% | S_DM:1- | S_DM:1503 | S_DM:404- | CTATA | 34 | 12 | 46 | S_DM:1- | S_DM:14 | 2445005 |
| | | ner | meianoga | 6 | | 234 | -1736 | 1441 | TAG | | | | 403 | 42-1736 | _L101 |
| | | | ster | | | | | | | | | | | | |
| 102 | hAT- | hAT | A. | 2,76 | 9.20% | hAT- | hAT- | hAT- | CTCAT | 0 | 1888 | 1888 | hAT- | hAT- | 2445005 |
| | 12_Crp | | mississip | 1 | | 12_Crp:1-16 | 12_Crp:274 | 12_Crp:67- | AAA | | | | 12_Crp:1- | 12_Crp:22 | _L102 |
| | | | piensis | | | | 6-2761 | 2262 | | | | | 66 | 63-2761 | |
| 103 | hAT- | hAT- | P. | 2311 | 9.40% | SEQ ID | SEQ ID | SEQ ID | SEQ ID | 292 | 50 | 342 | SEQ ID | SEQ ID | |
| | 1_PM | Charlie | marinus | | | NO: 83 | NO: 95 | NO: 107 | NO: 203 | | | | NO: 171 | NO: 183 | |
| 104 | Sen | PIF- | T | 2,22 | 9.60% | Senkushal:1 | Senkusha1:2 | Senkusha1:7 | TATAA | 0 | 84 | 84 | Senkusha1 | Senkusha1 | 2445005 |
| | kushal | Harbin | rubripes | 4 | | -18 | 207-2224 | 88-1708 | GTCA | | | | :1-787 | :1709- | _L104 |
| | | ger | | | | | | | G | | | | | 2224 | |
| 105 | Tc1- | TcMari | P. | 1,61 | 9.60% | Tc1- | Tc1- | Tc1- | TATAG | 4 | 19 | 23 | Tc1- | Tc1- | 2445005 |
| | 2_PM | ner | marinus | 0 | | 2_PM:1-28 | 2_PM:1583- | 2_PM:461- | TTCAC | | | | 2_PM:1- | 2_PM:139 | _L105 |
| | | | | | | | 1610 | 1390 | | | | | 460 | 1-1610 | |
| 106 | Harbin | PIF- | A. | 4,38 | 9.70% | N/A | N/A | Harbinger- | ATATA | 3 | 7 | 10 | Harbinger- | Harbinger- | 2445005 |
| | ger- | Harbin | mississip | 1 | | | | 2_AMi:461- | TAAG | | | | 2_AMi:1- | 2 AMi:18 | _L106 |
| | 2_AMi | ger | piensis | | | | | 1885 | A | | | | 460 | 86-4381 | |
| 108 | hAT- | hAT | mississip | 3,22 | 10.80 | hAT- | hAT- | hAT- | TAAG | 0 | 59 | 59 | hAT- | hAT- | 2445005 |
| | 2_Gav | | piensis | 2 | % | 2__Gav:1-16 | 2__Gav:3207 | 2__Gav:1044 | ATTTA | | | | 2_Gav:1- | 2__Gav:30 | _L108 |
| | | | | | | | -3222 | -3071 | TT | | | | 1043 | 72-3222 | |
| 110 | Tigger | TcMari | B. | 3,45 | 11.00 | Tigger2M1- | Tigger2f:34 | Tigger26:60 | TA | 7 | 1597 | 1604 | Tigget2f:1 | Tigget2£2 | 2445005 |
| | 2f | ner | acutorost | 5 | % | 19 | 37-3455 | 3-2462 | | | | | -602 | 463-3455 | _L110 |
| | | | rata | | | | | | | | | | | | |
| 111 | Tc1- | TcMari | D. rerio | 1,57 | 11.00 | Tc1- | Tc1- | Tc1- | TA | 36 | 309 | 345 | Tc1- | Tc1- | 2445005 |
| | 4_DR | ner | | 2 | % | 4_DR:1-51 | 4_DR:1522- | 4_DR:296- | | | | | 4_DR:1- | 4_DR:137 | _L111 |
| | | | | | | | 1572 | 1372 | | | | | 295 | 3-1572 | |
| 116 | Tigger | TcMari | B. | 2,67 | 11.90 | Tiggerl7:1- | Tiggerl7:26 | Tigger17:11 | TA | 1 | 273 | 274 | Tiggerl7: | Tiggerl7: | 2445005 |
| | 17 | ner | acutorost | 3 | % | 23 | 51-2673 | 3-2095 | | | | | 1-112 | 2096-2673 | _L116 |
| | | | rata | | | | | | | | | | | | |
| 117 | Tigger | TcMari | H. | 3,02 | 11.90 | Tigger3:1- | Tigger3:300 | Tigger3:463 | CTATA | 1 | 1489 | 1490 | Tigger3:1- | Tigger3:2 | 2445005 |
| | 3 | ner | sapiens | 9 | % | 23 | 7-3029 | -2307 | TAG | | | | 462 | 308-3029 | _L117 |
| 118 | hAT- | hAT | A. | 2,02 | 12.30 | hAT- | hAT- | hAT- | TA | 3 | 1 | 4 | hAT- | hAT- | 2445005 |
| | M_Crp | | mississip | 3 | % | 14_Crp:1-2 | 14_Crp:202 | 14_Crp:193- | | | | | 14_Crp:1- | 14_Crp:17 | _L118 |
| | | | piensis | | | | 2-2023 | 1779 | | | | | 192 | 80-2023 | |
| 119 | Marine | TcMari | A. | 1,98 | 12.30 | Mariner- | Mariner- | Mariner- | TTAA | n | 25 | 27 | Mariner- | Mariner- | 2445005 |
| | r- | ner | mississip | 4 | % | 9_Crp:1-19 | 9_Crp:1966- | 9_Crp:252- | | | | | 9_Crp:1- | 9_Crp:156 | _L119 |
| | 9_Crp | | piensis | | | | 1984 | 1559 | | | | | 251 | 0-1984 | |
| 124 | hAT- | hAT | A. | 5,87 | 12.80 | hAT- | hAT- | hAT- | TA | 0 | 566 | 566 | hAT- | hAT- | 2445005 |
| | 11_AM | | mississip | 1 | % | 11_AMi:1- | 11_AMi:585 | 11_AMi:879 | | | | | 11_AMi:1 | 11_AMi:3 | _L124 |
| | i | | piensis | | | 16 | 6-5871 | -3152 | | | | | -878 | 153-5871 | |
| 126 | Marine | TcMari | A. | 2,67 | 13.00 | N/A | N/A | Mariner- | TA | 2 | 6 | 8 | Mariner- | Mariner- | 2445005 |
| | r- | ner | mississip | 1 | % | | | 10_Crp:439- | | | | 10_Crp:1- | 10 Op:15 | _L126 | _L126 |
| | 10_Crp | | piensis | | | | | 1527 | | | | 438 | 28-2671 | | |
| 139 | hAT- | hAT-Ac | A. | 5670 | 13.80 | SEQ ID | SEQ ID | SEQ ID | SEQ ID | 1 | 302.2 | 3023 | SEQ ID | SEQ ID | |
| | 17_Cro | | mississip | | % | NO: 84 | NO: 96 | NO: 108 | NO: 196 | | | | NO: 172 | NO: 184 | |
| | c | | piensis | | | | | | | | | | | | |
| 140 | hAT- | hAT | mississip | 5,52 | 13.80 | hAT- | hAT- | hAT- | TA | 1 | 500 | 501 | hAT- | hAT- | 2445005 |
| | 4_AMi | | piensis | 1 | % | 4_AMi:1-14 | 4_AMi:5508 | 4_AMi:1477 | | | | | 4_AMi:1- | 4_AMi:37 | _L140 |
| | | | | | | | -5521 | -3756 | | | | | 1476 | 57-5521 | |
| 142 | Tigger | TcMar- | M.murinu | 2731 | 13.80 | SEQ ID | SEQ ID | SEQ ID | SEQ ID | 15 | 1430 | 1445 | SEQ ID | SEQ ID | |
| | 4 | Tigger | s | | % | NO: 85 | NO: 97 | NO: 109 | NO: 193 | | | | NO: 173 | NO: 185 | |
| 144 | Tigger | TcMar- | T | 2492 | 13.80 | SEQ ID | SEQ ID | SEQ ID | SEQ ID | 2 | 313 | 315 | SEQ ID | SEQ ID | |
| | 7 | Tigger | manatus | | % | NO: 86 | NO: 98 | NO: 110 | NO: 193 | | | | NO: 174 | NO: 186 | |
| | | | latirostris | | | | | | | | | | | | |
| 156 | Tigger | TcMari | P. | 2,71 | 14.20 | Tigger:1- | Tigger2:270 | Tigger2:603 | CTATA | 27 | 1155 | 1182 | Tigger2:1- | Tigger2:2 | 2445005 |
| | 2 | ner | paniscus | 8 | % | 19 | 0-2718 | -2486 | TAG | | | | 602 | 487-2718 | _L156 |
| 180 | hAT- | hAT | A. | 2844 | 14.50 | SEQ ID | SEQ ID | SEQ ID | SEQ ID | 0 | 9 | 9 | SEQ ID | SEQ ID | |
| | 19J7rp | | mississip | | % | NO: 87 | NO: 99 | NO: 111 | NO: 204 | | | | NO: 175 | NO: 187 | |
| | | | piensis | | | | | | | | | | | | |
| 183 | hAT- | hAT-Ac | A. | 6042 | 14.60 | SEQ ID | SEQ ID | SEQ ID | SEQ ID | 0 | 2162 | 2162 | SEQ ID | SEQ ID | |
| | 17B_C | | mississip | | % | NO: 88 | NO: 100 | NO: 112 | NO: 205 | | | | NO: 176 | NO: 188 | |
| | roc | | piensis | | | | | | | | | | | | |
| 188 | Tc1- | TcMari | x | 1,62 | 14.60 | Tc1- | Tc1- | Tc1- | TATTT | 0 | 7 | 7 | Tc1- | Tei- | 2445005 |
| | 13Xt | ner | tropicalis | 4 | % | 13_Xt:1-27 | 13_Xt:1598- | 13_Xt:381- | ATA | | | | 13_Xt:1- | 13_Xt:140 | _L188 |
| | | | | | | | 1624 | 1403 | | | | | 380 | 4-1624 | |
| 197 | hAT- | hAT | A. | 2,51 | 15.00 | hAT- | hAT- | hAT- | TA | 11 | 17 | 28 | hAT- | hAT- | 2445005 |
| | 1913_C | | mississip | 7 | % | 19B_Croc:1 | 19B_Croc:2 | 19B_Croc:3 | | | | | 19B_Croc: | 19B_Croc: | _L197 |
| | roc | | piensis | | | -15 | 503-2517 | 50-2095 | | | | | 1-349 | 2096-2517 | |
| 199 | MarsTi | TcMari | M. | 2,25 | 15.00 | MarsTigger | MarsTigger | MarsTigger | TTAA | 1 | 71 | 72 | MarsTigge | MarsTigge | 2445005 |
| | gger8 | ner | eugenii | 3 | % | 8:1-23 | 8:2231-2253 | 8:322-1950 | | | | | 1-8:1-321 | r8:1951- | _L199 |
| | | | | | | | | | | | | | | 225.3 | |
| 212 | Tigger | TcMari | C. | 2,40 | 15.60 | Tigger5:1- | Tigger5:238 | Tigger5:152 | TA | 1 | 124 | 12.5 | Tigger5:1- | Tigger5:2 | 2445005 |
| | 5 | ner | familiaris | 6 | % | 24 | 3-2406 | -2068 | | | | | 151 | 069-2406 | _L212 |
| 222 | hAT- | hAT-Ac | X. | 1114 | 16.00 | SEQ ID | SEQ ID | SEQ ID | SEQ ID | 0 | 141 | 141 | SEQ ID | SEQ ID | |
| | 10_XT | | tropicalis | 4 | % | NO: 89 | NO: 101 | NO: 113 | NO: 206 | | | | NO: 177 | NO: 189 | |
| 237 | hAT- | hAT | A. | 5,16 | 16.60 | hAT- | hAT- | | | | | | | | |
| | 6_AM | | mississip | 2 | % | 6_AMi:1-15 | 6_AMi:5148 | 6_AMi:2157 | | | | | 6_AMi:1- | 6_AMi:33 | 2445005 |
| | | | piensis | | | | -5162 | -3356 | | | | | 2156 | 57-5162 | _L237 |
| 245 | Mars | TcMari | M. | 1,81 | 17.30 | MarsFigger | MarsTigger | MarsTigger | TA | 1 | 319 | 320 | MarsTi gge | MarsTigge | 2445005 |
| | Tigger | ner | domestica | 3 | % | lc:1-22 | lc:1792- | lc:503-1813 | | | | | rlc:1-502 | rlc:1814- | _L245 |
| | 1c | | | | | | 1813 | | | | | | | 1813 | |
| 246 | Tigger | TcMari | B. | 1,18 | 17.40 | Tigger17c:1 | Tiggerl7c:1 | Tigger17c:1 | TA | 3 | 268 | 271 | Tiggerl7c: | Tigger17c: | 2445005 |
| | 17c | ner | acutorost | 8 | % | -23 | 166-1188 | 13-982 | | | | | 1-112 | 983-1188 | _L246 |
| | | | rata | | | | | | | | | | | | |
| 258 | Harbin | P1F- | A | 6,56 | 17.90 | N/A | N/A | Harbinger- | CTCTA | 0 | 29 | 29 | Harbinger- | Harbinger- | 2445005 |
| | ger- | Harbin | mississip | 8 | % | | | 1_AMi:4005 | GAG | | | | i amli- | 1_AMi:54 | _L258 |
| | 1_AM | ger | piensis | | | | | -5459 | | | | | 4004 | 60-6568 | |
| 260 | Tc1- | TcMari | X. | 1,61 | 18.10 | Tc1- | Tc1- | Tc1- | TA | n | 75 | 77 | Tc1- | Tc1- | 2445005 |
| | 14_Xt | ner | tropiealis | 2 | % | 14_Xt:1-214 | 14_Xt:1400- | 14_Xt:362- | | | | | 14_Xt:1- | 14_Xt:139 | _L260 |
| | | | | | | | 1612 | 1393 | | | | | 361 | 4-1612 | |
| 271 | Kanga | TcMari | V. pacos | 1,74 | 18.80 | Kangal:1- | Kangal:172 | Kanga1:240 | CTATA | 0 | 7 | 7 | Kangal:1- | Kangal:15 | 2445005 |
| | r | ner | | 5 | % | 22 | 4-1745 | -1511 | TAG | | | | 239 | 12-1745 | _L271 |
| 275 | Arthur | hAT | G. | 3,94 | 19.20 | Arthur1:1- | Arthurl:393 | Arthur1:115 | TA | 0 | 32 | 32 | Arthur1:1- | Arthurl:3 | 2445005 |
| | 1 | | variegatus | 7 | % | 11 | 7-3947 | 0-3711 | | | | | 1149 | 712-3947 | _L275 |
| 295 | Harbin | PIF- | mississip | 5,17 | 20.00 | Harbinger- | Harbinger- | Harbinger- | TA | 1 | 44 | 45 | Harbinger- | Harbinger- | 2445005 |
| | ger- | Harbin | piensis | 2 | % | 1_Crp:1-21 | 1_Crp:5152- | 1_Crp:576- | | | | | 1_Crp:1- | 1_Crp:237 | _L295 |
| | 1_Crp | ger | | | | | 5172. | 2375 | | | | | 575 | 6-5172 | |
| 314 | Zaphod | hAT | B. | 2,62 | 21.00 | Zaphod3:1- | Zaphod3:26 | Zaphod3:87 | TA | 0 | 1 | 1 | Zaphod3:1 | Zaphod3:2 | 2445005 |
| | 3 | | acutorost | 4 | % | 12 | 13-2624 | 8-2296 | | | | | -877 | 297-2624 | _L314 |
| | | | rata | | | | | | | | | | | | |
| 320 | Joey1 | TcMari | M. | 1,36 | 21.20 | Joeyl:1-27 | Joeyl:1339- | Joey1:255- | TA | 0 | 5 | 5 | Joey 1 1- | Joeyl:127 | 2445005 |
| | | ner | eugenii | 5 | % | | 1365 | 1271 | | | | | 254 | 2-1365 | _L320 |
| 331 | Zaphod | hAT | c. | 4,07 | 21.90 | Zaphod:1-11 | Zaphod:406 | Zaphod:101 | TTAA | 0 | 0 | 0 | Zaphod:1- | Zaphod:37 | 2445005 |
| | 3 | | syrichta | 9 | % | | 9-4079 | 0-3745 | | | | | 1009 | 46-4079 | _L331 |
| 342 | Harbin | PIF- | mississip | 5,32 | 23.10 | Harbinger- | Harbinger- | Harbinger- | TTAA | 1 | 16 | 17 | Harbinger- | Harbinger- | 2445005 |
| | ger- | Harbin | piensis | 9 | % | 3_AMi:1-21 | 3_AMi:5309 | 3_AMi:343- | | | | | 3_AMi1- | 3 AMr:20 | _L312 |
| | 3_AMi | ger | | | | | -5329 | 2007 | | | | | 342 | 08-5329 | |
| 348 | Harbin | PIF- | A. | 5,233 | 23.90 | N/A | N/A | Harbinger- | TA | 0 | 16 | 16 | Harbinger- | Harbinger- | 2445005 |
| | ger- | Harbin | mississip | | % | | | IB_Crp:311 | | | | | IB_Crp:1- | 1B_Crp:1 | _L348 |
| | IB_ | ger | piensis | | | | | -1342 | | | | | 310 | 343-5233 | |
| | CrP | | | | | | | | | | | | | | |
| 349 | MARW | TcMar- | D. yakuba | 1643 | 24.10 | SEQ ID | SEQ ID | SEQ ID | SEQ ID | 0 | 0 | 0 | SEQ ID | SEQ ID | |
| | OLENI | Tcl | | | % | NO: 90 | NO: 102 | NO: 114 | NO: 193 | | | | NO: 178 | NO: 190 | |
| 351 | Zaphod | hAT | G. | 3,592 | 24.40 | Zaphod2:1-9 | Zaphod2:35 | Zaphod2:82 | TA | 0 | 0 | 0 | Zaphod2:1 | Zaphod2.:3 | 2445005 |
| | 2 | | variegatu | | % | | 84-3592 | 2-3356 | | | | | ′-821 | 357-3592 | _L351 |
| | | | 8 | | | | | | | | | | | | |

* Except for where SEQ ID NOs are indicated in this table for 5′ TR, 3′ TR, transposase, LTF and RTF, the sequences of these corresponding elements are annotated as the locations of the nucleotides in the corresponding TE sequence as identified by the Repbase ID, the sequences of which are incorporated herein by reference. The Repbase database can be accessed at world wide web.girinst.org. The sequences of the TEs can also be accessed at NCBI (world wide web. ncbi.nlm.nih.gov) with the temporary NCBI identifier in the last column.

TABLE 2
Active transposable elements (TEs) as validated by the transposition assays.

| | | | | | | SEQ ID NOS | | | | | | Transfer | Transfer |
|---|---|---|---|---|---|---|---|---|---|---|---|---|---|
| TE | | | | Length | | | | Trans- | | | 5′TSD, | efficiency | efficiency |
| ID | TE name | Superfamily | Species | (bp) | Divergence | 5′TR | 3′TR | posase | LTF | RTF | 3′TSD | in 293T | in HeLa |
| 1 | hAT-2_AG | hAT | A. gambiae | 4,803 | 0.00% | 1 | 27 | 53 | 115 | 141 | 191 | 5.19% | 7.54% |
| 9 | HAT1_AG | hAT | A. gambiae | 3,702 | 0.10% | 2 | 28 | 54 | 116 | 142 | 192 | 5.28% | 0.87% |
| 14 | Tc1-8B_DR | TcMariner | D. rerio | 1,597 | 0.20% | 3 | 29 | 55 | 117 | 143 | 193 | *15.19% | *11.81% |
| 17 | hAT-6_PM | hAT | P. marinus | 2,391 | 0.20% | 4 | 30 | 56 | 118 | 144 | 195 | 3.16% | 1.38% |
| 22 | hAT-3_XT | hAT | X. tropicalis | 2,922 | 0.30% | 5 | 31 | 57 | 119 | 145 | 196 | 0.27% | 0.87% |
| 25 | Tc1-3_Xt | TcMariner | X. tropicalis | 1,614 | 0.40% | 6 | 32 | 58 | 120 | 146 | 193 | 9.62% | 3.69% |
| 28 | hAT-5_DR | hAT | D. rerio | 2,496 | 0.60% | 7 | 33 | 59 | 121 | 147 | 194 | 2.01% | 0.05% |
| 29 | Tc1-3_FR | TcMariner | T. rubripes | 1,595 | 0.70% | 8 | 34 | 60 | 122 | 148 | 193 | *19.99% | 10.61% |
| 32 | Tc1-5_Xt | TcMariner | X. tropicalis | 1,594 | 1.10% | 9 | 35 | 61 | 123 | 149 | 193 | 7.55% | 6.30% |
| 33 | Tc1-10_Xt | TcMar-Tc1 | X. tropicalis | 1606 | 1.30% | 10 | 36 | 62 | 124 | 150 | 193 | 1.64% | 2.43% |
| 35 | Mariner2_AG | TcMariner | A. gambiae | 2,000 | 1.50% | 11 | 37 | 63 | 125 | 151 | 193 | *17.28% | *9.39% |
| 36 | Tc1-1_Xt | TcMariner | X. laevis | 1,634 | 1.50% | 12 | 38 | 64 | 126 | 152 | 193 | *21.95% | *9.98% |
| 37 | Tc1-1_AG | TcMariner | A. gambiae | 1,413 | 1.60% | 13 | 39 | 65 | 127 | 153 | 193 | 11.18% | 6.43% |
| 39 | Tc1DR3_Xt | TcMariner | X. tropicalis | 1,231 | 1.70% | 14 | 40 | 66 | 128 | 154 | 198 | 1.26% | 3.12% |
| 41 | hAT-3_PM | hAT | P. marinus | 2,984 | 1.90% | 15 | 41 | 67 | 129 | 155 | 199 | 3.47% | 0.33% |
| 43 | Tc1-1_PM | TcMariner | P. marinus | 1,607 | 2.00% | 16 | 42 | 68 | 130 | 156 | 193 | 13.56% | 8.97% |
| 45 | Mariner- | TcMariner | A. | 2,121 | 2.10% | 17 | 43 | 69 | 131 | 157 | 193 | 6.79% | 1.96% |
| | 4_AMi | | mississippiensis | | | | | | | | | | |
| 46 | Myotis_hAT1 | hAT | M. lucifugus | 2,921 | 2.10% | 18 | 44 | 70 | 132 | 158 | 197 | 6.99% | 2.89% |
| 47 | hAT-7_PM | hAT | P. marinus | 2,499 | 2.20% | 19 | 45 | 71 | 133 | 159 | 200 | 7.40% | 4.34% |
| 51 | Tc1-11_Xt | TcMariner | X. tropicalis | 1633 | 2.50% | 20 | 46 | 72 | 134 | 160 | 193 | 4.45% | 1.96% |
| 52 | Tc1-16_Xt | TcMariner | X. tropicalis | 1635 | 2.80% | 21 | 47 | 73 | 135 | 161 | 193 | 9.88% | 7.87% |
| 54 | Tc1-4_Xt | TcMariner | X. tropicalis | 1596 | 3.30% | 22 | 48 | 74 | 136 | 162 | 193 | 14.79% | *6.80% |
| 56 | Tc1-15_Xt | TcMariner | X. tropicalis | 1581 | 3.50% | 23 | 49 | 75 | 137 | 163 | 193 | 13.76% | 3.84% |
| 59 | TC1_FR2 | TcMariner | T. rubripes | 1602 | 3.80% | 24 | 50 | 76 | 138 | 164 | 193 | 5.86% | 1.08% |
| 63 | hAT-9_XT | hAT | X. tropicalis | 5716 | 4.60% | 25 | 51 | 77 | 139 | 165 | 201 | 5.63% | 0.99% |
| 64 | Mariner- | TcMariner | X. tropicalis | 1840 | 4.60% | 26 | 52 | 78 | 140 | 166 | 193 | 8.56% | 3.13% |
| | 5_XT | | | | | | | | | | | | |
| 70 | Mariner- | TcMar-Tc2 | A. | 2479 | 5.10% | 79 | 91 | 103 | 167 | 179 | 193 | 10.27% | N/A |
| | 6_AMi | | mississippiensis | | | | | | | | | | |
| 82 | piggyBac- | PiggyBac | X. tropicalis | 8451 | 6.70% | 80 | 92 | 104 | 168 | 180 | 202 | 3.55% | N/A |
| | 2_XT | | | | | | | | | | | | |
| 86 | piggyBac- | PiggyBac | A. | 3224 | 7.00% | 81 | 93 | 105 | 169 | 181 | 202 | 7.36% | N/A |
| | 1_AMi | | mississippiensis | | | | | | | | | | |
| 90 | Mariner- | TcMar- | A. | 2292 | 7.50% | 82 | 94 | 106 | 170 | 182 | 193 | 10.58% | N/A |
| | 3_Crp | Tigger | mississippiensis | | | | | | | | | | |
| 103 | hAT-1_PM | hAT- | P. marinus | 2311 | 9.40% | 83 | 95 | 107 | 171 | 183 | 203 | 2.51% | N/A |
| | | Charlie | | | | | | | | | | | |
| 139 | hAT-17_Croc | hAT-Ac | A. | 5670 | 13.80% | 84 | 96 | 108 | 172 | 184 | 196 | 3.63% | 4.73% |
| | | | mississippiensis | | | | | | | | | | |
| 142 | Tigger4 | TcMar- | M. murinus | 2731 | 13.80% | 85 | 97 | 109 | 173 | 185 | 193 | 6.17% | 2.28% |
| | | Tigger | | | | | | | | | | | |
| 144 | Tigger7 | TcMar- | T. manatus | 2492 | 13.80% | 86 | 98 | 110 | 174 | 186 | 193 | 1.90% | N/A |
| | | Tigger | latirostris | | | | | | | | | | |
| 180 | hAT-19_Crp | hAT | A. | 2844 | 14.50% | 87 | 99 | 111 | 175 | 187 | 204 | 1.23% | N/A |
| | | | mississippiensis | | | | | | | | | | |
| 183 | hAT- | hAT-Ac | A. | 6042 | 14.60% | 88 | 100 | 112 | 176 | 188 | 205 | 0.77% | N/A |
| | 17B_Croc | | mississippiensis | | | | | | | | | | |
| 222 | hAT-10_XT | hAT-Ac | X. tropicalis | 11144 | 16.00% | 89 | 101 | 113 | 177 | 189 | 206 | 5.07% | N/A |
| 349 | MARWOLEN1 | TcMar-Tc1 | D. yakuba | 1643 | 24.10% | 90 | 102 | 114 | 178 | 190 | 193 | 6.31% | N/A |

The * before a transfer efficiency indicates an underestimation of the real value.

TABLE 3
Sequence similarities among the 14 identified hAT
superfamily transposases and control TE TcBuster

| | | | | | |
|---|---|---|---|---|---|
| TcBusterA2:K17 | 100.0% | | | | |
| 103_hAT-1_PM | 48.3% | 100.0% | | | |
| 17_hAT-6_PM | 36.9% | 40.8% | 100.0% | | |
| 46_Myotis_hAT1 | 35.6% | 39.8% | 48.1% | 100.0% | |
| 28_hAT-5_DR | 38.2% | 40.6% | 45.7% | 43.6% | 100.0% |
| 22_hAT-3_XT | 39.0% | 39.3% | 42.7% | 41.1% | 47.8% |
| 41_hAT-3_PM | 39.6% | 38.5% | 42.3% | 42.5% | 47.9% |
| 47_hAT-7_PM | 40.1% | 37.8% | 43.3% | 44.6% | 49.9% |
| 180_hAT-19_Crp | 17.7% | 18.5% | 18.9% | 19.7% | 20.6% |
| 1_hAT-2_AG | 23.0% | 23.3% | 22.3% | 24.3% | 25.8% |
| 9_HAT1_AG | 21.0% | 19.2% | 22.4% | 23.7% | 23.2% |
| 139_hAT-17_Croc | 22.6% | 20.1% | 18.6% | 21.1% | 23.2% |
| 183_hAT-17B_Croc | 22.0% | 19.6% | 18.7% | 20.8% | 22.5% |
| 63_hAT-9_XT | 25.0% | 22.9% | 20.3% | 22.7% | 24.3% |
| 222_hAT-10_XT | 22.9% | 21.1% | 20.9% | 23.1% | 23.3% |
| | TcBuster | 103_hAT- | 17_hAT- | 46_Myotis_hAT1 | 28_hAT- |
| | | 1_PM | 6_PM | | 5_DR |

| | | | | | |
|---|---|---|---|---|---|
| TcBusterA2:K17 | | | | | |
| 103_hAT-1_PM | | | | | |
| 17_hAT-6_PM | | | | | |
| 46_Myotis_hAT1 | | | | | |
| 28_hAT-5_DR | | | | | |
| 22_hAT-3_XT | 100.0% | | | | |
| 41_hAT-3_PM | 51.9% | 100.0% | | | |
| 47_hAT-7_PM | 53.4% | 56.1% | 100.0% | | |
| 180_hAT-19_Crp | 18.2% | 19.0% | 18.5% | 100.0% | |
| 1_hAT-2_AG | 22.5% | 24.2% | 23.8% | 24.9% | 100.0% |
| 9_HAT1_AG | 22.0% | 22.1% | 22.6% | 24.0% | 62.8% |
| 139_hAT-17_Croc | 21.9% | 21.2% | 21.7% | 24.5% | 38.8% |
| 183_hAT-17B_Croc | 21.2% | 20.7% | 21.1% | 24.2% | 38.3% |
| 63_hAT-9_XT | 20.2% | 20.9% | 21.6% | 27.6% | 38.9% |
| 222_hAT-10_XT | 19.9% | 22.9% | 22.6% | 26.1% | 36.9% |
| | 22_hAT- | 41_hAT- | 47_hAT- | 180_hAT- | 1_hAT- |
| | 3_XT | 3_PM | 7_PM | 19_Crp | 2_AG |

TABLE 4
Sequence similarities among the 14 identified piggyBac
superfamily transposases and control TE piggyBac

| | | | |
|---|---|---|---|
| 82_piggyBac-2_XT | 100.0% | | |
| piggyBac | 33.1% | 100.0% | |
| 86_piggyBac-1_AMi | 35.8% | 44.5% | 100.0% |
| | 82_piggyBac-2_XT | piggyBac | 86_piggyBac-1_AMi |

TABLE 5
Sequence similarities among the 22 identified TcMariner superfamily transposases and control TE SB100X

| | | | | | | |
|---|---|---|---|---|---|---|
| 56_Tc1-15_Xt | 100.0% | | | | | |
| 59_TC1_FR2 | 82.5% | 100.0% | | | | |
| 39_Tc1DR3_Xt | 43.0% | 44.4% | 100.0% | | | |
| 35_Mariner2_AG | 41.6% | 41.0% | 43.7% | 100.0% | | |
| 37_Tc1-1_AG | 39.3% | 41.7% | 40.2% | 55.0% | 100.0% | |
| 32_Tc1-5_Xt | 44.4% | 43.9% | 45.0% | 44.0% | 40.2% | 100.0% |
| 349_MARWOLEN1 | 42.7% | 42.7% | 44.8% | 47.0% | 43.8% | 42.7% |
| SB100X | 43.4% | 45.5% | 46.0% | 48.2% | 46.2% | 51.3% |
| 14_Tc1-8B_DR | 41.2% | 41.2% | 43.3% | 47.3% | 48.3% | 48.0% |
| 54_Tc1-4_Xt | 38.7% | 39.0% | 44.9% | 47.0% | 48.9% | 47.5% |
| 29_Tc1-3_FR | 39.0% | 40.2% | 42.2% | 50.0% | 50.2% | 47.2% |
| 25_Tc1-3_Xt | 39.6% | 41.3% | 43.1% | 50.0% | 51.1% | 47.5% |
| 43_Tc1-1_PM | 39.9% | 42.2% | 42.5% | 48.5% | 50.5% | 49.0% |
| 52_Tc1-16_Xt | 40.4% | 41.8% | 40.6% | 43.1% | 46.2% | 46.1% |
| 36_Tc1-1_Xt | 41.9% | 44.0% | 44.6% | 45.2% | 46.8% | 47.2% |
| 33_Tc1-10_Xt | 37.9% | 40.9% | 44.4% | 45.2% | 44.1% | 47.9% |
| 51_Tc1-11_Xt | 37.4% | 40.0% | 40.6% | 44.9% | 45.9% | 49.4% |
| 64_Mariner- | 24.6% | 24.3% | 17.9% | 20.4% | 22.2% | 19.8% |
| 5_XT | | | | | | |
| 70_Mariner- | 25.1% | 24.0% | 19.0% | 21.9% | 24.9% | 23.7% |
| 6_AMi | | | | | | |
| 142_Tigger4 | 27.5% | 26.3% | 21.9% | 25.4% | 27.0% | 25.6% |
| 144_Tigger7 | 28.7% | 28.7% | 24.2% | 25.4% | 25.2% | 26.7% |
| 45_Mariner- | 23.7% | 24.6% | 23.9% | 24.9% | 21.3% | 24.8% |
| 4_AMi | | | | | | |
| 90_Mariner- | 24.6% | 25.4% | 25.9% | 22.2% | 21.6% | 24.0% |
| 3_Crp | | | | | | |
| | 56_Tc1- | 59_TC1_FR2 | 39_Tc1DR3_Xt | 35_Mariner2_AG | 37_Tc1- | 32_Tc1- |
| | 15_Xt | | | | 1_AG | 5_Xt |

| | | | | | |
|---|---|---|---|---|---|
| 56_Tc1-15_Xt | | | | | |
| 59_TC1_FR2 | | | | | |
| 39_Tc1DR3_Xt | | | | | |
| 35_Mariner2_AG | | | | | |
| 37_Tc1-1_AG | | | | | |
| 32_Tc1-5_Xt | | | | | |
| 349_MARWOLEN1 | 100.0% | | | | |
| SB100X | 47.2% | 100.0% | | | |
| 14_Tc1-8B_DR | 44.5% | 63.0% | 100.0% | | |
| 54_Tc1-4_Xt | 46.9% | 63.0% | 69.8% | 100.0% | |
| 29_Tc1-3_FR | 45.7% | 63.6% | 71.6% | 76.8% | 100.0% |
| 25_Tc1-3_Xt | 46.6% | 61.9% | 71.3% | 80.6% | 86.8% |
| 43_Tc1-1_PM | 46.3% | 61.6% | 71.3% | 82.1% | 87.4% |
| 52_Tc1-16_Xt | 44.8% | 59.8% | 60.5% | 53.1% | 56.0% |
| 36_Tc1-1_Xt | 45.7% | 62.2% | 63.0% | 59.5% | 61.3% |
| 33_Tc1-10_Xt | 42.7% | 60.6% | 63.8% | 61.2% | 62.9% |
| 51_Tc1-11_Xt | 45.7% | 61.8% | 63.2% | 60.3% | 61.5% |
| 64_Mariner- | 18.2% | 23.2% | 21.1% | 19.6% | 19.1% |
| 5_XT | | | | | |
| 70_Mariner- | 20.9% | 26.7% | 24.6% | 24.0% | 24.0% |
| 6_AMi | | | | | |
| 142_Tigger4 | 23.6% | 26.4% | 25.4% | 24.6% | 26.4% |
| 144_Tigger7 | 23.3% | 28.7% | 24.9% | 27.6% | 25.8% |
| 45_Mariner- | 23.3% | 29.3% | 25.1% | 27.9% | 24.9% |
| 4_AMi | | | | | |
| 90_Mariner- | 24.8% | 27.3% | 23.7% | 24.9% | 22.3% |
| 3_Crp | | | | | |
| | 349_MARWOLEN1 | SB100X | 14_Tc1- | 54_Tc1- | 29_Tc1- |
| | | | 8B_DR | 4_Xt | 3_FR |

| | | | | | | | |
|---|---|---|---|---|---|---|---|
| 25_Tc1-3_Xt | 100.0 | | | | | | |
| 43_Tc1-1_PM | 88.0 | 100.0 | | | | | |
| 52_Tc1-16_Xt | 56.6 | 56.3 | 100.0 | | | | |
| 36_Tc1-1_Xt | 60.1 | 61.0 | 66.3 | 100.0 | | | |
| 33_Tc1-10_Xt | 64.1 | 63.5 | 57.4 | 64.7 | 100.0 | | |
| 51_Tc1-11_Xt | 61.8 | 60.0 | 60.3 | 65.9 | 70.9 | 100.0 | |
| 64_Mariner- | 19.9 | 19.9 | 21.4 | 22.9 | 21.8 | 19.4 | 100.0 |
| 5_XT | | | | | | | |
| 70_Mariner- | 24.0 | 24.0 | 24.6 | 25.8 | 20.6 | 21.2 | 56.3 |
| 6_AMi | | | | | | | |
| 142_Tigger4 | 24.9 | 24.0 | 24.9 | 24.0 | 22.1 | 23.5 | 29.1 |
| 144_Tigger7 | 27.0 | 26.4 | 24.9 | 27.6 | 23.5 | 24.1 | 28.6 |
| 45_Mariner- | 25.5 | 26.7 | 26.7 | 27.6 | 24.1 | 25.9 | 27.0 |
| 4_AMi | | | | | | | |
| 90_Mariner- | 23.8 | 23.2 | 24.6 | 23.5 | 22.1 | 24.4 | 27.7 |
| 3_Crp | | | | | | | |
| % | 25_Tc1- | 43_Tc1- | 52_Tc1- | 36_Tc1- | 33_Tc1- | 51_Tc1- | 64_Mariner- |
| | 3_Xt | 1_PM | 16_Xt | 1_Xt | 10_Xt | 11_Xt | 5_XT |

| | | | | | |
|---|---|---|---|---|---|
| 25_Tc1-3_Xt | | | | | |
| 43_Tc1-1_PM | | | | | |
| 52_Tc1-16_Xt | | | | | |
| 36_Tc1-1_Xt | | | | | |
| 33_Tc1-10_Xt | | | | | |
| 51_Tc1-11_Xt | | | | | |
| 64_Mariner- | | | | | |
| 5_XT | | | | | |
| 70_Mariner- | 100.0 | | | | |
| 6_AMi | | | | | |
| 142_Tigger4 | 31.0 | 100.0 | | | |
| 144_Tigger7 | 30.1 | 41.3 | 100.0 | | |
| 45_Mariner- | 29.2 | 40.1 | 50.5 | 100.0 | |
| 4_AMi | | | | | |
| 90_Mariner- | 27.8 | 41.8 | 46.7 | 55.4 | 100.0 |
| 3_Crp | | | | | |
| % | 70_Mariner- | 142_Tigger4 | 144_Tigger7 | 45_Mariner- | 90_Mariner- |
| | 6_AMi | | | 4_AMi | 3_Crp |

1. An engineered transposable element comprising, from 5′ to 3′:
a 5′ terminal repeat sequence (5′TR), a heterologous nucleic acid, and a 3′ terminal repeat sequence (3′TR);
wherein the 5′TR comprises a nucleic acid sequence selected from the group consisting of SEQ ID NOs: 1-26 and 79-90, a variant thereof, or a fragment thereof, wherein the 3′TR comprises a nucleic acid sequence selected from the group consisting of SEQ ID NOs: 27-52 and 91-102, a variant thereof, or a fragment thereof; and
wherein the engineered transposable element exhibits transposition activity that allows the heterologous nucleic acid to be inserted into a DNA of a cell.

2. The engineered transposable element of claim 1, wherein the 5′TR comprises a nucleic acid sequence that has at least about 90% sequence identity to a nucleic acid sequence selected from the group consisting of SEQ ID NOs: 1-26 and 79-90, and/or wherein the 3′TR comprises a nucleic acid sequence that has at least about 90% sequence identity to a nucleic acid sequence selected from the group consisting of SEQ ID NOs: 27-52 and 91-102.

3. (canceled)

4. The engineered transposable element of claim 1, further comprising a 5′ target site duplication sequence (TSD) flanking the 5′ of the 5′TR or a 3′TSD flanking the 3′ of the 3′TR, wherein the 5′TSD comprises a nucleic acid sequence selected from the group consisting of SEQ ID NOs: 191-206, a variant thereof, or a fragment thereof, and wherein the 3′TSD comprises a nucleic acid sequence selected from the group consisting of SEQ ID NOs: 191-206, a variant thereof, or a fragment thereof.

5. The engineered transposable element of claim 1, wherein the 5′TR comprises a nucleic acid sequence that has at least about 90% sequence identity to a nucleic acid sequence selected from the group consisting of SEQ ID NOs: 3, 8, 11, 12, 13, 16, 22, 23, 79, and 82, and the 3′TR comprises a nucleic acid sequence that has at least about 90% sequence identity to a nucleic acid sequence selected from the group consisting of SEQ ID NOs: 29, 34, 37, 38, 39, 42, 48, 49, 91, and 94.

6. The engineered transposable element of claim 1, wherein:
(a) the 5′TR comprises a nucleic acid sequence of SEQ ID NO: 3, and the 3′TR comprises a nucleic acid sequence of SEQ ID NO: 29;
(b) the 5′TR comprises a nucleic acid sequence of SEQ ID NO: 8, and the 3′TR comprises a nucleic acid sequence of SEQ ID NO: 34;
(c) the 5′TR comprises a nucleic acid sequence of SEQ ID NO: 11, and the 3′TR comprises a nucleic acid sequence of SEQ ID NO: 37;
(d) the 5′TR comprises a nucleic acid sequence of SEQ ID NO: 12, and the 3′TR comprises a nucleic acid sequence of SEQ ID NO: 38; or
(e) the 5′TR comprises a nucleic acid sequence of SEQ ID NO: 16, and the 3′TR comprises a nucleic acid sequence of SEQ ID NO: 42.

7. The engineered transposable element of claim 1, wherein the heterologous nucleic acid comprises a coding sequence.

8. The engineered transposable element of claim 7, wherein the heterologous nucleic acid further comprises a promoter operably linked to the coding sequence.

9. The engineered transposable element of claim 1, wherein the transposition activity of the engineered transposable element is higher than that of a piggyBac (PB) transposon, a Sleeping Beauty (SB) transposon, and/or a TcBuster (TB) transposon.

10. The engineered transposable element of claim 1, wherein the cell is an animal cell, a plant cell, an algal cell, a fungal cell, a yeast cell, or a bacterial cell.

11. The engineered transposable element of claim 1, wherein the cell is a mammalian cell.

12-13. (canceled)

14. The engineered transposable element of claim 1, wherein the transposition activity of the engineered transposable element is higher in healthy human cells than in cancer cells.

15. The engineered transposable element of claim 1, wherein the transposable element is present in a vector.

16. The engineered transposable element of claim 15, wherein the vector is a plasmid or a viral vector.

17. A gene transfer system comprising: i) an engineered transposable element of claim 1; and ii) a transposase, or a nucleic acid encoding h transposase.

18. The gene transfer system of claim 17, wherein the transposase comprises an amino acid sequence selected from the group consisting of SEQ ID NOs: 53-78 and 103-114, or a variant thereof.

19. A gene transfer system comprising: i) an engineered transposable element; and ii) a transposase, or a nucleic acid encoding h transposase, wherein the engineered transposable element comprises from 5′ to 3′:
a 5′TR, a heterologous nucleic acid, and a 3′TR,
wherein the engineered transposable element exhibits transposition activity that allows the heterologous nucleic acid to be inserted into a DNA of a cell, and
wherein the transposase comprises an amino acid sequence selected from the group consisting of SEQ ID NOs: 53-78 and 103-114, or a variant thereof.

20-24. (canceled)

25. A method of inserting a heterologous nucleic acid into a target nucleic acid, comprising:
contacting the target nucleic acid with the gene transfer system of claim 17, thereby inserting the heterologous nucleic acid into the target nucleic acid.

26. (canceled)

27. The method of claim 25, wherein the target nucleic acid is in a cell.

28-31. (canceled)

32. The method of claim 27, wherein insertion of the heterologous nucleic acid inactivates a gene of the cell.

33-39. (canceled)


SEQUENCE LISTING (206 sequences)

SEQ ID NO: 1 - DNA, 5 bp, Anopheles gambiae
taggc

SEQ ID NO: 2 - DNA, 3 bp, Anopheles gambiae
tag

SEQ ID NO: 3 - DNA, 201 bp, Danio rerio
cagcggggaaaataagtatttgacacatcagcatttttatcagtaaggggatttctaagtgggctactgacacaaaattcctaccagatgtagccatcaagccaaatattgaattcatacaaagaaatcagaacatttaagtatacaagttgagtcataataaataaagtgaaatgacacagggaataagtattgaacaca

SEQ ID NO: 4 - skipped

SEQ ID NO: 5 - DNA, 6 bp, Xenopus tropicalis
cagggg

SEQ ID NO: 6 - DNA, 210 bp, Xenopus tropicalis
cagtggaggaaataattatttgacccctcactgattttgtaagtttgtccaatgacaaagaaatgaaaagtctcagaacagtatcatttcaatggtaggtttattttaacagtggcagatagcacatcaaaaggaaaatcgaaaaaataactttaaataaaagatagcaactgatttgcatttcattgagtgaaataagtttttgaaccc

SEQ ID NO: 7 - DNA, 15 bp, Danio rerio
caggggtggcgaacc

SEQ ID NO: 8 - DNA, 131 bp, Takifugu rubripes
cagtgagagtaaaaagtatttgatcccttgctgattttgttggtttgtccactaataaagacatgatcattctatacttttaatggtagatgtattctaacatggagagacagaatatcaaaaagaaaatc

SEQ ID NO: 9 - DNA, 213 bp, Xenopus tropicalis
caaaccggattccaaaaaagttgggacactaaacaaattgtgaataaaaactgaacgcaatgatgtggaggtgccaacttctaatattttattcagaatagaacataaatcacggaacaaaagtttaaactgagaaaatgtaccattttaagggaaaaatatgttgattcagaatttcatggtgtcaacaaatcccaaaaaagttgggacaag

SEQ ID NO: 10 - DNA, 214 bp, Xenopus tropicalis
cagtggcttgcaaaagtattcggcccccttgaacttttccacattttgtcacattacagccacaaacatgaatcaattttattggaattccacgtgaaagaccaatacaaagtggtgtacacgtgagaagtggaacgaaaatcatacatgattccaaacattttttacaaataaataactgcaaagtggggtgtgcgtaattattcagccccct

SEQ ID NO: 11 - DNA, 18 bp, Anopheles gambiae
tacagtgtcggacaaatc

SEQ ID NO: 12 - DNA, 63 bp, Xenopus laevis
cagtggtgtgaaaaactatttgcccccttcctgatttcttattcttttgcatgtttgtcacac

SEQ ID NO: 13 - DNA, 13 bp, Anopheles gambiae
cactggtggacat

SEQ ID NO: 14 - DNA, 8 bp, Xenopus tropicalis
atatacac

SEQ ID NO: 15 - DNA, 16 bp, Petromyzon marinus
caggggtcaccaaact

SEQ ID NO: 16 - DNA, 129 bp, Petromyzon marinus
cagtgagggaaaaaagtatttgatcccctgctgattttgtacgtttgcccactgacaaagaaatgatcagtctataattttaatggtaggtttatttgaacagtgagagacagaataacaacaacaaaa

SEQ ID NO: 17 - DNA, 5 bp, Alligator mississippiensis
caggc

SEQ ID NO: 18 - DNA, 16 bp, Myotis lucifugus
cagtgatggcgaacct

SEQ ID NO: 19 - DNA, 3 bp, Petromyzon marinus
cag

SEQ ID NO: 20 - DNA, 157 bp, Xenopus tropicalis
cagtggatataaaaagtctacacacccctggtaaaatgtcaggttcctgtgctgtacaaaaatgagacaaagataaatcatttcagaactttttccacctttaatgtgacctataaactgtaccactcaattgaaaaacaaactgaaatcttttagg

SEQ ID NO: 21 - DNA, 215 bp, Xenopus tropicalis
cagtcatggccaaaattgttggcaccccagaaatttttccagaaaatcaagtatttctcacagaaaagtattgcagtaacacatgttttgctatacacatgtttattccctttgtgtgtattggaacagaacaaaaaagggaggaaaaaaagcaaattggacataatgtcacacaaaactccaaaaatgggctggacaaaattattggcaccctt

SEQ ID NO: 22 - DNA, 204 bp, Xenopus tropicalis
cagtgaggaacataagtatttgaacaccctgcgattttgcaagttctcccacttagaaatcatggaggggtctgaaattcacattgtaggtgcattcccactgtgagagacagcatttaaaaaaaaaattcaggaaatcacattgtatgatttttaaagaatgtatttgtattgcactgctgcacataagtatttgaacacctg

SEQ ID NO: 23 - DNA, 27 bp, Xenopus tropicalis
cactgctcaaaaaaataaagggaacac

SEQ ID NO: 24 - DNA, 29 bp, Takifugu rubripes
cactgctcaaaaaaattagaggaacactt

SEQ ID NO: 25 - DNA, 3 bp, Xenopus tropicalis
tag

SEQ ID NO: 26 - DNA, 28 bp, Xenopus tropicalis
ccgtattttccgcactataaggcgcacc

SEQ ID NO: 27 - DNA, 5 bp, Anopheles gambiae
gccta

SEQ ID NO: 28 - DNA, 3 bp, Anopheles gambiae
cta

SEQ ID NO: 29 - DNA, 201 bp, Danio rerio
tgtgttcaatacttattccctgtgtcatttcactttatttattatgactcaacttgtatacttaaatgttctgatttctttgtatgaattcaatatttggcttgatggctacatctggtaggaattttgtgtcagtagcccacttagaaatccccttactgataaaaatgctgatgtgtcaaatacttattttccccgctg

SEQ ID NO: 30 - skipped

SEQ ID NO: 31 - DNA, 6 bp, Xenopus tropicalis
cccctg

SEQ ID NO: 32 - DNA, 210 bp, Xenopus tropicalis
gggttcaaaaacttatttcactcaatgaaatgcaaatcagttgctatcttttatttaaagttattttttcgattttccttttgatgtgctatctgccactgttaaaataaacctaccattgaaatgatactgttctgagacttttcatttctttgtcattggacaaacttacaaaatcagtgaggggtcaaataattatttcctccactg

SEQ ID NO: 33 - DNA, 15 bp, Danio rerio
ggttcgccacccctg

SEQ ID NO: 34 - DNA, 131 bp, Takifugu rubripes
gattttctttttgatattctgtctctccatgttagaatacatctaccattaaaagtatagaatgatcatgtctttattagtggacaaaccaacaaaatcagcaagggatcaaatactttttactctcactg

SEQ ID NO: 35 - DNA, 213 bp, Xenopus tropicalis
cttgtcccaacttttttgggatttgttgacaccatgaaattctgaatcaacatatttttcccttaaaatggtacattttctcagtttaaacttttgttccgtgatttatgttctattctgaataaaatattagaagttggcacctccacatcattgcgttcagtttttattcacaatttgtttagtgtcccaacttttttggaatccgggttg

SEQ ID NO: 36 - DNA, 214 bp, Xenopus tropicalis
agggggctgaataattacgcacaccccactttgcagttatttatttgtaaaaaatgtttggaatcatgtatgattttcgttccacttctcacgtgtacaccactttgtattggtctttcacgtggaattccaataaaattgattcatgtttgtggctgtaatgtgacaaaatgtggaaaagttcaagggggccgaatacttttgcaagccactg

SEQ ID NO: 37 - DNA, 18 bp, Anopheles gambiae
gatttgtccgacactgta

SEQ ID NO: 38 - DNA, 63 bp, Xenopus laevis
gtgtgacaaacatgcaaaagaataagaaatcaggaagggggcaaatagtttttcacaccactg

SEQ ID NO: 39 - DNA, 13 bp, Anopheles gambiae
atgtccaccagtg

SEQ ID NO: 40 - DNA, 8 bp, Xenopus tropicalis
gtgtatat

SEQ ID NO: 41 - DNA, 16 bp, Petromyzon marinus
agtttggtgacccctg

SEQ ID NO: 42 - DNA, 129 bp, Petromyzon marinus
ttttgttgttgttattctgtctctcactgttcaaataaacctaccattaaaattatagactgatcatttctttgtcagtgggcaaacgtacaaaatcagcaggggatcaaatacttttttccctcactg

SEQ ID NO: 43 - DNA, 5 bp, Alligator mississippiensis
gcctg

SEQ ID NO: 44 - DNA, 16 bp, Myotis lucifugus
aggttcgccatcactg

SEQ ID NO: 45 - DNA, 3 bp, Petromyzon marinus
ctg

SEQ ID NO: 46 - DNA, 157 bp, Xenopus tropicalis
cctaaaagatttcagtttgtttttcaattgagtggtacagtttataggtcacattaaaggtggaaaaagttctgaaatgatttatctttgtctcatttttgtacagcacaggaacctgacattttaccaggggtgtgtagactttttatatccactg

SEQ ID NO: 47 - DNA, 215 bp, Xenopus tropicalis
aagggtgccaataattttgtccagcccatttttggagttttgtgtgacattatgtccaatttgctttttttcctcccttttttgttctgttccaatacacacaaagggaataaacatgtgtatagcaaaacatgtgttactgcaatacttttctgtgagaaatacttgattttctggaaaaatttctggggtgccaacaattttggccatgactg

SEQ ID NO: 48 - DNA, 204 bp, Xenopus tropicalis
caggtgttcaaatacttatgtgcagcagtgcaatacaaatacattctttaaaaatcatacaatgtgatttcctgaattttttttttaaatgctgtctctcacagtgggaatgcacctacaatgtgaatttcagacccctccatgatttctaagtgggagaacttgcaaaatcgcagggtgttcaaatacttatgttcctcactg

SEQ ID NO: 49 - DNA, 27 bp, Xenopus tropicalis
gtgttccctttatttttttgagcagtg

SEQ ID NO: 50 - DNA, 29 bp, Takifugu rubripes
aagtgttcctctaatttttttgagcagtg

SEQ ID NO: 51 - DNA, 3 bp, Xenopus tropicalis
cta

SEQ ID NO: 52 - DNA, 28 bp, Xenopus tropicalis
ggtgcgccttatagtgcggaaaatacgg

SEQ ID NO: 53 - protein, 595 aa, Anopheles gambiae
MASNVATSSSIWDHFTRNGTKAKCRYCYNEIAFTKGSTSNLKRHMKAKHPSISLERQHLASTTSPSTQNSEPGPSCSSRPPNLISNFFKQPMNSETKRKLDMMLLKLICKDCLPLSIVESEAFKTFVGCLNQNYDLPTRKNVSNALLPSIYNEILVKVQGEVRNATSIALTTDGWTNVNNTSFLGLTAHFIDNDYKLRSCLLECSEISLSHSGQNIAAWIKEVIIKYEIQDKIVGIVTDNAANMKSAARELEFNHVTCFAHSLHLIVKDAIKKSIISTVDEVKRIVMYFKKSPKATNELADTQSKFNLPNLKLKQDVPTRWNSTYDMLNRFYKNKIAIVACADKLNTLDPIKIDWAILEHSLNALKIFDVATNMVSAEKNITVSHVGLLSKMIIRKLNETDYTIPELGNLVTHLKEGVKKRLEIYSNNQIIAKSMLLDPRIKKQGFHEEPLKYRETYELIIQELIPFQTPSMNAQEPHNIDNEANLLLGEFITWVNNAECEIESPIELAKNELNSFLKIRNIDIKNDPLEWWRIHSSKYPSIYALAKTIICIPGTSVPCERLFSKAGQIYSDKRSRLHPKKFKEIIFIQQNVDKF

SEQ ID NO: 54 - protein, 603 aa, Anopheles gambiae
MMAPTNATTSPVWDHFSPVETGAKCLYCLKVFKYTKGTTSNLKRHLNLVHKTVPYLKQKQPIPQTINIDDEAGPSAVNFQPSNQYFNSNMSIQGYLKKPINSETKKVLDRMLLDLICKECLPFNLVESEIFKKFVYTLNPNYIMPTRKSLSNALLPSVYNQEFEKAKEKLSTAKAIAITSDGWTNLNQISFFALTGHYIDENCKLSSILIECSEFENPHSGRNIANWIQGTLNKFDIEDKIVAMVTDNASNMKAASTELNFCHIPCFAHTLNLIVRDAIKKSVLPVVEEVKRVVMLFKKSPKASQMLADTQKKLNLDQLKMIQEVSTRWNSGYDMLNRFYKNKIALLSCADSLKMKISLESHDWEAIEQIVRVLKYFYSATNIVSAQKYITISHVGLLCNVLLTKTSQFRNDEDIAENIQNLVALLIEGLQNKLKIYRSNEQILKSMILDPRIKQLGFQDDVEKFKNICESIISELLPLQKPAVEVEKVVKKVSKDVDMLFGDLLKNKGAQNYKTPRQIAENELHQYLSVENIDLENDPLLWWKEHQVLYPSLYTLAMSTLCIPGTSVPCERLFSKAGQIYSEKRSRLAPKKLQEILFIQQNA

SEQ ID NO: 55 - protein, 341 aa, Danio rerio
MMGKNKELSQDLRSLIVEKHFDGNGYRRISRMLNVPVSTVGAIIRKWKKHKFTINRPRSGAPRKIPVRGVQRIIRRVLQEPRTTRAELQEDLASAGTIVSKKTISNALNHHGIHARSPRKTPLLNKKHVEARLKFAKQHLEKPVDYWETIVWSDESKIELFGSHSTHHVWRRNGTAHHPKNTIPTVKFGGGSIMVWGCFSARGTGRLHIIEGRMNGEMYRDILDKNLLPSTRKLKMKRGWTFQQDNDPKHKAKETMKWFQRKKIKLLEWPSQSPDLNPIENLWRELKIKVHKRGPRNLQDLKTVCVEEWARITPEQCRRLVSPYKRRLEAVITNKGFSTKY

SEQ ID NO: 56 - protein, 645 aa, Petromyzon marinus
MAKRKKDDEYRTFQDEWTEEFAFVERAGSAVCLICNDKITSLKRSNVKRHFDTRHATFASKYPAGDSRKKACQELLSRVQASQQQLRVWTRQGDYNSASFAGSLAIVRNGKPFTDGEYAKTFMLDVANELFDDLPNKDKIIKRIQDMPLSARTVHDRTIVMANKVEETQVKDINAAPFFSLALDESTDVSHLSQFSVIARYAVGDTLREESLAVLPLKGSTRGEDLFKSFMEFAQEKSLPMDKLLSVCTDGAPCMVGKNKGFVALLREHENRPILSFHCIIHQEALCAQMCDRQFGEVMSLVIRVINFIVARALNDRQFKTLLDEVVNNYPGLLLHSNVRWLSRGKVLSRFAACLNEIRTFLEMKGVGHPELADTEWLLKFYYLVDLTEHLNQLNVKMQGIGNTVLSLQQAVFAFENKLELFITDLETGRLLHFEKLSQFKDACTASEPIQNLDLHQLAGCTSSLLQSFKARFGEFREHTRLFKFITHPNECSLNTADLNYIPGVSVRDFEAEVADLKASDMWVNKFKSLNEDLERITRQKAELASKHMWTEMKKLQPEDQLILKTWNALPVTYHTLQRVSIAVLTMFGSTYACEQSFSHLKNIKSNLRSRLTDESLNACMKLNLTKYQPDYKDISKSMQHQKSH

SEQ ID NO: 57 - protein, 612 aa, Xenopus tropicalis
MTSSQPAVKRKIDDEHRQFQEKWEMQYFFVEHRGIPTCLICAEKVAVHKEYNLKRHYSTKHAEECAKYQGDERAKRVASLKACLMRQQDFFKKATKENVASVQASYMVSEMIAKAGKPFTEGEFVKKCMLQVASIICPEKKGQFSKISLSANTVAERISDMSSDIYHQLCEKAKCFDAYSVALDESTDITGTAQLTIYVRGVDCNFELTEELLTIIPMHGQTTANEIFHHLCDAIENAGLPWKRFVGIITDGAPSMTGRKNGLVALVKKKLEEEGIEEEAIALHCIIHQQALCSKCLPCDNVMSVVVKCVNQIRSRGLTHRRFRAFLEEMGSEYGDVLYFTEVRWLSRGNVLKRFFELREEVKAFMEKNGKAVSELSDHKWLMDLAFLVDITQRLNVLNKMLQGQGQLVSAAYDNVRAFSTKLVLWKSQLSQTNLCHFPACKELVDAGIPFSGEKYVDAIFKLEKEFDHRFADFKTHRATFQIFVDPFSFDVQDAPPVLQMELIDLQCNSDIKAKFREMSGKADTHVQFLRELPPSFPELSRMFKRTMCLFGSTYLCEKLFSTLNFNKSKYRSRLNDDHLQAILRVSTASSLKPNVVQICEKKRCQVSGSKE

SEQ ID NO: 58 - protein, 340 aa, Xenopus tropicalis
MGKTKELSKDVRDKIVDLHKAGMGYKTISKKLGEKVTTVGAIVRKWKEHKMTINRPRSGAPRKISPRGVSMILRKVKKHPRTTREELVNDLKLAGTTVTKKTIGNTLHRNGLKSCRARKVPLLKKAHVQARLKFANEHLNDSVSDWEKVLWSDETKIELFGINSTRCVWRKKNAAYDPQNTVPTVKHGGGNILLWGCFSAKGTGQLIRINGKMDGAMYREILNDNLLPSARKLKMGRGWVFQHDNDPKHTAKATKEWLKKKHIKVMEWPSQSPDLNPIENLWRELKLRVAQRQPRNLRDLEMICKEEWTNIPPKMCANLVINYKKRLTSVLANKGFSTKY

SEQ ID NO: 59 - protein, 616 aa, Danio rerio
MAESGKRTAKRKYEDERRTFLSEWEDLYFFVERNGKPFCLICQTSLSHFKASNLERHFTSLHSAVAREFPKGSELRKHKVKTLKGQAEKQTQLFRKFTKHSETVTLASYQLAWNIARAKKPYLEGEFVKKCLSDAVAILCPENENLKRSVKDLQLSRHTVEQRISDIDNSVETHLLSDLQKCQYFSIALDESCDVQDKPQLAIFVRFVSEDCTIREELLDIVPLKDRTRGIDLKETLMTVVEKANLQLSKLTAIVTDGAPAMLGSERGLVGLCKADDRFPAFWTFHCIIHQEHLVSKKLNLDHIMKPVLEIVNFVRTHALNHRQFKNLIDELDEDLPSDLLFHCAVRWLSRGHVLSRFFELLNPVKLFLAEKHKEYPELHDPQWISDLAFLVDVLHYLNGLNVDLQGKLKMLPDLVQSVFAFVNKLKLFKTHLQKRDYTHFPTLLKASGQEAEVVKGSTARYATLLENLEQSFEERFSNLRQKRQQITFLINPFTAESGCLKAPLVEDEAASQLEMIELSEDDRLKSVLREGTVEFWKIVPVERYPNVKQAALKLLSMFGSTYVCESLFSTLKLVKSKHRSVLTDTHVKELLRVATTEYEPDLKKIVETKECQVSH

SEQ ID NO: 60 - protein, 340 aa, Takifugu rubripes
MGKTKELSQDLRDRIVDLHKSGMGYKNISKLLGIKVTTIGAIVRKFKKYNMTINRPRSGAPQKISPRGVAMIMRTVRNRPATTQQELVNDLQAAGTKVTRKTIGNTLRRNGLKSCSARKVPLLKKAHVEARLKYANDHLKDAQSDWEKVLWSDETKIELFGLNSTRHVWRKKNAAYDPRNTVPTVKHGGGNIMFWGCFSAKGTGLLHRITGKMDGAMYRAVLRDNLLPSARKLKMGRGWVFQHDNDPKHTAKATKEWLKKNHIKVMEWPSQSPDLNPIENLWRELKVRVAKRQPTNLNDLERICKEEWAKIPPDMCANLVVNYNKRLTAVLANNGFATKY

SEQ ID NO: 61 - protein, 358 aa, Xenopus tropicalis
MIGYKKSFSEWQCLSEAKMGRGSPIPTMLRRKIVEQYQKGVTQRKIAKILHLSSSTVHNIIRRFRESGTISVRKGQGRKTILDARDLRALKRHCTTNRNATVKEITEWAQEYFQKPLSVNTIHRAIRRCQLKLYSAKKKPFLSKIHKLRRFHWARDHLKWSVAKWKTVLWSDESRFEVLFGNLGRHVIRTKEDKDNPSCYQRSVQKPASLMVWGCMSACGMGSLHVWKGSINAEKYIQVLEQHMLPSRRHLFQGRPCIFQQDNARPHSASITTSWLRRRRIRVLKWPVCSPDLSPIENIWRIIKRKVRQRRPKTIEQLEACIRQEWESIPIPKLEKLVSSVPRRLLSVVRRRGDATQW

SEQ ID NO: 62 - protein, 339 aa, Xenopus tropicalis
MKSKEHTRQVRDKVIEKFKAGLGYKKISKALNIPRSTVQAIIQKWKEYGTTVNLPRQGRPPKLTGRTRRALIRNAAKRPMVTLDELQRSTAQVGESVHRTTISRALHKVGLYGRVARRKPLLTENHKKSRLQFATSHVGDTANMWKKVLWSDETKMELFGQNAKRYVWRKTNTAHHSEHTIPTVKYGGGSIMLWGCFSSAGTGKLVRVDGKMDGAKYRAILEENLLESAKDLRLGRRFTFQQDNDPKHKARATMEWFKTKHIHVLEWPSQSPDLNPIENLWQDLKTAVHKRCPSNLTELELFCKEEWARISVSRCAKLVETYPKRLAAVIAAKGGSTKY

SEQ ID NO: 63 - protein, 333 aa, Anopheles gambiae
MGRSAHSTQQQRLDIKRLSAAGYSQRKIAEILGRSKTFVYNALHSTGTKIPTGRPRKTSARDDARMTRLCKADPFKSARAIRDELQLSVSDRTVQRRLFENNLVGRNPRKVPLLRRCHVQARLQFAREHYDWAGENLNKWRNVLWSDESKVNLVGSDGKRFVRRPKNTAYRPQYTLKTVKHGGGNIMVWACFSWYGVGPIFWIKDIMDQHRYLNIIQTVMLPHAEWEMPLKWQFMHDNDPKHTAKAVKKWFVDQKIDVMNWPAQSPDLNPIENLWKIVKAKLPPAGSRTKEKLWKHIENAWYSIPPSTCKSLVESMPKRMRAVIRNNGHATKY

SEQ ID NO: 64 - protein, 340 aa, Xenopus laevis
MPRSKEIQEQMRTKVIEIYQSGKGYKAISKALGLQRTTVRAIIHKWQKHGTVVNLPRSGRPTKITPRAQRQLIREATKDPRTTSKELQASLASIKVSVHDSTIRKRLGKNGLHGRFPRRKPLLSKKNIKARLNFAKKHLNDCQDFWENTLWTDETKVELFGRCVSRYIWRKSNTAFQKKNIIPTVKYGGGSVMVWGCFAASGPGRLAVIDGTMNSTVYQKILKENVRPSVRQLKLKRSWVLQQDNDPKHTSKSTSEWLKKNKMKTLEWPSQSPDLNPIEMLWHDLKKAVHARKPSNKAELQQFCKDEWAKIPPERCKRLVASYRKRLIAVIAAKGGPTSY

SEQ ID NO: 65 - protein, 332 aa, Anopheles gambiae
MGRGKHCTPEERKHIQGLYRENVPIKTICKAFGRSRTFVDNAIRSEATGKSTGRPRKTTADVDAQIVEMIRADPFKTCTRIKQELGLQVSAKTVSRRLHAAGFCARRPRKVRKLLPHHVEARIRFAEEHLAASIFWWSKIIFSDESRINLDGSDGIKYVWRFPNQAYHPKNTIKTLSHGGGHVMVWGCFSWHGTGPLFRINGTLNSEGYRKILSRKMLPYARQQFGDEEHYIFQHDNDSKHTSRTVKCYLANQDVQVLPWPALSPDLNPIENLWSTLKRQLKNQPARSADDLWTRCKVMWERIPRSECRNLIGDMAKRCQEVIANNGHQIDR

SEQ ID NO: 66 - protein, 346 aa, Xenopus tropicalis
MGKKGDLSNFERGMVVGARRAGLSISQSAQLLGFSRTTISRVYKEWCEKGKTSSMRQSCGRKCLVDARGQRRMGRLIQADRRATLTEITTRYNRGMQQSICEATTRTTLRRMGYNSRRPHRVPLISTTNRKKRLQFAQAHQNWTVEDWKNVAWSDESRFLLRHSNGRVRIWRKQNENMDPSCLVTTVQAGGGGVMVWGMFSWHTLGPLVPIGHRLNATAYLSIVSDHVHPFMTTMYPSSDGYFQQDNAPCHKARIISNWFLEHDNEFTVLKWPPQSPDLNPIEHLWDVVERELRALDVHPTNLHQLQDAILSIWANISKECFQHLVESMPRRIKAVLKAKGGQTPY

SEQ ID NO: 67 - protein, 602 aa, Petromyzon marinus
MATKKRKVDAECRVFNEEWGVKYFFVETKDQKASCVICSESVAVLKEYNLRRHYETKHLSTYSKFSGKLRSEKYESMKRGLETQRNLFTRKFAENESVTRTSYKIVHKMAERGKPFTDGNFIKECMMEAANDLCPERADLFGSISLSASSVVRRTEELGENIVLQIREKARNLLWYSLALDESTDLSSTSQLLVFIRGVNLDFQITEELASVCSMHGTTTGKDIFMEVQKTLQDYNLQWNQLRGVTVDGGKNMAGVRKGLVGQIRTQLEDLQIPGALFIHCIIHQQALCGKDLDISCVLKPVVSAVNFIRGHALNHRQFQAFLEEMDSDFCDLPYHTAVRWLSCGKVLFRFYKLRNEIDVFLTEKDRADPQLSDPTWLSKLSFLVDITSHMNELNLKLQGKDNLVCDLYRIIKGFRRKLSLFEAQLEGENFSHFHCFKEFCATIAEDVNLDFPKKIIRDLKKHFLKRFSDLDRIESDILLFQNPFDCNLDDVPVELQLELIDLQANDLLKEKHREGKLVEFYRCLPDVEFPKLKKFAAGMASVFGTTYVCEQTFSKMKYVKSTHRTRLTDEHLKAILLIGCSNSKPNIDDILKAKRQFHKSH

SEQ ID NO: 68 - protein, 340 aa, Petromyzon marinus
MAKTKELSKDVRDKIVDLHKAGMGYKTIAKQLGEKVTTVGAIIRKWKKHKRTVNLPRPGAPCKISPRGVAMIMRTVRNQPRTTREDLVNDLKAAGTIVTKKTIGNTLRREGLKSCSARKVPLLKKAHIHARLKFANEHLNDSEDNWVKVLWSDETKMELFGINSTRRVWRRRNAAYDPKNTIPTVKHGGGNIMLWGCFSAKGTGQLHRIKGTMDGAMYRQILGENLLPSARALKMGRGWVFQHDNDPKHTAKATKEWLKKKHIKVLEWPSQSPDLNPIENLWRELKVRVAKRQPRNLNDLEKICKEEWDKIPPEMCANLVANYKKRLTSVIANKGFATKY

SEQ ID NO: 69 - protein, 550 aa, Alligator mississippiensis
MSAKRKSMTSPGGSAKKQRKAIDLEMKMKIINDYEAGKKVKAIARDLELAHSTISTILKDKDRVKEAVQASTGFKAIITRQRKGLIHEMEKLLAIWFDDQIQKRMPMSLLIIQAKARSIFETLKAREGEESTETFTASRGWFQRFRRRFNVHNRSISGEAASADVEAAEKFVDQFDEIIEKGGYRPEQIFNVDETGLFWKKMPERSYIHKEAKAMPGFKAFKDRVTLLLGGNVAGFKLKPFLIHRSENPRALKQVSKHTLPVYYRANSKAWMTQALFEDWFINCFIPSVKHYCLEKGVPFKIILLLDNAPGHPQHLDDLHPDVKVVYLPKNTTAILQPMDQGAIATFKVYYLRATFSKAVAATESDEVTLRDFWKSYNILHCIKNIESAWEGVTEKCMQGIWKKCLKVFVNNFEGFDKDEHVDVINKKIVELANVLNLDVEVKDIEELVEYVEGELTNEDLIELEAQQHLEEEEEEEEERMEEVQKKFTVNGLAGVFSKVNAAILELEGMDPNVERFTKVERQMNELLRCYCEIYEEKKKKKRKLQSRPH

SEQ ID NO: 70 - protein, 643 aa, Myotis lucifugus
MENQKNKKARLNDKGSGSSSSRPFQDIWTEMYGIVDRNNRSLCILCNETVVSRTWNVKRHFETNHCQLLKKSIDERKEYISRQLHLYKSQSNSIIKFVRCSTNLTSASYCIAHSIAQHGKALSDGEFIKETFLRCAPALFHDMKNKDDIIKRISELPLSRNTIKDRIIDLNKNVQHQLKKDLNSCKYFSISLDETTDVTSNARLAIIARYSDGLTMREELIKLESVPISTSGNEICKVVIKTFSDLNIDISKIVSVTTDGAPNMVGKNVGFLKLFMEAIRHPLVPFHCIIHQEVLCAKSGFSELNDLMSVVTKIVNFIASRPLHKREFSALLQEVDSTYSGLLMFNNVRWLSRGKVLERFVECFEEITVFIENKDLANFPQINDHKWVSNLMFFTDLSVHMNELNLKLQGFGKSIDVMFGYIKSFESKLKIFKRDIETKTYKYFPRIKKYFEKATTTSVMESILMYQNIVNSLFEQFSERFDQFRSLEQTIKIIKYPDVVVYSTLELKDFQWMQIDDLEMQLADFQGSIWTHVFVDLRSKLENLERNRLNNEEECNYEQEILSAWNRVPDTFSDIKNLAMALLTIFSSTYFCETLFSALNNIKTNKRNRLTDEVSGACLALKCTKYEPAIEELADKLQHQKSH

SEQ ID NO: 71 - protein, 602 aa, Petromyzon marinus
MSGSRKRKVDNECRVFNTEWTTKYFFTEVQSKAVCLICRETVAVFKEYNISRHFATKHANYASKQSTQERAATAQRLAANLQTQQHFFHRQTAIQESTTKASFLVAFEIAKASKPFSEGEFVKECMVQTADILCPEIKSKFEKVSLSRRTVTRRVELIDENIASQLNKKSDSFELYSLALDESTDVKDTAQLLIFIRGIDDSFAITEEFLTMESLKGTTRGEDLYNQVSAVIERMKLPWSKLVNVTTDGSPNLTGKNVGLLKRIQNKVKEENPDQDLIFLHCIIHQESLCKSVLQLNHVVNPAVKLVNFIRARGLQHRQFITFLEETDADHQDLLYHSRVRWLSLGKVLQRVWELKEDIIAFLELMGKSDEFPELSDKNWLSDFAFAVDIFSHMNELNVKLQGKDQFVHDMYKHVKAFKSKLTLFSRQIANKSFAHFPTLAMQEEAPRNAKKYSKSLEDLHGEFCRRFSDFENIEQSLQLVSCPLSQDSETAPQELQLELIDLQSDSVLKEKFNSVKLNDFYASLNRATFPNLRRTAQKMLTLFGSTYVCEQTFSVMNANKARHRSKLTDQHLRSILRIATTKITPDLDALAKMGDQQHCSH

SEQ ID NO: 72 - protein, 339 aa, Xenopus tropicalis
MVHRELPKHQRDLIVKRYQSGEGYKRISKALDIPWNTVKTVIIKWRKYGTTVTLPRTGRPSKIDEKTRRKLVREATKRPTATLKELQEYLASTGCVVHVTTISRILHMSGLWGRVARRKPFLTKKNIQARLHFAKTHLKSPKSMWEKVLWSDETKVELFGHNSKKYVWRKNNTAHHQKNTIPTVKHGGGSIMLWGCFSSAGTGALVKIEGIMNSSKYQSILAQNLQASARKLNMRRNFIFQHDNDPKHTSKSTKEWLHRKKIKVLEWPSQSPDLNPIENLWGDLKRAVHRRCPRNLTDLECFCKEEWANLAKSKCAMLIDSYPKRLSAVIKSKGASTKY

SEQ ID NO: 73 - protein, 344 aa, Xenopus tropicalis
MDNRKRRRELSEDLRTKIVEKYQQSQGYKSISRDLDLPLSTVRNIIKKFATHGTVANLPGRGRKRKIDERLQRRIVRMVDKQPQTSSKEIQAVLQAQGASVSARTIRRHLNEMKRYGRRPRRTPLLTQRHKKARLQFAKMYLSKPQSFWENVLWTDETKIELFGKAHHSTVYRKRNEAYKEKNTVPTVKYGGGSMMFWGCFAASGTGCLECVQGIMKSEDYQRILGRTVEPSVRKLGLRPRSWVFQQDNDPKHTSKSTQKWMATKRWRVLKWPAMSPDLNPIEHLWRDLKIAVGKRRPSNKRDLEQFAKEEWSKIPGERCKKLIDGYRKRLISVIFSKGCATKY

SEQ ID NO: 74 - protein, 340 aa, Xenopus tropicalis
MGKTKELSKDTRDKIVDLHKAGKGYGAIAKQLGENRSTVGAIVRKWKRLKTTVSLPRTGAPCKISPRGVSLMIRKVRNQPRTTREELVNDMKRAGTTVSKVTVGRTLRRHGFKSCIARKVPLLKSSHVQARLKFANDHLDDPEEAWEKVMWSDETKVELFGLNFTRRVWRKNKDELHPKNTIPTVKHGGGNIMLWGCFSAKGTGRLHCIKERMNGAMYCEILSNNLLPSVRALKMGRGWVFQHDNDPKHTARITKEWLRKRHIKVLEWPSQSPDLNPIENLWRELKLRVAQRQPRNLTDLEEICVEEWAKIPVAVCANLVKNYRKRLTSVIANKGFCTKY

SEQ ID NO: 75 - protein, 341 aa, Xenopus tropicalis
MRRSLQPTQMAQVVQLIQDGTSMRAVARRFAVSVSVVSRAWRRYQETGQYIRRRGGGRRRATTQQQDRYLRLCARRNRRSTARALQNDLQRATNVHVSAQTVRNRLHEGGMRARRPQVGVVLTAQHRAGRLAFAREHQDWQIRHWRPVLFTDESRFTLSTCDRRDRVWRRRGERSAACNILQHDRFGSGSVMVWGGISLGGRTALHVLARGSLTAIRYRDEILRPLVRPYAGAVGPGFLLMQDNARPHVAGVCQQFLQDEGIDAMDWPARSPDLNPIEHIWDIMSRSIHQRHVAPQTVQELVDALVQVWEEIPQETIRHLIRSMPRRCREVIQARGGHTHY

SEQ ID NO: 76 - protein, 341 aa, Takifugu rubripes
MVRYLDATEVAQVVQLLQDGTSIRAVARRFAVSPSTVSRAWRRFQETGSYSRRAGQGRRRSLNPQQDRYLLLCARRNRMSTARALQNDLQQATGVNVSDQTIRNRLHEGGLRARRPVVGPVLTARHRRARLAFAIDHQNWQLRHWRPVLFTDESRFNLSTCDRRERVWRCRGERYAACNIIQHDRFGGGSVMVWGGISLEGRTDLYRLDNGTLTAIRYRDEILGPIVRTYAGAVGPGFLLVHDNARPHVARVCRQFLEDEGIDTIDWPPRSPDLNPIEHLWDIMFRSIRRRQVDPQTVQELSDALVQIWEEIPQDTIRRLIRSMPRRCQACVQARGGHTNY

SEQ ID NO: 77 - protein, 1073 aa, Xenopus tropicalis
MPGKGRPPRRGTRGRAAMISCGPRKLPSFPKALTLNSQNAEEVVDWLTQHTPSSTVSNFATTSSSSSAMGTPRTTSSTTAAPSSLESEELFTHEFVELSDAQPLLPEEDEGDEDVTPDIIIQAGNTTEMDIRCDEVPAAAVFCELSEEIDASEENDDEEIDILWVPTRREQEEDSSDGETESQRGRRRIRLRRSRDSSQGTVGQQHESAPVVSQPTHPPTSTVTARKPTSKGSAVWHFFNVCASDKSAVICNECSQKLSLGKPNTHLGTTAMRRHMNAKHKALWEQHLKGSSQTLSHPPSAPASYCSTSAVLDPSQPPSTPPSTLTTSSCSSAPSQVSVRAMFERKKPMPPSHPLARRLTAGLSALLARQLLPYQLVDSEAFHQFVAIGTPQWKVPSRNFFSQKGIPHLYQHVQSQVTASLSFSVGAKVHMTTDTWSSKHGQGRYVTYTAHWVNLVMAGKQGMRGSTTVELVSPPRVACGSATTSTPPLLSNSSSKSSSNSSSNLASSSASSSSAAVSSSTPVHPQLHIGYSTCQVRRCHAVLGMTCLESRNHTGSALLSSLHSQADRWLTPHQLKIGKVVCDNGSNLLAALRLGNLTHVPCMAHVLNLIVQRFVSKYPGLQDILRQSRKVSGHFRRSYTAMARLADIQRRHNLPVRRLICDSQTRWNSTLMMFERLLQQQRAVNEYLFELGGRTGSAELGIFFPRYWVLMRDACRLMRPFDEVTNMVSRTEGTISDLIPFAFFLERAVRRVTDDAVDQRDQEHDFWAESPERSQAPAATQGEVSEVESEEEGGFVEAEDQQEQASRGACGHLLWSPGLVRGWGEETVVDEDVVLDNEEGEMDTSASNLVRMGSFMLSCLLKDPRIKRLKEKDLYWVATLLDPRYKHKVAEMLPTYHKSERMLHFQTSLQNMLYNAFKGDVTPHSRGRGAGNPPTSTPARTMHFGHSVTSDMQSFFSPRQRQDPSGSTLRERLDRQVADYLALTADIDTLRSDEPLDYWVRRLDLWPELSQFAINLLSCPASSVLSERTFSAAGGIVTEKRTRLGHKSVDYLTFIKMNEAWISEGYCTPEDLF

SEQ ID NO: 78 - protein, 429 aa, Xenopus tropicalis
MAPKKRHAYEAQFKLQAASYAVVNGNRAAAKEFNINESMVRKWRKQENELRQVKKTKQSFRGNKARWPQLEDQLEQWVIEQRTAGRSVSTVTIRLKATTIAQDLEIEHFQGGPSWCFRFMKRRHLSIRARTTVAQQLPADYKEKMAIFRTYCSNKITDKKIQPNHITNMDEVPLTFDIPVNHTVEIKGTSTVSIRTTGHEKSAFTVVLSCHGNGQKLPPMVIFKRKTLPKEKFPAGVIVKANQKGWMDEEKMREWLREVYVKRPDGFFHTSPSLLICDSMRAHLTATVKKQVKQMNSELAIIPGGLTKELQPLDVGVNRAFKVKLRTAWERWMTDGEHTFTKTGKQRRASYATICEWIVDAWAKVSAITVVRAFAKTGIIAEQPPGNDTGNETDSDNDEREPGMFDGEIAQLFNSDTEDEDFDGFVGED

SEQ ID NO: 79 - DNA, 12 bp, Alligator mississippiensis
ccgtattttctc

SEQ ID NO: 80 - DNA, 6 bp, Xenopus tropicalis
cccttt

SEQ ID NO: 81 - DNA, 18 bp, Alligator mississippiensis
ccttcatacgttcccatg

SEQ ID NO: 82 - DNA, 13 bp, Alligator mississippiensis
cagtagaaccccg

SEQ ID NO: 83 - DNA, 16 bp, Petromyzon marinus
cagtggttcttaacct

SEQ ID NO: 84 - DNA, 15 bp, Alligator mississippiensis
tagggctgtgcgaaa

SEQ ID NO: 85 - DNA, 9 bp, Microcebus murinus
caggttgag

SEQ ID NO: 86 - DNA, 23 bp, Trichechus manatus
cagtagtccccccttatccgcgg

SEQ ID NO: 87 - DNA, 5 bp, Alligator mississippiensis
taggg

SEQ ID NO: 88 - DNA, 4 bp, Alligator mississippiensis
tagg

SEQ ID NO: 89 - skipped

SEQ ID NO: 90 - DNA, 216 bp, Drosophila yakuba
tacagtggcgagcaaaactgagtgcatgttcacaactctcactttggccatcaataaaaacacaaccgcttatgcaaattcaataattttttttatattattaatcttaattaatttataacttattttatgagaaaaaacaaatttagcatatatattcaaagacttagaataaaaaaactaaataaactcacataaactcaattttgcacgttt

SEQ ID NO: 91 - DNA, 12 bp, Alligator mississippiensis
gagaaaatacgg

SEQ ID NO: 92 - DNA, 6 bp, Xenopus tropicalis
aaaggg

SEQ ID NO: 93 - DNA, 18 bp, Alligator mississippiensis
catgagaacggatgaggg

SEQ ID NO: 94 - DNA, 13 bp, Alligator mississippiensis
cggggttctactg

SEQ ID NO: 95 - DNA, 16 bp, Petromyzon marinus
aggttaagaaccactg

SEQ ID NO: 96 - DNA, 15 bp, Alligator mississippiensis
tttcgcacagcccta

SEQ ID NO: 97 - DNA, 9 bp, Microcebus murinus
ctcaacctg

SEQ ID NO: 98 - DNA, 23 bp, Trichechus manatus
ccgcggataaggggggactactg

SEQ ID NO: 99 - DNA, 5 bp, Alligator mississippiensis
cccta

SEQ ID NO: 100 - DNA, 4 bp, Alligator mississippiensis
ccta

SEQ ID NO: 101 - skipped

SEQ ID NO: 102 - DNA, 216 bp, Drosophila yakuba
aaacgtgcaaaattgagtttatgtgagtttatttagtttttttattctaagtctttgaatatatatgctaaatttgttttttctcataaaataagttataaattaattaagattaataatataaaaaaaattattgaatttgcataagcggttgtgtttttattgatggccaaagtgagagttgtgaacatgcactcagttttgctcgccactgta

SEQ ID NO: 103 - protein, 431 aa, Alligator mississippiensis
MPKRKSYTFDFKLSVIDYAKKHGNRAAGREFSVDEKSVREWRKEEDILKTLNPRKRARRGKHCKWPTLEANLKEWVVAQRESNKVVSTIAIRQKARVMANEMKITDFGGGCNWVHKFMHRNNLAVRSRTTVGQKLPDDWEEKLTKFRDFVRKECRVHNLCPADIINMDEVPISFDVPATRTVDQRGKKTIAISTTGHERTNFTVVLACTASGVKLKPMVIFKRVTMPREKIPNGVAVICNKKGWMNEDIMKEWTERCFRTRQGGFFAPKSLLIFDAMAAHKHSSVQKQINKAGAHIAVIPGGLTCKLQPLDISVNHSFKCFMRKEWEKWMSQGVHSFTPSGKQRRATYTEVCNWIVAAWRAVKPSSIINGFFKAGILDGASGEESDYTDSEDYADELDDAAFVDAYFGESSYDTDFEGFPASEKEEAPNWV

SEQ ID NO: 104 - protein, 589 aa, Xenopus tropicalis
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLPRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHY

SEQ ID NO: 105 - protein, 626 aa, Alligator mississippiensis
MRRQRIFSAEACLAQVTGDDSDTSTDEILSQGSEDADFVPDMGDCSEQEDHASTEASSSSDEEELVPQAEVMPTLPVGKGRGPARGRRGRPRRATAGRGAEPLPPPPENIADDDTFRGRNGRVWGKSPQLIHHRRTAKDIVHESAGPTRESNKDTVVETFHLFLSQDILTVIIRETNREAERRFREWNDSHPENRKEWKKLDETELKAYIGLLLLSGLYHGGHEALEELWEQRTGRPCFRATMSLKRFRQITNYLRFDNKLTREERRAVDKLAAFRDIWTQFVEQLRKWYIPGIHLTVDEQLIAFRGRCPFRQYMPSKPAKYGLKIWWCCDAATSFPLSGDIYLGKQPEAPRQGNLGTQVIEKLVHPWYKTGRNIVGDNFFTSTDLAERLLQQDLTYVGTIRKNKADIPNEMQANRRRETFSSIFAFDGELTLVSYVPKKSKVVLLLSSMHHDLSVGGEQRKPDIIHDYNKNKGGVDNLDHLVRMTTCRRKINRWPMTLFFNMLDVAAIASLIIWLGHNANWKQRSHLRRRRFFLQELGEQLIDGQIKRRMMDTRNLSKSVKSGMTALNYKLPQRTPVEPSASMSGRCAFCPRTDDRKTRKRCEDCKRFACVHHSTSLLLCEECNI

SEQ ID NO: 106 - protein, 627 aa, Alligator mississippiensis
MRRPSQMELRAAWVPKQDSGFSLSVAIVFKMSASNTDSTSASASTRKRKAITLEEKLEVVKRYERGEKTKEIRCATGLSESTLRTIRDNAEKIKESCKSATRLSTARVSLTRSAIMEKMERMLTTWIEHQNQEHVPLSTQVIQAKARSIYEVLKRDDPEAKPFNASAGWFDRFKARHGFHNLKLTGEAAAADKSAADKFPSILQAAIEEGSYDPRQVFNLDETGLFWKRMPSRTFISVEERTAPGFKAAKDRCTLLLGANAAGDFKLKPLMVYHAENPRALKGYAKGHLPVYWRANRKGWVTGSLFEDYFSSLLHLELRAYCDRQNLPFKILLLLDNAPGHPPSLGDLSENIKVLFLPPNTTSLIQPMDQGVIRAFKSYYLRRTFKKLINETESKEKANVKEFWKAFNIKMAIDIIGDAWNDVTQQCLNGVWKNIWPAVVNDFRGFAADEFISDARHEIVEMAKSVGFEQVDEENVAELLDSHREELSNEDLLELDRERHEEEEPMEENERPSPRVLTMKGMADAFKLLDGYLAFFDENDPNRERSAKVARLMKEANACYTELYREKRRCSVQSSLDKFFRKRDSTISTQSTDSQPSTSGGEPATSGGEISPPSFDVFDLDEDSAPH

SEQ ID NO: 107 - protein, 602 aa, Petromyzon marinus
MSGKRRKWSDEYVQHGFSCITERDGSERPICMICNAKLSNSSLAPAKLKEHFLKLHGDGEYKNTTLAEFRVKRARFDERATLPALGFVPVNKPILTASYEVAYLIAKQGKPHTIGETLVKPAALKMANLILGTAAEGKLSQIPLSNDTVSDRIEDMSKDILAQVVADLISSPAKFSLQLDETTDVSDLSQLAVFVRYVKDDMIKEEFLFCKPLTTTTKAADVKKLVDDFFRDNNLSWDMVSAVCTDGAPVMLGRKSGFGALVKADAPHIIVTHCILHRHALATKTLPPKMAEVLKTVVECVNYVRKSALRHRIFSELCKEMGSEFEVLLLHSNIRWLSRGQVLNRVFAMRVELALFLQEHQHCHADCFKNPEFILILAYMADIFAALNHLNQQMQGGGVNIMEAEEHLKAFQKKLPLWKRRTENNNFANFPLLDDCASKIEDVSGIGDISVSTELKQTIATHLDELAKSLDGYFPTRESYPAWVRQPFTFAVETADVNDEFLDEIIEIQQSQVQQQLFRTTTLSTFWCRQMNKYPVIAKKALDFFIPFVTTYLCEQSFSRMLDIKTKKRNRLCCENDMRVALAKVKPRISELISQRQQQKSH

SEQ ID NO: 108 - protein, 757 aa, Alligator mississippiensis
MTVSSTPISLPMLSLPVPEEELELDLDLSAIAREILGKEGESSEFVLHTPQVSPRARSPSPEGTSEEAEVEVAEASGSAESAPPVAVPLPAEEGEKAASAHAPAPQKRAGSVVWDHFELADDPTYAICLHCRAKTSRGKGTKHMSTTGMLMHLRRHHPLALAPPQPGTSGSVPKGKSPSRSKAPVPPKQRQATLEQWGKGGQKGGRVARASEITQSIGEMLAVDGQPFSLVERPGFRRLMALVAPSYQVPARTTFSRTVVPSLYEACREYLREELRKAGPQVALHFTSDIWSSRGGDHAYLSLTGHWCDQSGRRWALLQAEVMDESHTATEIMGAMNRLVQGWLVGQGELTRGFMVTDNGANMVKAVRDANFVGIRCVAHKFHLIVRDALEGDRAAGDGASTTSKLISKCRKVAGYFHRSIKGGKMLRDKQAELNIPQHKIMQDVETRWNSTYLMLERLVEQQKAIHEMALLGEIGISGPLNRAEWDTISQILVVLKPFLEATETLSAGDALLSQVIPVVRELQSQMESFQGINVPGWGKPLSPDVQALVRRLKEGIRKRLDPLRSSTVHVLAATCDPRVKGSVCGSQSLNQWTEVLVKRVREAERRRRGDVEEGDPLSRASTPSTSQSPPPPQALPMWAKGMASMVGSRGTRPHHQAGSAQALVAAYLAEDVEPLLCDPLAYWASRSQMWPDLATVAREHLSCPPTSVPSERVFSIAGDVVTPHRTSLDPGLVEQLVFLKVNLPLLGFPKLQVQTE

SEQ ID NO: 109 - protein, 554 aa, Microcebus murinus
MSKRPADTPMGNSDKKKRKHLCLSIAQKVKLLEKLDSGVSVKRLTEEYGVGMTTIYDLKKQKDKLLKFYAESDEQKLMKNRKTLHKAKNEDLDRVLKEWIRQRRSEHMPLNGMLIMKQAKIYHDELKIEGNCEYSTGWLQKFKKRHGIKFLKICGDKASADHEAAEKFIDEFAKVIADENLTPEQVYNADETSLFWRYCPRKTLTTADETAPTGIKDAKDRITVLGCANAAGTHKCKLAVIGKSLRPRCFQGVNFLPVHYYANKKAWITRDIFSDWFHKHFVPAARAHCREAGLDDDCKILLFLDNCSAHPPAEILIKNNVYAMYFPPNVTSLIQPCDQGILRSMKSKYKNTFLNSMLAAVNRGVGVEGFQKEFSMKDAIYAVANAWNTVTKDTVVHAWHNLWPATMFSDDDEQGGDFEGFRMSSEKKMMSDLLTYAKNIPSESVSKLEEVDIEEVFNIDNEAPVVHSLTDGEIAEMVLNQGDRDNSDDEDDVVNTAEKVPIDDMVKMCDGLIEGLEQRAFITEQEIMSVYKIKERLLRQKPLLMRQMTLEETF

SEQ ID NO: 110 - protein, 642 aa, Trichechus manatus
MAPKRKSSDAGNSDMPKRSRKVLPLSEKVKVLDLIRKEKKSYAEVAKIYGKNESSIREIVKKEKEIRASFAVAPQTAKVTATVRDKCLVKMEKALNLWVEDMNRKRVPIDGNVLRQKALSLYEDFSKGSPETSDTKPFTASKGWLHRFRNRFGLKNIKITGEAASANEEAAATFPAELKKLIKEKGYHPKQVFNCDETGLFWKKMPNRTYIHKSAKEAPGHKTWKDRLTLVLCGNAAGHMIKPGVVYRAKNPRALKNKNKNYLPVFWQHNQKAWVTAILFMEWFHQCFIPEVKKYLEEEGLEFKVLLIIDNAPGHPESVCYENENVEVVFLPPNTTSLLQPLDQGIIRFVKATYTRLVFDRIRSAIDADPNLDIMQCWKSFTIADAITFIKAAMDELKPETVNACWKNLWSEVVNDFKGFPGIDGEVRKIIHAARQVGGEGFADMLDEEVEEHIEGHREVLTNEELEELVESSTEEEEDEEETEAEPAMWTLPKFAEVFRIAQTLKDKIMEYDPRMERSIKVTRMITEGLQPLQQHFDELKRKRQQLPITMFFQKISAKNLQLSRIPNHRHRLLLTSNHRHRHGSMIQDHPKQMILLLTYRQKVNSSLTLRHNAYVIHLTSSHHVGIVSSHIITRRRVSTVQ

SEQ ID NO: 111 - protein, 582 aa, Alligator mississippiensis
MNAKKSSKTAKYVSALDRARQYPAGTLHADGGRLFCTSCNVTLDVSRKTSIDRHLESEAHMKRKAAAEAEGQSKKQATVSSLFKRTTESSLARREAAFSLVEAFAAANIPLEKLDHPKLRDYLNQNVPNAGSFPRANKLRQDYLPAVIASHVQSTKAALAEYHSFSIVADEATDPQDNYVLHILFVPGSWTDRDLPVFQTEPVHLTSVNFSTVSQAIVKVIVSYGVDFNKISAFLSDNAMYMCKAFSDVLQGMLPNAVHVTCNAHILSLVSDIWRTQFPEVDSLVSSFKKAFKHCPGRKVRYRESVASQSGDSHGTVPLPPEPVLTRWNSWFNAVRYHAKHMQHYQQFVSEELEITPSTQCMLKLRDLLGRGDIQDQVTFVADNATRFMELLTWFEGRQAVIHHAHNKLMDLISWVEDRASQELSSCPRENQRRRDLFQAIAVKLNQYYRYDPSLPPRFRQPAAPFLSAVRILDPNQVPLLSCDPQLLKAIPGWGKEHDNERAAYVNFVKECQMPVPVPVFWDSVCDRFPHLYALARRCLTIPTNSVDAERAISVYGQVFTPQRQRMSPATAAGCSMLAFNT

SEQ ID NO: 112 - protein, 754 aa, Alligator mississippiensis
MTVSSTPISSPSVSLPVPEEELDLEAGDLSALAKEILGTEGESEFVLHTPQSSPRARSPSPEGTSEEVEVVEASGSAESAPPVVVPLPAEEGDKAGSPPSTQKRGGSVVWDHFEVADDPKYAICQHCRRQISRGKEVKHFTTTAMLLHLRRQHPLALAPPQPGTSGSVPKGKSPSRSKAPVPTKQRQATLERWGKAADKGGRVARASEVTQSIGEMLALDGQPFSLVERPGFRRLMALVAPSYQVPARTTFSRTVVPSLYEACREYLREELRKAGPQVALHFTSDIWSSRGGDHAYLSLTGHWCDQSGRRWALLQAEVMDESHTAREIMVAMNRMVQGWLVGQGELTRGFMVTDNGANMVKAVRDANFVGIRCVAHKLHLIVRDALEGDRAAGDGAVTTTQLISKCRKVAGYFHRSIKGGKMLRDKQAELSVPQHKIMQDVETRWNSTYLMLERLVEQQKAIHEMALLGEIGISGPLNKAEWDTISQILVVLKPFLEATETLSAGDALLSQVIPVVRELENQMEKFQGIDVPGWGKPLSPEVQALVKRLKEGIKKWLDPLRSSTVHVLAGMCDPRVKGSMCTGSTKTLDHWTEVLVNRVREAEGQRRGDVEEGDPLSRASTPSTSQSPPRQPLPMWAKGMASMLGSRRTRPQPRAGSAEASVATYLAEDVEPLDCDPLAYWATRSQMWQDLATVAREHLSCPPTSVPSERVFSIAGDVVTPHRTSLDPGLVEQLVFLKVNLPLLGFPELHLQTE

SEQ ID NO: 113 - protein, 1156 aa, Xenopus tropicalis
MTGRGRGRKGGMGGKGGEGGRKGGVGGEGGRKGGVGGEGGVGGKGGLGGRSRGAGCGQEPSTRAMPSLGNIGQQPQQQSQQQQQQLQQQQVAGRSGATGSRKTLPDFFATTSRPIQSQQAEAVMDWLTQATSSAADSSEQQEVASPASSVIRGMLSPLLFDPEVDFSPDTQDLFDDQTGMGGAFLSDEEEVYVAQHTTSAVGDVGQGPLMAGQQADEPDVDVDAGSDLGEDVEDDGDRSWVPAQEDNSSSSEGELCVVVSDADEEPAPKKQKTPPTTARGGRQQGSTAPGSTGSGRMGRDLQPSGPQPSTAGKVVARTSAVWAFFTCQSQDPSVAVCHLCRQKVRRGQTGSHMGTSALSSHMKRHHRMTWEQHQSGRAPGGSGASCPPPPIPQGTGASSPIPVAREEDSEAHSWRRPLCSSASSAAPSVSSSSLLPSAQPLSRQVSLPQFLGRKAPLSSSHPQVQRLNACLAKLLAVQLLPYQLVDAAPFRQLMACAAPNWRIPSRHYFARKAVPALHQQVVDNVSLSLDYAVGGRVHCTTDTWTSRHGQGRYISYTAHWVTLMSAGEGAGSRTPLRLEVPPRGVKGKPHTATSSSSSSTMDEPPLKRPRSYASVQYKRCQAVLQLLCLGERSHTAPELHAAFQTQVQRWFTPRQLQAGNVVCDNGRNLLAALHLGRLTHVPCFAHVLNLVVQHFLKSYTGLGVVLEKARRVCGHFRRSPTASASLSRMQRQHSLPPHRLICDLPTRWNSTLHMVERLVEQRWAVSNYLLEHSARGTQGANLGYFSTEQWQQMRQLCRVLAPFEQATRFVSRDNACVSDIIPLVFLLKRTLDGLLEEGDVPEEEECRPRRQVEGAARHDEEDMPNEEDEGEEDWVPAEQGEQGPRHPTTPAVVRGWEDTEQGEEAGDDLLHLQGSQEEVGRGHLFYMAAHMQTCLRSDSRICAIKDREDYWVATMIDPRYKEKMAQFLVPSQRERRVGQLKRALCAKLMEAFPQPDTQASTTPHIQQRQESSSSSSSAARGGGNLMDVWRSFFEPRQAAAGLVSTQSHHQQRLDNMVADYMGSVSVQDAMHTDDDPMNFWVSRLDQWPELAQYALEVLACPPASVLSERVFSAAGGVVTEKRTRLSTGSVDRLSFIKMNEAWISGDIHVPIADIRD

SEQ ID NO: 114 - protein, 334 aa, Drosophila yakuba
MRKLSGEIQNNIVSLTESGNSTRQIAERLGISQSAVVSIQKRRKLTPKPQVSGRKKLLKDSDARLMMSEMRKNKNLTPKGACLAINKNVSEWTARRALQDIGYMSIVKKNKPALSDKNVKARLKFAKDHKNWTIDDWKRVVWSDESKFNRFQSDGKQYCWIRPGDRVQRHHVKQTVKHGGGNIMVWGCFTWWHIGPLQLVEGIMKKEDYLRILQTNLPNYFDKCAYPEKDIIFQQDGDPKHTAKIVKEWIGKQHFQLMEWPAQSPDLNPIENLWSIVKRRLGQYDSAPKNMGDLWERVAVEWSRIPQDILRNLVESMPKRVTEVIVNKGLWTKY

SEQ ID NO: 115 - DNA, 2687 bp, Anopheles gambiae
taggcatgggctaattgtgcgagacctatgccgcacactcagttcactcaagtgtgcggctgaccaccgcacgcgaaaaagtgtacgggtaggtcttccgcacgcataaggaatgacttcggttgtgactggttgaacgaaaaaaaaaacattaattttagtcgactgtgttcaacagtcaaataaaaaatcgatcacactctgagcgagaaagaatacgcaaattgttagcgtatttagttgggtcagtcgcactaaaagaactcctgtccaagactgacatggaacgagaaatacactcatacccgttgggtcagtcgcacactgccgcacgcgtgttcagtttggtcagtcgcacacacagctgcacaaaaagaaattctgtcgcacactgccgcacgtttgctcagttcgttcattcgcacacagccgcacaaaaagaacttctggcgcacactgccgcacgcgtgttcagttgggtcagtcgcacacagccgcacaaaaagaacttctgccgcacactgccgcacgtttgctcaattcgttcattcgcacacagccgcacaaaaagaacttctgccgcacgctgccgcacgtgaggtcagttgggtcagtcgcacacagccgcacaaaaagaacttctgccgcacactgccgcacgtttgctcaattcgttcattcgcacacagccgcacaaaaagaacttctgccgcacgctgccgcacgtgaggtcagttgggtcagtcgcacacagccgcacaaaaataactcacactgactacgctagtaatatgctcgttctctctgcgctgtcggatcagtcgcacacagtcatacaaagaactgctaccgcacactgtcctgatgaaacacagggtttgatgtctttttggttgtttcttcattttttggatttatctcacattttattgcatgcttcccaaattattttggactcattccacaatttattatatgtttctcatataattttgtattcgttttgtgatttattcttaccatcccagttgttttgaaacaaataattgaacaaattttatgaaacgttaaataatcgtgatccataaaccctgtaataggagacaaaattggtatcaatagcatcttcttccttctcattttctgctgattgttttttacgtagcaccagacattcttagcgtggggaactgtgtggtattgatatgtatcgaatgtaagagcaaggagggaaaaacgtacactaggtattcgctagttgaatgatttgaaatgtggatcatttttttaagttgcatggtcgatgtgagatttataaatcgatgatttttaatcttgaaatattcaccacatgcaaaatcaaaaaatctccaaatctttatattcttttgctttttatcatttaaaaaaatatgccaattcaaaatgcaatacatgtcacacattacaatttcaattagcgagctcaaactgtaccatcccacgatgttttattcgaacaccattactagtgaggcatgcttttcgcacactaagttgatgagtgcaatatgtatataaaagaacgtcaagtattcggaaagtggcttacccattatgaatgagtccctcgtttgttggtgttgtatgaataatattgcagtaggtaacaaagtgaaatagtattgaagtgcgtttccggtatcagagttttagctttgcattgtcgttgcgattcctattttggttgtgtatcgaatcagtcgcattaaaggaaagtacccagtttgaaaattcataacgcgtaaatactttttctgtggaagaatcggcagggataacctgataactaattaaaggtaagttgctttattatttttaacttttaacaagatgagaaaaaatgacaaatcgtcatttattcttttttttatttcataaaaaaaatcctagttcagtggtcgagaaagtacggcaatccgtttacgatttttgccagcattttcactccttaacagccccttgctattgtaggagtattcaaaatctcccacagtttcacgccttcatctgaaaatccggtttggaaatttactgacatttgtatagaatttagctgtctatggcgtcaagttaaccgacgttttttttctagttcagtggtcagcaaactttgtggtgaaaagagccaaattctatgaaaatgttggtagatttcgatatgaagagccaaatttgaaaaccaagcatacaattgagttaaccactgtcctcatttggaccttccttccacaaatgacattcaaagccaaaactattcaatgttttggtcacaccagtgacaactatgatcgactgacttttcatgatgactgagtaacttttaatcgaagtaatagatttaatcattatagtaaaatttacatcatcaattaaaaataatgatctacaatttatcaataattaaagtttcctcaaaataccaaagcacagtgatagtattacgtttattttatttgaataatattgctataattataatttacaacaattgctgacgggactggaactataccaaaacgttctttaattaaaaaaaaactgttcgttttagcagtcatactgttaaagccatttaggctgttagataattgaacaaatatttaaaattttatttttgattaatgttgtaatatgttttagttaag

SEQ ID NO: 116 - DNA, 1472 bp, Anopheles gambiae
tagagttgtgcctcaagaaccagaactgtacgattcattcaattcatcctaaagaacgaacgacctacgttcatctaatcaacgttcatcgattctttataaattataatgtattgagttgttcatgaaaataacccgggtaggaacccgtcgaaatgaagaacgtcctagtacgccaggtcgttcattccccccatacaaatatgaacgtgaatcgtatgaccaggacgttcacgaaaagaacgtcctacccgtcgaaataaagaacgtcctagcaagccaggtcgttcattccccccattcaaaatgaacgtgaatcgtatgaccaggacgttcacgaaaagaacgtcctacccgtcgaaataaagaacgtcctagcaagtcaggtcgttcattttttcccatacaaatatgaacgtgaatcgtatgaccaggtcgttcattctcctgatacaaaaatgcgctgtcccacatgatagacatgtgtcgatgaaacaaaatcagcatactttctcactcagcccaacaaaacatccgatctggcattctgcactggctttctgcggagagcacagtacaagataagagagcggatagaatgtttcgttgatctacatcgttcactctctttctgatcctcggagacgatgccgcttctgcaatatgcgctctctttttcttcccgctgcaatactttcagctgatagttaccgagagaacgatcacagagatagagagtcaaaaaaatgacgaggacggggggtggtatgtgggatgagtttttttacctacttgtgagcgaggtacagtcaaagttcaataattgaacgaaattttggtttgtatatttttcgatggatatttgatagtaggttgatagatcaactaaaaagcatgcctttatattagtgaatctaatttcatagaaaatcgcaaaactgtcgcctataatgaaaaaaaaacttcatttattttgtgcaggaaagaacgtgaaaatttatcaccaaacgtgacactaaccaaacagtgagctgccaatcgaagttcaattagtgagtttgaactgtactagtcgttttgtttgagagagaaatgaccttctcgcgctttgaggaagagaggaaaattacactaatacgtttatggtcactaaatccatatgataaatacatagatcacgtgtgttcggttcgttgttgtataggtgattttgaccgttatttttgaatacgttcgatttcttcaatgtgttgtaggcttatggtaataaacattgctgaaaccgtgattgtattgataaattgatctttgatatgccatttgttgtgatcttttgacttgtaaaccacaaattgatctacgctccatcgaaataagtgaaaatttaataattgcacggcgatcaaaggtaacattagtcttggtagttaaaaacaaaaacataaagtgttgtatgaaacatttccacagat

SEQ ID NO: 117 - DNA, 364 bp, Danio rerio
cagcggggaaaataagtatttgacacatcagcatttttatcagtaaggggatttctaagtgggctactgacacaaaattcctaccagatgtagccatcaagccaaatattgaattcatacaaagaaatcagaacatttaagtatacaagttgagtcataataaataaagtgaaatgacacagggaataagtattgaacacatgaagataacaaggtgcaaaatggcatagaaagtcaggagatctgtcagtattgagagaaaaaccctgctccctatcagtactaattgatatcagctgctttagtcctaattgatggcctataaaggcttctcattactcaggaggcacacaggaaagacttc

SEQ ID NO: 118 - DNA, 287 bp, Petromyzon marinus
cacaggggtcgggaacctatggctcgcgagccagatgtggctcttttgatgattgcatatggctcgcagataaaacaaaatattctcacttgttttaatccatccatcgatttttcactgcgcatgtcctgttcagggctgcaggagcaattgtccattattttaattggatgatactggcctacgtttgccgttgctgggtaaaacaggtaaacgcccccttttgaatcagtctgagactgcgtctgctcggtcattagctcaaagctaacccttcgacgaagaag

SEQ ID NO: 119 - DNA, 877 bp, Xenopus tropicalis
caggggtctcaaactcacggcccgcgggccatttgcggccctccatccaatattttgcggcccgcaccaaggggagctggcgcagaagtcgggctccgtcagcagaagcgctggcagcggcggcagaagcaccccagttaccaggggcggcataaaaagccgcacctggtaactttaagagccgaatttccggtttttaaaccggaaattcggctcttctagtgcagagagcgcaattgcgctctctgcatctagcgatctcaccgccggcagtgtcagtcagaatgtcgggctccatcagcagaagcgccggcagaagcgccggcggcagaagcccccagttaccaggggcggcataaaaagccgcacctggtaactttaagagccgaatttccggtttttaaaccggaaattcggctcttctagtgcagagagcgcaattgcgctctctgcactagcgatctcaccgccggcatccaggagtgtcagtaaagcgcttccgggccagcgttgcgtccctcctaagccacgcctgctgcaagttttttctgttgtgggagtacagtccctgcctagcgccaaatttggatctgctgaatctgtccctgctgatctgtctgaagcctgtcgttgcgttcgcctctggtatgattcaagggaccatttctgtcaaatttgaggtacgtgcctgtttcctctgtgggtgaatatcgggaatgctgggagttgtagttcggttgcagtagctcctgcttagcagctaattgtgcacacagaacgttgtgccttgcattatatcccttgaaagccaattagaattagaacagacacaagggagagaagtttagtacagaattaggttttgtcatacttcatc

SEQ ID NO: 120 - DNA, 365 bp, Xenopus tropicalis
cagtggaggaaataattatttgacccctcactgattttgtaagtttgtccaatgacaaagaaatgaaaagtctcagaacagtatcatttcaatggtaggtttattttaacagtggcagatagcacatcaaaaggaaaatcgaaaaaataactttaaataaaagatagcaactgatttgcatttcattgagtgaaataagtttttgaacccctaccaaccattaagagttctggctcccacagagtggttagacacttctactcaattagtcaacctcattaaggacacctgtcttaactagtcacctgtataaaagacacctgtccacagaatcaatcaatcaagcagactccaaactctccaac

SEQ ID NO: 121 - DNA, 472 bp, Danio rerio
caggggtggcgaacccgcggctcgcgagccgcttgcggctcttttactgctcttgtgcggctcaacctctcgcgcgatttaacatacattaaccaattgactaacgccccattcacacggtgcttctgcgtctctttctgcttttgtgcggctcaacctcttgcgcgatttaacatataataaccaattgactaatgccccattcacacggggcttcagcgttgctcgctgcagaagctgggagtagctcaacttttcaagcgcttacggacgcgttagccaatcagatcgctgtatgcaaatacacctgctagacagtggcctattgctgactgaattttattttctgacgcttccatgacgatcgtttcagctctaacttcagacacgccttcagtcaagggttgacactgaagccccgtttgattggggcgcaacacgagtgacgtgcagttcgttaatatcaatcgtc

SEQ ID NO: 122 - DNA, 356 bp, Takifugu rubripes
cagtgagagtaaaaagtatttgatcccttgctgattttgttggtttgtccactaataaagacatgatcattctatacttttaatggtagatgtattctaacatggagagacagaatatcaaaaagaaaatcaagaaaataactttaaagaatttattttaatttatttgtttttcattgagggaaataagtatttgatcccctagtatgcattaggagttctggctttcacagaccagttagacgctcccaaacaacttgttacctgaattgaagacaccaggtctaactaatcacctgtatgtaagacaactgttcacagaatcagacaatcagacagattccaaactctccacc

SEQ ID NO: 123 - DNA, 297 bp, Xenopus tropicalis
caaaccggattccaaaaaagttgggacactaaacaaattgtgaataaaaactgaacgcaatgatgtggaggtgccaacttctaatattttattcagaatagaacataaatcacggaacaaaagtttaaactgagaaaatgtaccattttaagggaaaaatatgttgattcagaatttcatggtgtcaacaaatcccaaaaaagttgggacaagtagcaataagaggctggaaaaagtaaatttgagcataacgaagagctggaagaccaaataacactaattaggtcaattggcaac

SEQ ID NO: 124 - DNA, 369 bp, Xenopus tropicalis
cagtggcttgcaaaagtattcggcccccttgaacttttccacattttgtcacattacagccacaaacatgaatcaattttattggaattccacgtgaaagaccaatacaaagtggtgtacacgtgagaagtggaacgaaaatcatacatgattccaaacattttttacaaataaataactgcaaagtggggtgtgcgtaattattcagccccctttggtctgagtgcagtcagttgcccatagacattgcctgatgagtgctaatgactaaatagagtgcacctgtgtgtaatctaatgtcagtacaaatacagctgctctgtgacggcctcagaggttgtctaagagaatattgggagcaacaacacc

SEQ ID NO: 125 - DNA, 623 bp, Anopheles gambiae
tacagtgtcggacaaatcaataggaccacttatcaatttattattcacttatcatatttcatgacttaaagtgaatcaatctttaccaaactttcatgaaagcactattagaatgtttactttaagaatgtttttaaagtttggtgaaaagttaagaaacaaaaaagttatgataagaaatgtaaaaataacccaaaaatcatgcgccaaaataataggaccagttcagaaaattttaaaataattatcatttattattgttaaaacacagtttttgatgttattgagttgtcggataacttaacttaaattagtgaactaatgcgtattcattttttttgtagaaacgcttagcgcagtggcaattcgctgtgtctttaattaacatgctctaatttactaatgcaagaatattacggcactcgcttaagggttttgagctccctttgcactgagagacgaaatagtggtgagtaccttcgtctatgggtataaaagcgcgtcacagcgctggatcgcttacactgtgttctaagccgttgcctagaacacttcacacgatccattagctcgtgtatttttgggtgcactacttcaagcgcttgattgaaggaattccaacg

SEQ ID NO: 126 - DNA, 393 bp, Xenopus laevis
cagtggtgtgaaaaactatttgcccccttcctgatttcttattcttttgcatgtttgtcacacttaaatgtttctgctcatcaaaaaccgttaactattagtcaaagataacataattgaacacaaaatgcagtttttaaatgaaggtttacgttattaagggagaaaaaaaactccaaatctacatggccctgtgtgaaaaagtgattgccccccttgttaaaaaataacttaactgtggtttatcacatttcaattttcaatttcaatatcaatttctgtagtcacccccaggcctgattactgccacacctgtttcaatcaagaaatcacttaaataggagctacctgacacagagaagtagaccaaaagcacctcaaaagctagacatc

SEQ ID NO: 127 - DNA, 271 bp, Anopheles gambiae
cactggtggacataaaaataggaaaaaaaaattttgtgtgttttttttgcatacactaggtgtgtcatcgccaacagttcgaggcgtactgaatttgcaatagccgaattttgccttttatactctcatttttggtgcgctacttatctgagttttgagtgccggcagcgttttgcggcagtataaaaggagagccaaaccgtgttgtgcttcattcttccatcagtggtcaaggtgaaaggttgctatttaaaaattccacaaaatcatc

SEQ ID NO: 128 - DNA, 153 bp, Xenopus tropicalis
atatacactcaccaaaaggattattaggaacacctgttcaatttctcattaatgcaattatctaatcaaccaatcacatggcagttgcttcaatgcatttaggggtgtggtcctggtcaagacaatctcctgaactccaaactgaatgtcaga

SEQ ID NO: 129 - DNA, 872 bp, Petromyzon marinus
caggggtcaccaaactacggcccgcgggccgatccggcccgcgacgtcagtccatcaggcccgccgacggtacttgaatttggttgataaaacgtatggaacacgagacgttgtgatacaatttaaatttaaatattatgtttagaatactaggagatcttcacgcgtgcggagacctcctgactgctatgtgccttaaatttactattttaattttaaattattgtatctagattttcttgtttattgtaatgtttgcttatttcgcgccatcccaagtccttccccggctcgtggtagctccgcctacgctctgacgttaccatggtttccatggcaacgtcatggcaacacgttaccatggtttccatggcaacacacacaaacacgcatctttcccgcccccagattttccggctgtcaacacaggtccgacgctccagaaatatttacttctttttgcctgtcatccattttataggtatgtaacaaggatttctttgtttgacagtgataggtaagtgaactacaagtgtgattagaggtcataaagtaacaaacatagaataacaatgccatagaagcctcgtgttattacgaaaatattatccccagctcagaaaaattccccggtcaaggtgttaatcgagctggtctctaggctgtttatttaggcccgccctatttatatattttgtaaaccttattatgtgatatattctattaccaaaggtacctataaactttatatgtacctttggtgagccgtagcttcaccagtgacccctgaatcctctgcttttcttgtgtttcaataggaaacaattcaatgtctttttcattttcagatcccgttttcttcacccttcatt

SEQ ID NO: 130 - DNA, 368 bp, Petromyzon marinus
cagtgagggaaaaaagtatttgatcccctgctgattttgtacgtttgcccactgacaaagaaatgatcagtctataattttaatggtaggtttatttgaacagtgagagacagaataacaacaacaaaaatccagaaaaacgcatgtcaaaaatgttataaattgatttgcattttaatgagggaaataagtatttgaccccctctcaatcagaaagatttctggctcccaggtgtcttttatacaggtaacgagctgagattaggagcacactcttaaagggagtgctcctaatctcagcttgttacctgtataaaagacacctgtccacagaagcaatcaatcaatcagattccaaactctccacc

SEQ ID NO: 131 - DNA, 87 bp, Alligator mississippiensis
caggcgctcctcacttaacgacctacctgtttaacgaccgcgcggacttacgaccgcgcggtcgtggaaacggaagtactgtacaag

SEQ ID NO: 132 - DNA, 699 bp, Myotis lucifugus
cagtgatggcgaacctatgacacgcgtgtcagaggtgacacgcgaactcattttgggtgacacgcatcgacagagacaaaaaattgcataattcaaatatttgtctctcgcaacggccagagttgggcatcactcgagtaatattattactcacgttactcatgatcaataatgcaattctaaaaccggatgtaaacatgtacatcggtgttgcgtagcaaagatacatagtgaaaacatccaagatacattgttccaatattgacattaatataaaaaggttaagaattgcactataaagagattaatctttactcatgacattttatttttactcgagtgacaaaatgcccaattctggaaacggcctcgttattgtcaacatgtgcctcgggtaagcttcccgtgcgttcgggtgcattcggcaaacggacgcacttgatcgccatgttcgttaggagacaggagtgggagggttagaagaaaaagatatacagtgcaacagtgctcgacgagacattctgcgtgtcggataattgattacagcaccatatatttgttggttatacatactatctttgaattgttttgcaccaggagtcagtgcaacaacactgcaaaaagttaataggtaagaaacctatcaaccttttataaaaaacgccgtgatttatatttatttatttttgcagccttgtg

SEQ ID NO: 133 - DNA, 301 bp, Petromyzon marinus
cagacatgggcaaagtacggcccccgggccgtatacggcccgttgggctttttaatccggcccgccgaacttgtccaaatattattattaaattaaattttacctttctcctgtaatgcccccatttccccattagatggcgcactttgaccgggagtgtggtgtatagggctcgaaagggcacatgatctcgcaatgcattgtgggccgtcagcgccattttcagggtttgtttacattgctgagttagatacggtgctcccttcactttttcgctttgccctgtgagcccttctgtaaa

SEQ ID NO: 134 - DNA, 394 bp, Xenopus tropicalis
cagtggatataaaaagtctacacacccctggtaaaatgtcaggttcctgtgctgtacaaaaatgagacaaagataaatcatttcagaactttttccacctttaatgtgacctataaactgtaccactcaattgaaaaacaaactgaaatcttttaggtggagggaagaaaaccaaaaaaactaaaataatgtggttgcataagtgtgcacaccctcttctaactggggatgtagctgtgttcagaattaagcaatcacattcaaaatcatgttaaataggagtcagcatacacctgccatcatttaaagtgcctctgattaaccccaaataaagttcagctgctctagttggtctttcctgacatttttttagtcgcatcccacagcaaaagcc

SEQ ID NO: 135 - DNA, 382 bp, Xenopus tropicalis
cagtcatggccaaaattgttggcaccccagaaatttttccagaaaatcaagtatttctcacagaaaagtattgcagtaacacatgttttgctatacacatgtttattccctttgtgtgtattggaacagaacaaaaaagggaggaaaaaaagcaaattggacataatgtcacacaaaactccaaaaatgggctggacaaaattattggcaccctttcaaaattgtggataaataagattgtttcaaacatgtgatgctcctttaaactcacctggggcaagtaacaggtgtgggcaatataaaaatcacacctgaaagcagataaaaaggagagaagttcacttagtctttgcattgtgtgtctgtgtgtgccacactaagc

SEQ ID NO: 136 - DNA, 357 bp, Xenopus tropicalis
cagtgaggaacataagtatttgaacaccctgcgattttgcaagttctcccacttagaaatcatggaggggtctgaaattcacattgtaggtgcattcccactgtgagagacagcatttaaaaaaaaaattcaggaaatcacattgtatgatttttaaagaatgtatttgtattgcactgctgcacataagtatttgaacacctggcaatcagcaagaattctggctctcaaagacctgttactctgcctttaaaaagtccacctctactccactcattaatctaaattagtagcacctgtctgagctctttaaagacacctgtccaccccacagtcagtcagactccaactactacc

SEQ ID NO: 137 - DNA, 297 bp, Xenopus tropicalis
cactgctcaaaaaaataaagggaacacttaaacaacacaatgtaactccaagtcaatcacacttctgtgaaatcaaactgtccacttaggtagcaacactgattgacaatcaatttcacatgctgttgtgcaaatggaatagacaacaggtggaaattataggcaattagcaagacatccccaataaaggagtggttctgcaggtggtgaccacagaccacttctcagttcctatgctttctggctgatgttttggtcacttttgaatgctggcggtgctttcactctagtggtagc

SEQ ID NO: 138 - DNA, 318 bp, Takifugu rubripes
cactgctcaaaaaaattagaggaacactttttaatcagagtatagcaacctatcagtcaaacttctgggatattgatctggtcagttaagtagcagagggggttgttaatcagtttctgctgctttgttgttaatgaaatcaacaacaggagcatcagaggggcaacaatgagacggcccccaaaacaggaatggctttccaggttgaggccactgatacttttttccctcctcatctcttttggctgattttttactgactgactttctactgctgtagttattcatttggcttggatcaacatctctgttgatagc

SEQ ID NO: 139 - DNA, 1052 bp, Xenopus tropicalis
tagggatgtagcgaactgttcgccggcgaactaattcgcgcgaacatcgggtgttcgcgaacccgcaagttcgcgaacttttggcgtatgttcgccatttgggttcgccttagctggcgccaaattttgacctctcaccccagaccagcagatacatggcagccaatcaggcagctctccctcctgggccacccccccccccttggaccactcccttccatatataaaccgaagcccagcagccattttacattctgcctgtgtgtgcttgaagagatagtgtagggagagagctgtgcattgatttgagggagagtttaagtaggttttctggctagtaatctactttctactgctctgtatttgtgtggggagaggaagctgtgcattgatttgagggagagtttaagtaggttttctggctagtaatctactttctactgctctgtatttgtgtggggagaggaagctgtgcattgatttgagggagagtttaagtaggttttctggctagtaatctacttctactgctctgtatttgtgtggggagaggagctgtgtattgatttgaggcagagtttaagtaggttttctggctagtaatctactttctactgctctgtatattttgtctaaataacaatttgtctaaataacaataataattccgtgtccagaaacatcacctgagtgacggttttccaccagcaataatatattccgtatccactactgtatacgttgcccttgcaggccttgttgcccggtgtctgcaaccaagtgccacctagctgtgtgagctttttcacaatctgtctaaataataataattccgtgtccagaaacatcacctgagtgacggttttccaccagcaataatatattccgtatccactactgtatacgttgcccttgcaggccttgttgcccggtgtctgcaaccaagtgccacctagctgtgtgagctttttcacaatctgtctaaataacaataataattccgtgtccagaaacatcacccaagttgttgttgttttgtaaaaataaaaaaa

SEQ ID NO: 140 - DNA, 303 bp, Xenopus tropicalis
ccgtattttccgcactataaggcgcacccaagagccttaaattttctcaaaaatcgaccgtgcgcctaataatacagtgcgccttatgtgtgcactgagttgttgtgcgactttggtaagcgctccgcttgattgactgtcggaccatttcccgctgacacagggacgtaatacgtacactacatatgttggcagcgataaaccaatcagagaacattacgtaatacgtgcagtacgtacgcttacctctgtcacgcctccggtaggtatactaccagtatactgcaaaacaaccccccgaaa

SEQ ID NO: 141 - DNA, 328 bp, Anopheles gambiae
aaaattatttagtttttggattgaatttaggtaagcctaagaatgaaataaattgttagtgtgtatgatatattttttataacttttctcttaaactaaaaatttataaattagaattgtaacaccatggactaaacatctcaacaaatacataaaagtaaagttttgttcgagcatccgcattatttatttaaaataaaagacagttcaaaatttaattctttgtgtgaccgacagtgcgactgacctaccgcacgcgttcagttcctcatactgaacgaactacaccgcacgcgttcactgaaaagaatcaaattgcccaagccta

SEQ ID NO: 142 - DNA, 418 bp, Anopheles gambiae
tcaaaacgtcgttgcaaaatttgtaacatggagtatatgaaataattgttttgtaatgcactttttgtaattaagtaagacttgaattatctagatgtaagtgttatttttgtaatataaaaaatgaattcaaaatggtgttaaaatgtgttggttgccttaatcataatagatgcatcacttaataacataattaggaatacacgagttttttagttcagtgactaagaactgttatgcttattatatgaaactgttaacaaacacccataatatgaaactatcgcgaaataagatcgatttaccatactgtggttagttgacgtgaacgattgaatcgcgattcacgctcagttcatgttaatgaaagaaccggtaaattcacgttcatggtaatgaatcgaattgcccaacccta

SEQ ID NO: 143 - DNA, 207 bp, Danio rerio
gtaaagtgtgttcaatacttattccctgtgtcatttcactttatttattatgactcaacttgtatacttaaatgttctgatttctttgtatgaattcaatatttggcttgatggctacatctggtaggaattttgtgtcagtagcccacttagaaatccccttactgataaaaatgctgatgtgtcaaatacttattttccccgctg

SEQ ID NO: 144 - DNA, 166 bp, Petromyzon marinus
tggtgagtgttataacaacattatttaaaagaataaattcagaggcttattatactgacatttagttgaattcacttttaaaatatattatatggctctcactgaaataaatttccaaattttttgctttcatggctctcttagtcaaaaaggttcccgacccctg

SEQ ID NO: 145 - DNA, 206 bp, Xenopus tropicalis
gcaaaagatgccatgttcagaagaactgttcatgatcttcactcaatgttctattcatgttcaggacacttcatgttcagaagaaataattaaaactgttaataatgacatttgagtacttttttttgtaaaatcccttatgcggcccagcctcatcctgactttgcctcctgcggcccccaggtaaattgagtttgagccccctg

SEQ ID NO: 146 - DNA, 226 bp, Xenopus tropicalis
gtctttttttgttagagggttcaaaaacttatttcactcaatgaaatgcaaatcagttgctatcttttatttaaagttattttttcgattttccttttgatgtgctatctgccactgttaaaataaacctaccattgaaatgatactgttctgagacttttcatttctttgtcattggacaaacttacaaaatcagtgaggggtcaaataattatttcctccactg

SEQ ID NO: 147 - DNA, 173 bp, Danio rerio
gtcaagtaaaatgaggtaggctaatttaaatccatgaaaactattgcgttatataattaggtgtataagttattgtgacagatatactgttgtttgcgcatctgttgtgtggctctttgcagtgataaagttttttttttggctcatcatgccaaacaggttcgccacccctg

SEQ ID NO: 148 - DNA, 216 bp, Takifugu rubripes
gtgcttttggatagagggatcaaatacttattaccctcaatgaaaaacaaataaattaaaataaattctttaaagttattttctggattttctttttgatattctgtctctccatgttagaatacatctaccattaaaagtatagaatgatcatgtctttattagtggacaaaccaacaaaatcagcaagggatcaaatactttttactctcactg

SEQ ID NO: 149 - DNA, 220 bp, Xenopus tropicalis
aaatggccttgtcccaacttttttgggatttgttgacaccatgaaattctgaatcaacatatttttcccttaaaatggtacattttctcagtttaaacttttgttccgtgatttatgttctattctgaataaaatattagaagttggcacctccacatcattgcgttcagtttttattcacaatttgtttagtgtcccaacttttttggaatccgggttg

SEQ ID NO: 150 - DNA, 217 bp, Xenopus tropicalis
ctcagggggctgaataattacgcacaccccactttgcagttatttatttgtaaaaaatgtttggaatcatgtatgattttcgttccacttctcacgtgtacaccactttgtattggtctttcacgtggaattccaataaaattgattcatgtttgtggctgtaatgtgacaaaatgtggaaaagttcaagggggccgaatacttttgcaagccactg

SEQ ID NO: 151 - DNA, 375 bp, Anopheles gambiae
atgctaccaagttagcccaggataaggctgttttgttattttctttagtttttcaaatgtacgggctggccacattgcgaattaaaagccggagctgtaattatttataggcaataatgataaatggttaaaataataaaattttctatactggtcctattattttgtcgcatgatttttggggttttttcaacatttcttataataacttttttgtttgttaacttatcaacaaactttcaaagcagactttaagccaacatccgaaaagtgctttcatgaaaatttggtaaatattgattcactttaggtaatgaaatgtgataagtgaataaaacattattaagtggtcctattgatttgtccgacactgta

SEQ ID NO: 152 - DNA, 218 bp, Xenopus laevis
gttcagggggcaattactttttcacacagggccatgtaggtttggattttttttctccctaaataataaaaaccctcatttaaaaactgcattttgtgtttacttgtgttatctttgactaatagttaaatgtgtttgatgatcagaaacattttgtgtgacaaacatgcaaaagaataagaaatcaggaagggggcaaatagtttttcacaccactg

SEQ ID NO: 153 - DNA, 143 bp, Anopheles gambiae
aatgtgttttcgctcagtggaacaccgcaacacctcctcccaacaaccacttaaacagtccttttcagggctaccaaatatttctgacaagaactatgtctttcggtcacagaaaaaagttcctatttttatgtccaccagtg

SEQ ID NO: 154 - DNA, 37 bp, Xenopus tropicalis
tatggtgttcctaataatccttttggtgtgtgtatat

SEQ ID NO: 155 - DNA, 303 bp, Petromyzon marinus
ccttttttgctgcttagtttgtgagattcggatatgtgcacactaggtctgatgtaactatctttttgctaacgtcgccttctttgataactttttagtaaataaatatgttggttgtctttgttttttgtgcattgcacgccactcacccatgattaacatggaatgctaaacttagtttttcgtcttttttccccgagctttcgttctggcttgcaagtattgaacagaatgattatgcggcccgcgctcgtcattctttcagttatccggcccactgtgaaaaaagtttggtgacccctg

SEQ ID NO: 156 - DNA, 216 bp, Petromyzon marinus
gtcatgttttgcagaggggtcaaatacttatttccctcattaaaatgcaaatcaatttataacatttttgacatgcgtttttctggattttgttgttgttattctgtctctcactgttcaaataaacctaccattaaaattatagactgatcatttctttgtcagtgggcaaacgtacaaaatcagcaggggatcaaatacttttttccctcactg

SEQ ID NO: 157 - DNA, 381 bp, Alligator mississippiensis
cgggattcttctcgaaagctacacccaggaatccacactttgaaggattcgatgactaaaatgttaatctgaatgtctttttttataactttaatttttttggtacatcatttttataattttcgctgattgtatttttatgttctgtaatatgtaagagttatgcaaattaaaacaaagttatcgagttggagtttcctttagaccttttctttattcagaataaataaaaacgattacctgtatttacagtatgcaaacctaagtaatgtatattagagctgatttcctctattcaggggcggctcctcgcttatcgaccaattcgcttaacgacctagtcgtgggaacggaactcggtcgttaagtgaggagtgcctg

SEQ ID NO: 158 - DNA, 290 bp, Myotis lucifugus
tttctagttttcgtaccatttgattatcattgtttttattattattttttcctaaatgtacattgtttttctgtttttgttcattttatacatattttttggttgatttttctttgttaaatggcatttaaatatataaaataaatatcaaaaatataaatcttttttttactatggttgcaaatatcaaaaaaaaattatgaatatttctatatgtgacacggcaccagagttaagttagggtttttcaaaattttgacacgccgagctcaaaaggttcgccatcactg

SEQ ID NO: 159 - DNA, 389 bp, Petromyzon marinus
aactgaaagtgatgatgtgttgctgcagcgaggttggagccaaatctccgacacacagcctgcgtggctgctctaacctgttaatgtagataataaagaagttcaagtgaggtcattggtttgggcgcaggcctcagatgttttcatgatgtttgatcagtcacgttacgacatgtttttgtattgttcatgttcgtgcaatttgttaaattgccttgcaaataaatgctttgctacctgttaaaggccaaatcctttcatgtaatgatttttacatgtcatttatattagttcacacaaacactccatccatctgttcctggcccggcccctctgtcaaattttagaacccattgtggcccgcgagtcaaaaaatttgcccacccctg

SEQ ID NO: 160 - DNA, 219 bp, Xenopus tropicalis
tttaagggtgtgcacacttatgcaaccacattattttagtttttttggttttcttccctccgcctaaaagatttcagtttgtttttcaattgagtggtacagtttataggtcacattaaaggtggaaaaagttctgaaatgatttatctttgtctcatttttgtacagcacaggaacctgacattttaccaggggtgtgtagactttttatatccactg

SEQ ID NO: 161 - DNA, 218 bp, Xenopus tropicalis
gttaagggtgccaataattttgtccagcccatttttggagttttgtgtgacattatgtccaatttgctttttttcctcccttttttgttctgttccaatacacacaaagggaataaacatgtgtatagcaaaacatgtgttactgcaatacttttctgtgagaaatacttgattttctggaaaaatttctggggtgccaacaattttggccatgactg

SEQ ID NO: 162 - DNA, 216 bp, Xenopus tropicalis
cactgattttctcaggtgttcaaatacttatgtgcagcagtgcaatacaaatacattctttaaaaatcatacaatgtgatttcctgaattttttttttaaatgctgtctctcacagtgggaatgcacctacaatgtgaatttcagacccctccatgatttctaagtgggagaacttgcaaaatcgcagggtgttcaaatacttatgttcctcactg

SEQ ID NO: 163 - DNA, 258 bp, Xenopus tropicalis
gcctcattttgacttgttttaaggacattacatcaaagttggatcagcctgtagtgtttttttccactttaattttgagtgtgactccaaatccagacctccatgggttgataaatttgatttccattgataatttttgtgtgattttgttgtcagcacattcaactatgtaaagaacgaagtatttaataagaatatttcattcattcagatctaggatgtcttatttttgtgttccctttatttttttgagcagtg

SEQ ID NO: 164 - DNA, 258 bp, Takifugu rubripes
gaatcattttgagttgctacaatgacattttggcaaaatggaccagtctgctgcatcattttttcactttcatttttggggtgtctttgatttcccccctctatagggtgatcattttcatttctatcaaattatgtggcatcattttgttactaatacatcacctactttttatcaggaaagatattcaagatcattttcccccccgtttagatctgatgtgttttcgaagtgttcctctaatttttttgagcagtg

SEQ ID NO: 165 - DNA, 1442 bp, Xenopus tropicalis
ctgcccatgcagctgtccttctctgcacgccgcttgacaccacacacagctgtcctttagcgtcctcctccaccaccgtacaaactagggtgcaaatcctactggcttaattttaagccaaactttttggacaggtaaatcctgaatttttctgtcttctgtgcttggcacgctatcttagtttttttaaagggtttgcctggggcatggttatgataccatctatctaagatgttttttctgtcttctgtgcttggcaccctatcttagttctttgaaagggtttgcctggggcatggttatgataccatctagctttgatgttttttctgtcttctgtgcttgtcaccctatcttagttctttgaaagggtttgcctggggcatggttatgataccatctagctttgatgttttttctgtcttctgtgcttggcaccctatcttagttctttgaaagggtttgcctggggcatggttatgataccatctatctttgatgttttttctgtcttctgtgcttggcacgctatcttagttttttgaaagggttctgcctggggaatggttatgataccatctatctaacatattttttctgtcctctgtgcttcagtggctgcgacaacaaaaatacaaactttttcaacatttatctaacatattttttctgtcctctgtgcttcagtggctgcaacaaaaaaaacataatttttcaggaatgtacacattcctgatttttcagggttctgcaacagcggcaaaatcgtatcttttatggtcaccacaggtgatcaaaaaggtaggacaaaactgggcccacactgcagaatcagtgttttttggttcacgtcactgtacattgaattacctctgcctgaccgtgcacgtgcgcacaagcacggtgactgctaaacacaccactacagaaatattcccaccgacaggacgaacgtcctggaggtgacaagcaactagtaaaaactattattcgctcacttgacagtatcattaaagctttttgcgttttttttcgttgcagtaaacgcggcgttttgtctttgcgtgtgaaccggccgtaacctttacacgacttgattggcatgtagacgccggactttttaaagcagtttattacataagtttaggaatgtagtgtgatttctgccctttacagcacaaaacgcaacgctgtgtcaacaacgtatttttcagagaaatttttgcccttgatccccctcctgcatgccactgtccaggtcgtggcaccctttaaacaactttaaaatcagttttctggccagaaatggcttttctaggttttaaagttcgccttcccattgaagtctatggggttcgcaaagttcgcaaaagttcgcactttttggcggaagttcgcgaacgggttcgcgaactttttttgtgaggttcgctacatctcta

SEQ ID NO: 166 - DNA, 247 bp, Xenopus tropicalis
ttaacaaataatgtgagtgtattgttaatacaaatacaaagttcaactaaactcttgctttagttaccgttaccggtactttttttttgttgaataaagttcaactaaactcactgttttgcttccgttactttagcatgcgccttataatccggtgcgccttatatatgtaataagtacagaaatagaccccgtaattgagactgcgccttataatccggtgcgccttatagtgcggaaaatacgg

SEQ ID NO: 167 - DNA, 655 bp, Alligator mississippiensis
ccgtattttctcacatatagcccgcacactgatataacctgcaccaatgtataacccacggaaaattgtattttcggaaaatagtctgtgtatagaccgcacccatgtattccctgcacctgatagtgtcggtaccgggggcgattttaagcctattaagcctgagaggtgctctgcctactacaggcctttatacagtaacgtttagtcgttaattattaccagaatgttcgccatttgagcgctaattattactgtcaatttgtaacatctgctcaagtgtgtttagaaactccagtcagtaaagacgaccgactgcctacgaatttaccccgacctccctatctcactaatttcatgtatagttttagctacacgtagaatgtattatagataacagcatttctaacttaattttagtatagttaatggtagaatacagtaataaaataagcgaaatcggttgttgtccacatgccttaattagtaagcaacatatgaagctgaaacgaatgaccttgtggtaggttgttgtgagtattcagacttgcaaaagacatgtgaagtctcttttaaatctggacttacgcgattcctggggtaattacacgactaccgcagggttcgatcctacccttgcatacttcatttgaaa

SEQ ID NO: 168 - DNA, 1894 bp, Xenopus tropicalis
ccctttgcctgccaacaccgtacggcgtacgtgttggcaaggcaatgcactgagtgccagcgacgtaccttgtacgtcttcctgcttgtggcgatgtggatactgatcggcggcttcttcctctttcaaaagccgccgatcgctgcaggatcccctaggcaacgagcgctgggggctggaagtgacgctgttgcgtcagatgaccccaagaaggaccccagagcagcggctacgtcatagacatgtgcctgctgctggggtatttaaactcagccccccagcgccgccctctgcctctctgctggtcctggatgttgctggagccctcctgctgtgtgctgctgttctcctgcggtccttggctgtgagatttgctgtgcctgccctcgggtagtccctttgcttttacattacacactcactactcacttactacacacttactacacacttactacacatttttacattttagtactttacattactttttttttgcagatttacactaactacacttactaacttacatttgcactcactacacttacacacatacacttacacacatacacttacacttacacacatctataaaaaaagctttttgtgtctttgtgtcattgtttttgtccaaaaaaacctgtttatttggtgttgtgcccttttattcactgtttgttatttcatttatttgttcactgttatttgttcattgccttttattgtgattttattgattattcttttctgcgttgctcttttgttatatacttttgcttgttaccatcatttctgattattagtgattttattgtttgagttttgctttgctcttattacttgtttttggttgtactttgcattttcagttttatattactgttcattgctggttgttttgattttcagttattttattgaatcttgaggtttgcattgttttttataactggttctaacttgtttttgcttgtgctttgaatttatagttttttattcctaatcattactggttctcttgttattgatttgtgatattttgaccactagttactactaattgcattttatttgattgtaagttctttggctagtctgatagggtttttgttccagaaagttccagagagttcagggagttgaagagttcagggggttgagcttatagcattttttttttcttggtctgatttgtttatattatcagttgggtttttttctccagtctctgccttgcctgttgcagtagttttacaagtatcccattttattgctttattgcttttattactttattatatatttttgcttgttaccaccatctcattgttgtttgagttttgctttgacttctgactagttttacttctgactagttcttgcttgtacttctagttttatattgcttttcattactagttcaattgattttcagtgattttattttgaattttgagttttgcttggttctaattactgttcattgcttgattttgctctaattactagttgtaacttgttcttctttgtactttggattttcagttttatattgctgttcattgctcttgattttcagtgattttattttatctttattttgcattgttgttataactgcctctaacttgacctctagttctttggctagtctgatagggttttttgttccagaaagttccagggagttgggagggagttcaagggggttgagcttattagcagttttttttttcttggtctggtttgtttatattatcagttgggttttttttctccagtctctgccttgcctgttgcagtagttttacaagcaatctttttttctggcttgattgttagttctttgtcactgagttttagattagcctgtacatattttgtttgcaac

SEQ ID NO: 169 - DNA, 992 bp, Alligator mississippiensis
ccttcatacgttcccatggggtaatttcctaccccagacgcatattttgtgcagtatcttaaagttaattaattctaaaaaatattctttttaggacattgtcactggcaccgcagtgcacaatatccccaaagaacagccagcttacatgaatggtttgctctctgtggatgaaaaactgcccagggtaacatgttaccccaggcgtgaatgtgtttttccatatggggtaacaacttaccccaagcccatgttaacgcattggggtaacttgttaccccaatgcaatttctgtataaatgacaggcctagacctgtttggccactttgggtggatgcatctgaaaggtcagcattcattcacacctcattcatgtccacttcaggtatccatggtaactaatggatggccatcctccactctgcttttgcagaagatgtggtaatcccttttgctaaccactttgtttgcaggctgcaaggtcatcaaaacaccctgccctttgccaaaaaaacccaaaaccctttgtcacacccaaatccacctctgctctcttttctcatggcaaaactccgcctcacctgcccctttcataacatacaagctagtttgccctgcctttgttggaagtgaggacaaggagcaaagggacagcagagaagaaatagaacaacatcatactgcaatacagcgtacactatggagtccccttcaacggtaagggcttgacaccccccatgccccttttacctgctctcaataagcccctgcatgtgtggtttccaaacagcatccacccacatttggcttgcctgcaaagggtggatgctggtctctggcttctgtgcccaggaggagagggtgctacctgcgctgccatagggcagggcggaattgcttctgtgacagcataggtggacaggcttgtctttgattgttatttatatgcattcagcagtaatcatttttttttttttttctgcagtccagg

SEQ ID NO: 170 - DNA, 121 bp, Alligator mississippiensis
cagtagaaccccgttttacgcgtattcgacttacccgagtctcgcgtttacgcgaattaatattagcacccaagcagaaaaatacaccccgagacgcgcgtcattcgcgtatatacgcgag

SEQ ID NO: 171 - DNA, 281 bp, Petromyzon marinus
cagtggttcttaacctgggttcgatcgaaccccaggggttcggtgagtcgatctcaggggttcggcagagcccccgccacggaggtccggacacacttgaattattgtgtaaatttgtgatgacacacacggcccgcttggccatcactggctgcagatgatcatgctacactgcttggccaatcagcgctgcggggaatttagtgcgtactcagtagtcaacgtgtgactgttgttgtcgtacgtcgtgtgatttctttaatattttatccatactaact

SEQ ID NO: 172 - DNA, 1159 bp, Alligator mississippiensis
tagggctgtgcgaaatttcgccggctgtttcgtttcgacgctgtttcgactcgtttcgagctcgaaacagcgaaatcgaaacgaaacgaagggcttcgaaacagcctcgaaacgaaacgagggcagtcgaaacgtttcgaaagtttcgaaacgtttcgagtttcgaagctgaagctgggcttgcttgcagggaaacagaaagtaaggagagggagggggaagaggggggaaggactgctctgatattgactgtgtagctggccagtgactgtgatccttccctgaggtcccttccaatcccagtgctctgggggagggatggaaaaacagcataaaaagcagcattgtttgaactgagctgctttctgccttggggtcagttactgagctgtgtcttccagcagctcagcagcgtcagacaacaaaggctaccattcatttcctacttgctagcctagcaagtgggattcatcaataccttttgctttctttctatacccttttattttattattatattcattcattattcctttcaatttattcctcccccaaaatcctctttttgtttttgttagtctatttcgattcttttcccccagaaaagaaatctgtgctttctctcacagtgtgcattccattcactgtgcagggaagcacagcataattatattgcatattataatatagaatttatacaggaacacatacatacatttatatattttattatttattacataaatacatacatacatacatacatacattacataaatacacatacatatattatctacatatattacataaatcagattacattacagaagaacacacagattttccagcacaccaaggcaaagacaagacactatgaaacacaccatgaagcgcgctggaaaggcagctggcaagccttcagggaggggcggaagagggggtagagggagagctgtaagttccccccccccccccaaactgagacgcagcctctttcctacaacaagcaggcatgaggcttccactagtacaggcagcaaggagaggggagcagtgccagtgccactgcctgtgcctgagtctgcatcccctccaagagcaccagccataacccccccccaccaccaccacgacaacaagcagcaccagcaccagc

SEQ ID NO: 173 - DNA, 408 bp, Microcebus murinus
caggttgagcatccctaatccgaaaatccgaaatccgaaatgctccaaaatccgaaactttttgagcgccgacatgacgccacaagtggaaaattccacacctgacctcatgtgacgggtcgcagtcaaaacgcaggcgcacaacacacagtttattcagcgtccccaagggaaaaaagaccctcccagcccccttcagctgcgatatatcttttccgcgcacgcccagattcccccacgcaagcacgcccacaaagggtaataaaatggcacgtgtgcaggccggacgcgccaacggcaggttccccacgatgccccacatggggccaagacctacgtgcattactcactgtgttttttttgttttttgcttattctctgctctgtggtgtaaagatattgttgaaa

SEQ ID NO: 174 - DNA, 319 bp, Trichechus manatus
cagtagtccccccttatccgcggtttcgctttccgcggtttcagttacccgcggtcaaccgcggtccgaaaatattaaatggaaaattccagaaataaacaattcataagttttaaattgcgcgccgttctgagtagcgtgatgaaatctcgcgccgtcccgctccgtcccgcccgggacgtgaatcatccctttgtccagcgtatccacgctgtatacgctacccgcccgttagtcacttagtagccgtctcggttatcagatcgactgtcgcggtatcgcagtgcttgtgttcaagtaacccttattttacttaata

SEQ ID NO: 175 - DNA, 352 bp, Alligator mississippiensis
tagggacctaccgaaatcgcgatttcgcgattttcgcggaaaccgcgatttcgggcggtctccgcgaaaaagcgcgggctccgcgaaaaagcgcgggctcgctgcgggggggaaagcaaaagaaagctgcaagaacagaaaagggcgggaagccctggcgtcctgaagcacggggggagggaggaggggggagagggagcaaatcggaggctgagcccctgacgtaggctcagtccaggagccagtcaggggccagccagggaagcgggagggttttggcgccggcttttaaagcgcccgcaggcatgctgctgcagcatgcatgctcgcttccgcctgaggaagagcaggg

SEQ ID NO: 176 - DNA, 992 bp, Alligator mississippiensis
tagggctgtgcgaagcttcggttgccgttcgattcggagaagattcggcccgattcggaggccgaatcaccgaatccgaatcgaatcgggaggccaaaaaaccctccgaatcgattcggagcctccgaaacgattcggaaaagattcggagattcggaaggcggtctttcaggggaacagaaactgagaagagggagggggagcagggggggggaaagactgacatctgtagctggccagtgaccgtgatctttccctgagtccccttccaatcacagtgctctgggggagggacgtagaaacagcatataagcctctgtctgcagcctttctgtcccttttgcgagctgtttctgcctgagcaacagcgtctgaaaggtactggactggcttggcttcctagtcagtctcctgctagtttctgttcttggaaaggattggatacagcttactaattacccttgcctagaatccctatccaatccatttctttcaacttattcttcccccaaaaacctcttatttgttcttgttttttcccccacactttaaaagaaagctgtgttttccctggcagtgtgatccatcactgtgcaggaaagcacatacattacacacactcacacatacagtaaacagagaagaagaagagatattaattattaaacacacacacacacagagaagacacaacagagaagaaagaacacacacacacacacacacacacacagacacattttccagcacaggaaagattaatatgaagcgtgctggaaaggcaggtgccaggtcttctggcaggggagggagaggggctagagggggtagggggagagctgcaagttccccccccccccaaacatagacgcatcctctttcctggcagccatgagtcttctgctgccggtgggagcaaggaggtgggagcagtacctgtccctgcacctgcaccacctcccctaacaccagaaatagccaccagcaccagc

SEQ ID NO: 177 - DNA, 729 bp, Xenopus tropicalis
tagggatgagcgaaaaaattcgccagcgtcggtttcgcggcgaacttttgcgtttcgccgccggcgaaatgtttcgcgaaacggcggcaaaaattcgccgtgcggctcgtttcgccgcgcggcagaattttgacgcacgtccaaaattttttgtcgcgcggttatttttggcgcgcgcggccaaatttggtcgtgcgcgagcaatttttgccgcgagcggccaattttagtcgcgcgcgggcaatttttgacgtgtgcgccgatttttccgccgtgcgacaaaaaaaaaaacgcgcgacaaacggaaacgtcattttagccttgcccatttcttccacatcagccaattagattggagccctcccactgtccatataaggaggtgcagaggcggccattactcagtgcgtttttggtagtggatagagtaggagctgaggtgtgtgaggtgttgtctgtttagctagaggtaggatttgttagagctttctgtgagtcagtgcaagtggaagttgtggatttcagttcactgctttccctcttctgcttgcctgctcccctgtgagacccaagagcccttgttagggctacgtttgtctgtctttgtttgtgtgtgtgtatcccttgtgggtgcacacttctggggtttagtcttttatttatttatccctaccttcccccaaatatccaacccccccctatttttttttcttttttagttggtccagg

SEQ ID NO: 178 - DNA, 394 bp, Drosophila yakuba
tacagtggcgagcaaaactgagtgcatgttcacaactctcactttggccatcaataaaaacacaaccgcttatgcaaattcaataattttttttatattattaatcttaattaatttataacttattttatgagaaaaaacaaatttagcatatatattcaaagacttagaataaaaaaactaaataaactcacataaactcaattttgcacgttttaagtcttgtaactgtttttgattttaacatctcttatcagtataaatcggccagatttaccgttatatttcttttaatgctattttcggtggtcattgattagttaaaagcgtgtttttgcagagcaaagttctttagggtgaataaatataaataattagtgataaaatccttaaa

SEQ ID NO: 179 - DNA, 528 bp, Alligator mississippiensis
atgttatttagacatttatatgttttaattttcattaaagactgttttaacatttgcaataaaaaatgtgattttgtagcataagttttttactattaatgttgtgatagcgcacctttagccagtagttaagcaacacttccctgcttttcctatctggcatcttacttactttttgagctacattaacagtttaaaaaataccagtacatttaacaatccatatcttttatttataaattgaaacaaactatatacatttgtatttataattcagtcaatttaatgaatttgacttctttccatcggcgttgggtggaccagctatagcactcatatcgtcatcgtccagtatgtcgtataacctggaaatactattcaaacaattattaatgcacttattaacttctgctacacttgtttttgcataaatggctctgcatagcccgcacccttgtatagcccgcacccatccgtcatgtttgtaaaaacttatataacccgcggactatatgcgagaaaatacgg

SEQ ID NO: 180 - DNA, 4787 bp, Xenopus tropicalis
aaggtttataaattgtgtattgatttggattggatttatagtaagcattggtatcccactttgtgcaaaatttatatagtatactttgatttatgtattcagggcagttgtggttattttggaattttcagtaccctaatatattgcttattatggatggcactgggggtatagccaattgaggtgtaaggtaacgcacagggcgcctacacatttgtggtgtgtgatgattatgatgattcatcttgcctgctgatgttttcatctcccctgctgatgccacaaagttttttttctactgacacctcataagctgcccttgctatctgtttttttggacaatatttcatattttactagtttattgggtctccctgaaatcttgttttttgttatgggctccagcttttttgtaaagtgatttttgcatgtttagggtttttgttttgcagttgtgaaagtagttttgctgatggtttgctgatagatggggacagtttgcatttcaatactaatcaactattatctactaatcaactaattcaatgctaattgtagtctttctctttattatggatattagtactgagctgaaggcataacagtgaaaggaacagggaacttgattgacaacttactgaggatatctttaattttgggaagcatatattttcttcttagtcacctagcaccacaatttagcaaaaagctaaatgcttacatcaggggtcgtcaaactttttaagtgctgggtccttccgctctccgctgggtctttccgctctccgctgcgtccatctgctctccgcctcgcatcatattggggaacaggtaaattatcttggggggccgtatcaagcccaagggccgtagtttgaggatgtctggcttacatgaaccgcaagcgtgatcagacttgacatcacctctttagctaatgcaagctttattgttagcatctcaaatgctcacaattcagtgaagtacacatgaaacgaaagggccgaaagatgctcaactaaatagataaacgtaagtagaatttatttggggctaaattgggcagggtgttttagtcattattgcaatttcaaggagaaatctaactttggatctttttttatcctttattttaggctaaactgcaaagttcatcggagaactgtgtccacaattctatatgtagggaagcaaggatggttttcctgaatagctgcaggtgagcactatcaaggaacatatagtttgtgggggtattttccaggtaaggggggtgttaagcagtaccaccgctccctatacgcagagtacacactctgcgtagggcaccaactcccaggggggcacccagacagtaccgccgctccctacgcagagtgcgcactctgcgcaacaacataccaagtcttcgccagcgccagtcagcgccagcggctccttctctcttatgctgcggcaacaggcccttttataaggttgcgcccgtgcgtatgacgtcacacatcagcgacgaggcgcaaccttatagaagtgcctgttgccgcggcataagagagaaggaggcgccgacgatcactggtctgttgggggtactttctgtgggggcactgtgcgggctggctaatgtctatggggggtactgtgtatgaggcaattgggggtactgtttttgggggcactgtttatgggggcaattgggggtactttgtatgggggcactttgtgtgggggctactgtctatggagggtactgtttatgggggcaattgggggtactttctatgggggcactgtgtgtggggtctactgtctatggggggtactgtgtatgaggcaattgggggcactttgtgtgggtgctactgtctatggggggtactgtttatgggggcaattggttctattttggcccctatagtaggtgtttttttttttttttttttttctcggtaatatttgggggagggggcaccaaagtaaatttcacccatttggccagcagcggccctggtgttaagactcaaaatctagcagatatgcatttatagcatagccaccagatttagctattgccctagttttagggtgtttggtgggtgtgtcttttgatactcacatatgtggggtatcgtttgattcagaagaagctgaagattgatattgagaaggttttttgtagttgtcacggcaattttagggagaattttgactttggatctttttttttcttcatttcaggccaaaccgcaaatttctacgaagaactgcgtccacagtttttcatgtaggggaacaaggatggtaccgctgaatagctgcaggtgtgcactttcaagaaatatatggtttgtgggggctattttacaggtagggggtgttttgactaaaaaactgcaaggagtgcacttagagagtagccccaaactttccagctgaaattgctcgtatgtattgcccctgttttggggtgtttggtggccccgtctttaggtgcacacttgcatgtggggtatcgttttgttcgggagaatttgcagattgatattgagcaggttttttgtagttgtcatggcaattttggggagaaatttaactttggatctttttttttcttcatttcaggccaaactgcaaacttctacgaagaactgcgtccacagtttttcatgtaggggaacaagcatggcaccgttgaatagctgcaggtgtgcactttcaagaaatatatggtttgtgggggctatttcacaggtagggggtgttttgactaaaaaactgcaaggagtgcacttagagagtagccccaaactttccagctgaaattgctcgtatgtattgcccctgttttggggtgtttggtggccccgtctttaggtgcacacttgcatgtggggtatcgttttgttcgggagaatttgcagattgatattgagcaggttttttgtagttgtcatggcaattttggggagaaatttaactttggatctttttttttcttcatttcaggccaaactgcaaacttctacgaagaactgcgtccacaatttttcatgtaggggaacaaggatggcaccgctgaatagctgcaggtgtgcactttcaagaaatatatggtttgtgggggctattttacaggtagggggtgttttgactaaaaaactgcaaggagtgcacttagagagtagccccaaactttccagctgaaattgctcgtatgtattgcccctgttttggggtgtttggtggccccgtctttaggtgcacacttgcatgtggggtatcgttttgttcgggagaagttgcagattgatattgagcaggttttttgtagttgtcatggcaattttggggagaaatttaactttggatctttttttttcttcatttcaggccaaactgcaaacttctacgaagaactgcgtccacaattttccatgtaggggaacaaggatggcaccgctgaatagctgcaggtgtgcactttcaagaaatatatggtttgtgggggctattttacaggtagggggtgttttgactaaaaaactgcaaggagtgcacttagagagtagccccaaactttccagctgaaattgctcgtatgtattgcccctgttttggggtgtttggtggccccgtctttaggtgcacacttgcatgtggggtatcgttttgttcgggagaagttgcagattgatattgagcaggttttttgtagttgtcatggcaattttggggagaaatttaactttggatctttttttttcttcatttcaggccaaactgcaaacttctatgaagaactgcgtccacagttttccatgtaggggaacaaggatggcaccgctgaatagctgcaggtgtgcactttcaagaaatatatggtttgtgggggctatttcacaggtagggggtgttttgactaaaaaactgcaaggagtgcacttagagagtagccccaaactttccagctgaaattgctcgtatgtattgcccctgttttggggtgtttggtggccccgtctttaggtgcacacttgcatgtggggtatcgttttacttgtgagaacttgttctttcatattttacttcatttgaaaatttttattggatatttttttctcaaattcacttttgtctctgtgactttgatggcatttcataaaataaaaaaaatcccaaaaagtattgttgaatttcgtagtgatctgtatgacgccaactgcatttagagcaaaaaactaccccagacccaaaaacctagaggtgtgtagtttctaaaaatacctaacttatggaggtctttcacttctatcattagatataccatgttaacatagagatgcgcttatcggttttgtttcaatgtaaaattgtagaaaatgttgttttattattggggtgtcttctgggcaagaaaagtgggataccaatacatatttggtattggtggattcagcagaatcggggcttttatgaataataaaatttttgtcagtaaatgtaatttttcttgaaaaaaaaacacaaaataaaaacatatttttatttattttattttttttttacatatttcactcaaaatttttgttacatctcctgaaaaagtacattttttttttacagtgttcaagtccaattcgctccggaaaaaacgatatataatttgccttatttcatgtaggctttcttgtcaaaaaacctaagcaaatgtaatgagtgcaaaatgtctcaaaattgcttggcagtagatgttcgcttttagggcaaattggctggcagtgaaaggg

SEQ ID NO: 181 - DNA, 351 bp, Alligator mississippiensis
ttaaaacagttaggaaatgtttttggaagtaattgttagtttgaaaacagtgtgaactgcagacttagcaaattgttcattcacgttttgaaactctaaaaaaagtttaaattttgttcatagttctagttaaataatattttgggttcaaatcatcataaatactctttcaaacaatctgtatgtttaataaatagtttttggtacatttatactgttgtcactggttccattttgttcatgcgggtggggtaacaagttaccccatgagaccaacaatgtcgaattgcggggtaacaagttaccccatgagaacaacaacggttacggtttcatgagaacggatgaggg

SEQ ID NO: 182 - DNA, 287 bp, Alligator mississippiensis
tggatggattgaagattgttgcatgtccattttgtatttttttattattttacttataatgtaaatgaataaatatgtttacaatacgtttttgtaacataataaataaagtattggttgaaaaaggttgtatttggtgcctttggtgtcataaatgtgaggtccaaggaacgcaacccctcacttttctattatttcctatgggaaaattcacctcgagttgcgcgaattcgacatacgcgaggtttctcaggaacgcaatatacgcgtaagtcggggttctactg

SEQ ID NO: 183 - DNA, 221 bp, Petromyzon marinus
tttccaggtatgtaatttgttgtgagtttatgcattgtgttggttttattctttgaacaaggtaatgttcatgcacggctcattttgtgtaccagtaaaaaacatatctatgtcttgaatttgaaaaaaatcacattttatttttcactaaagaggggttcggtgaatgcgcatatgaaactggtggggttcggtacctccaacaaggttaagaaccactg

SEQ ID NO: 184 - DNA, 2237 bp, Alligator mississippiensis
gtgccaccctcctcactccgtccgcacctatttccttttttcattttttaaaaaccatttaatgccccgctctcagagctggagggggcactcactgcatcaccagccagcaggggaagaacatgtccctgctgagctcccgtgccaggacgaacttggaggtatgggctggggctatggggaaggaggaggccccaggtcctgggcatgaccactaaaactgcactctgccagggtcgggaggcctggtgggactgccggcggcgcggcagccccaggaaatgccaggaccgaggatccccctggtgaaaggcgggtaggaggcgccataacctccagccaccacacgcacgcacacagtcctggctggggaaagcccagacccgtcacgtctttgacaggcctgaggcttgctggttgcagtggagttgtgaggctccaggtgagctcagcttagctgcctgggatgccagctttcgggttgaaaaacccttcgtcaggctgaggaagcacctgcagttggtgtgtgtgctgttcctggatggaaggaatagtaaagaagccagaggctggcatgcaatgcaggcaagaaagccagtcagtgaaaatggaaatggaggcgtcagggggtgagggacaggctggggtgggggggggaagggggatgtagcagcgcaggtaaaagtggagaggtacctggggagtcagatgtccggcaggttgtagtgtgtcagaattccaatgtctatattgagtccatgagtttctgtacctaggaggttgatgaagtgcagttcataggctcgtctgtgaaaagtggtttgtaaattccctttgaggatcaggcctgagagattggagacaggtggtgttctgttcttgtctttgatagattttcggtgtgcgctcattctggtgcgcagttgttgcttggtgtctcctacgtattttccatcagggcacttggtgccctgatggagatatgtattgtattgtgaagatatctccatactggagatattgttggcaggttttgcgtgtcttgtcatggcatggcctggatccattcggtgtgttttgggctgtaggaagttggcttctggtgatgaggttagcgaggttcagtgcttgtttgaatttacaaaccacttttcacagacgagcctatgaactgcacttcatcaacctcctaggtacagaaactcatggactcaatatagacattggaattctgacacactacaacctgccggacatctgactccccaggtacctctccacttttacctgcgctgctacatcccccttcccccccaccccagcctgtccctcaccccctgacgcctccatttccattttcactgactggctttcttgcctgcattgcatgccagcctctggcttctttactattccttccatccaggaacagcacacacaccaactgcaggtgcttcctcagcctgacgaagggtttttcaacccgaaaccttgctaagatatatttctctaactattcagttggtctactaaaagataccagattagattgacccaaagaaccttgtctgccagtgccacgtccttagaccaacacccctagcacctgtccccggtcaggcgacaggcgggcacatctaagccccagggctggggctggcagggagagttctctggcagctgctggagaagaagctcctgcaggaaaggaaagaaaggctctgtgacagtttggctgcctgagatgctgctggtgctactgtgctgcttactattttacctgttgttatttaaagtggtggaccgggctgaggctgtaggggaggaggagacctcattggggcacactaccgtgtcgtgtagaggccccagcgccagtgagggcgcgccttaacacacacacaaagtcacacatgtcacgagttttgcttgtcagcagcttgaggtgcagtttcccagaaacccctgcacctatctccttgaaacttggcaggcttcgtggcctcagcaggggctaccatccctgctgttttcatccgaatcaggcaagaaatgacaaagttataggcagtttcgtgattccccattatagcctatgggcgaaacgtcgaaacagcgtcgaaacagcgaaacagtttcgacgaaacgaaacggaacgaaactcgaaacgaaacactgtcccgtcgaaacggcgaaacggaagtcgaaacgaaacggtgctgtttcgcacagcccta

SEQ ID NO: 185 - DNA, 658 bp, Microcebus murinus
aaagccatccagcagaatgcctcctcatccctagaggacccacttcctggtccctcaactgcttctgatgtttcttctcacctaaaaaataaaatacagtgtacagtaaccttttaatcaaaacacagcatcgtaggtggagactgaaagcctgccgttgtttgttgttgctgttgtttaacagctgatacaggtattctggtgatgctactgtgctgcttagttaccctgaacacattattttttcactgtattaatggtatgtcatattttttactgttaagtacttatgtgtgaataagtgtaagaaaatgattgcttatcggtagcatataaattcagagtcaggaatgatggtgatgccaaacaaccacagattgtccacatgggtggctgagatagtgacacctttgctttctgatggttcaatgtacacaaactttgtttcatgcacaaaattatttaaaatattgtataaaattaccttcaggctatgtgtataaggtgtatatgaaacataaatgaatttcgtgtttagacttgggtcccatccccaagatatctcattatgtatatgcaaatattccaaaatccgaaaaaatccgaaatccgaaacacttctggtcccaagcatttcggataagggatactcaacctg

SEQ ID NO: 186 - DNA, 244 bp, Trichechus manatus
gatattttgagagagagaccacattcacataacttttattacagtatattgttataattgttctattttattattagttattgttgttaatctcttactgtgcctaatttataaattaaactttatcataggtatgtatgtataggaaaaaacatagtatatatagggttcggtactatccgcggtttcaggcatccactgggggtcttggaacgtatcccccgcggataaggggggactactg

SEQ ID NO: 187 - DNA, 743 bp, Alligator mississippiensis
catgaactgcctgtctcaaggttagcattcagtttcagtaaactcctttttataatgtcctttaataaagtgtatatagttggtattagttacaattcatgttgtgcatagtacagctttattttcataagatatttcagtttagagtttaattaagtgcttagatggtattagttaataaagtgtatatagttggtattaataaagtgtatgtagttggtattagttaataaagtgtatatagttggtattagttgcaattcatggtgtgcatagctttattttaataagatatttcattttagagcttaattaagtgcttagactgcttaataacgcgtcaattaatggcttccgcgggcttaatcccctgattaagcccgcggaagccattaattgacgttattattaagcagtctaggacgcggttaatggcttccgcgggcttgatcaagcccgcggaagccattaaccgcgtcttagactgcttaataacgcgtcaattaatggcttccgcgggcttaatcaggggattaagcccgcggaagccattaattgacgcgttagcaagccgcggtttggggccatctggcaatctgcggggaggggcgggactaccggggcccctcggccaatcagaggcgtgggggggcccgccgcgctcggcccgcgaaaatggcgcccggccccgcgatctgcccgcgaaatcggccggcagccgcctcgagttcggtaggccccta

SEQ ID NO: 188 - DNA, 2786 bp, Alligator mississippiensis
gtgccaccctcctcactccgtccgcacctatttcttttttttaaccatttaatgccccgctctcagagctggaggcggcactcactgcatcaccagccagcaggggaagaacatgtccctgctgagctcccgtgccaggacgaacttggaggtatgggctggggctatggggaaggaggaggccccaggtcctgggcatgaccactaaaactgcaccctgccagggtcgggaggcctggtgggactgccggcggcgcggcagccccaggaaatgccaggaccgaggatctccctggtgaaaggcgggtaggaggcgccttataacctccagccaccacacgcacgcacagtcctggctggggaaagcccagacccgtaagatctttgagaggcctgaggcttgctggttgcagtggagttgtgaggctccaggtgagcttagctgcctgggatgccatctgcgaggcatgggctttcgggtttaaaacccttcgtcaggctgaggaagcatctagttggtgtgtgtgtgctcttcctggatggaaggaatagtaaagaagccagaggctggcatgcaatgcaggcaagaaagccagtcagtgaaaatggaaatggaggcgtcagggggtgagagacaggctggggtaggggggaagggggatgtagcaggtaaaagtggagaggtacctgctctggggagtcagatgtcaggcaggttgtagtgtgtcataaatccaatgtctatattgagtccatgagtttctgtatctaggaggttgatgaagtgaagttcataggcccgtctgtgaaaagtggtttgtaaattccctttgaggatcaggactgagagactggagacagagtggttttcttgtgagaaatgtgcccccacaggtagttgggtattcttgtctgtgatagattctggtgcgcagtttgtgggggtactggagatacgttggcaggttttgcatttcttgtcatggcatggtctggatccattcggtgtgttttgggctgtaggaagttggcttctggtgatgaggttagcgaggttcggtgcttgtttgaatttacaaaccacttttcacagacgggcctatgaacttcacttcatcaacctcctagatacagaaactcatggactcaatatagacattggatttatgacacactacaacctgcctgacatctgactccccaggtagcaggtacctctccacttttacctgctacatcccccttcccccccccaccccagcctgtctctcaccccctgacgcctccatttccattttcactgactggctttcttgcctgcattgcatgccagcctctggcttctttactattccttccatccaggaagagcacacaccaactagatgcttcctcagcctgacgaagggtttttaaacccgaaagcttgcaaagaagaatttctccaactattcagttggtctaataaaagatatcagattgaccctaagaaccttgtctgggctatgtccttagaccaacacggctacaacctacaccccttaacacacacaagtgacacgagttttgctttcagcagtttgaggtgcagtttcacagaaacccctgcaccactctgcctgaaacctggcaggcctccctcatgcccgcacgaggggctatgtatgtggcaggacaggtgctaatgagggcggccatttaatgccccgctctcagagctggaggcggcagtttagtttctcaccagccagcaggggaagaacatctccctgctgagctcccgtgccaggaagaacttggaggtataagggctggggctatatggggaggaggaggccccacatcatcctgggcatgacctaaaactgcaccctgccagggtagggaggcctgggtgggactgccggtggcgcggcagccccaggaaatgccaggaccgcgaacctccctggtgaaaggcgggtaggagcaggaggcacctgataacctccagccaccgcacagtcctggctggggaaagcccagacctgtaagatctttgagaggcctgaggcttactggttgcagtggagttgtgagggtgagaacaggggctaccatccctgcagttttcaccccgctgcgccttaacacacacacaaagtcacacgtcacgagttttgcttgtcagcagcttgaggtgcagtttcccagaaacccctgcacctatctccttgaaacctggcaggcttcgtggcctcagaaggggctaccatccctgcagttttcaccccgctgcgccttaacacacacacagtcacacgtcacgagttttgcttgtcagcagtttgaggtgcagtttcccagaaacccctgcacctatctccttgaaacctggcaggcttcgtggcctcagaaggggctaccatccctgcagttttcaccctaatgcgccttaacacacacacgtgacacgagttttgcttgtcagcagcttgaggtgcagtttcccagaaacccctgcacctatctccttgaaacttggcaggcttcatgccctcagaaggggctaccatctctgccgttttcatccgaatcagccacaaaatgacaaagttataggtatttcagtgattccccattatagtctatggccgaatctccgaatctctccgaatcggcgccgaatcttccgaagccgattcggccgaatcgattcgggacagtgatccgaatctccgaattgaatcactgtcctccgaatcggccgaatccgaatcaaatacttccctattcgcacaggccta

SEQ ID NO: 189 - DNA, 6944 bp, Xenopus tropicalis
cctcctctccttcctcctctcctcccagatgtaccctcctctctcaattgctgccttatactcccctaataatctagctgctgctgctgctgccactatatcatctcatatcatattctaatttgtggcctatcaagggtccttacaatgttgctactctttcttaaattctaatttcttctaatttggggtctgaaatggctcctaattatattgtggctactcctgcaaaatatcatctactttggggctggaaatggctcctaattatattgtggctactcctacaaaatatcatctactatggggctggaaatggctcctaattatatcgttgctacttctgctaaaatatggaccaatgtcttctaatttgtggtctgttgttttctaataacctgctgctgctgctgctgccacagtatcatctaatttcttctaattatggcctatcaaggctacttgcaatgttgctacttcggcaaaatatcatctactatggggctggaaatggctcctaattataccgttgcgacttctgctaaaatatggaccaatgtcttctaatttgtggtctgttgttttctaataacctgctgctgctgctgccacagtatcgtctaatttcttctaattatggcctatactggctacttgcaatgttgctacttcggcaaaatatcatctactatggggctggaaatggctcctaattataccgttgctacttctgctaaaatatggaccaatgtcttctaatttgtggtctgttgttttctaataacctgctgctgctgctgccacagtatcgtctaatttcttctaattatggcctatactggctacttgcaatgttgctacttcggcaaaatatcatctactatggggccggaaatggctcctaattataccgttgctacttctgctaaaatatggaccaatgtcttctaatttgtggtctgttgttttctaataacctgctgctgctgctgccacagtatcgtctaatttcttctaattatggcctatcaaggctacttgcaatgttgctatgcctgcaagtatcatctactatggggctggaaatggctcctaattataccgttgcgacttctgctaaaatatggaccaatgtcttctaatttgtggtctgttgttttctaataacctgctgctgctgcttggttggtttgaaccaagatcagctgctggcgataattgccatttgaattctaaacttgtgctgaggtaaacatcgttggtctgtaggcacaagaattttccgtgctgcaatttgcgccgtctgatcccaaaagcctgtgctgaggtaaaacagtgatggtatataggcacaaggtttgaaccaagatcagctgctggcgataattgccatttgaattctaaacttgtgctgaggtaaacatcgttggtctgtaggcacaagaattttccgtgctgcaatttgcgccgtctgatcccaaaagcctgtgctgaggtaaaacagtgatggtatataggcacaaggtttgaaccaagatcagctgctggcgataattgccatttgatttgaaaagtgagagatctatgctgttgtaatgggaagatcttttattagagaagcgccgtggaattgaaaaacttatgccgaggtaaaaatcagtggtatgtaggcaaaaagcttcaattcctaatatttaattttatgtaagctctaattggtaatttagtttggtcattggatgcttttgatttttacggaggaggtgggggagtttgatgcattggtaaactcccttccccctcctccccttttcgaatgtagaagcattgtattgccaactgcctgtgctgaggtaaaaacgttggtatgaaagcacaaggcataaagtgatgatattgtctctgccttctaggtgtttagtgataactgctgtcgatcattttataggtgctcagtaacaggctgagattgaccattgcggcaggagtaactgtacattaaaagttatttagtccctatgtagtggacaattatatgtggaacattttgaagctaagtaattgcaatattgattgatgatggcaaataaataaataaacaattaaaaaaagaaatcaaataaataaataatgaaaataatttacttcacacaagtttattataaataaagatatatttggtgacatctatagaaatacatttataatacaataataatacaataatttacaatccttatgttttcattcagggtgaccccattacttctagtttggggggtgtaagattggcagcagtttcagtttccccataaaagtcaatgggtgaaatttggctgttgttgactttaagtcattcaacaaatgttgctgtgtaattcggggtgaccccatgattatgttattcaagtttggggggtgtagcttcaaagctgtaagagtggcagcagtttgaaaatcttccctgtcaaagtcaatgggaaaattggggggttcagaggggcgccacaaaaagacgggggcgggatcgcttagaaaagcacaagcaacctggtccgctatagggtgaggaagtgtgtggagtttgggtgttgtatccctaaaactgtaggaggagtagcgtttagaaaatggggggcgctaagaataagaagaaaaagcgaaagaatcagctgatgtcgaagaacaacccaacataataataaagttcctgctgctgctgccgcaatatcatcaaatttcgtctggtttggggtctgttaaggctcctaataatgtacttgtattgaaggcttgcatttcattataattaattgaattatttaatggaggtgcaagccaaaaaagcataatggttctgaggctcattcaatacaatagcagaatagtcaaaattgtcacttacgtgtcaaactagaggcctgtgcgtaaaaattcacacgtgcacgtcaaaatatatgcttgcgcgaccaaatgtacattttcgcgacaaaatataaccttgcgcgaccaaatgtacaatttcgcgaccaaatatacgcttgcgcgaccaaatgtacacctacacgactaaatatacgcttgcgcgaacaaatgtacgtttcgcgacgaaatattcgtgcgcgcgacaagcccggagcgcgacgaattcgtcgctcgcacggtaatttgcgcgcatgcgcgttaccggcgcgcgcatacgtacgcggcgcgcgcgtcaatagggtgtgtccctagtataaatacctggtgccctgccctgtgaccagtaggactggaaaactttgcaaggcgtggacatggctggacctgggaacatgaatgaggcgcagctgcggtactttatatccttcctgcaccgggagggatatgacaatatcccagcagggactcccgggatccaatccctccgcagggggattatcaggagactgcggcggcgcctcagacgggatcaccaggtcaatcttagtgtccgcgtcttgcagcgtttgtggagtgacgtcaaaagacgtcacacagagcttgtggaagagctgaggggagaagtggaaggtagattttattaaaaattaatataaagatttttcttctaaattaccactcacttgtttcctatttaatttgctatttttgagcaaaaatgtgtttgtgaaaatactttaacattaggattggaacaatagccataattgcagattatttaaatagagattatctcttaatggtgtttcccatcccataactgccatacttatagaggccctgccccatacctgccatgcttatagataccctgccccataccttctgtaaggtttggacccacacatacacagaggcttttgctataggctttattttacaaactgttaggctgccagctctgctcacatggctatgacaccataatggctcacatgactgtcataccatagtgactatgatactatctggtttgctggaaagggcgccctctaatgtcagcagtgttgcatagcactaaacattaacaaaacataacaaacatcttataacagggttgctgttgaacactacacctgccatgcttatagatgccctgccccatacctgccatgcttatagatgccctgccccatacctgccatgcttatagatgccctgccccatacctgccatgcttatagatgccctgccccatacctgccatgcttatagatgccctgccccatacctgccatgcttatagatgccctgccccatacctgccatgcttatagatgccctgccccatacctgccatgcttatagatgccctgccccatacctgccatgcttatagatgccctgccccatacctgccatgcttatagatgtccccgccccatacctgccatgcttatagataatggttttcctgtagttacatgacacatctcagcctgtatcatttgttcggtacaaaagcaagtgctgtatttctcaaactgaaattctttcattgccagacgaatggctacaggtggatcagggtgcaaatggggcagaggcagaggcagaggcaccaccggctcctgcacaggatccccagcttgccgatgatgaggcagaggcagcacccccagcagcaccaccagcagcgccagaagcacccccaggagcacgtgatgctgcctcccaaacaggggggacaaatttatttttggacctcctacaagaggtccgccaggaggtggcccttatgagggaggacatgggccttatgaggcaagacatggcccttatggggcaggatgtggcccttatgcggcaggacttaagggacattcaggaggctatcctgtctggttctcctcctcctcctcctcctcctcctcctcctcctcctcctcctgcttttgtgtaagtgtttatgtgcagtgcaccgttgtgcacttttgttccattaaaatattcatggttttcaatcttcaacatttgaattttatttcatttaactactgatcagatgatacttgaagttgtgtaacattacatagtattttagtagattcattacatttacacaaaaatatacagtatattcatgttaatgtgggatacaaagcacccagatggtatctgttcatatttattattagttgtggctgtgtttgtaagctagtagtaacatataaaatatagattgtacttttaggtacttttttagtgcagtagtagataccattaactgtcaaccagaagtaatgtaaaaaatcacagacatgtttttaagaaactggcaaaatgggagccttttttcacttgggagaaaacacttgtagcatagcggataggaggttctctaacaatgtagggcatgtgtgtagcaggcagtatactcagccaaactgcatgggaattggtcttcccattgactgcaatgcaattcagcgaattttgcaacctgtttgggaattttgccacaaagcaaaacatggcagattaacttaaagggtggttcacatttaagttaattattagcatgtagtagaatggccaattataagcatagtaacatagtaagataggttgaaaaaagacatacgtccatcacgttcaaccttaatacctatatataacctgcctaactggttaattaagagtaaggcaaaaaaccccatctgaagcctctctaatttgccgcagaggggaaacaattccttcctgactccaagatggcaatcggaccagtccctggatcaacttgtactgagagctatctcccataaccctgtattccctcacttgtactgagagctatctcccctacccctgtattccctcacttgtactgagagctatctcccatacccctgtattccctcacttgtactgagagctatctcccctacccctgtattccctcacttgtactgagagctatctcccataaccctgtagtgatactaagcaactttacaattggtcttcattatttatttgttcacagtttttgaattgtttgccatcttcttttaactgccattccaactctatgttagcaagcccatggttgctaaggtaatttggaaactagcaaccagataacaggaaccgcatgcgttaatttgcacatagaaataatactaacgcatgattcacacttagacgcgctaaatatcgcattaggctatgcgaaaattaacccctacttggggcaggcggtaattatagaaaagtgcagtaaatgagcttttggcaacacaatatggactttgcagtgggatttattcaagtctgtgttggccccagagtgatgcagccgccagtttgcagggaaatgggcattttcagaacagtagttttccgaaagtaatgccgtgtatggctaatatggcgtgcgtttttttgcacacggcgactatttgtgcgcgatgcgttgatgtgatgcgtccaatatagcacgaaaatattcttcgcgacttaaataacacacacagcatcgcgataaaatttttaacgcatgctagaaatagcgctcgttttaacgcacttaagtgaatcggccctcataaataaaaaaaatgaagaccaattgcaaattgtctcagaatatctctctctacatcagctcgggggcaacatgctgctcaccaaccccttggatgttgctctcagtgaaaaaggtgcaacccacaaacaaaaccacctgtagaatttgtgcgctcgctgcgcggcgaagtagctatgtcacacggtgaaattttcgccgcgcgtacagttattttcgcgcgatggaaaattccgccgcgcgacgataatggtggcgccaaatttatttcgccgcgcgcgacaaaaattacgccgcacaaaaattacgctgcgcgacacattagtttcgctaatttttcgccgtttcgctaattttttcgccgtttcgcgaataattcggcgaaacgggacaaattcgctcatcactg

SEQ ID NO: 190 - DNA, 244 bp, Drosophila yakuba
caataatgtagttattaagttttgattgaaacgtgcaaaattgagtttatgtgagtttatttagtttttttattctaagtctttgaatatatatgctaaatttgttttttctcataaaataagttataaattaattaagattaataatataaaaaaaattattgaatttgcataagcggttgtgtttttattgatggccaaagtgagagttgtgaacatgcactcagttttgctcgccactgta

SEQ ID NO: 191 - DNA, 8 bp, Anopheles gambiae
gtatggac

SEQ ID NO: 192 - DNA, 8 bp, Anopheles gambiae
atgtgaac

SEQ ID NO: 193 - DNA, 2 bp, Danio rerio
ta

SEQ ID NO: 194 - DNA, 8 bp, Danio rerio
gtctatac

SEQ ID NO: 195 - DNA, 6 bp, Petromyzon marinus
ttagag

SEQ ID NO: 196 - DNA, 8 bp, Xenopus tropicalis
ctgtatag

SEQ ID NO: 197 - DNA, 8 bp, Myotis lucifugus
gtctagag

SEQ ID NO: 198 - DNA, 2 bp, Xenopus tropicalis
at

SEQ ID NO: 199 - DNA, 8 bp, Petromyzon marinus
ctctagac

SEQ ID NO: 200 - DNA, 8 bp, Petromyzon marinus
gtctagac

SEQ ID NO: 201 - DNA, 8 bp, Xenopus tropicalis
atcatcat

SEQ ID NO: 202 - DNA, 4 bp, Xenopus tropicalis
ttaa

SEQ ID NO: 203 - DNA, 8 bp, Petromyzon marinus
ctctagag

SEQ ID NO: 204 - DNA, 8 bp, Alligator mississippiensis
tttataat

SEQ ID NO: 205 - DNA, 8 bp, Alligator mississippiensis
ctatatag

SEQ ID NO: 206 - DNA, 8 bp, Xenopus tropicalis
attaatag